(19) United States
(12) Patent Application Publication
(10) Pub. No.: US 2025/0359423 A1
(43) Pub. Date: Nov. 20, 2025

(54) ORGANIC COMPOUND AND ORGANIC ELECTROLUMINESCENT DEVICE, AND ELECTRONIC APPARATUS THEREOF

(71) Applicant: SHAANXI LIGHTE OPTOELECTRONICS MATERIAL CO., LTD., Shaanxi (CN)

(72) Inventor: Fumin YUE, Shaanxi (CN)

(21) Appl. No.: 18/870,948

(22) PCT Filed: Sep. 26, 2023
(86) PCT No.: PCT/CN2023/121782

§ 371 Date: Dec. 2, 2024

(30) Foreign Application Priority Data
Feb. 21, 2023 (CN) 202310146580.4

(51) Int. Cl. H10K 50/11; C07C 211/54; C09K 11/06; H10K 50/15; H10K 85/60

(52) U.S. Cl. CPC H10K 50/11; C07C 211/54; C09K 11/06; H10K 50/15; H10K 85/622; H10K 85/626; H10K 85/633; H10K 85/636; H10K 85/654; H10K 85/6572; H10K 85/6574; H10K 85/6576; C09K 2211/1014

(57) ABSTRACT

The present application relates to the technical field of organic electroluminescence, and provides an organic compound, and an organic electroluminescent device, and an electronic apparatus thereof. The organic compound has a structure represented by formula I. The organic electroluminescent device prepared by taking the compound as a hole transport layer material has good photoelectric performance.

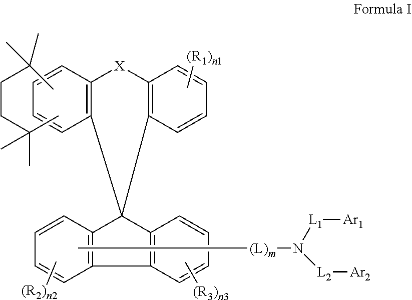

### CROSS-REFERENCE TO RELATED DISCLOSURES

The present disclosure claims the priority of Chinese patent disclosure No. 2023101465804 filed on Feb. 21, 2023, which is incorporated herein by reference in its entirety as a part of this disclosure.

### FIELD OF THE INVENTION

The present disclosure relates to the technical field of organic electroluminescence, and specifically to an organic compound and an organic electroluminescent device, and an electronic apparatus thereof.

### BACKGROUND OF THE INVENTION

Organic electroluminescent devices (OLEDs) are devices prepared by depositing a layer of organic material between two metal electrodes through spin coating or vacuum vapor deposition. A classic three-layer Organic electroluminescent device includes a hole transport layer, a light-emitting layer, and an electron transport layer. The holes generated by the anode passing through the hold transfer layer combine with the electrons generated by the cathode passing through the electron transport layer to form excitons in the light-emitting layer, and then emit light. Organic electroluminescent devices can adjust the emission of various required light by changing the material of the light-emitting layer as needed.

At present, commercial products based on OLED light-emitting and display technology have been industrialized. Compared with liquid crystal display technology, OLED display technology has many advantages such as self light-emission, free-radiation, lightweight, thin thickness, wide viewing angle, wide color gamut, color stability, fast response speed, strong environmental adaptability, and the ability to achieve flexible display. Therefore, OLED display technology is receiving increasing attention and corresponding technological investment.

At present, many existing technologies have disclosed that aromatic amine compounds can be used as hole transport materials or auxiliary hole transport layer materials in OLED devices to adjust the transport and injection of charge carriers into the organic light-emitting layer. However, it is still necessary to continue developing new materials to further improve the performance of electronic devices.

### SUMMARY OF THE INVENTION

The objective of the present disclosure is to provide an organic compound and an organic electroluminescent device and an electronic apparatus thereof. The use of the organic compound in organic electroluminescent devices can improve the performance of the devices.

According to a first aspect of the present disclosure, there is provided an organic compound having a structure shown in Formula I:

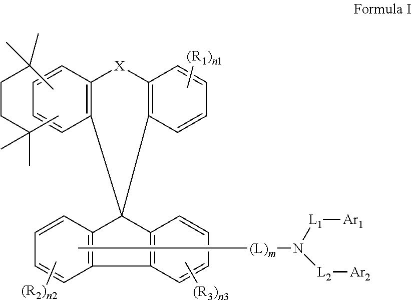

wherein,
X is selected from a single bond, C(R₄R₅), O, and S;
R₄ and R₅ are the same or different, and are each independently selected from a hydrogen, an alkyl having 1 to 10 carbon atoms, and an aryl having 6 to 20 carbon atoms;
R₁, R₂, and R₃ are the same or different, and are each independently selected from a deuterium, a cyano, a halogen group, an alkyl having 1 to 10 carbon atoms, a haloalkyl having 1 to 10 carbon atoms, a deuteroalkyl having 1 to 10 carbon atoms, a deuteroaryl having 6 to 12 carbon atoms, a trialkylsilyl having 3 to 12 carbon atoms, an aryl having 6 to 20 carbon atoms, a heteroaryl having 3 to 20 carbon atoms, and a cycloalkyl having 3 to 10 carbon atoms;
m is the number of sequentially linked L, and m is selected from 0, 1, and 2;
n₁ is the number of R₁, and n₁ is selected from 0, 1, 2, 3, and 4; when n₁ is greater than 1, any two R₁ are the same or different; optionally, any two adjacent R₁ form a ring;
n₂ is the number of R₂, and n₂ is selected from 0, 1, 2, 3, and 4; when n₂ is greater than 1, any two R₂ are the same or different; optionally, any two adjacent R₂ form a ring;
n₃ is the number of R₃, and n₃ is selected from 0, 1, 2, 3, and 4; when n₃ is greater than 1, any two R₃ are the same or different; optionally, any two adjacent R₃ form a ring;
L, L₁, and L₂ are the same or different, and are each independently selected from a single bond, a substituted or unsubstituted arylene having 6 to 30 carbon atoms, and a substituted or unsubstituted heteroarylene having 3 to 30 carbon atoms;
Ar₁ and Ar₂ are each independently selected from a substituted or unsubstituted aryl having 6 to 30 carbon atoms and a substituted or unsubstituted heteroaryl having 3 to 30 carbon atoms;
substituent(s) in L, L₁, L₂, Ar₁ and Ar₂ are the same or different, and are each independently selected from a deuterium, a cyano, a halogen group, an alkyl having 1 to 10 carbon atoms, a haloalkyl having 1 to 10 carbon atoms, a deuteroalkyl having 1 to 10 carbon atoms, a trialkylsilyl having 3 to 12 carbon atoms, an aryl having 6 to 20 carbon atoms, a heteroaryl having 3 to 20 carbon atoms, and a cycloalkyl having 3 to 10 carbon atoms; optionally, any two adjacent substituents in Ar₁ form a saturated or unsaturated 3-membered to 15-membered ring; optionally, any two adjacent substituents in Ar₂ form a saturated or unsaturated 3-membered to 15-membered ring.

According to a second aspect of the present disclosure, there is provided an organic electroluminescent device, comprising an anode and a cathode disposed opposite to each other, and a functional layer disposed between the anode and the cathode; the functional layer contains the organic compound as described above.

According to a third aspect of the present disclosure, there is provided an electronic apparatus, comprising the organic electroluminescent device described in the second aspect.

The parent nucleus structure of the compound in the present disclosure is a structure of a dibenzo(penta- or hexa-)membered ring-spiro-fluorene, in which the benzene ring of the dibenzo(penta- or hexa-)membered ring is fused with a tetramethylcyclohexyl

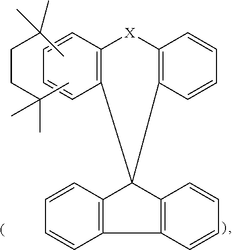

wherein the planes of the dibenzo(penta- or hexa-)membered ring and fluorene are perpendicular to each other. The polyalkyl substituted cycloalkyl fused on a dibenzo(penta- or hexa-)membered ring can further adjust the steric hindrance of compound molecules, effectively avoiding the intermolecular stacking, and improving the forming film property of the compound. On another hand, an aromatic amine group is linked to the non-fused fluorenyl of the parent nucleus, in this case the fused cyclic alkyl in the parent nucleus is far away from the N atom in the aromatic amine, thereby reducing the influence of the electron-rich alkyl on hole injection and improving the hole injection ability of the compound. Therefore, the compound of the present disclosure was vapor deposited as the material of a hole transport layer into organic electroluminescent devices can not only improve the luminous efficiency of the devices, but also enhance the film-forming property of the material, thereby significantly extending the lifetime of the devices.

### BRIEF DESCRIPTION OF THE DRAWINGS

The drawings are used for a further understanding of the present disclosure and constitute a part of the specification, and together with the following detailed embodiments, are used to explain the present disclosure, but do not constitute a limitation of the present disclosure.

FIG. 1 is a schematic structural diagram of an organic electroluminescent device according to an embodiment of the present disclosure.

FIG. 2 is a schematic structural diagram of an electronic apparatus according to an embodiment of the present disclosure.

### LIST OF REFERENCE SIGNS

| | | | |
|---|---|---|---|
| 100: Anode | 200: Cathode | 300: Functional | 310: Hole |
| | | layer | injection layer |
| 321: Hole | 322: Electron | 330: Organic light- | 340: Electron |
| transport layer | blocking layer | emitting layer | transport layer |
| 350: Electron | 400: Electronic | | |
| injection layer | apparatus | | |

### DETAILED DESCRIPTION OF THE EMBODIMENTS

Exemplary embodiments will now be described more comprehensively with reference to the accompanying drawings. The exemplary embodiments, however, can be implemented in a variety of forms and should not be interpreted as being limited to the examples set forth herein. On the contrary, these embodiments are provided to make the present disclosure more comprehensive and complete, and to convey the concepts of these exemplary embodiments fully to those of ordinary skill in the art. Features, structures, or characteristics described herein can be combined in one or more embodiments in any suitable manner. In the following description, many specific details are provided to give a full understanding of the embodiments of the present disclosure.

In a first aspect, the present disclosure provides an organic compound having a structure shown in Formula I:

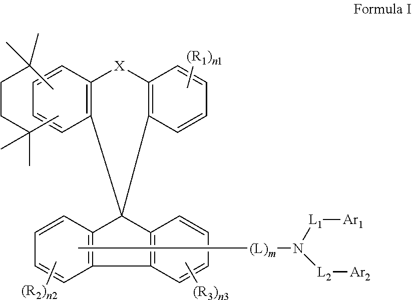

wherein,
X is selected from a single bond, C(R₄R₅), O, and S;
R₄ and R₅ are the same or different, and are each independently selected from a hydrogen, an alkyl group having 1 to 10 carbon atoms, and an aryl having 6 to 20 carbon atoms;
R₁, R₂, and R₃ are the same or different, and are each independently selected from a deuterium, a cyano, a halogen group, an alkyl having 1 to 10 carbon atoms, a haloalkyl having 1 to 10 carbon atoms, a deuteroalkyl having 1 to 10 carbon atoms, a deuteroaryl having 6 to 12 carbon atoms, a trialkylsilyl having 3 to 12 carbon atoms, an aryl having 6 to 20 carbon atoms, a heteroaryl having 3 to 20 carbon atoms, and a cycloalkyl having 3 to 10 carbon atoms;
m is the number of sequentially linked L, and m is selected from 0, 1, and 2;
n₁ is the number of R₁, and n₁ is selected from 0, 1, 2, 3, and 4; when n₁ is greater than 1, any two R₁ are the same or different; optionally, any two adjacent R₁ form a ring;
n₂ is the number of R₂, and n₂ is selected from 0, 1, 2, 3, and 4; when n₂ is greater than 1, any two R₂ are the same or different; optionally, any two adjacent R₂ form a ring;
n₃ is the number of R₃, and n₃ is selected from 0, 1, 2, 3, and 4; when n₃ is greater than 1, any two R₃ are the same or different; optionally, any two adjacent R₃ form a ring;
L, L₁, and L₂ are the same or different, and are each independently selected from a single bond, a substituted or unsubstituted arylene having 6 to 30 carbon atoms, and a substituted or unsubstituted heteroarylene having 3 to 30 carbon atoms;
Ar₁ and Ar₂ are each independently selected from a substituted or unsubstituted aryl having 6 to 30 carbon atoms and a substituted or unsubstituted heteroaryl having 3 to 30 carbon atoms;
substituent(s) in L, L₁, L₂, Ar₁ and Ar₂ are the same or different, and are each independently selected from a deuterium, a cyano, a halogen group, an alkyl having 1 to 10 carbon atoms, a haloalkyl having 1 to 10 carbon atoms, a deuteroalkyl having 1 to 10 carbon atoms, a trialkylsilyl having 3 to 12 carbon atoms, an aryl having 6 to 20 carbon atoms, a heteroaryl having 3 to 20 carbon atoms, and a cycloalkyl having 3 to 10 carbon atoms; optionally, any two adjacent substituents in Ar₁ form a saturated or unsaturated 3-membered to 15-membered ring; optionally, any two adjacent substituents in Ar₂ form a saturated or unsaturated 3-membered to 15-membered ring.

In the present disclosure, the terms “optional” and “optionally” mean that the subsequently described event or circumstance may or may not occur. For example, “optionally, any two adjacent substituents form a saturated or unsaturated 3-membered to 15-membered ring” includes scenarios where any two adjacent substituents form a ring, as well as scenarios where any two adjacent substituents exist independently without forming a ring. “Any two adjacent” can include having two substituents on the same atom, and can also include having one substituent on each of adjacent atoms; among them, when there are two substituents on the same atom, the two substituents can form a saturated or unsaturated spiro ring with the atom they are linked to together; when two adjacent atoms each have a substituent, these two substituents can be fused into a ring.

In the present disclosure, the expression “each . . . independently“may be used interchangeably with the expressions” . . . respectively independently“and” . . . each independently”, and all these expressions should be interpreted in a broad sense. They can not only mean that, for same symbols in a different group, the selection of a specific option for one of the symbols and the selection of a specific option for another one of the symbols do not affect each other, but also mean that for same symbols in same groups, the selection of a specific option for one of the symbols and the selection of a specific option for another one of the symbols do not affect each other. For example,

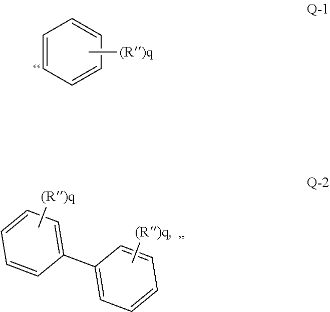

in which each q is independently 0, 1, 2, and 3, and each R″ is independently selected from a hydrogen, a deuterium, a fluorine, and a chlorine”, which means that the Formula Q-1 represents q substituents R″ on the benzene ring, and each R″ can be the same or different, with no mutual influence between the options for each R″; Formula Q-2 represents that there are q substituents R″ on each benzene ring of biphenyl, and the number q of R″ substituents on the two benzene rings can be the same or different, with no mutual influence between the options for each R″.

In the present disclosure, the term “substituted or unsubstituted” means that the functional group defined by the term may or may not have a substituent (hereinafter referred to as Rc for ease of description). For example, “substituted or unsubstituted aryl” refers to an aryl with the substituent Rc or an aryl without a substituent. Among them, the above substituent, i.e., Rc, may be, for example, a deuterium, a halogen group, a cyano, a heteroaryl, an aryl, an a trialkylsilyl, an alkyl, a haloalkyl, and a cycloalkyl, etc. The number of substituents may be one or more.

In the present disclosure, “more” refers to two or more, such as 2, 3, 4, 5, and 6, etc.

In the present disclosure, the number of carbon atoms of a substituted or unsubstituted functional group refers to the number of all carbon atoms.

The hydrogen atom in the structure of the compound in the present disclosure includes various isotope atoms of hydrogen, such as hydrogen (H), deuterium (D), and tritium (T).

The “D” in the structural formula of the compound in the present disclosure represents a deuterium substitution.

In the present disclosure, an aryl refers to any functional group or substituent derived from an aromatic carbon ring. An aryl may be a monocyclic aryl (e.g., phenyl) or a polycyclic aryl. In other words, an aryl may be a monocyclic aryl, a fused aryl, two or more monocyclic aryls linked by carbon-carbon bond conjugation, a monocyclic aryl and a fused aryl linked by carbon-carbon bond conjugation, or two or more fused aryls linked by carbon-carbon bond conjugation. That is, unless otherwise specified, two or more aromatic groups linked by carbon-carbon bond conjugation may also be regarded as an aryl in the present disclosure. Among them, a fused aryl may include, for example, a bicyclic fused aryl (e.g., naphthyl), a tricyclic fused aryl (e.g., phenanthryl, fluorenyl, anthryl), etc. The aryl does not contain heteroatoms such as B, N, O, S, P, Se, and Si. Examples of an aryl include, but are not limited to, a phenyl, a naphthyl, a fluorenyl, a spirobifluorenyl, an anthryl, a phenanthryl, a biphenyl, a terphenyl, a triphenylene, a perylenyl, a benzo[9,10]phenanthryl, a pyrenyl, a benzofluoranthryl, and a chrysenyl, etc.

In the present disclosure, “an arylene” refers to a divalent group formed by further removing one or more hydrogen atom(s) from an aryl.

In the present disclosure, a terphenyl includes

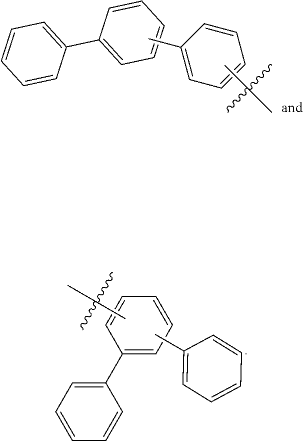

In the present disclosure, the number of carbon atoms of a substituted or unsubstituted aryl (arylene) may be 6, 8, 10, 12, 13, 14, 15, 16, 17, 18, 19, 20, 21, 22, 23, 24, 25, and 30. In some embodiments, a substituted or unsubstituted aryl is a substituted or unsubstituted aryl having 6 to 30 carbon atoms; in other embodiments, a substituted or unsubstituted aryl is a substituted or unsubstituted aryl having 6 to 25 carbon atoms; in other embodiments, a substituted or unsubstituted aryl is a substituted or unsubstituted aryl having 6 to 18 carbon atoms; in other embodiments, a substituted or unsubstituted aryl is a substituted or unsubstituted aryl having 6 to 15 carbon atoms.

In the present disclosure, a fluorenyl can be substituted by one or more substituent(s). In a case that the above-mentioned fluorenyl is substituted, the substituted fluorenyl may be:

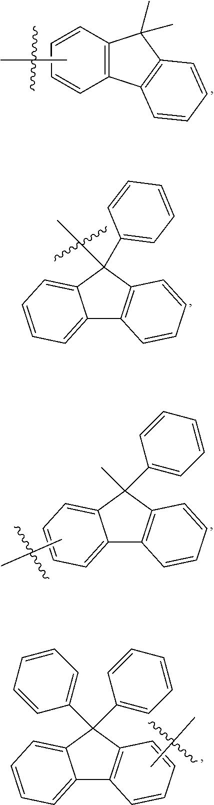

etc, but is not limited thereto.

In the present disclosure, an aryl as a substituent of L, L₁, L₂, Ar₁ and Ar₂ is, for example, but not limited to, a phenyl, a naphthyl, a phenanthryl, a biphenyl, a fluorenyl, and a dimethylfluorenyl, etc.

In the present disclosure, “a heteroaryl” refers to a monovalent aromatic ring containing 1, 2, 3, 4, 5, and 6 heteroatoms or a derivative thereof. The heteroatoms may be one or more selected from B, O, N, P, Si, Se, and S. A heteroaryl may be a monocyclic heteroaryl or a polycyclic heteroaryl. In other words, a heteroaryl may be a single aromatic ring system, or multiple aromatic ring systems linked by carbon-carbon bond conjugation, with any of the aromatic ring systems being an aromatic monocyclic ring or a fused aromatic ring. For example, a heteroaryl may include, thienyl, furyl, pyrrolyl, imidazolyl, thiazolyl, oxazolyl, oxadiazolyl, triazolyl, pyridyl, dipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, N-phenylcarbazolyl, N-pyridylcarbazolyl, and N-methylcarbazolyl, etc, but not limited to thereto.

In the present disclosure, “a heteroarylene” involved refers to a divalent group or a multivalent group formed by further removing one or more hydrogen atom(s) from a heteroaryl.

In the present disclosure, the number of carbon atoms of a substituted or unsubstituted heteroaryl (heteroarylene) may be selected from 3, 4, 5, 6, 7, 8, 9, 10, 11, 12, 13, 14, 15, 16, 17, 18, 20, 21, 22, 23, 24, 25, 26, 27, 28, 29, and 30. In some embodiments, a substituted or unsubstituted heteroaryl is a substituted or unsubstituted heteroaryl having a total carbon atoms of 12 to 18. In other embodiments, a substituted or unsubstituted heteroaryl is a substituted or unsubstituted heteroaryl having a total carbon atoms of 5 to 12.

In the present disclosure, a heteroaryl as a substituent for L, L₁, L₂, Ar₁ and Ar₂, includes, but is not limited to, a pyridyl, a carbazolyl, a dibenzothienyl, a dibenzofuranyl, a benzoxazolyl, a benzothiazolyl, and a benzimidazolyl.

In the present disclosure, a substituted heteroaryl may mean that one or more hydrogen atom(s) in the heteroaryl are replaced by a group such as a deuterium, a halogen group, a cyano, an aryl, a heteroaryl, a trialkylsilyl, an alkyl, a cycloalkyl, and a haloalkyl, etc.

In the present disclosure, an alkyl having 1 to 10 carbon atoms may include a straight-chain alkyl having 1 to 10 carbon atoms or a branched-chain alkyl having 3 to 10 carbon atoms. The number of the carbon atoms of an alkyl may be for example, 1, 2, 3, 4, 5, 6, 7, 8, 9, and 10. Specific examples of an alkyl include, but are not limited to, a methyl, an ethyl, a n-propyl, an isopropyl, a n-butyl, an isobutyl, a tert-butyl, a n-pentyl, an isopentyl, a neopentyl, and a n-hexyl, etc.

In the present disclosure, a halogen group may be for example, a fluorine, a chlorine, a bromine, and an iodine.

In the present disclosure, specific examples of a trialkylsilyl include, but are not limited to, a trimethylsilyl, and a triethylsilyl, etc.

In the present disclosure, specific examples of a haloalkyl group include, but are not limited to, a trifluoromethyl.

In the present disclosure, the number of carbon atoms of a cycloalkyl having 3 to 10 carbon atoms may be, for example, 3, 4, 5, 6, 7, 8 and 10. Specific examples of a cycloalkyl include, but are not limited to, a cyclopentyl, a cyclohexyl, and an adamantyl.

In the present disclosure, the number of carbon atoms of a deuteroalkyl having 1 to 10 carbon atoms may be, for example 1, 2, 3, 4, 5, 6, 7, 8 and 10. Specific examples of a deuteroalkyl include, but are not limited to, a trideuteromethyl.

In the present disclosure, the number of carbon atoms of a haloalkyl having 1 to 10 carbon atoms may be, for example 1, 2, 3, 4, 5, 6, 7, 8 and 10. Specific examples of a haloalkyl include, but are not limited to, a trifluoromethyl.

In the present disclosure, a ring system that is formed of n atoms is n-membered ring. For example, a phenyl is a 6-membered ring. A 3-membered to 15-membered ring refers to a cyclic group having 3 to 15 ring atoms. For example, a 3-membered to 15-membered ring is a cyclopentane, a cyclohexane, a fluorene ring, and a benzene ring, etc.

In the present disclosure,

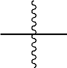

refers to the chemical bond that linked to other groups.

In the present disclosure, a single bond

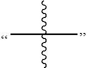

extending from a ring system involved in the non-positional linkage bond, which represents that one end of the linkage bond can be linked to any position in the ring system through which the bond passes, and the other end is linked to the rest of the compound molecule. For example, as shown in Formula (f) below, the naphthyl represented by Formula (f) is linked to other positions of the molecule through two non-positional bonds passing through the two rings, which indicates any of possible linkages shown in Formulae (f-1) to (f-10):

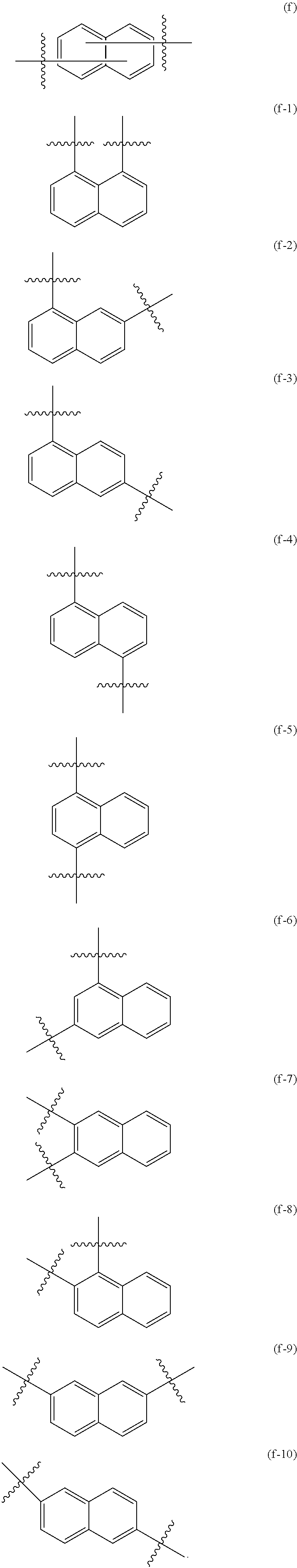

As another example, as shown in Formula (X′) below, the dibenzofuranyl represented by Formula (X′) is linked to other positions of the molecule via a non-positional linkage bond extending from the center of a side benzene ring, which indicates any of possible linkages shown in Formulae (X′-1) to (X′-4):

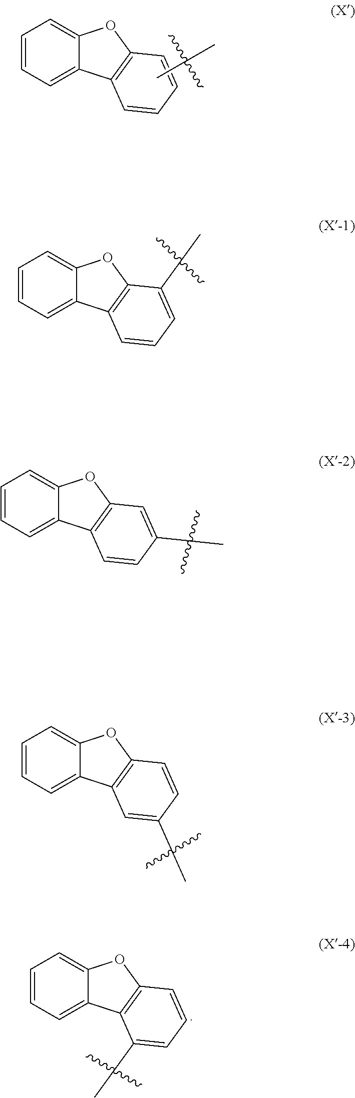

The non-positional substituent in the present disclosure refers to a substituent linked by a single bond extending from the center of the ring system, indicating that the substituent can be linked to any possible position in the ring system. For example, as shown in Formula (Y) below, the substituent R′ represented by Formula (Y) is linked to a quinoline ring via a non-positional linkage bond, which indicates any of possible linkages shown in Formulae (Y-1) to (Y-7):

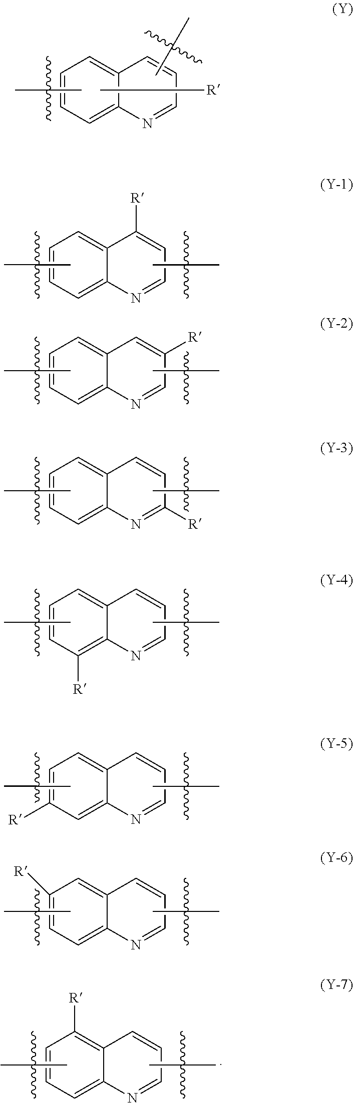

In some embodiments, the organic compound has the structure shown in any one of Formulae I-1 to I-3:

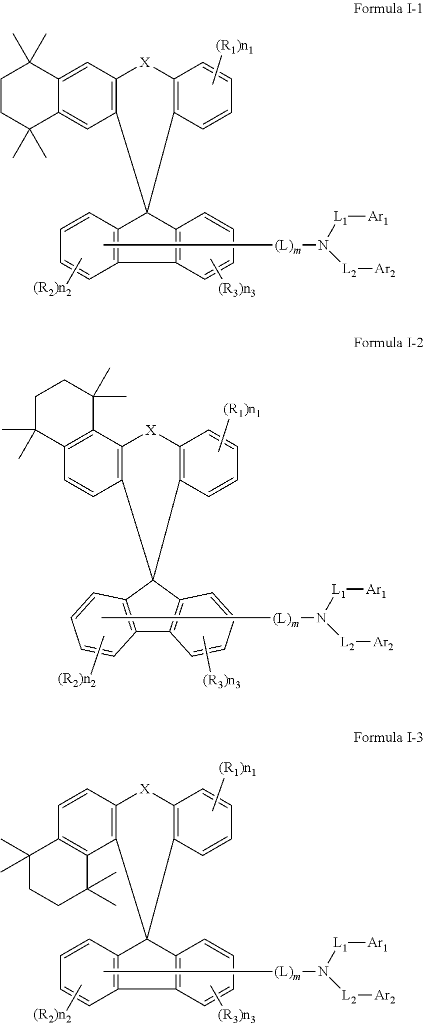

wherein, X, L, L₁, L₂, Ar₁, Ar₂, R₁, R₂, R₃, n₁, n₂, n₃, and m have the same definitions as those in Formula I.

In some embodiments, the organic compound has the structure shown in any one of Formulae I-a to I-l:

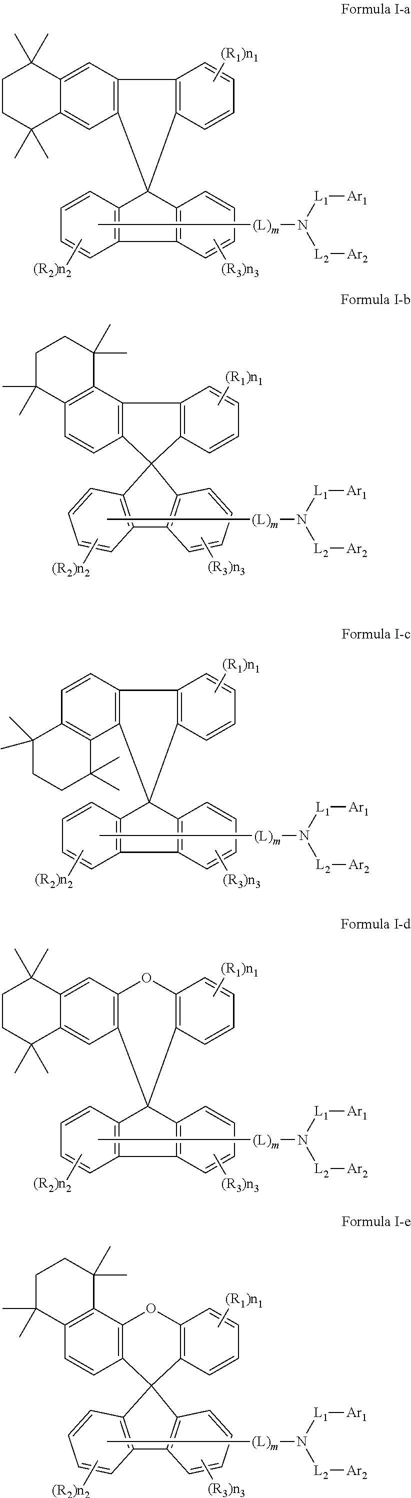

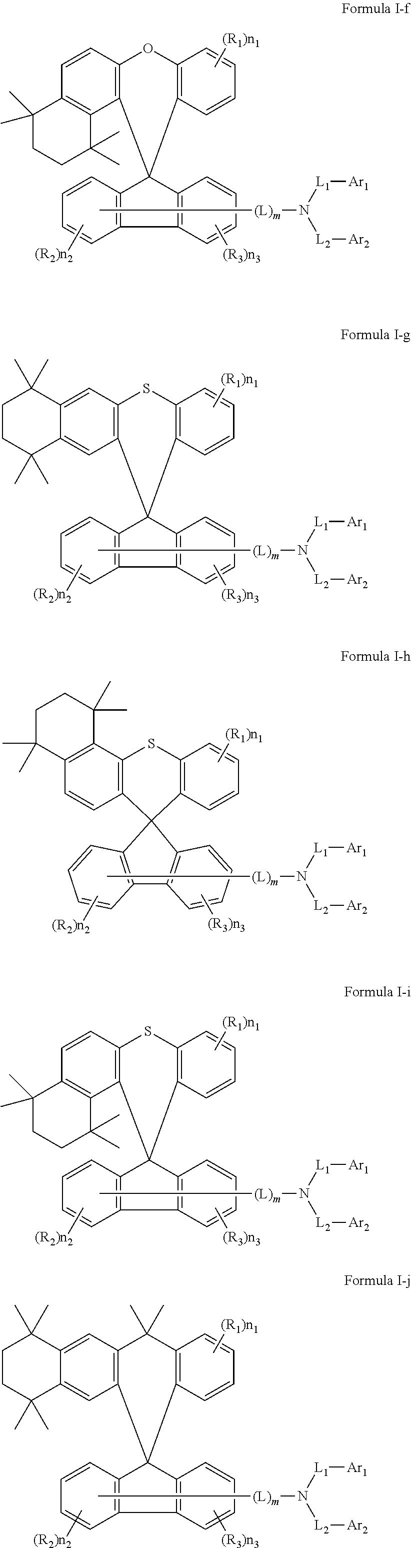

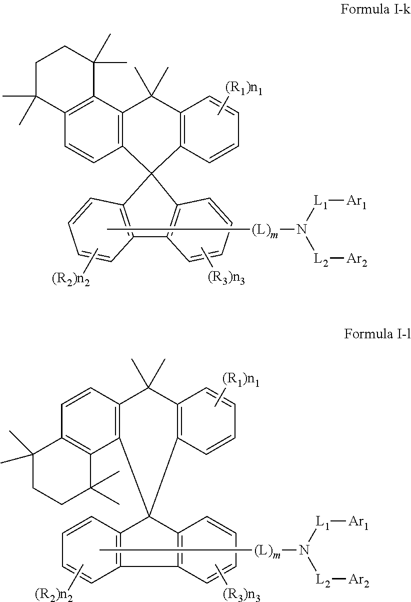

wherein L, L₁, L₂, Ar₁, Ar₂, R₁, R₂, R₃, n₁, n₂, n₃, and m have the same definitions as those in Formula I.

In some embodiments, X is selected from a single bond, C(R₄R₅), O, and S.

Optionally, R₄ and R₅ are each independently selected from a hydrogen, an alkyl having 1 to 6 carbon atoms, and an aryl having 6 to 15 carbon atoms.

Optionally, R₄ and R₅ are each independently selected from a methyl, an ethyl, a n-propyl, an isopropyl, and a phenyl.

In some embodiments, X is selected from a single bond and O.

Optionally, R₁, R₂, and R₃ are the same or different, and are each independently selected from a deuterium, a fluorine, a cyano, a methyl, an ethyl, an isopropyl, a tert-butyl, a cyclohexyl, a phenyl, a deuterophenyl, and a naphthyl.

In some embodiments, Ar₁ and Ar₂ are each selected from a substituted or unsubstituted aryl having 6 to 25 carbon atoms, and a substituted or unsubstituted heteroaryl having 5 to 18 carbon atoms; for example, Ar₁ and Ar₂ are each independently selected from a substituted or unsubstituted aryl having 6, 7, 8, 9, 10, 11, 12, 13, 14, 15, 16, 17, 18, 19, 20, 21, 22, 23, 24, and 25 carbon atoms, and a substituted or unsubstituted heteroaryl having 5, 6, 7, 8, 9, 10, 12, 13, 14, 15, 16, 17, and 18 carbon atoms.

Optionally, substituent(s) in Ar₁ and Ar₂ are the same or different, and are each independently selected from a deuterium, a fluorine, a cyano, a trimethylsilyl, an alkyl having 1 to 5 carbon atoms, a cycloalkyl having 5 to 10 carbon atoms, a haloalkyl having 1 to 5 carbon atoms, an aryl having 6 to 12 carbon atoms, and a heteroaryl having 5 to 12 carbon atoms. Optionally, any two adjacent substituents in Ar₁ form a saturated or unsaturated ring having 5 to 13 carbon atoms. Optionally, any two adjacent substituents in Ar₂ form a saturated or unsaturated ring having 5 to 13 carbon atoms.

In some embodiments, Ar₁ and Ar₂ are each independently selected from a substituted or unsubstituted phenyl, a substituted or unsubstituted naphthyl, a substituted or unsubstituted biphenyl, a substituted or unsubstituted fluorenyl, a substituted or unsubstituted phenanthryl, a substituted or unsubstituted terphenyl, a substituted or unsubstituted dibenzofuranyl, a substituted or unsubstituted dibenzothienyl, a substituted or unsubstituted carbazolyl, and a substituted or unsubstituted spirodifluorenyl.

Optionally, substituent(s) in Ar₁ and Ar₂ are each independently selected from a deuterium, a fluorine, a cyano, a trimethylsilyl, a trifluoromethyl, a methyl, an ethyl, an isopropyl, a tert-butyl, a phenyl, a naphthyl, a dibenzofuranyl, a dibenzothienyl, and a carbazolyl.

In some embodiments, Ar₁ and Ar₂ are each independently selected from a substituted or unsubstituted group W; the unsubstituted group W is selected from the group consisting of the following groups:

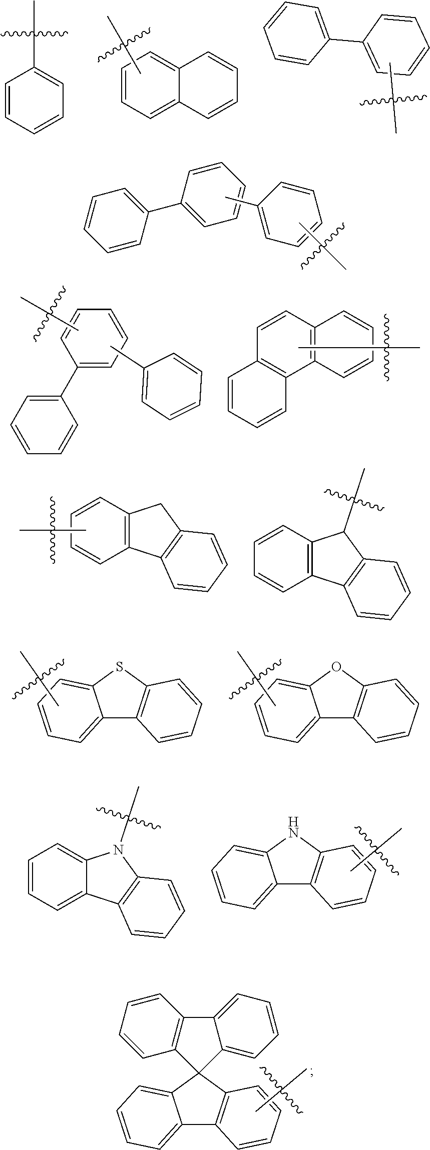

the substituted group W has one or more substituent(s), and each substituent is independently selected from a deuterium, a fluorine, a cyano, a trimethylsilyl, a trifluoromethyl, a methyl, an ethyl, an isopropyl, a tert-butyl, a phenyl, a naphthyl, a dibenzofuranyl, a dibenzothienyl, and a carbazolyl, and when the number of the substituents in group W is greater than 1, each substituent may be the same or different.

In some embodiments, Ar₁ and Ar₂ are each independently selected from the group consisting of the following groups:

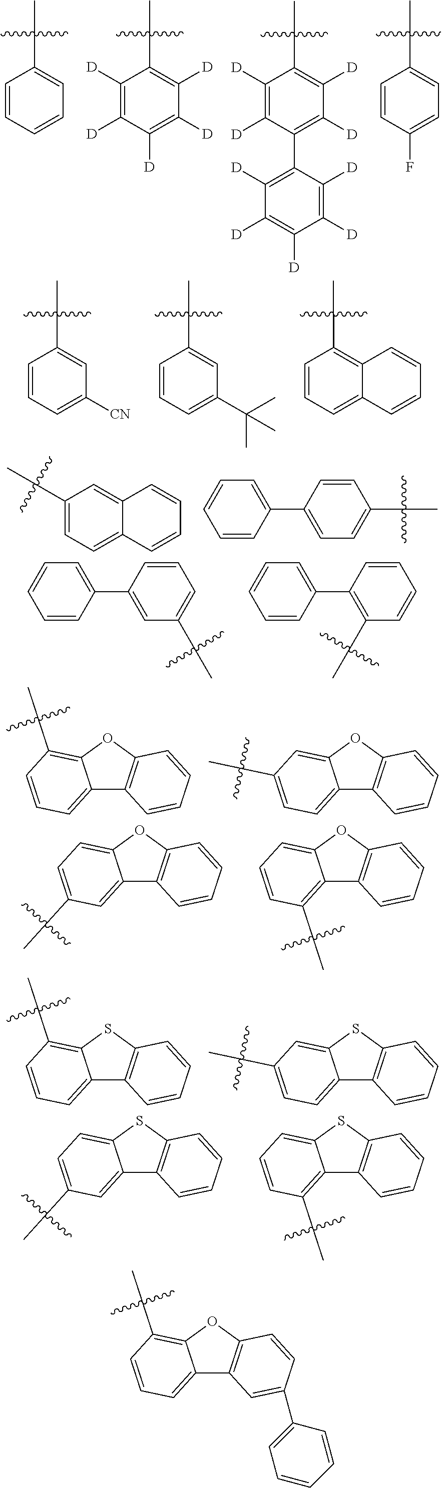

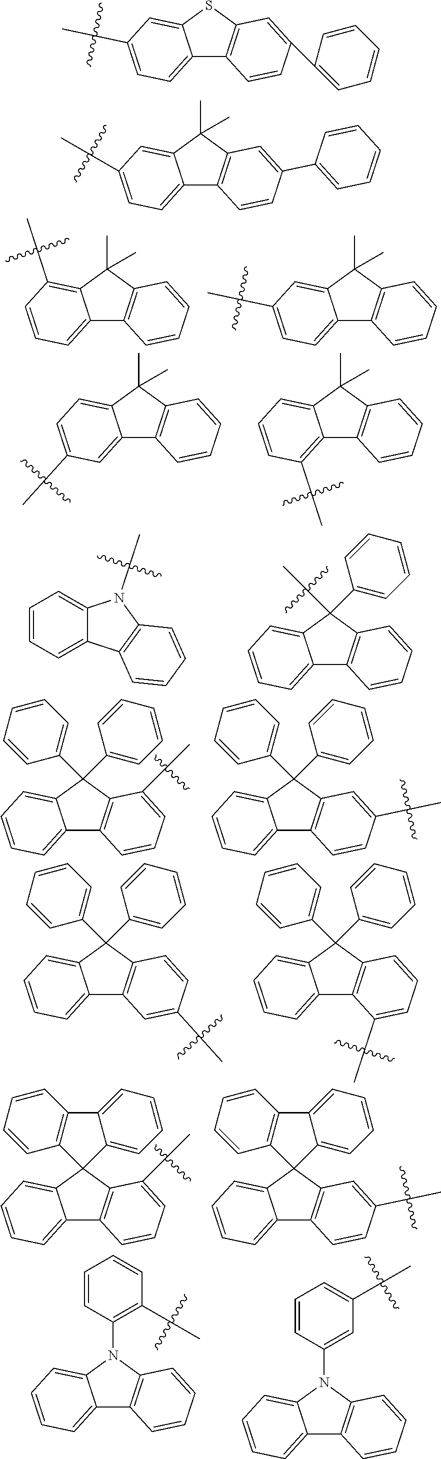

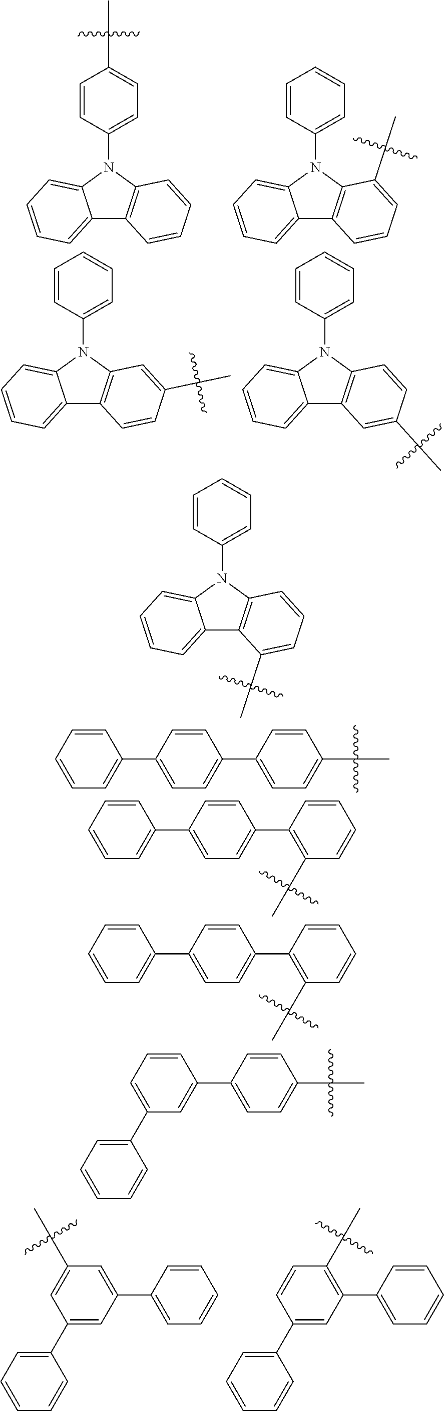

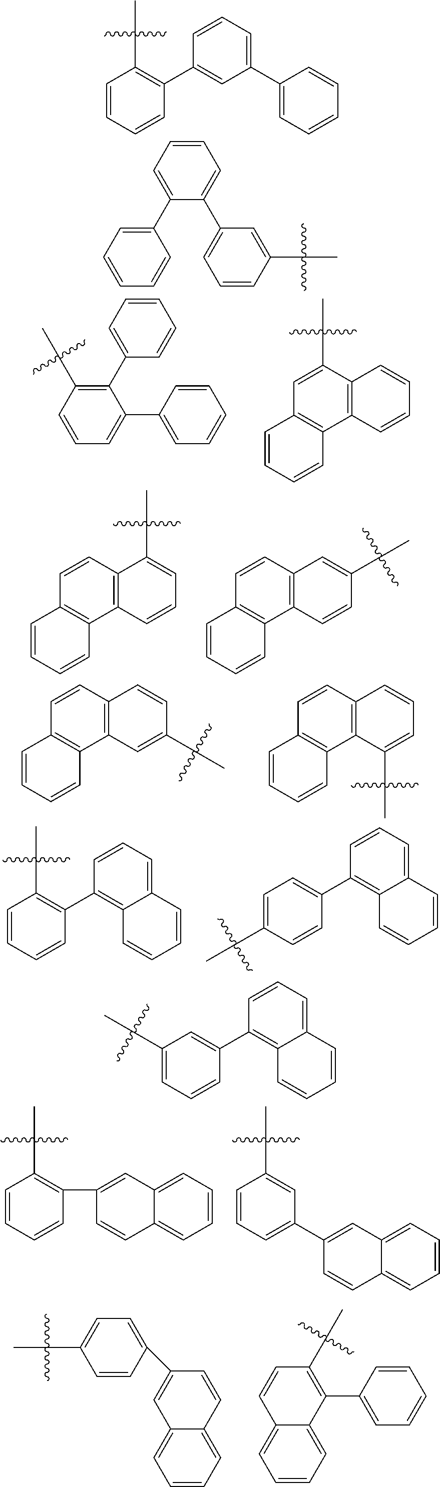

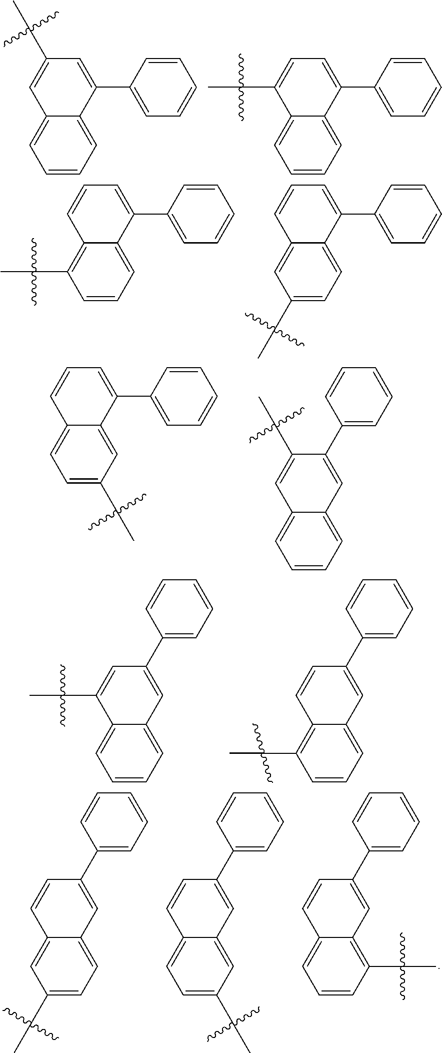

In some embodiments, L, L₁ and L₂ are the same or different, and are each independently selected from a single bond, a substituted or unsubstituted arylene having 6 to 18 carbon atoms, and a substituted or unsubstituted heteroarylene having 5 to 18 carbon atoms. For example, L, L₁, and L₂ are each independently selected from a single bond, a substituted or unsubstituted arylene having 6, 7, 8, 9, 10, 11, 12, 13, 14, 15, 16, 17, and 18 carbon atoms, and a heteroarylene having 5, 6, 7, 8, 9, 10, 11, 12, 13, 14, 15, 16, 17, and 18 carbon atoms.

Optionally, substituent(s) in L, L₁, and L₂ are each independently selected from a deuterium, a halogen group, a cyano, an alkyl having 1 to 5 carbon atoms, a haloalkyl having 1 to 5 carbon atoms, a deuteroalkyl having 1 to 5 carbon atoms, a trialkylsilyl having 3 to 8 carbon atoms, an aryl having 6 to 10 carbon atoms, and a heteroaryl having 5 to 12 carbon atoms.

In some embodiments, L, L₁, and L₂ are each independently selected from a single bond, a substituted or unsubstituted phenylene, a substituted or unsubstituted naphthylene, a substituted or unsubstituted biphenylene, a substituted or unsubstituted terphenylene, a substituted or unsubstituted fluorenylene, a substituted or unsubstituted carbazolylene, a substituted or unsubstituted dibenzothienylene, and a substituted or unsubstituted dibenzofuranylene.

Optionally, substituent(s) in L, L₁ and L₂ are each independently selected from a deuterium, a fluorine, a cyano, a trimethylsilyl, a trideuteromethyl, a trifluoromethyl, a methyl, an ethyl, an isopropyl, a tert-butyl, a phenyl, and a naphthyl.

In some embodiments,

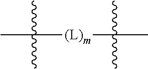

is selected from a single bond, and the group consisting of the following groups:

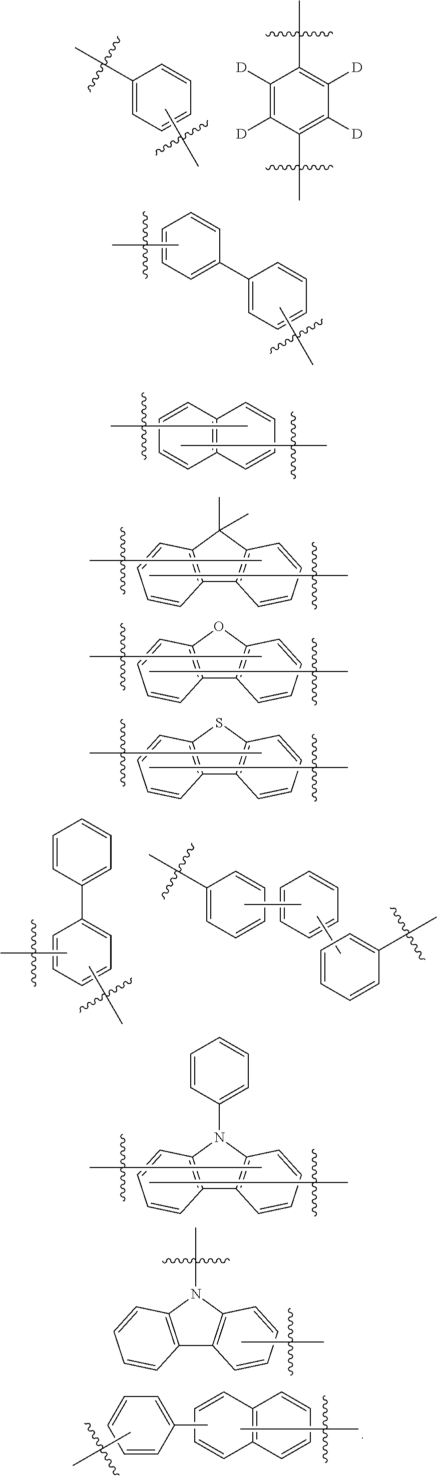

In some embodiments, L₁ and L₂ are each independently selected from a single bond, and the group consisting of the following groups:

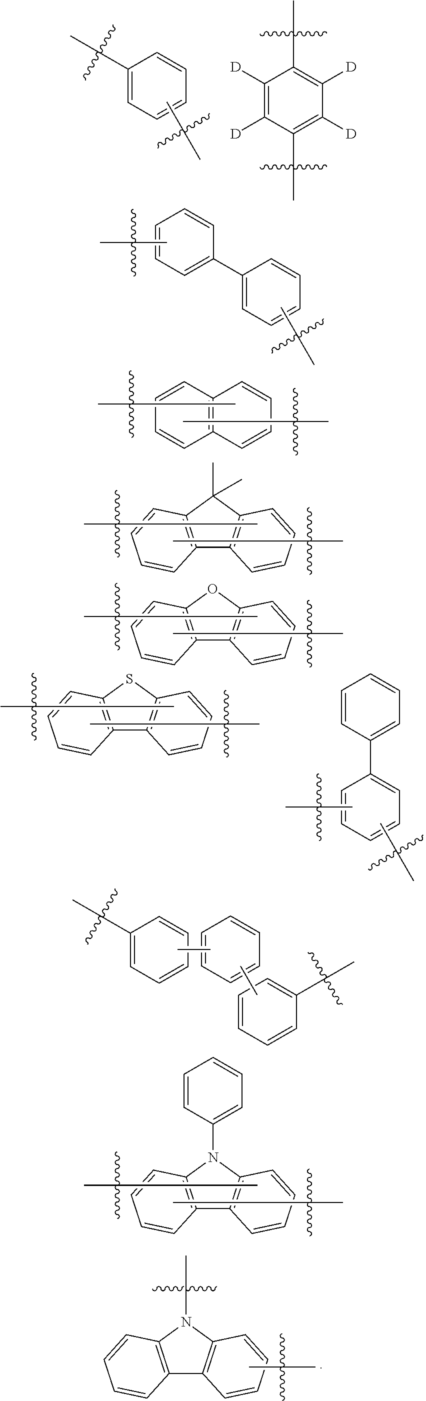

In some embodiments, each L is independently selected from a single bond, a substituted or unsubstituted phenylene, a substituted or unsubstituted naphthylene, a substituted or unsubstituted biphenylene, a substituted or unsubstituted fluorenylene, a substituted or unsubstituted carbazolylene, a substituted or unsubstituted dibenzothienylene, and a substituted or unsubstituted dibenzofuranylene.

Optionally, substituent(s) in L are each independently selected from a deuterium, a fluorine, a cyano, a trimethylsilyl, a trideuteromethyl, a trifluoromethyl, a methyl, an ethyl, an isopropyl, a tert-butyl, a phenyl, and a naphthyl.

In some embodiments, each L is selected from a single bond, and the group consisting of the following groups:

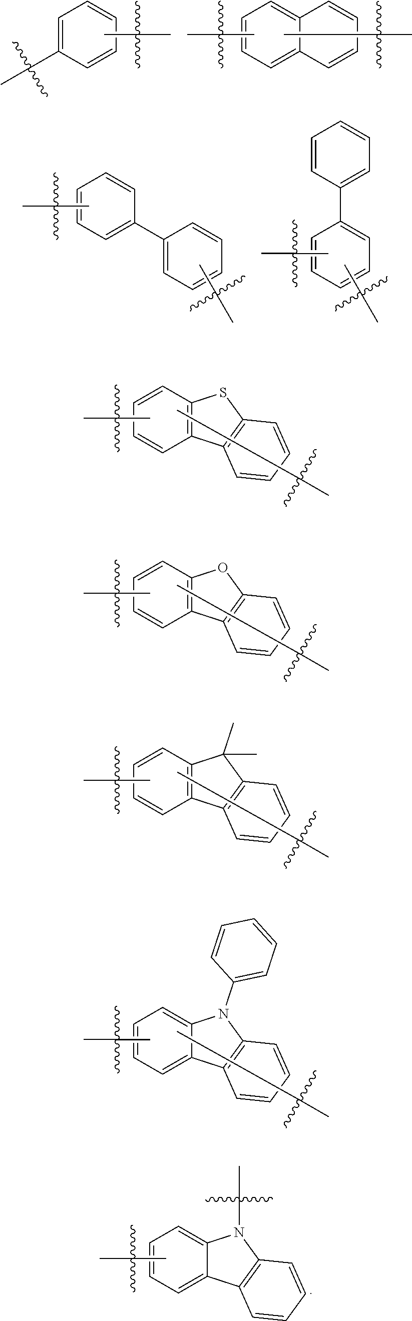

In some embodiments,

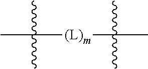

is selected from a single bond, and the group consisting of the following groups:

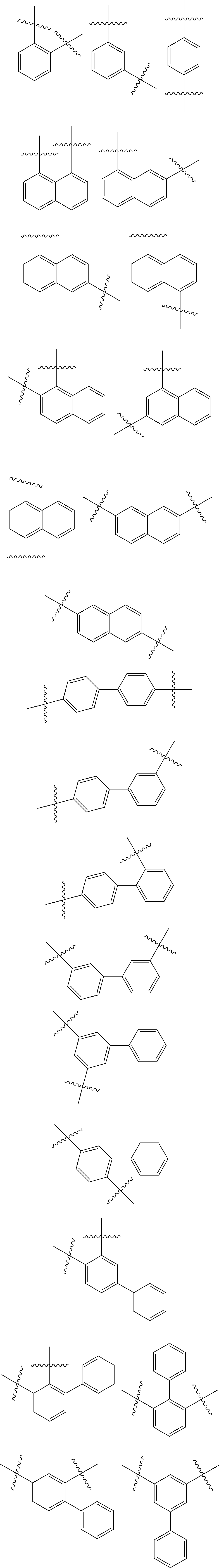

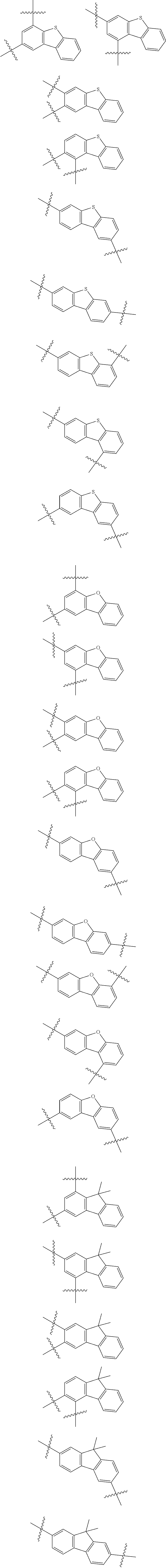

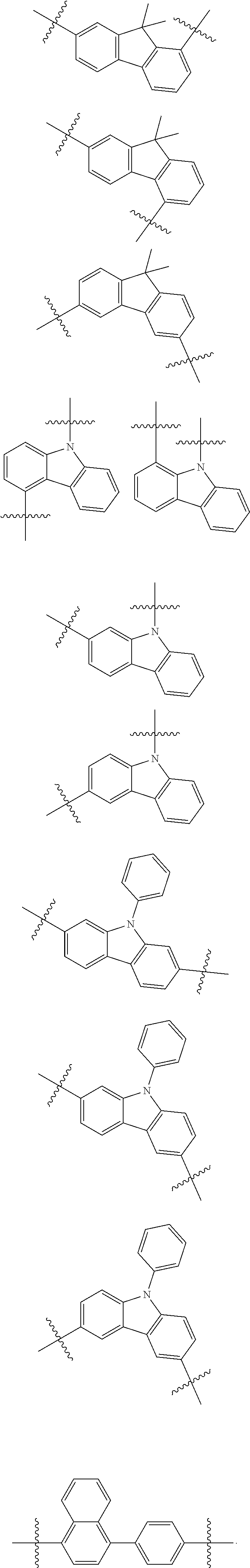

In some embodiments, L₁ and L₂ are each independently selected from a single bond, a substituted or unsubstituted phenylene, a substituted or unsubstituted naphthylene, a substituted or unsubstituted biphenylene, and a substituted or unsubstituted carbazolylene.

Optionally, substituent(s) in L₁ and L₂ are each independently selected from a deuterium, a fluorine, a cyano, a trimethylsilyl, a trideuteromethyl, a trifluoromethyl, a methyl, an ethyl, an isopropyl, a tert-butyl, a phenyl, and a naphthyl.

In some embodiments, L₁ and L₂ are each independently selected from a single bond, and the group consisting of the following groups:

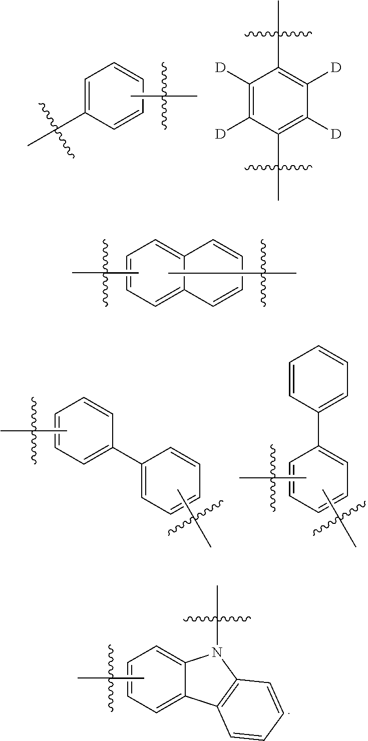

In some embodiments, L₁ and L₂ are each independently selected from a single bond, and the group consisting of the following groups:

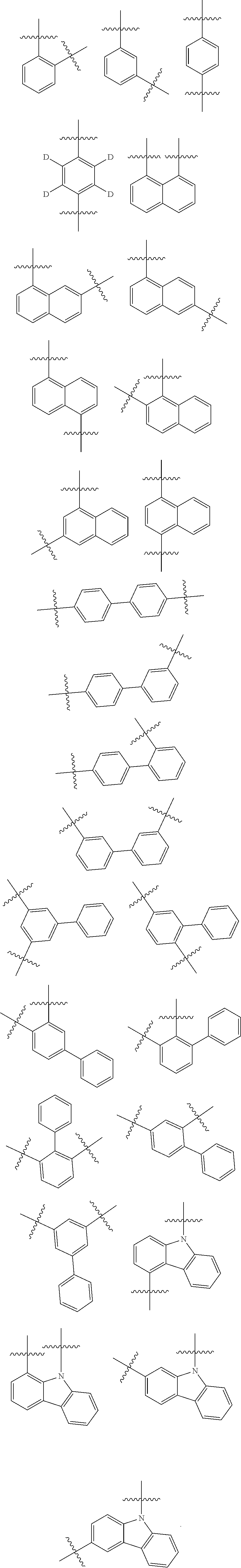

In some embodiments,

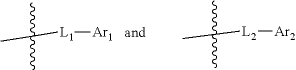

are each independently selected from the group consisting of the following groups:

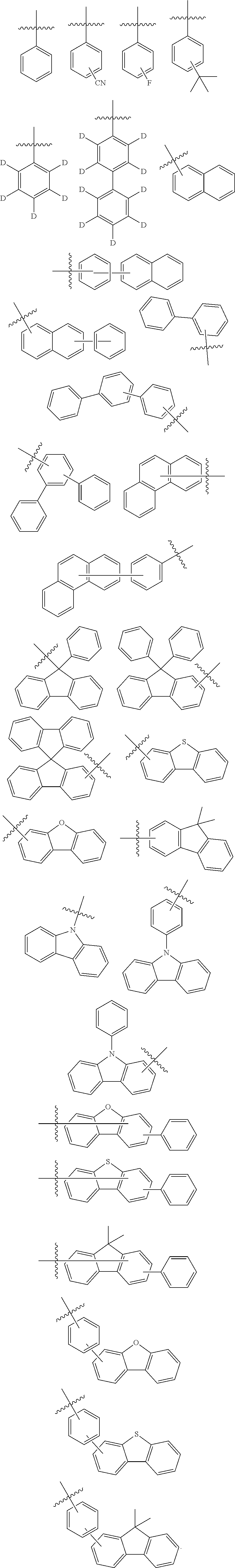

Specifically, the organic compound may be selected from the group consisting of the following compounds:

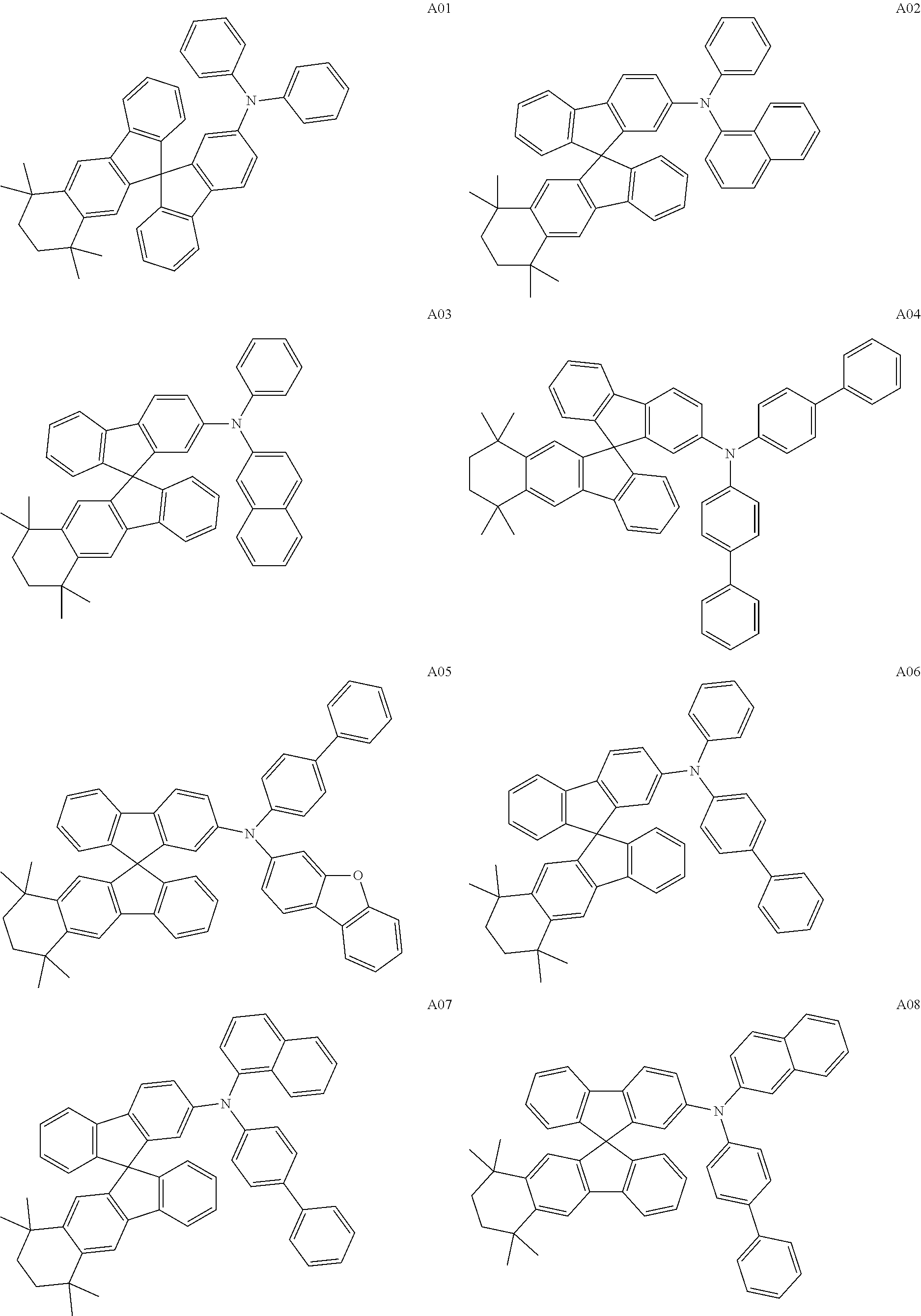

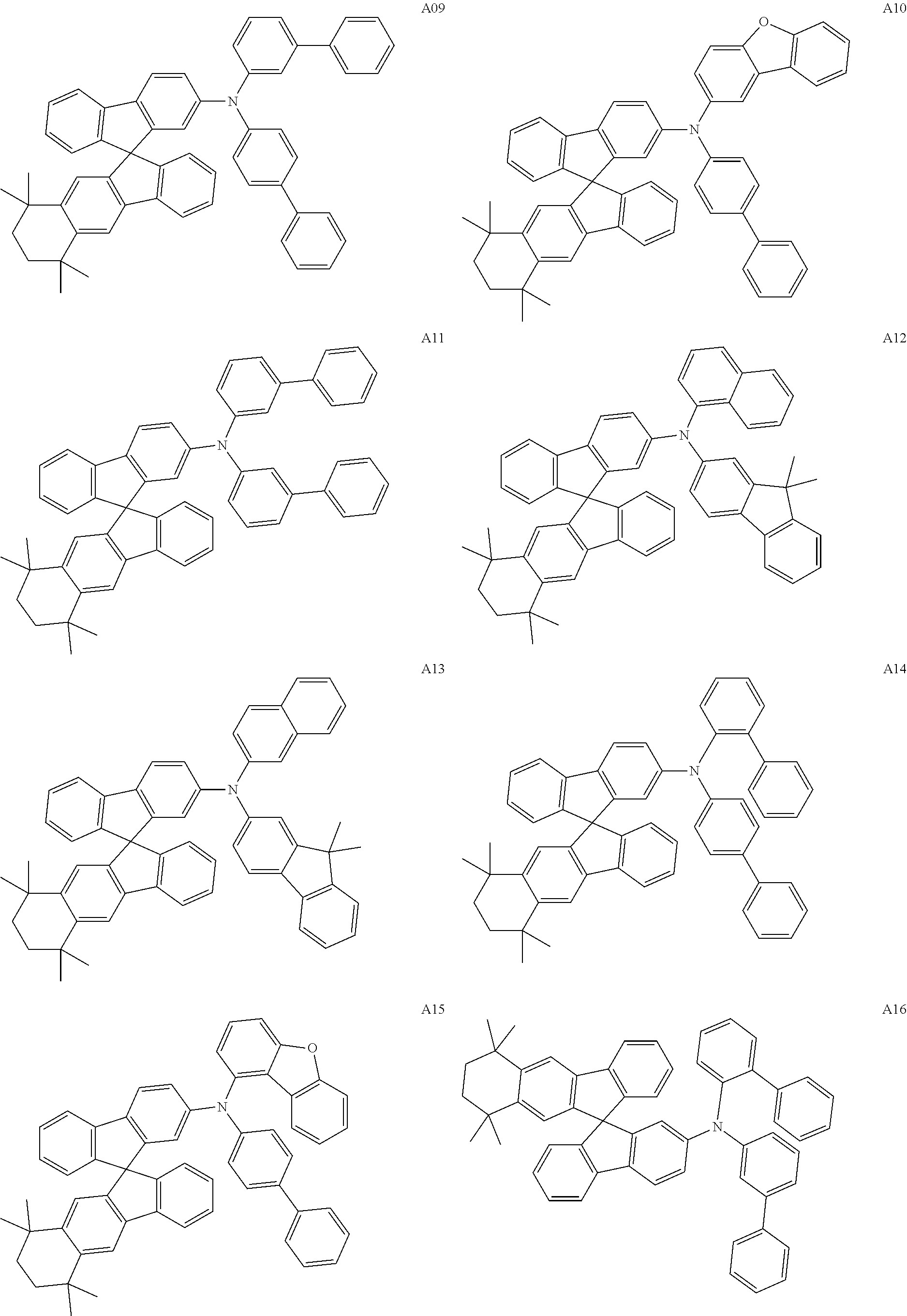

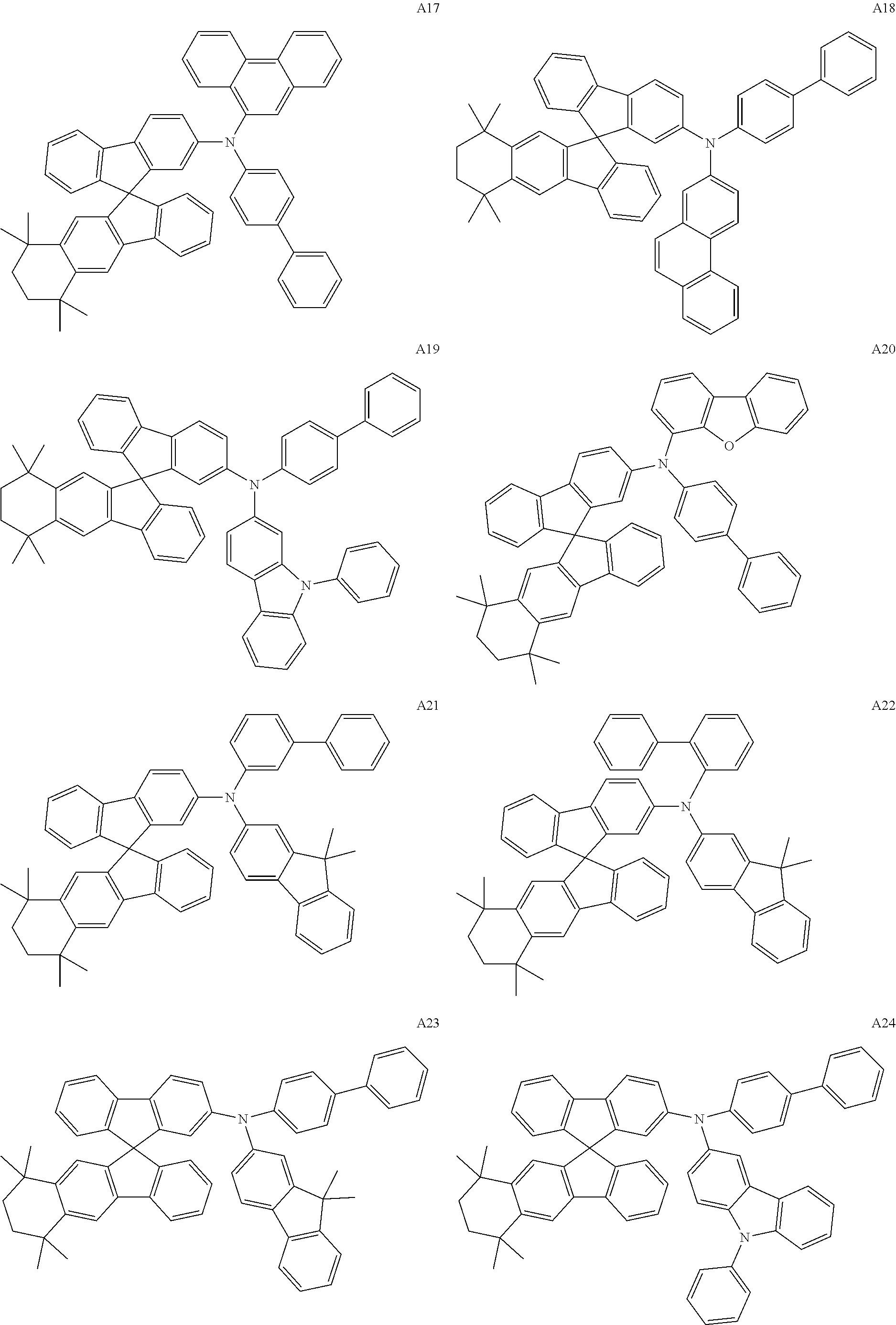

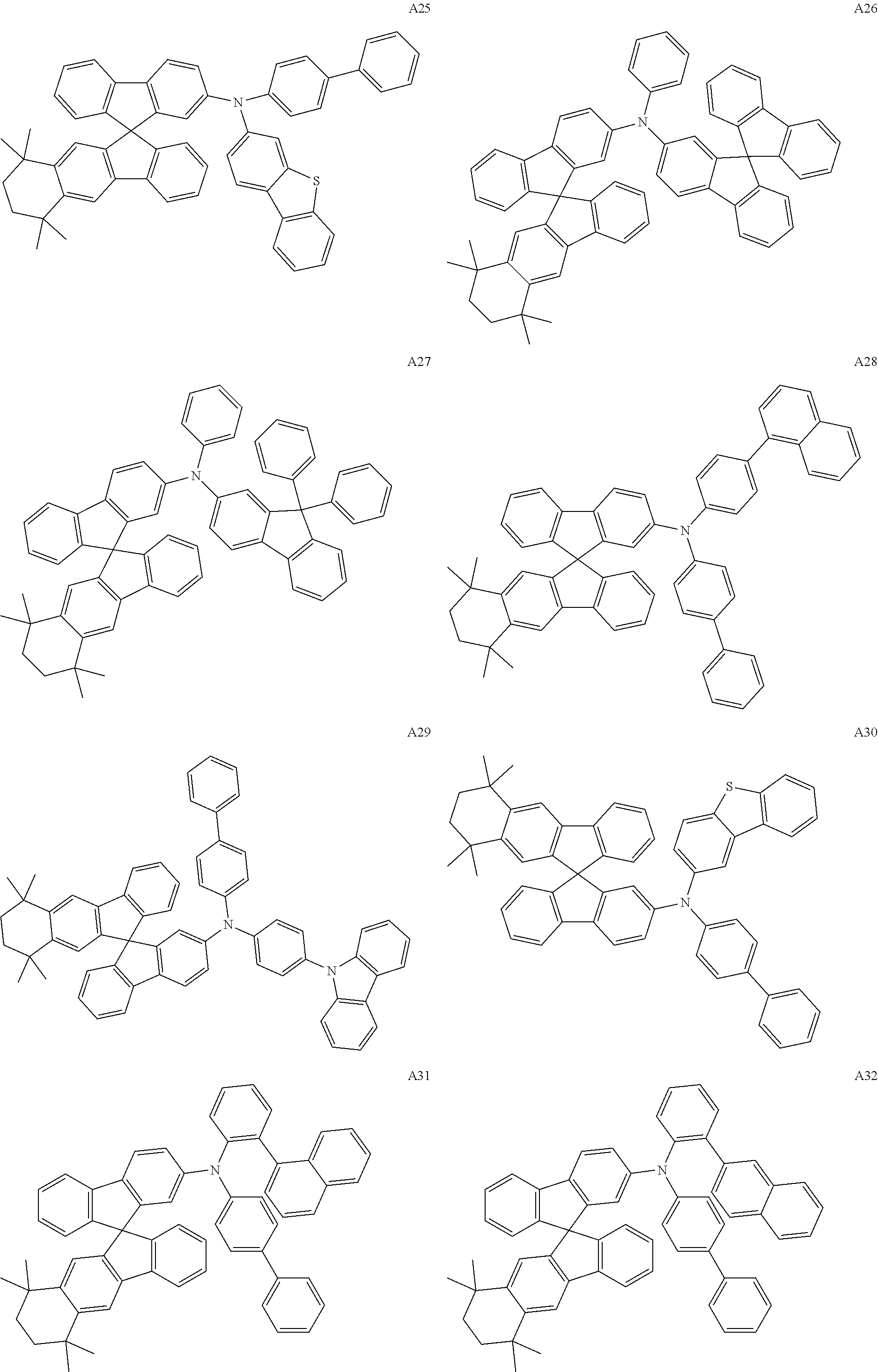

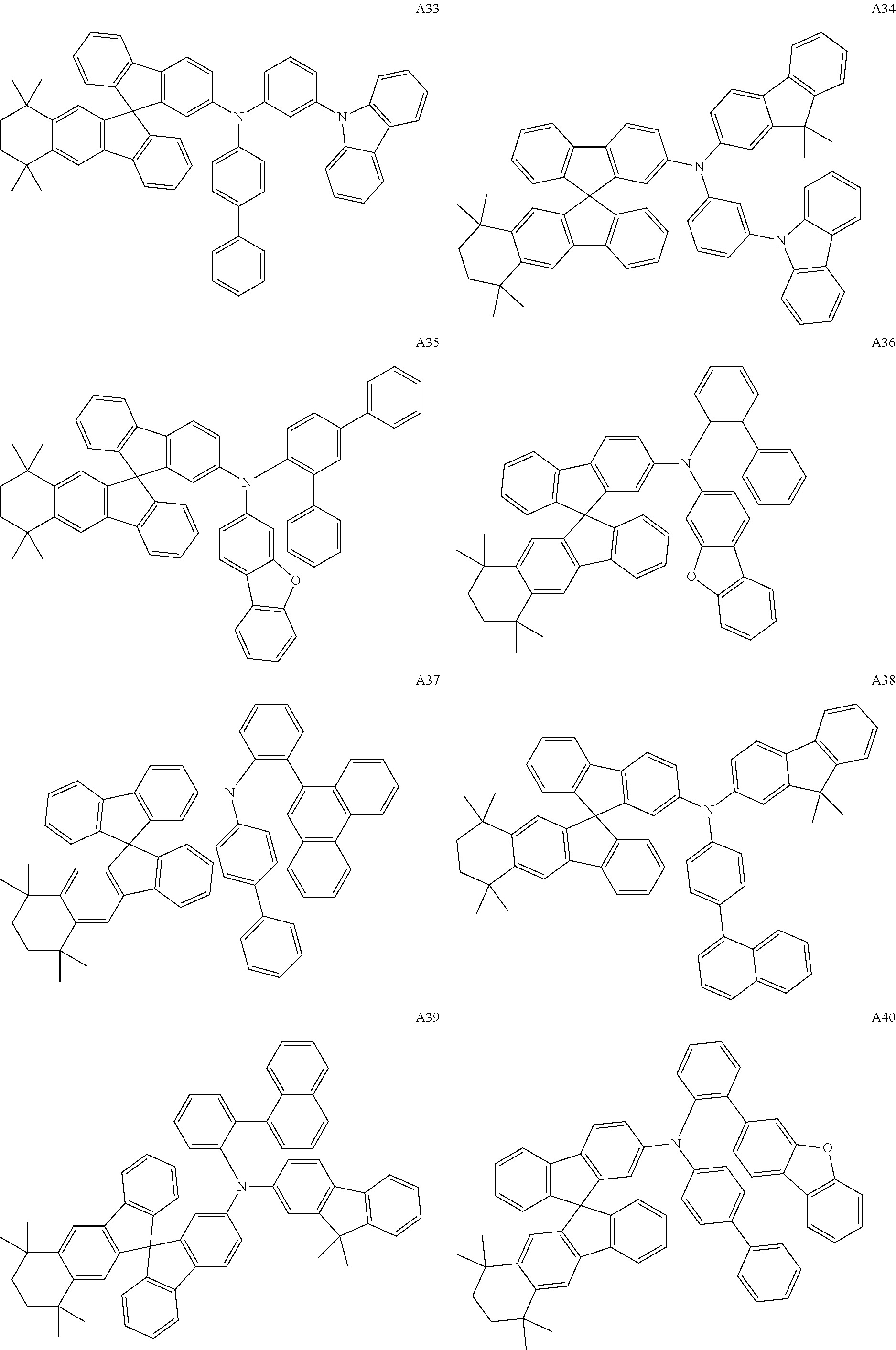

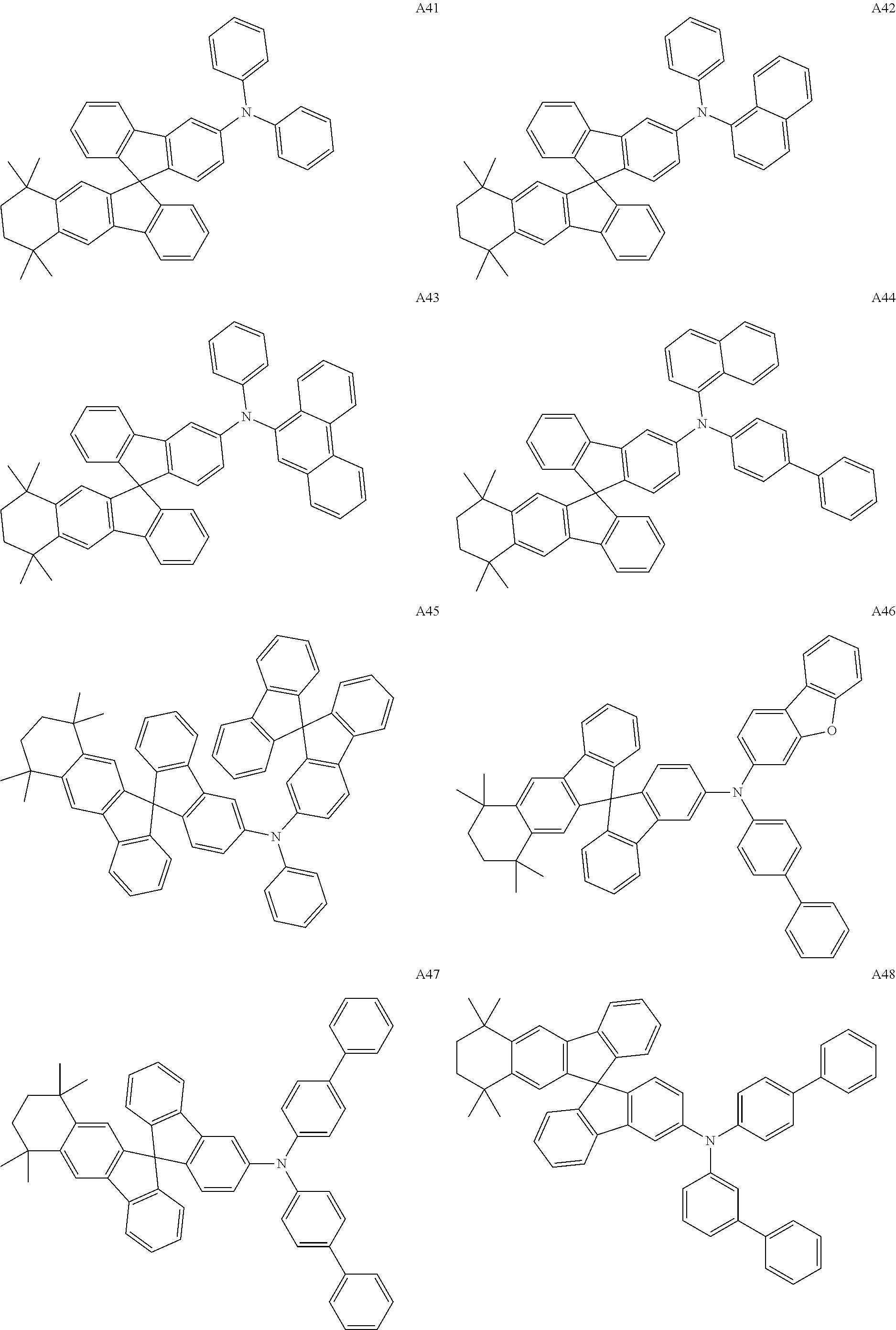

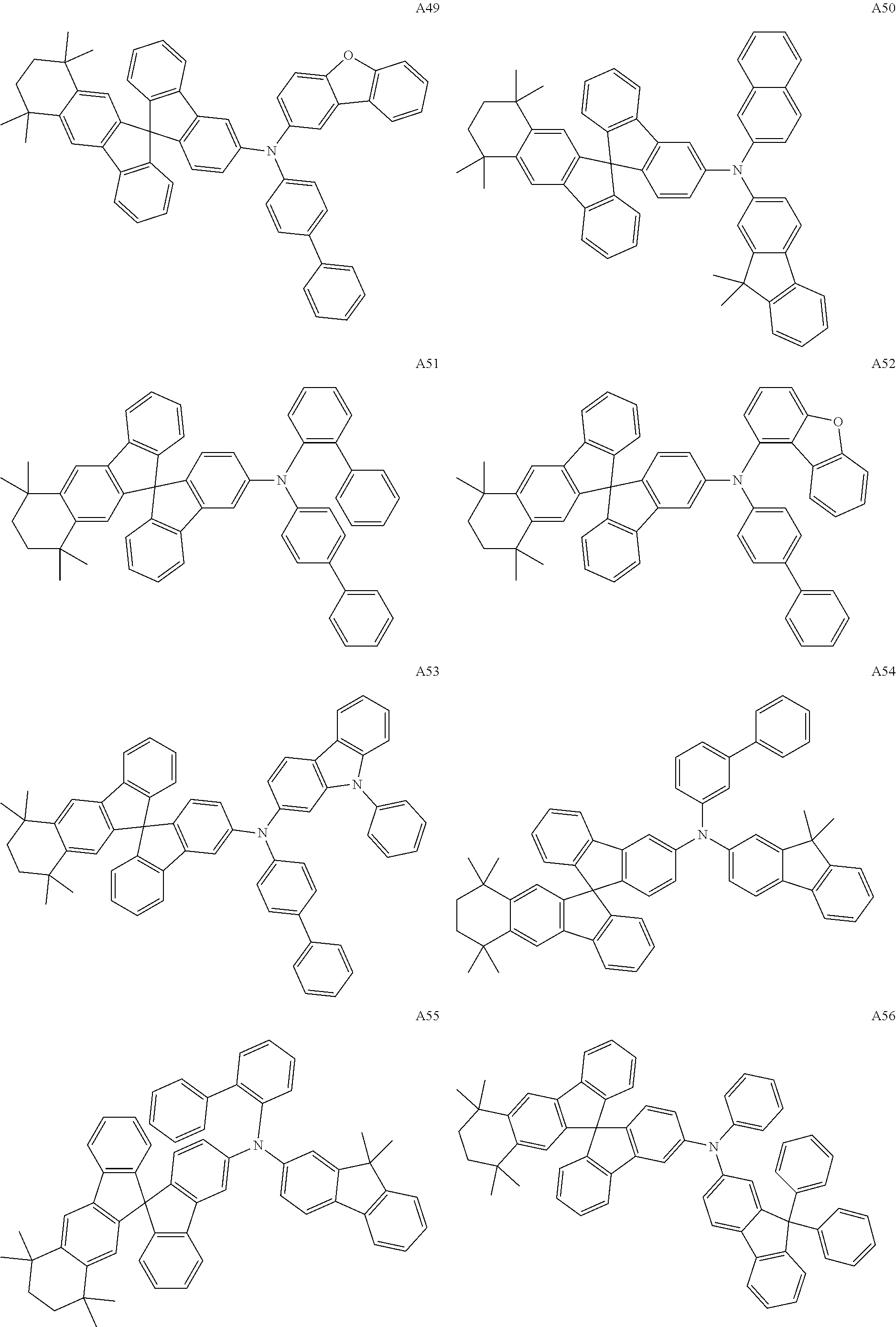

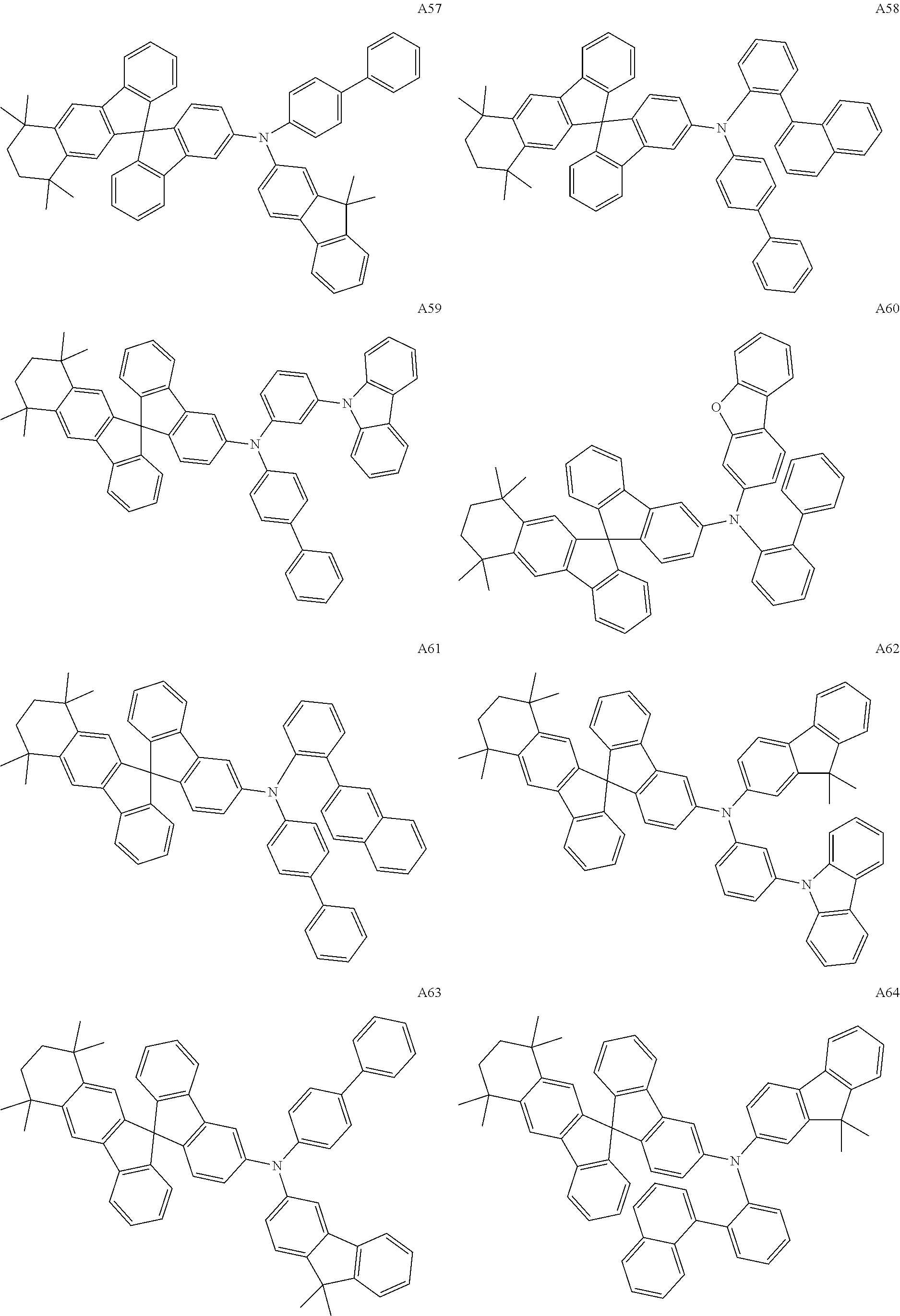

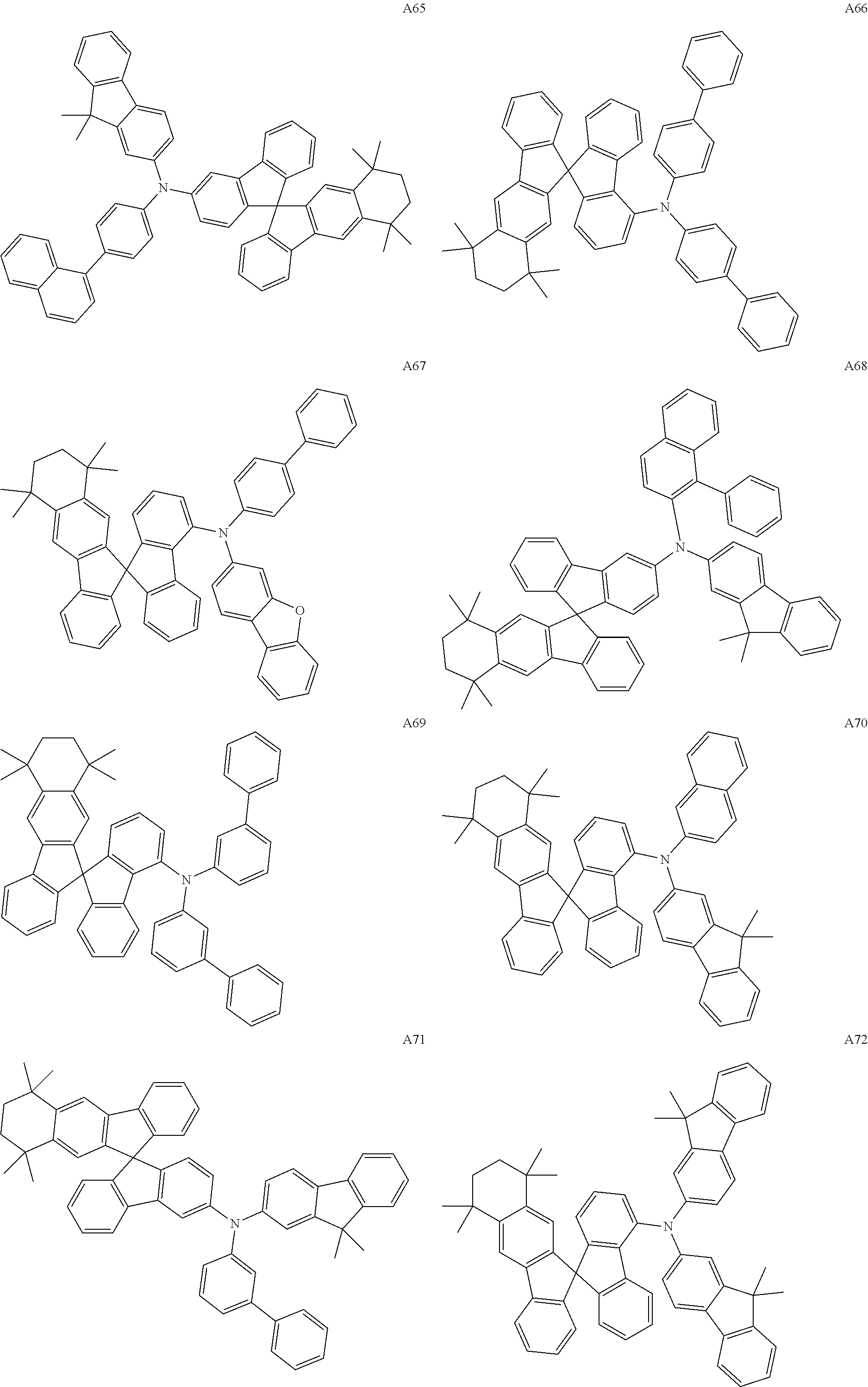

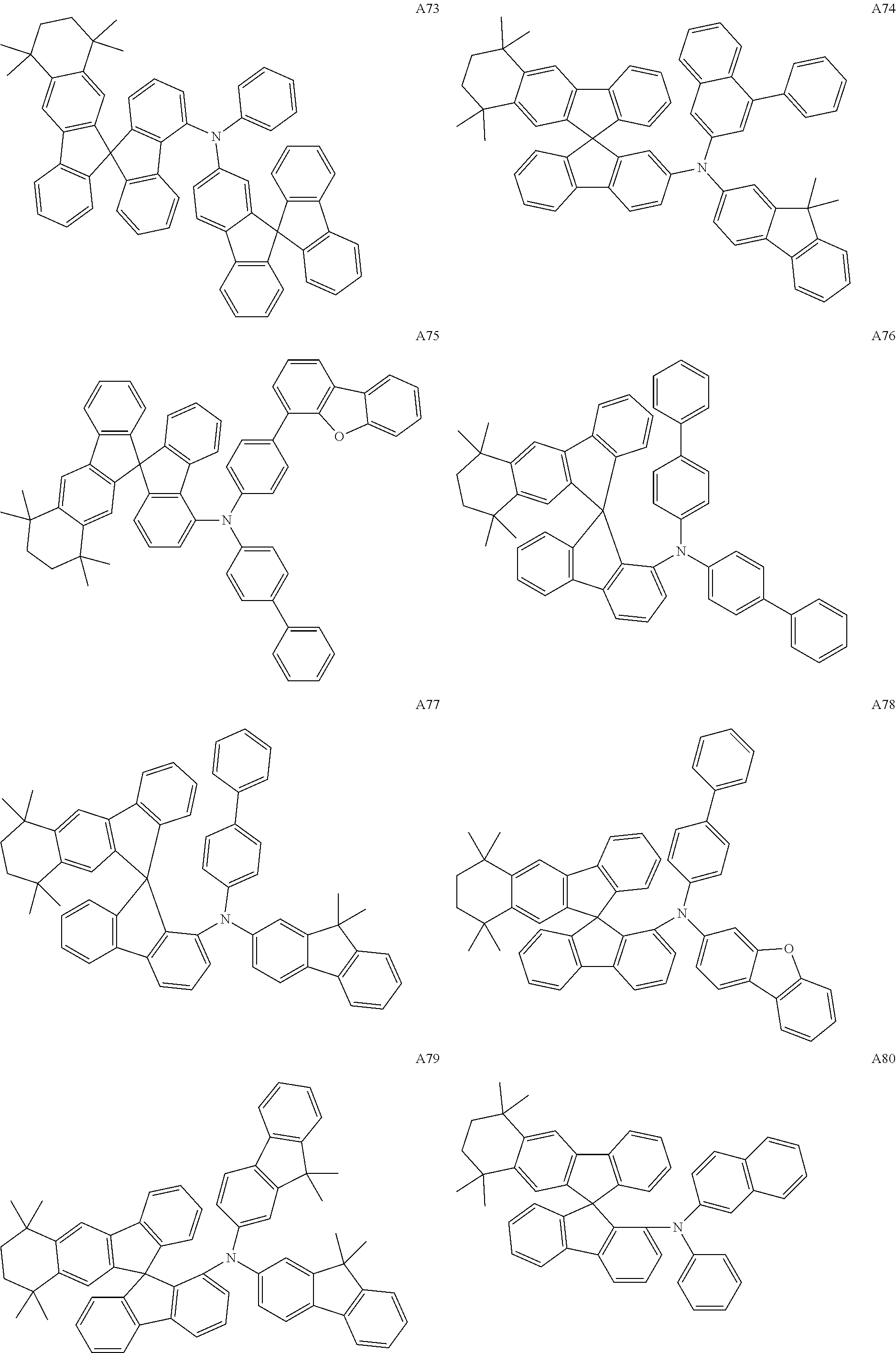

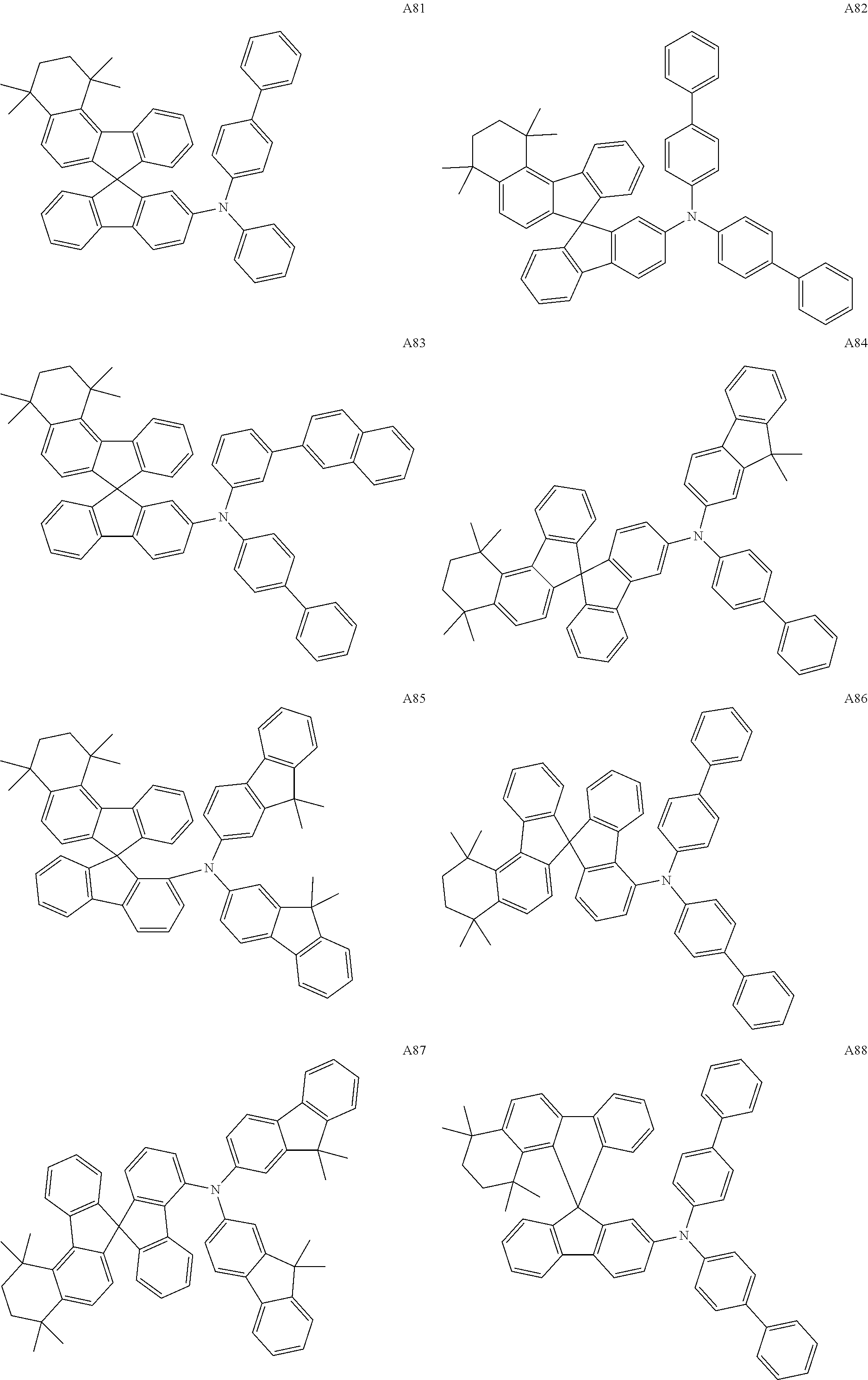

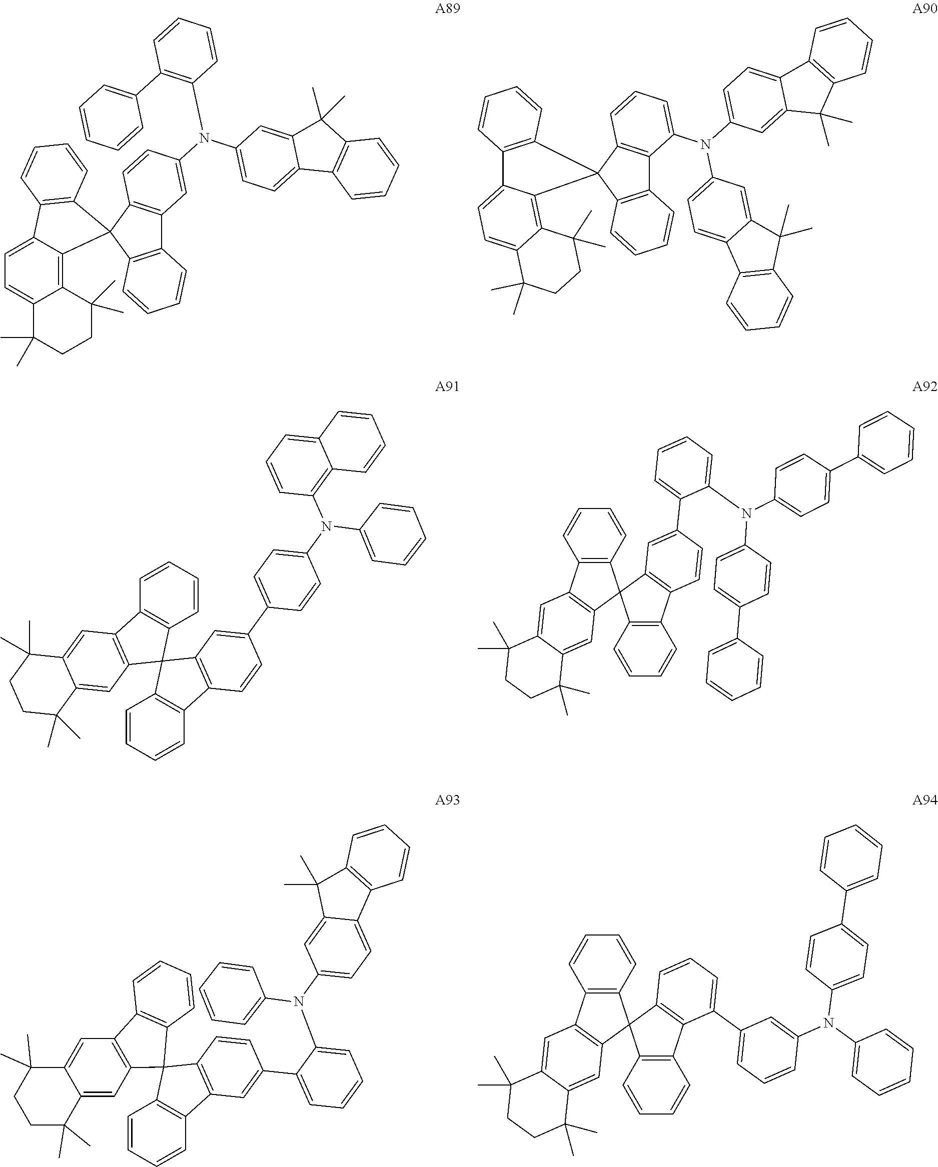

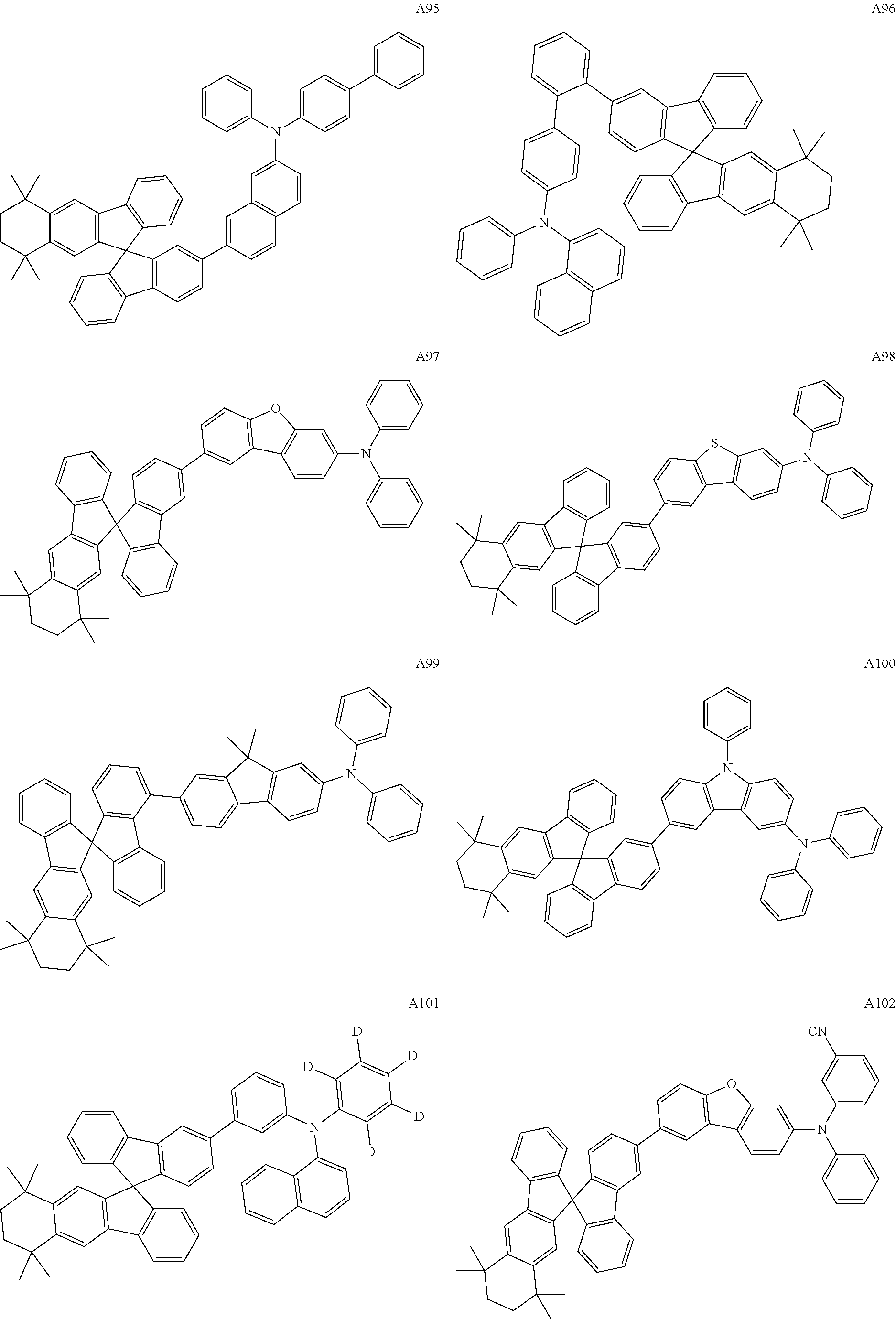

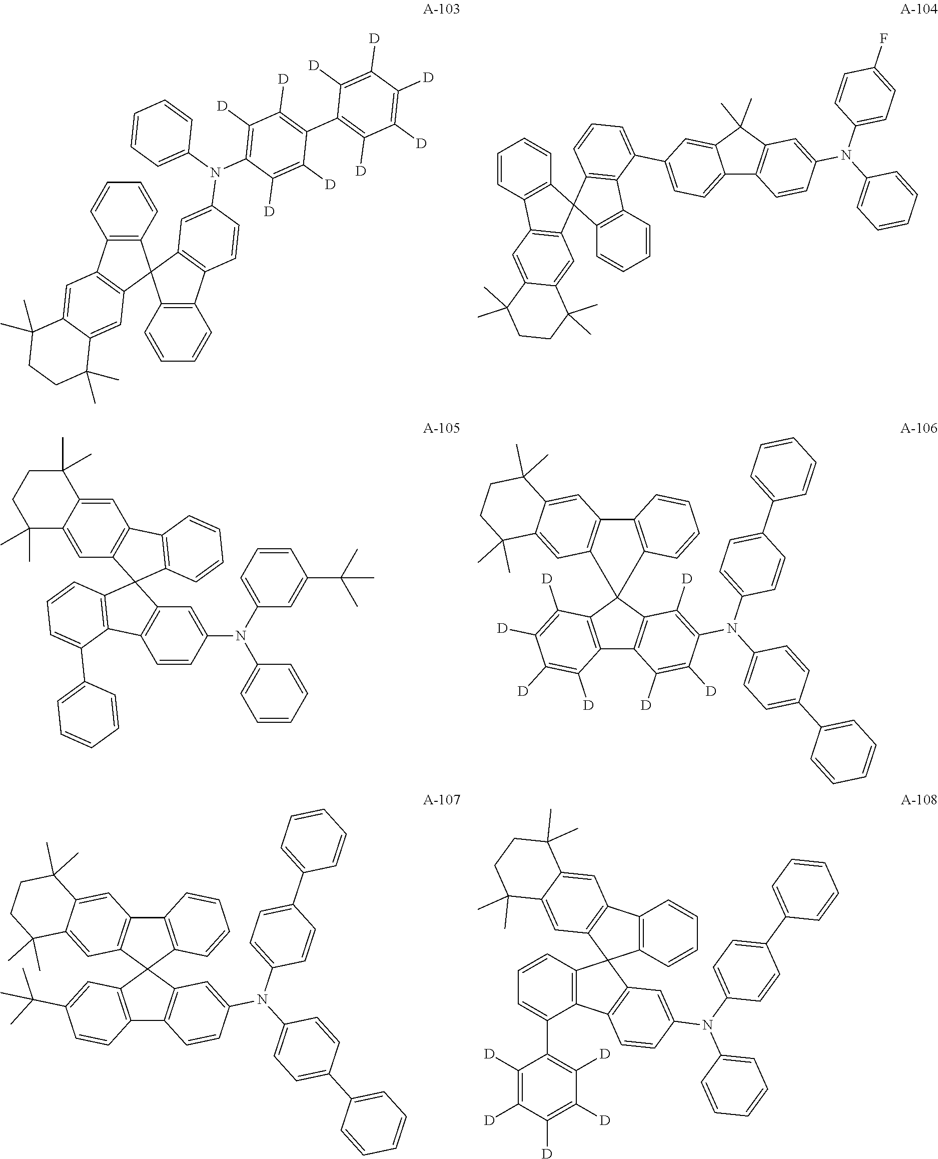

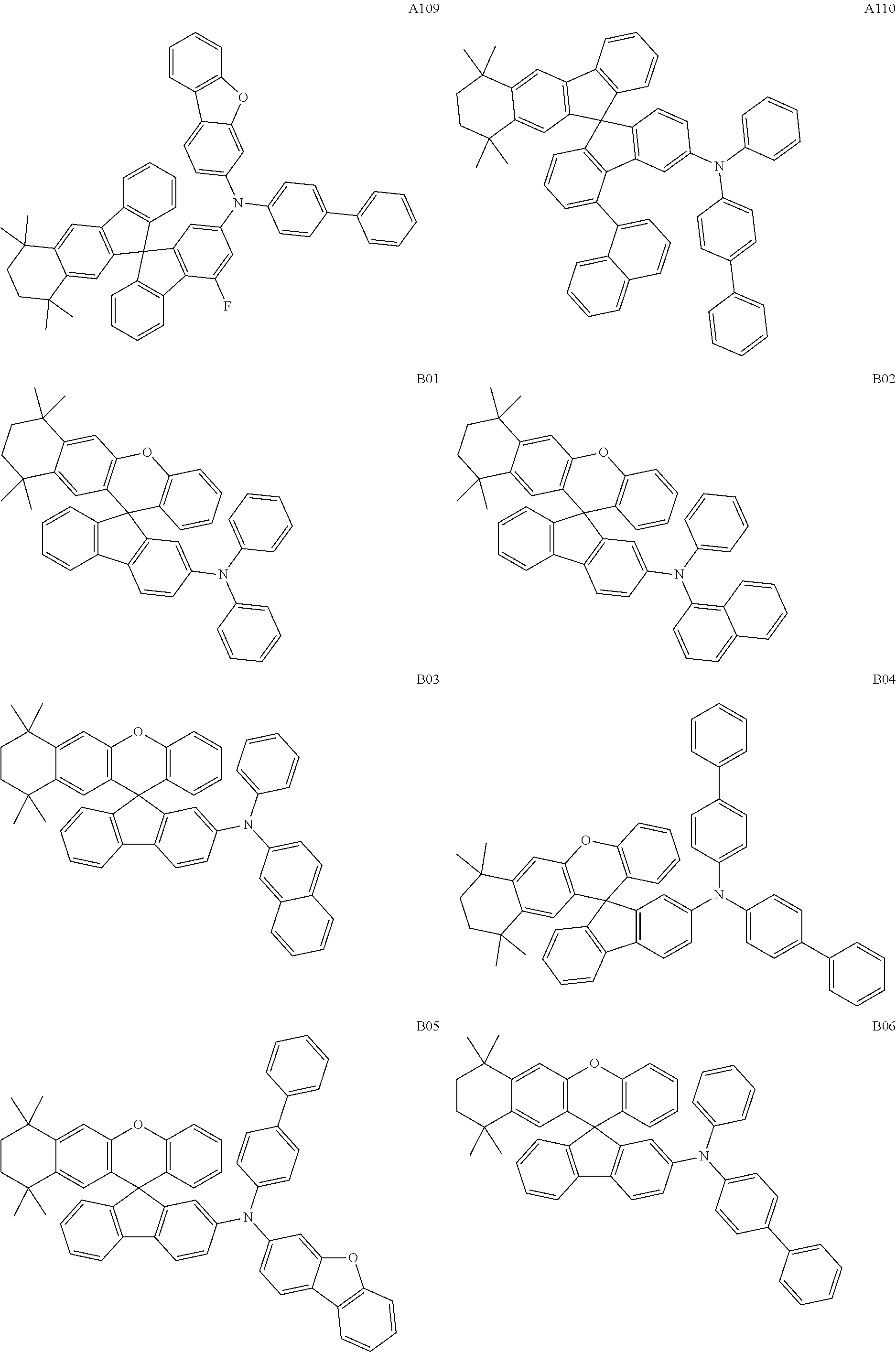

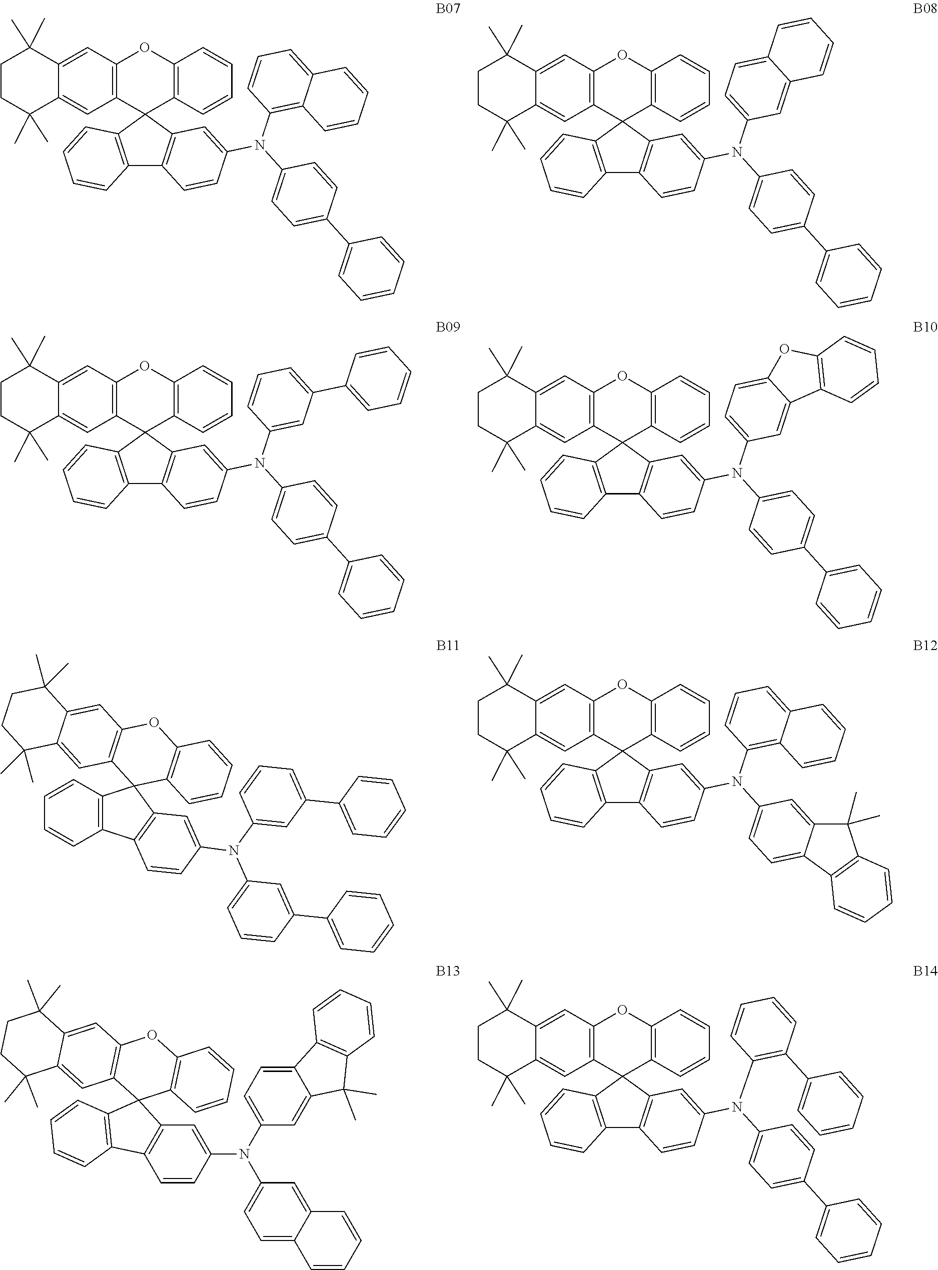

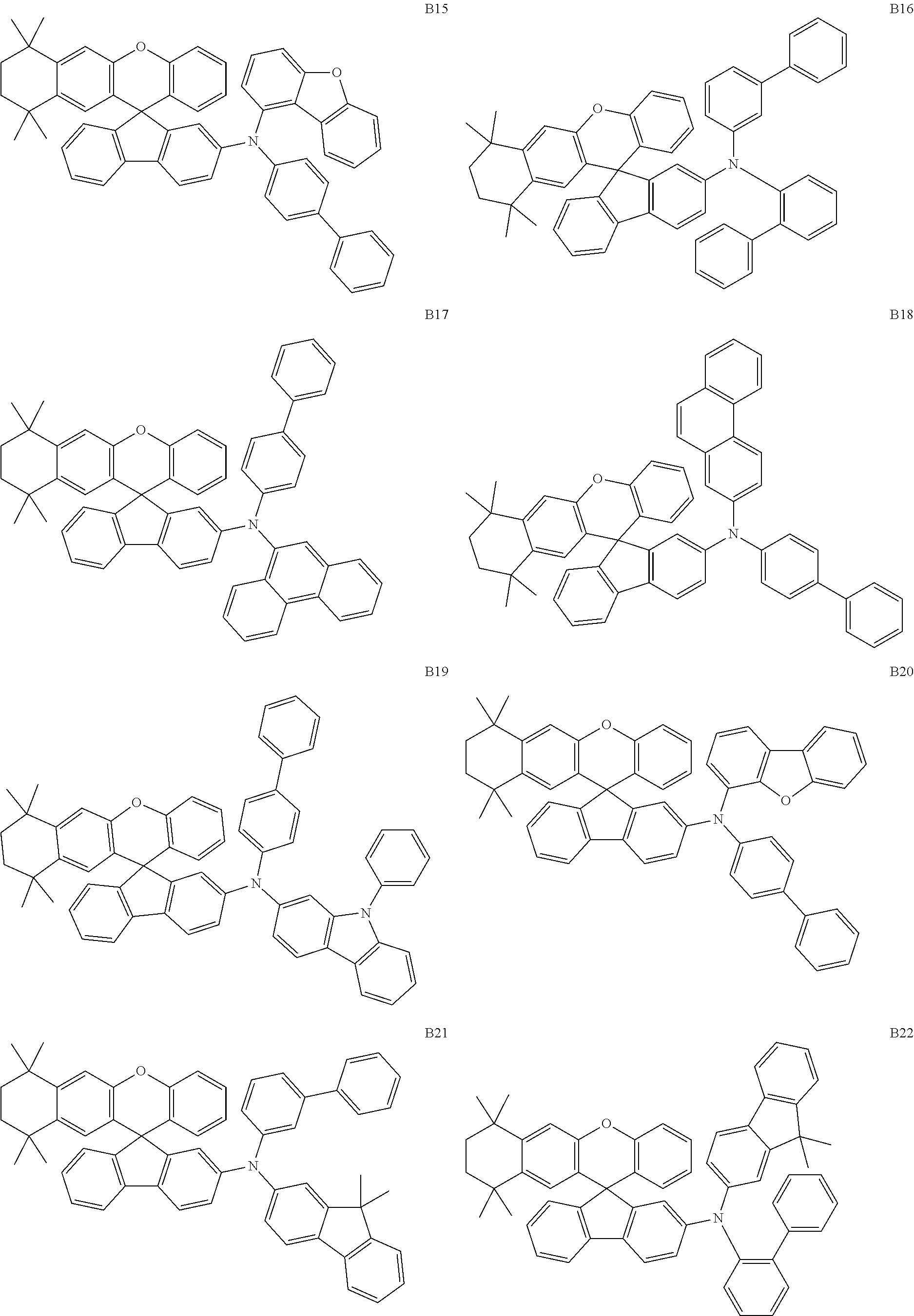

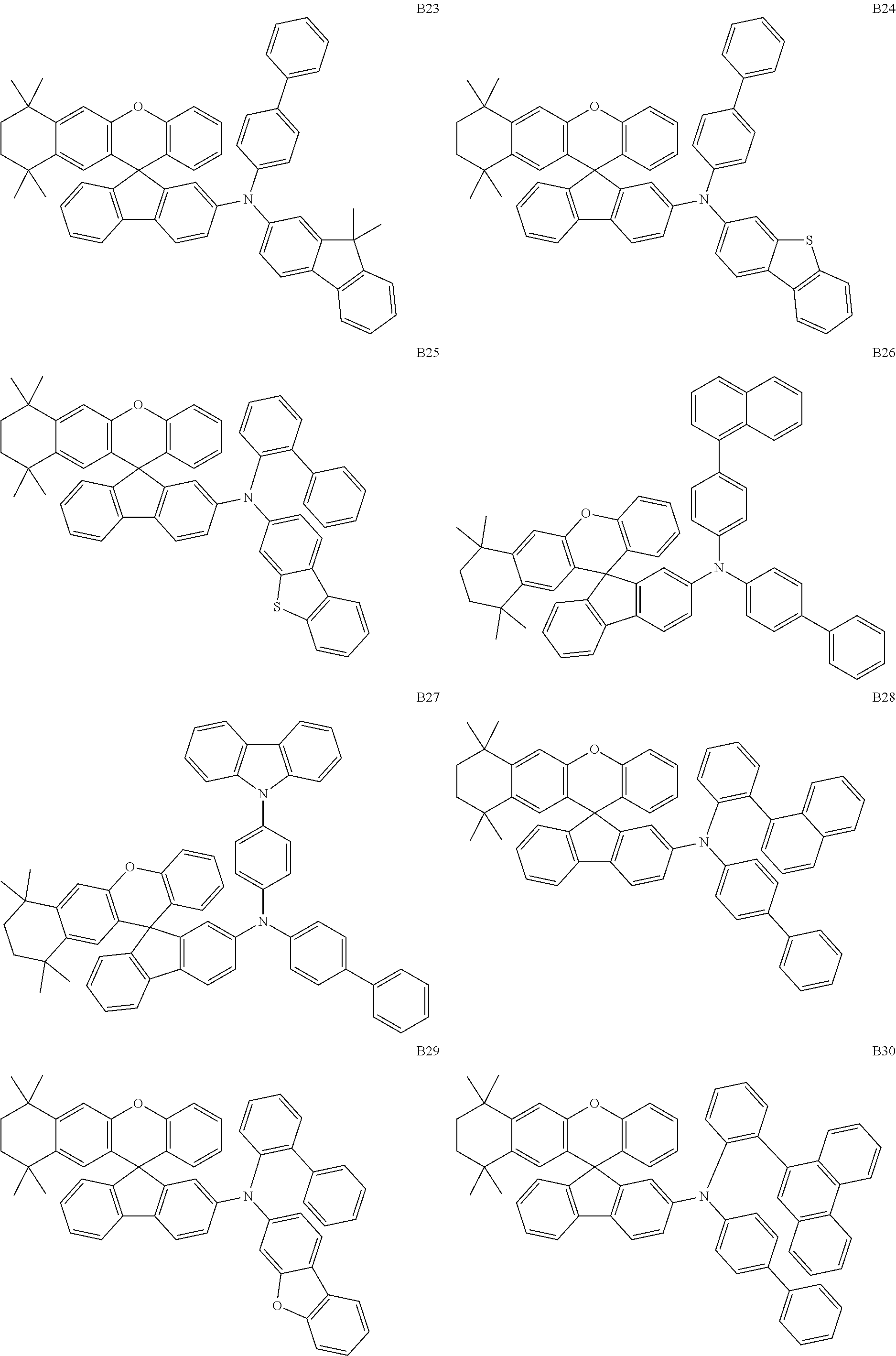

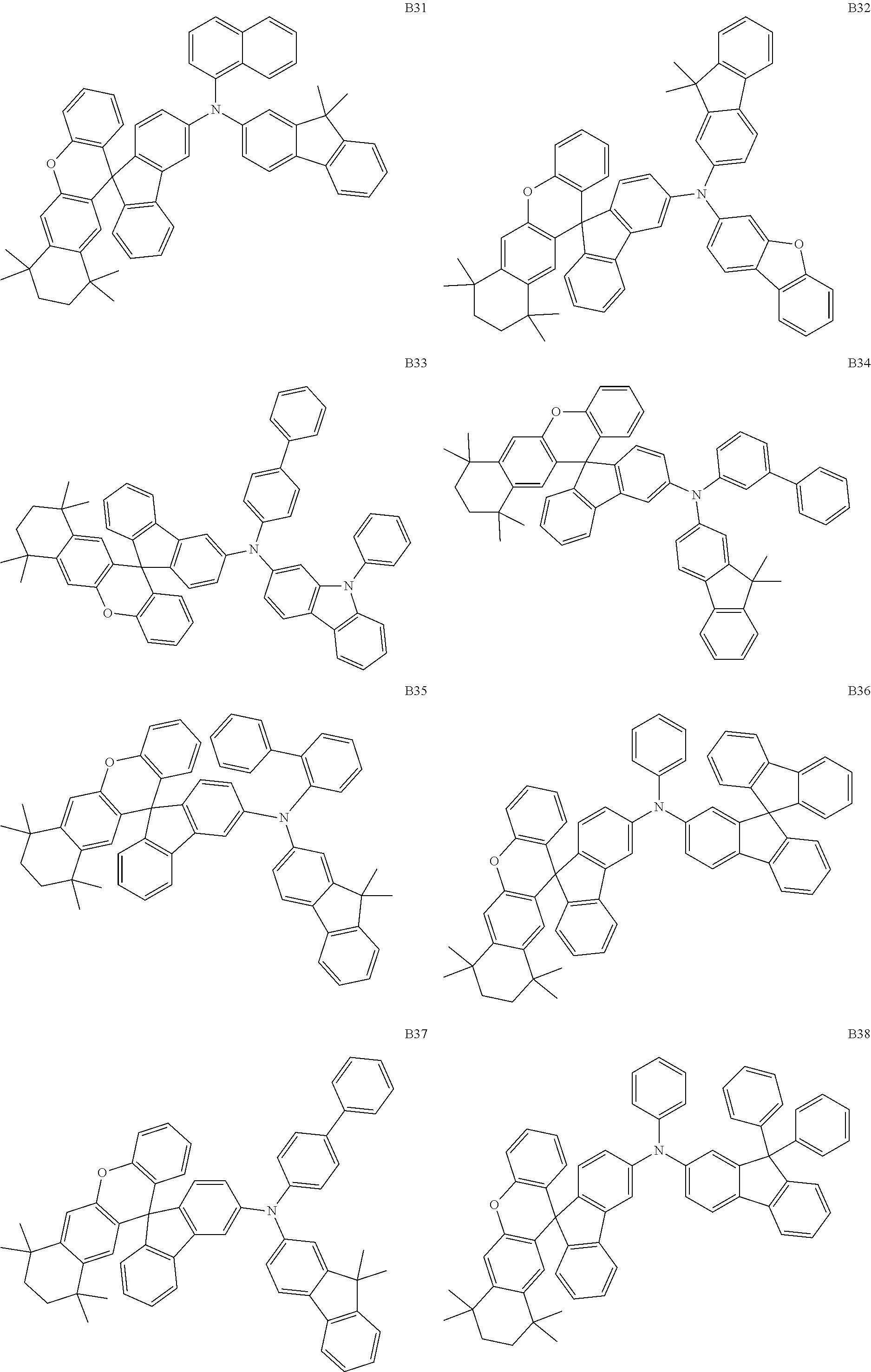

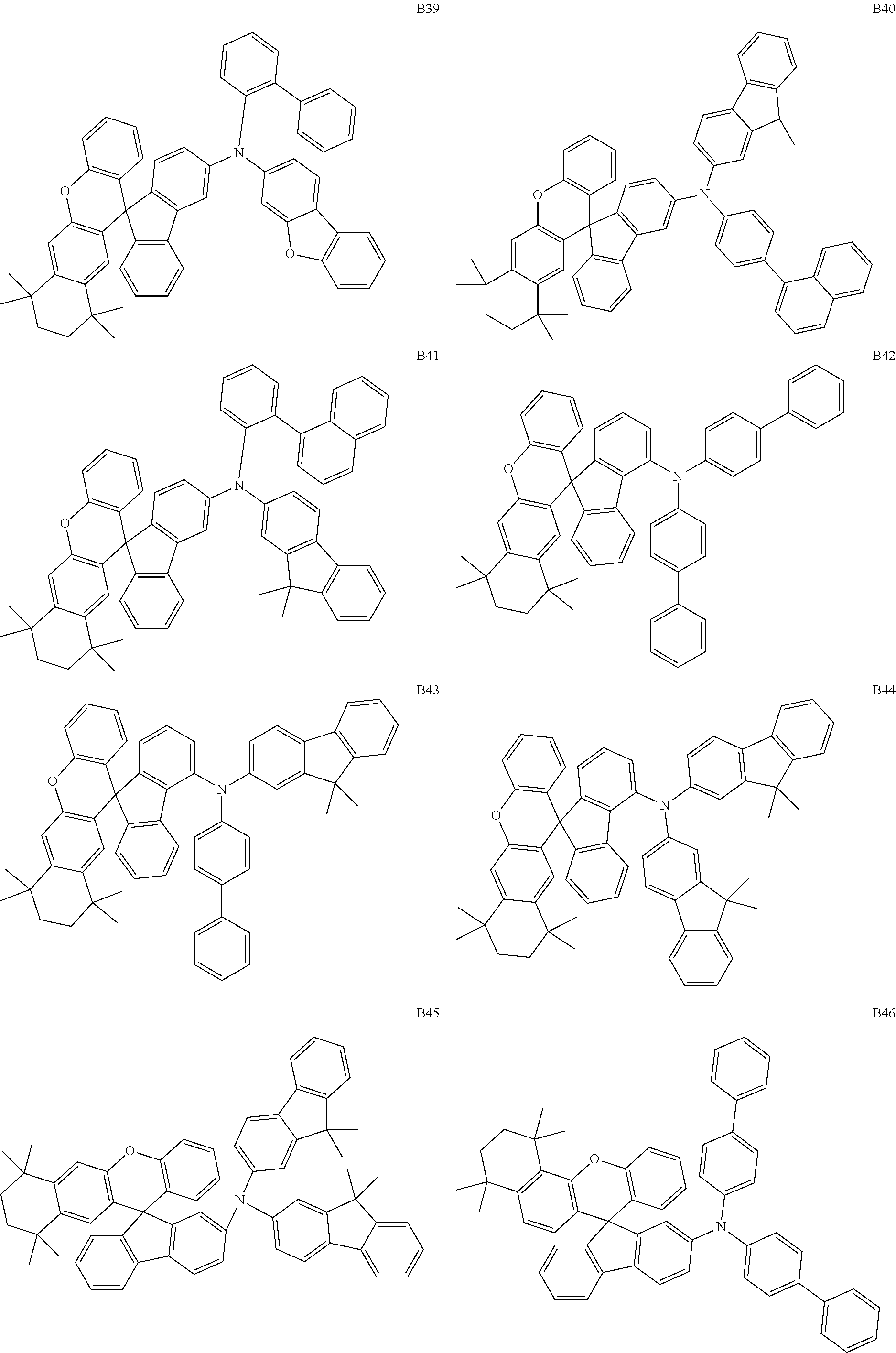

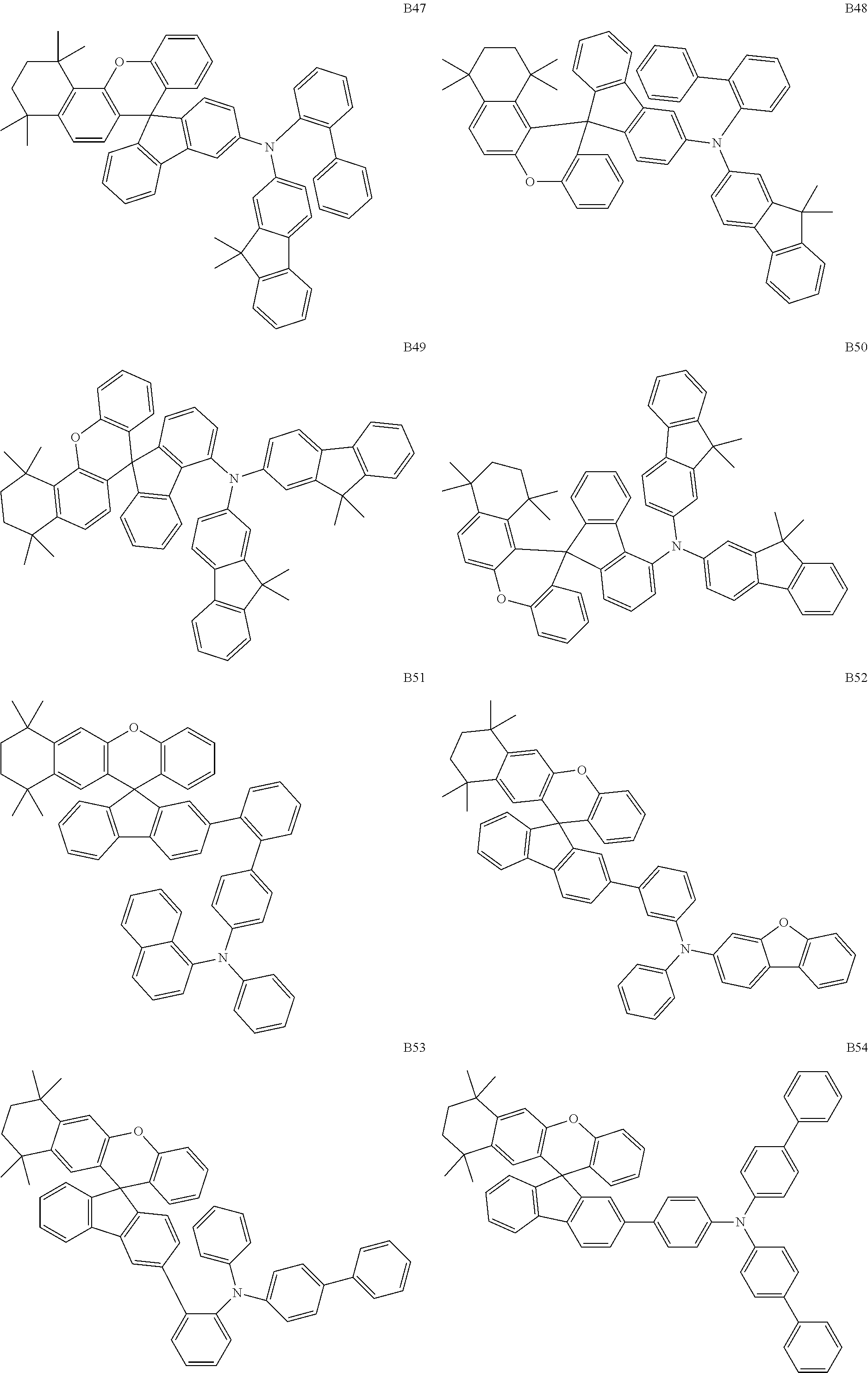

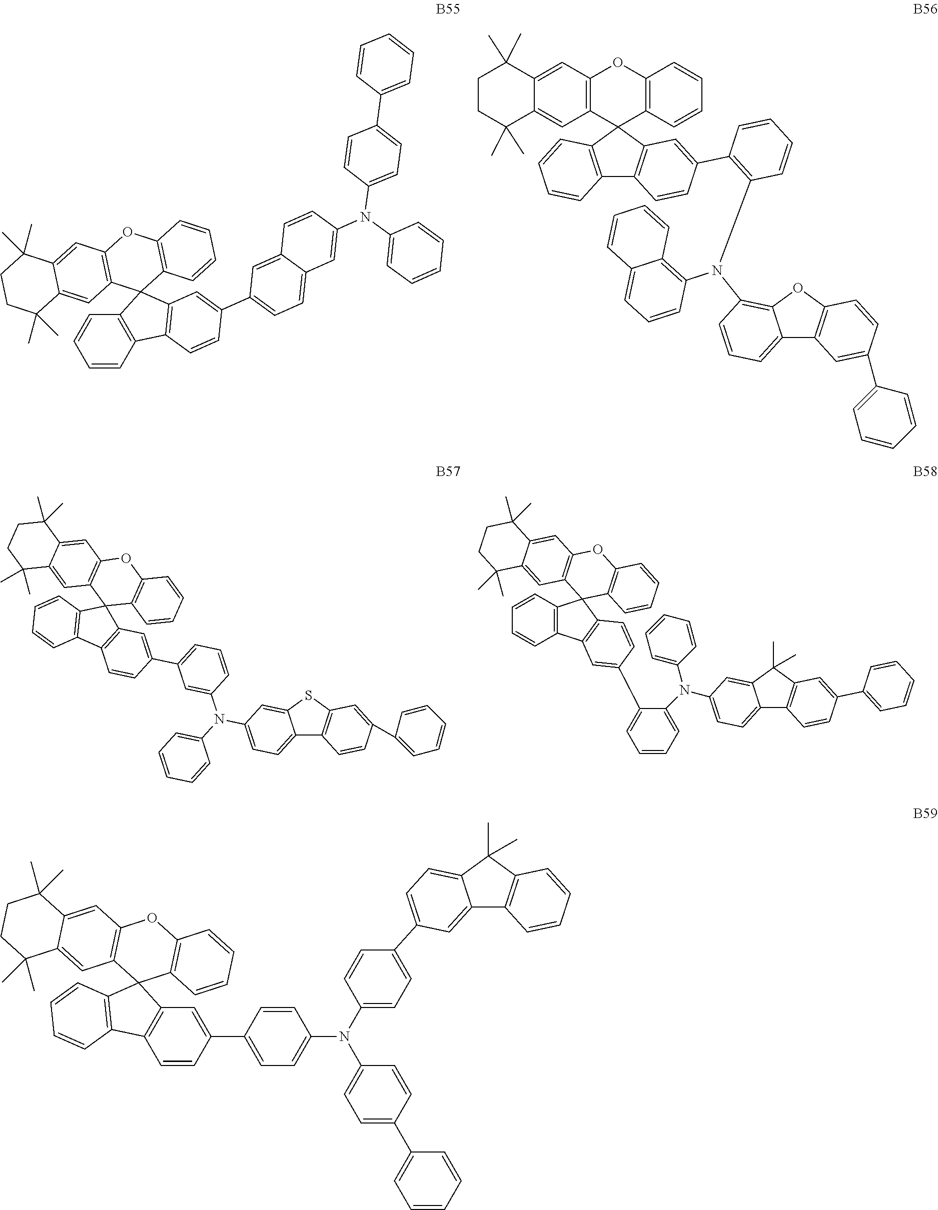

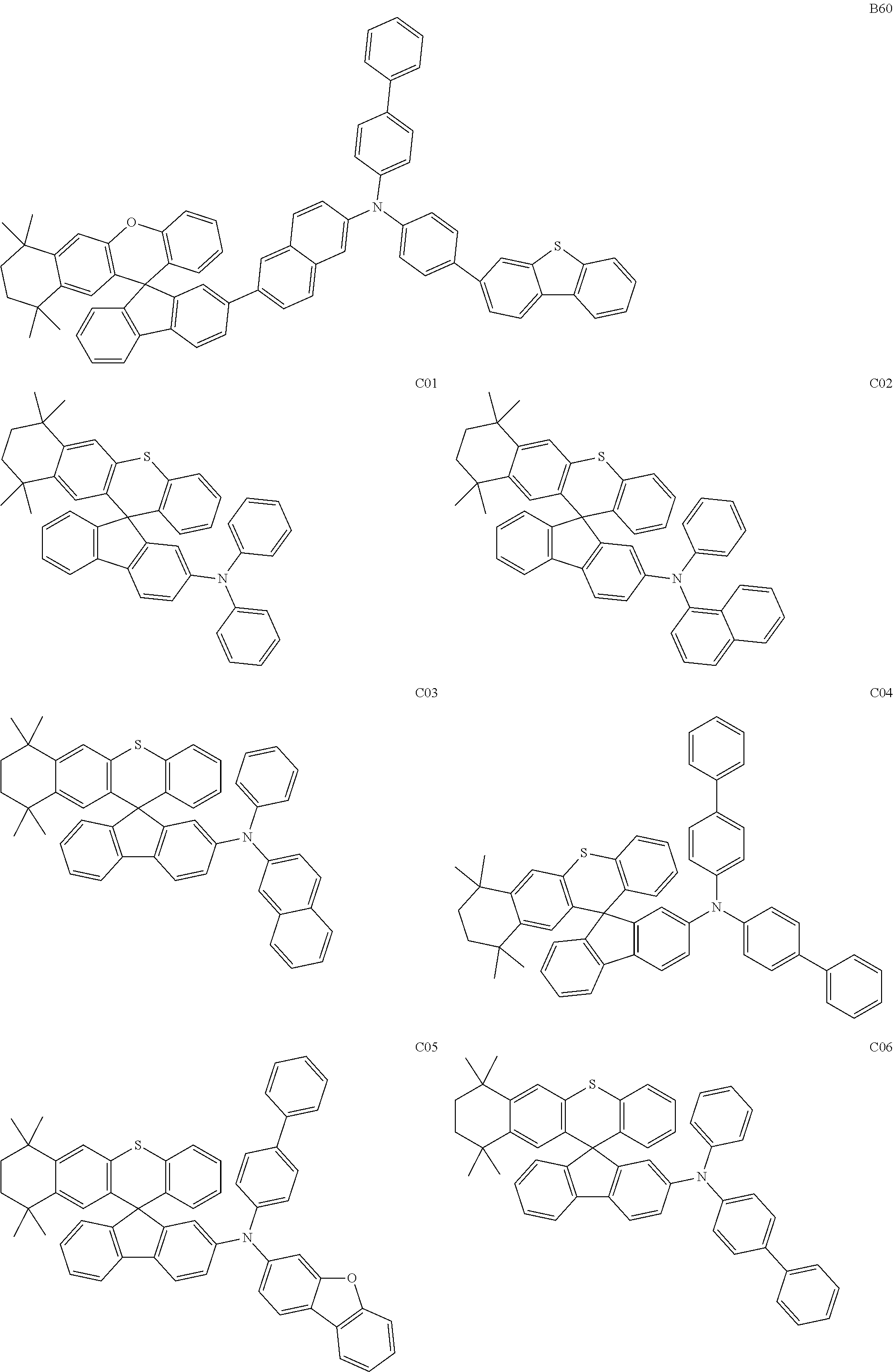

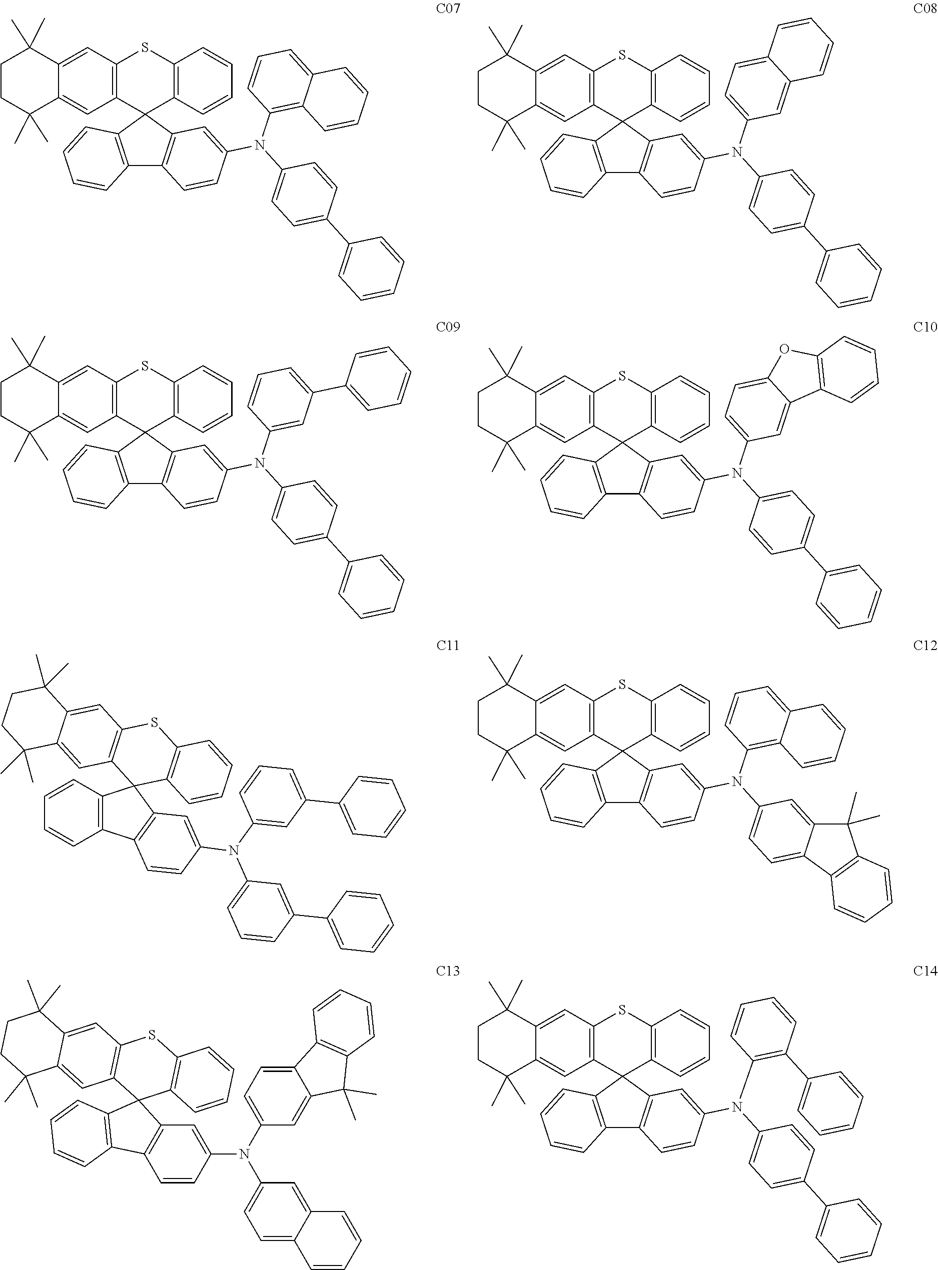

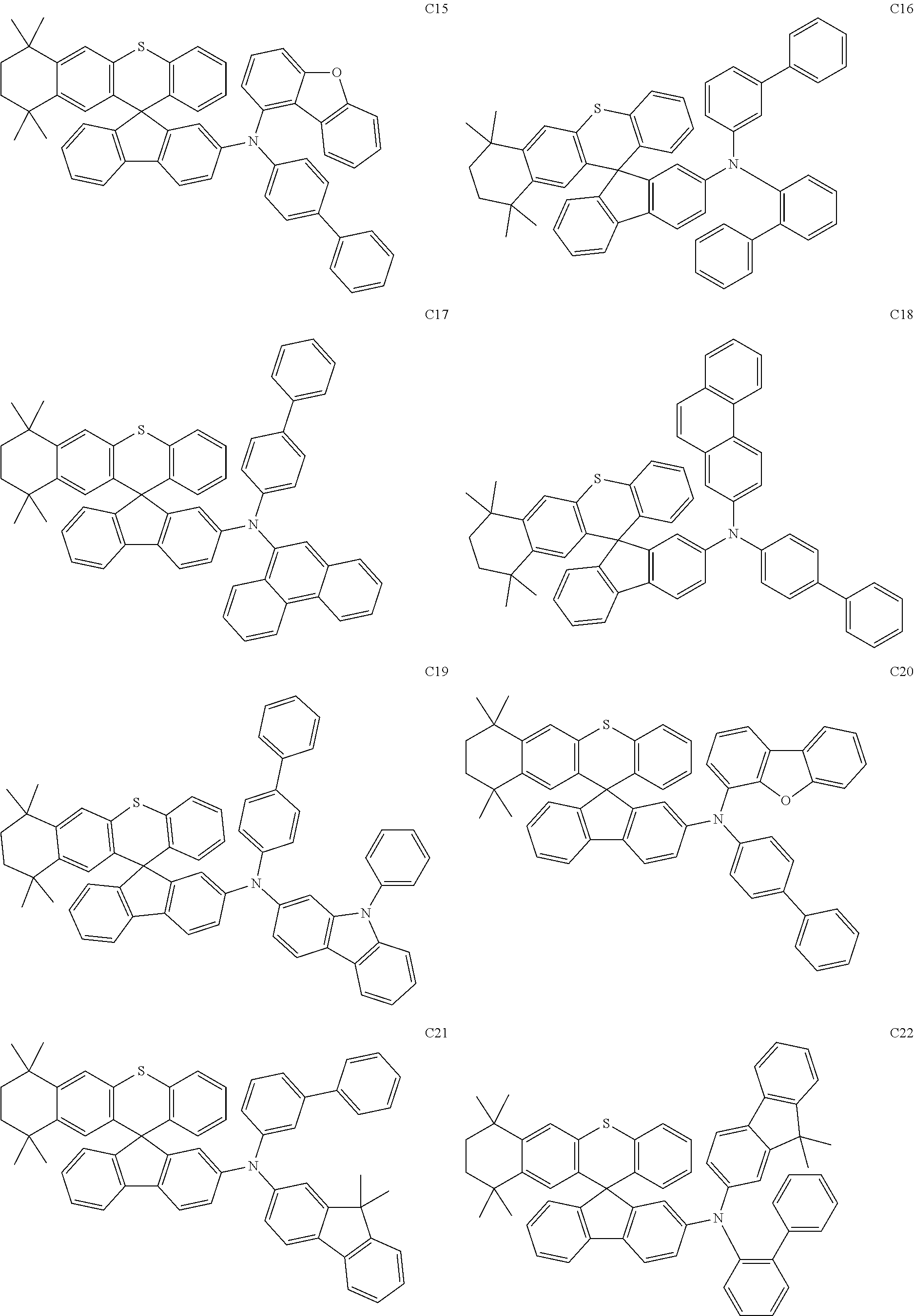

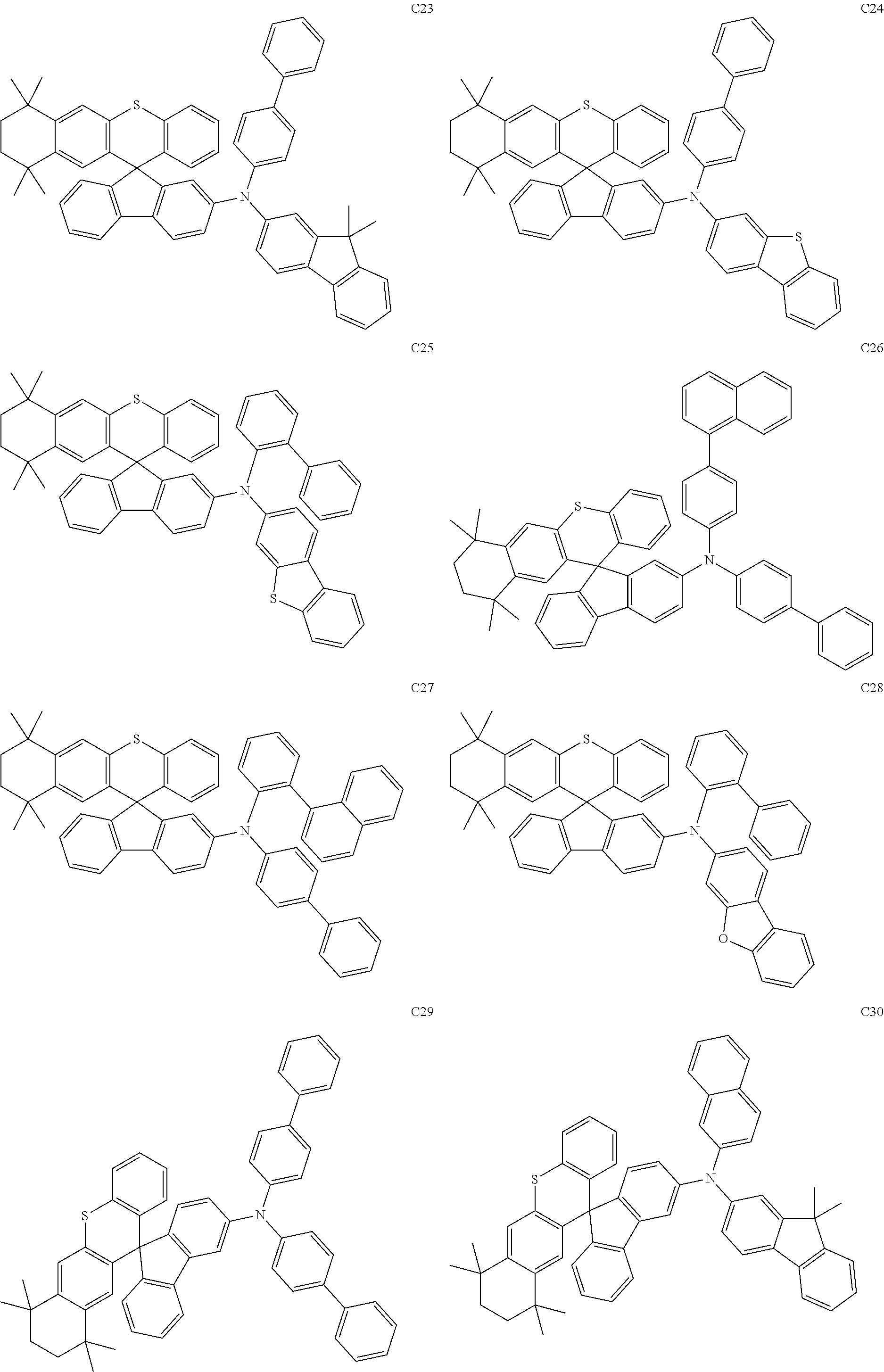

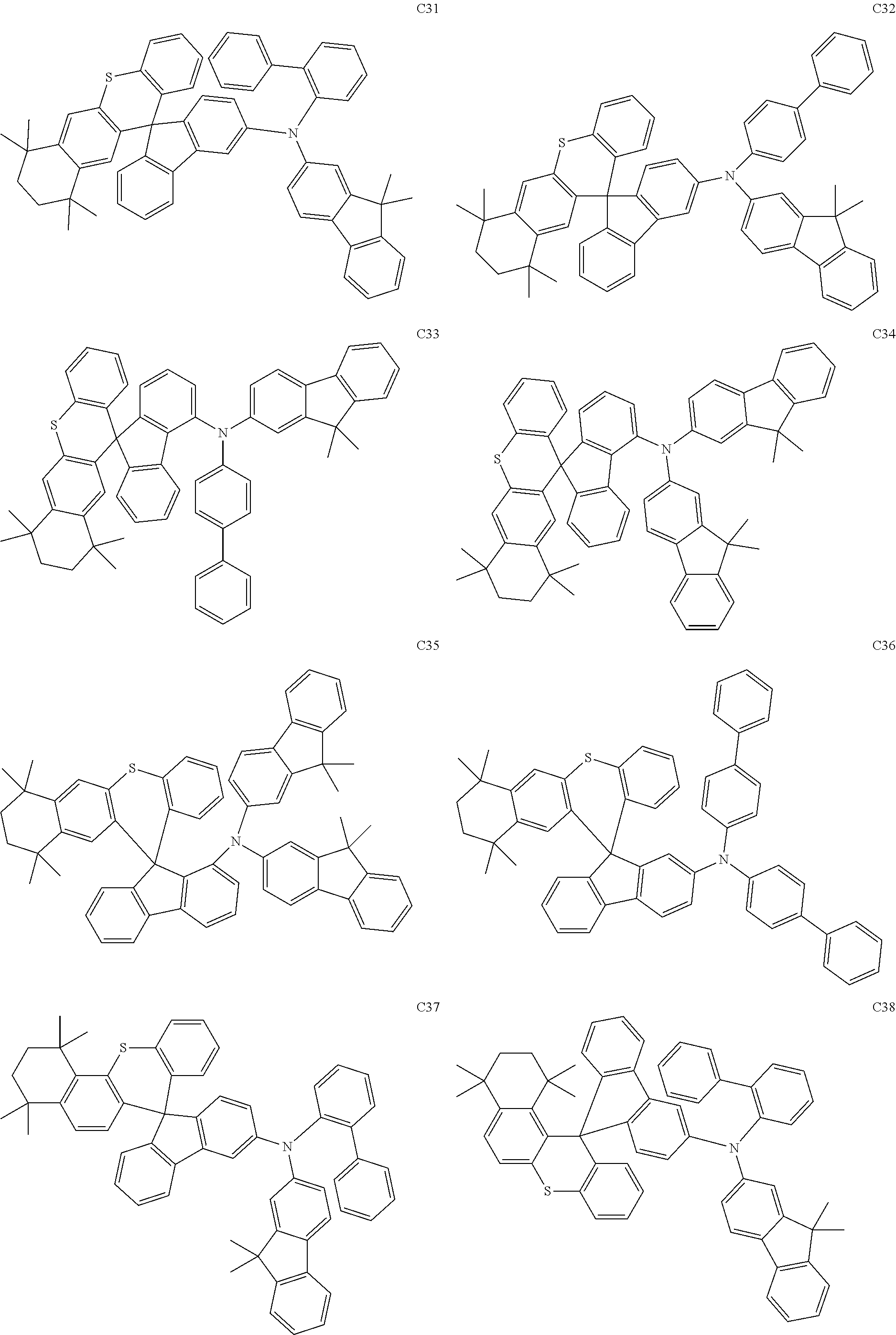

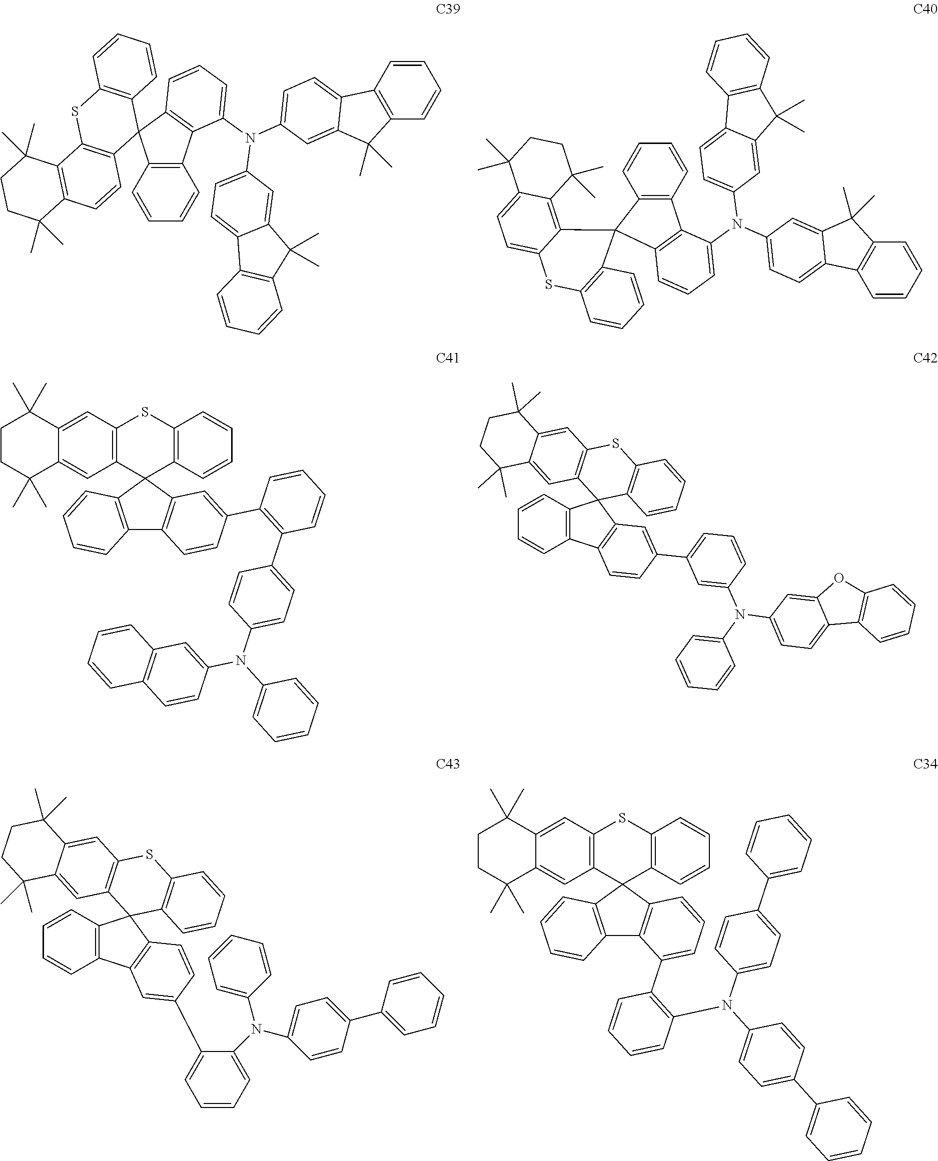

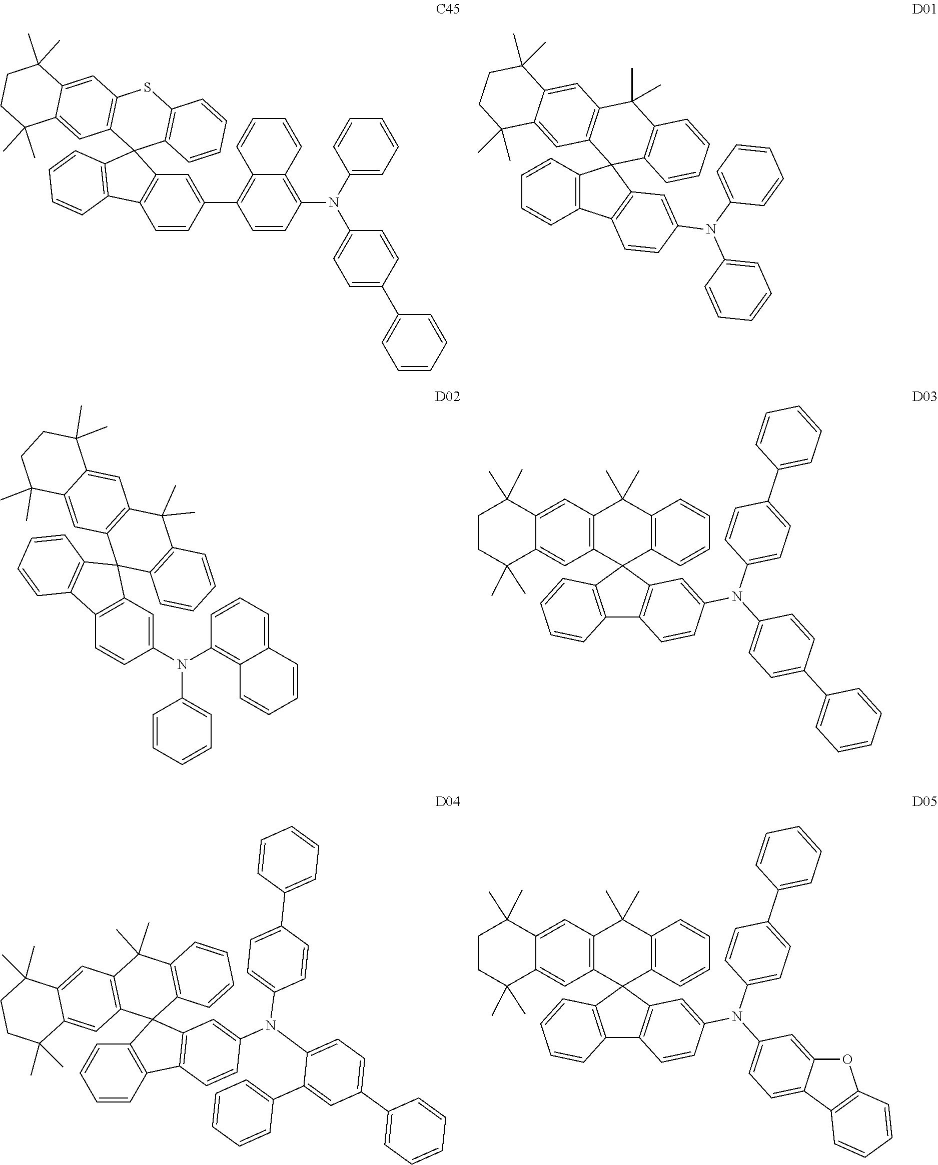

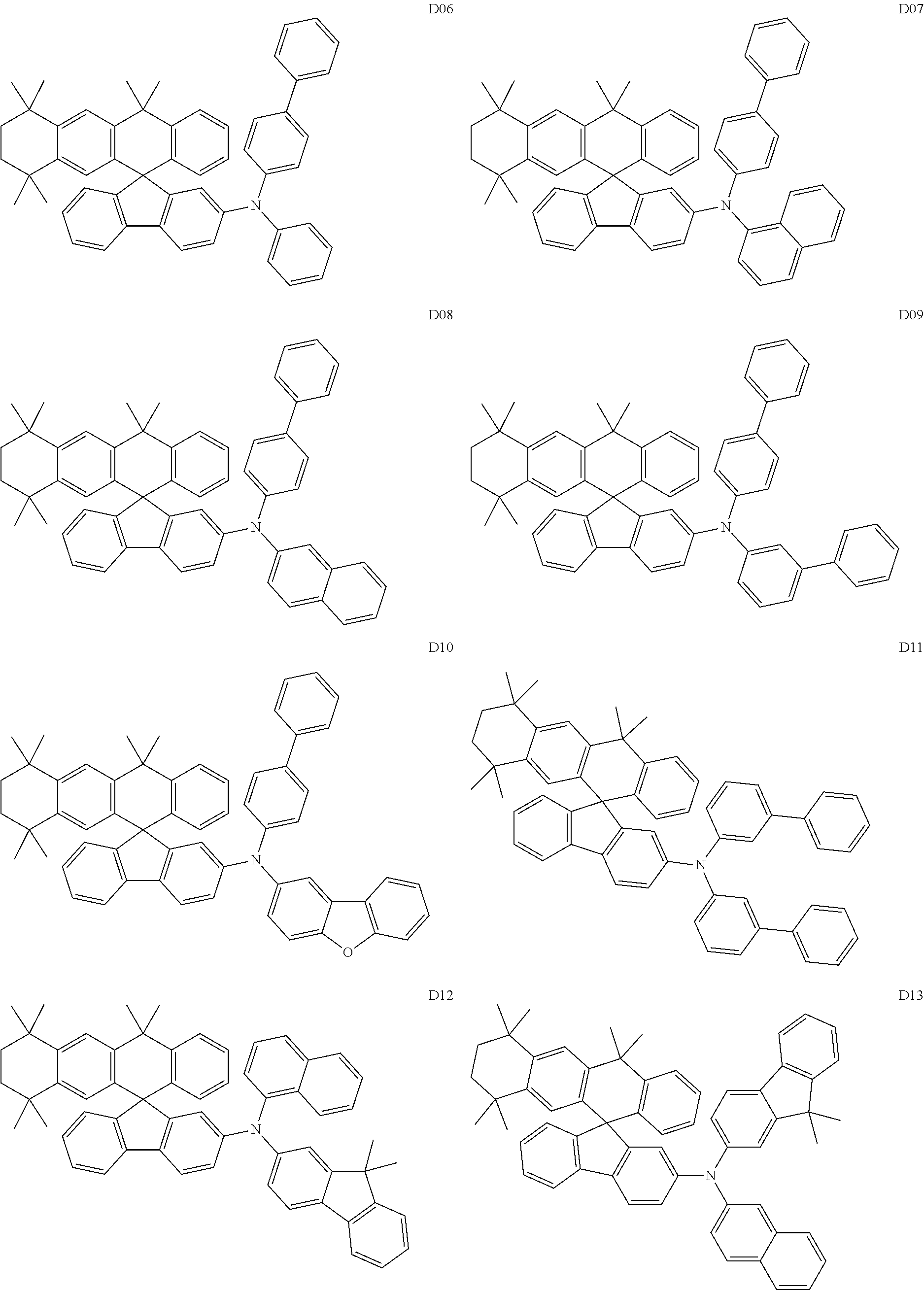

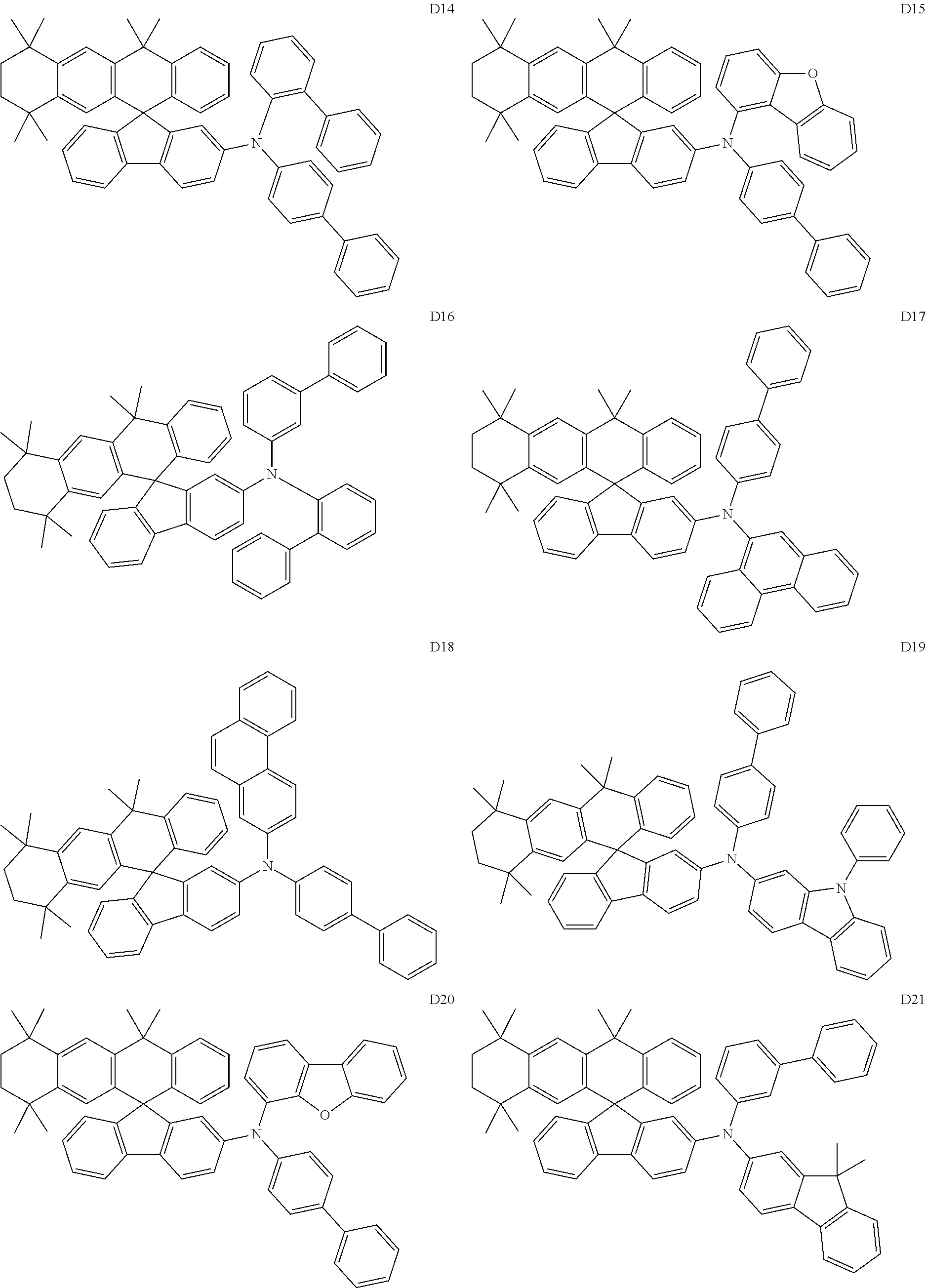

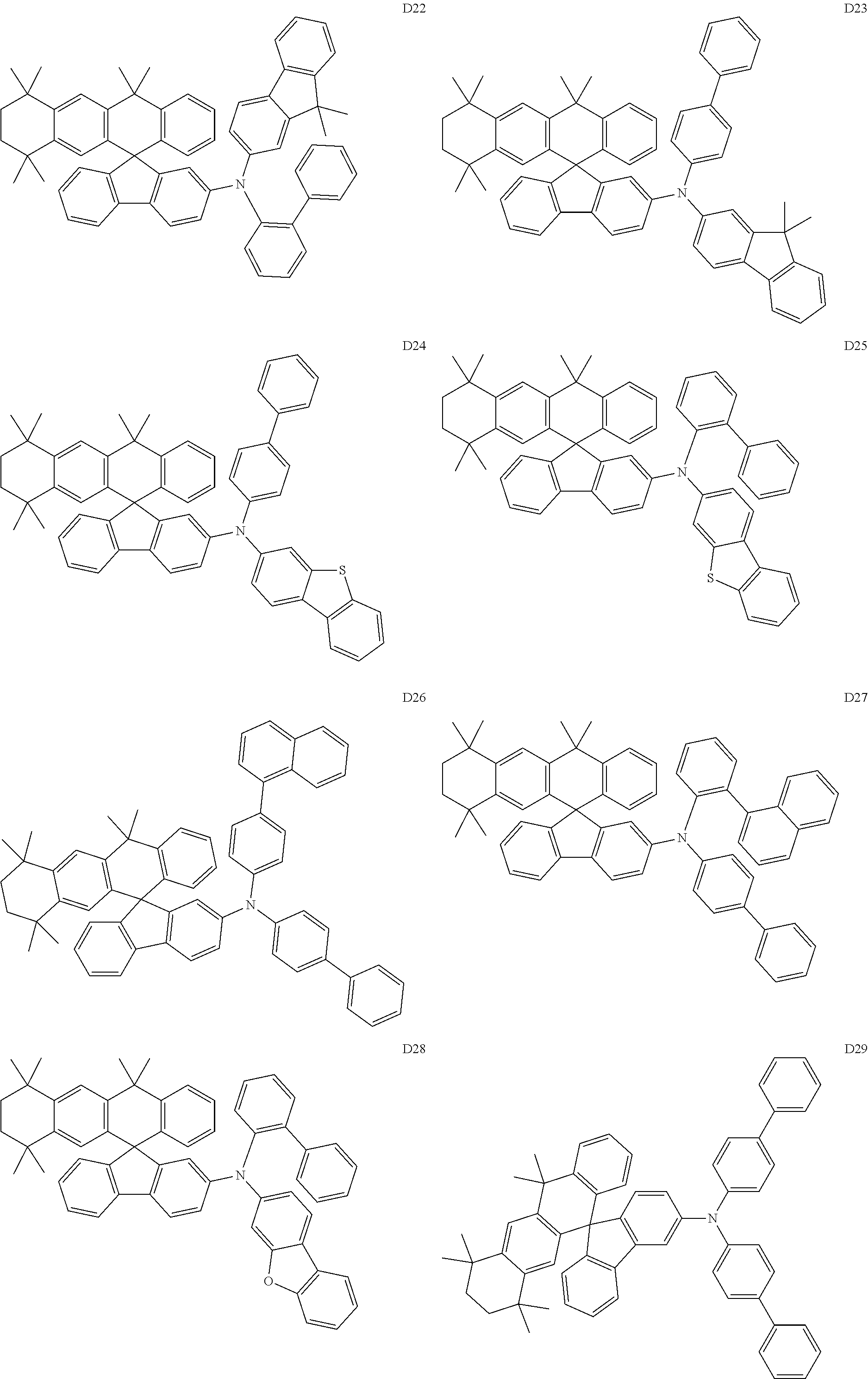

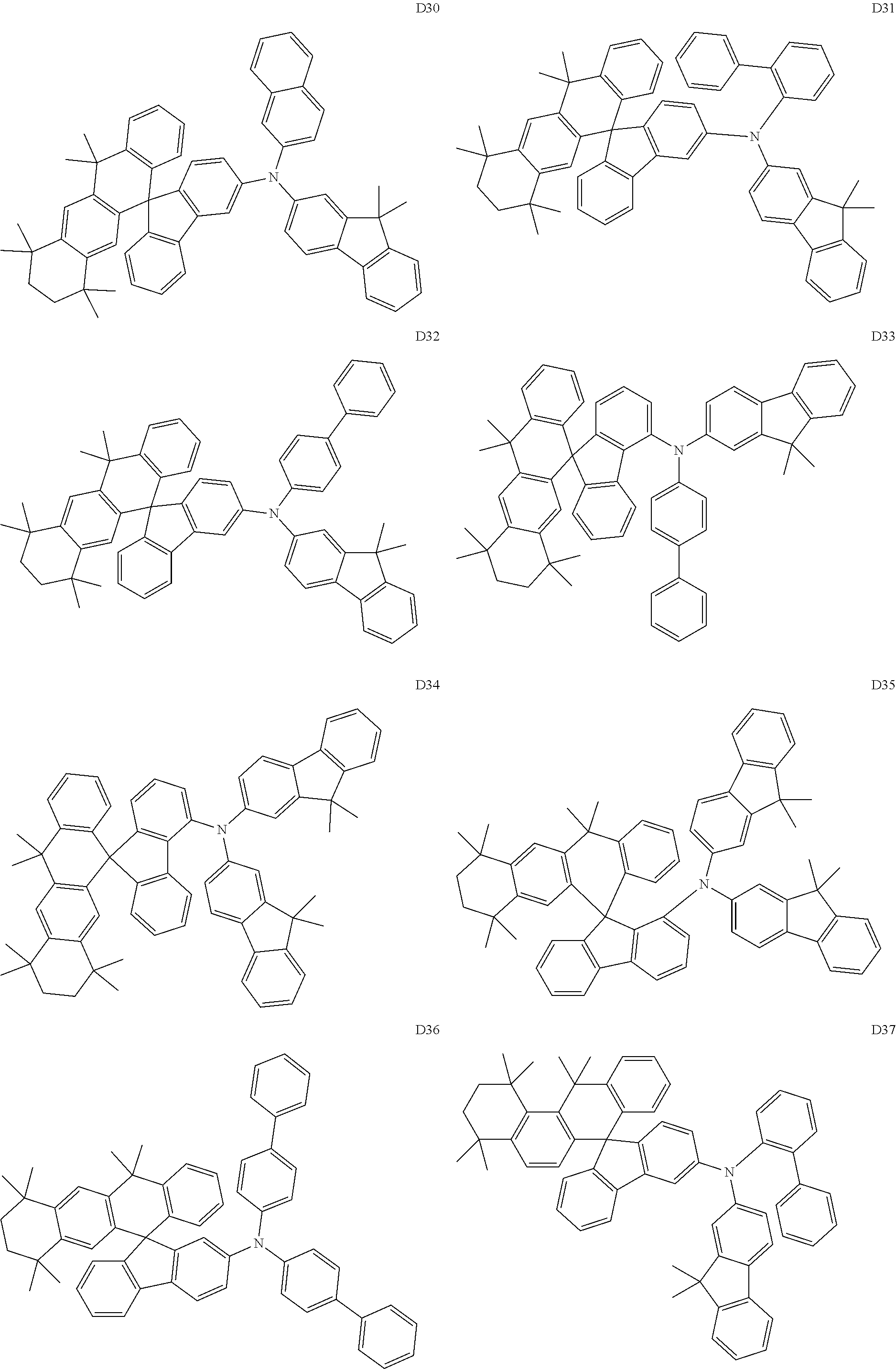

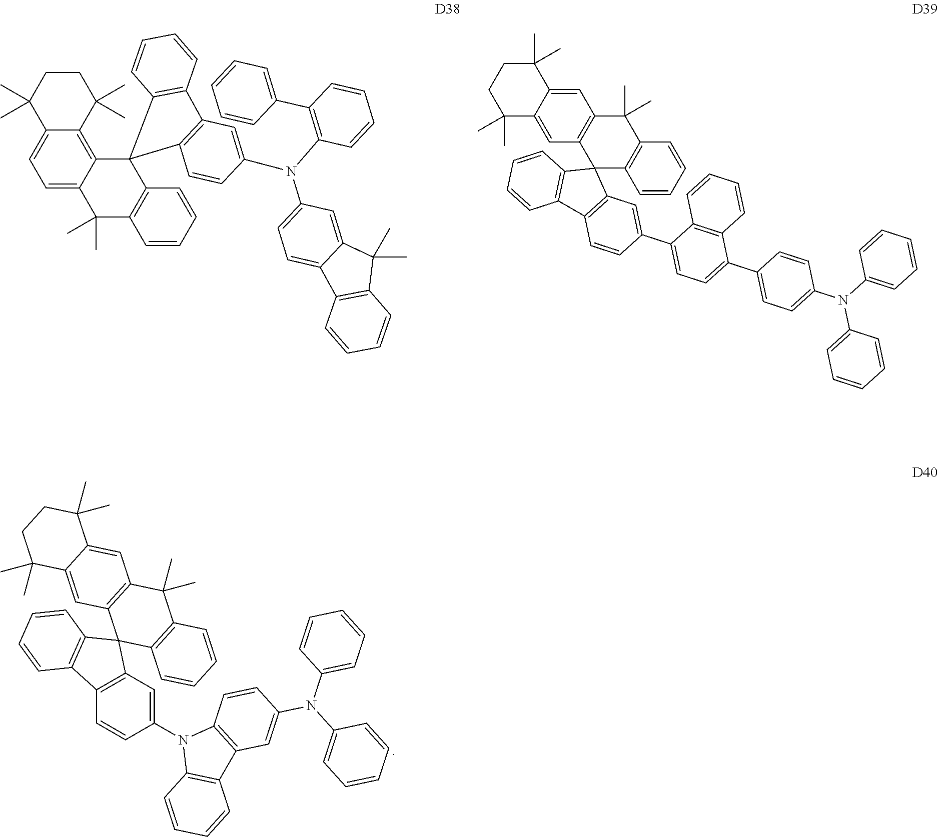

In a second aspect, the present disclosure provides an organic electroluminescent device comprising an anode, a cathode, and a functional layer disposed between the anode and the cathode; wherein the functional layer comprises the organic compound described in the first aspect of the present disclosure.

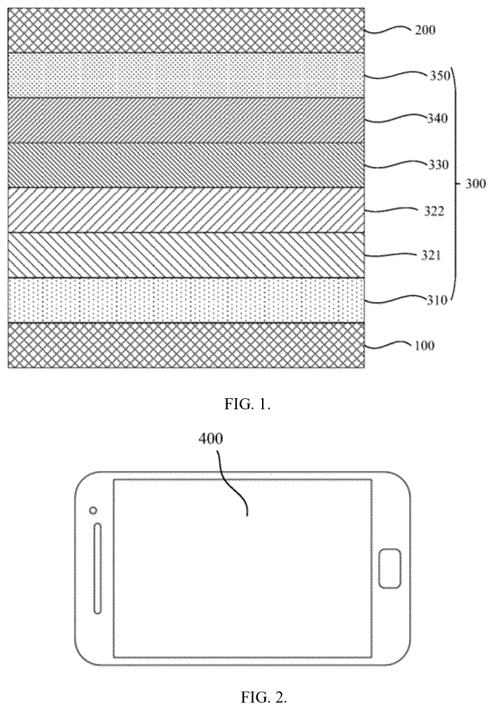

According to one embodiment, the structure of the organic electroluminescent device is shown in FIG. 1, which includes an anode 100 and a cathode 200 disposed opposite to each other, as well as a functional layer 300 disposed between the anode 100 and the cathode 200; the functional layer 300 contains the organic compound provided in the present disclosure.

Optionally, the functional layer 300 includes a hole transport layer 321 disposed between the anode and the organic light-emitting layer. Among them, the hole transport layer 321 contains the organic compound provided in the present disclosure. The hole transport layer 321 either may be composed of the organic compound provided in the present disclosure or may be collectively composed of the organic compound provided in the present disclosure and other materials.

In a specific embodiment of the present disclosure, the organic electroluminescent device may include an anode 100, a hole transport layer 321, an electron blocking layer 322 (also known as “a hole auxiliary layer”), an organic light-emitting layer 330 as an energy conversion layer, an electron transport layer 340, and a cathode 200 stacked in sequence. The organic compound provided in the present disclosure can be applied to the hole transport layer 321 of an organic electroluminescent device, effectively improving the luminous efficiency, lifetime, and thermal stability of the organic electroluminescent device, and reducing the driving voltage of the organic electroluminescent device.

In the present disclosure, the anode 100 comprises an anode material, which is alternatively a large work function material contributing to injection of holes into the functional layer. Specific examples of the anode material include: metals such as nickel, platinum, vanadium, chromium, copper, zinc and gold, or alloys thereof, metal oxides such as zinc oxide, indium oxide, indium tin oxide (ITO), and indium zinc oxide (IZO); combinations of metals and oxides, such as ZnO:Al or SnO₂:Sb; and conductive polymers such as poly(3-methylthiophene), poly[3,4-(ethylene-1,2-dioxy) thiophene](PEDT), polypyrrole, and polyaniline, but are not limited thereto. Optionally, a transparent electrode comprising indium tin oxide (ITO) as the anode is included.

Optionally, the hole injection layer 310 may be further provided between the anode 100 and the hole transport layer 321 to enhance the ability to inject holes into the hole transport layer 321. The hole injection layer 310 may choose to use a benzidine derivative, a starburst arylamine-based compound, a phthalocyanine derivative or other materials. It is not particularly limited in the present disclosure. The material of the hole injection layer 310 is selected from the following compounds or any combination thereof, for example;

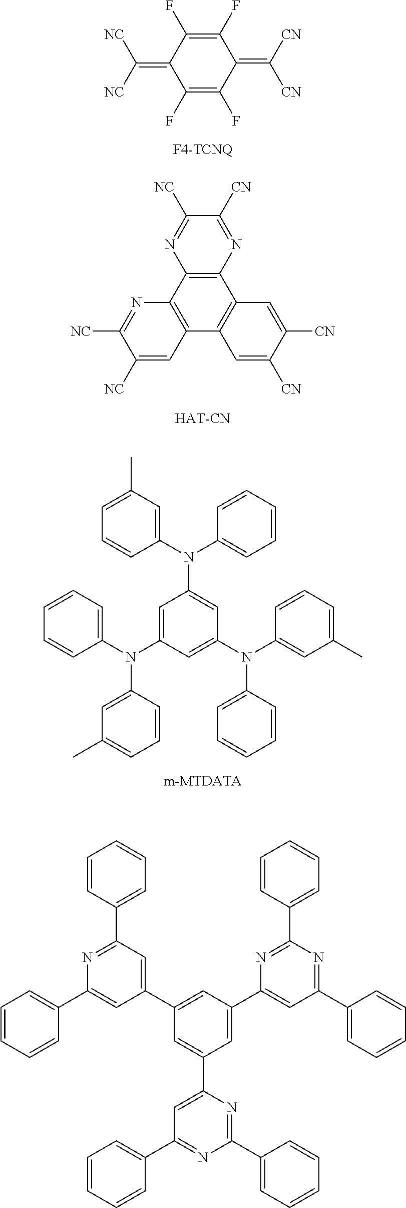 [SMILES: N#CC(C#N)=c1c(F)c(F)c(=C(C#N)C#N)c(F)c1F ; N#Cc1cc2c3cc(C#N)c(C#N)nc3c3nc(C#N)c(C#N)nc3c2cc1C#N ; Cc1cccc(N(c2ccccc2)c2cc(N(c3ccccc3)c3cccc(C)c3)cc(N(c3ccccc3)c3cccc(C)c3)c2)c1 ; c1ccc(-c2cc(-c3cc(-c4cc(-c5ccccc5)nc(-c5ccccc5)n4)cc(-c4cc(-c5ccccc5)nc(-c5ccccc5)n4)c3)cc(-c3ccccc3)n2)cc1]

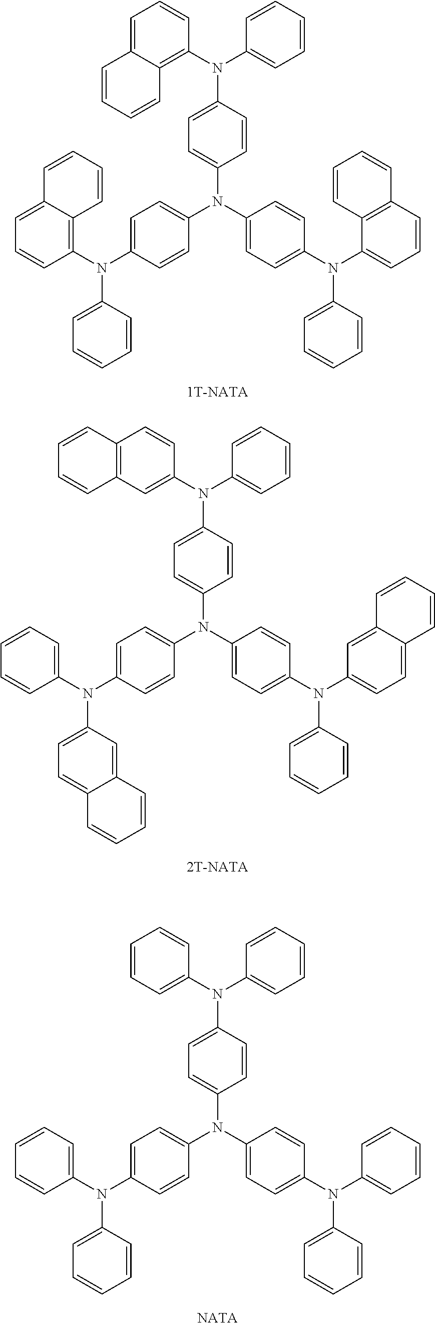

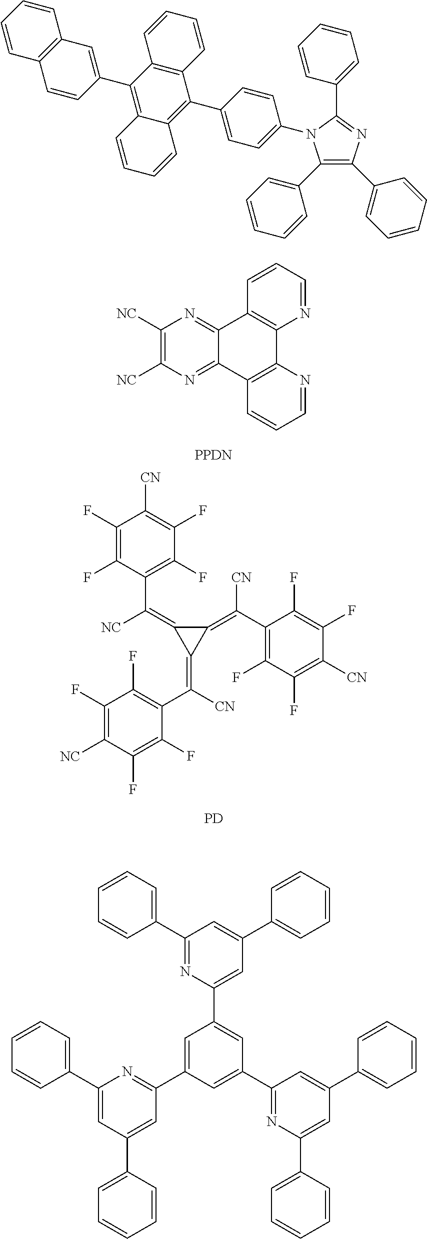

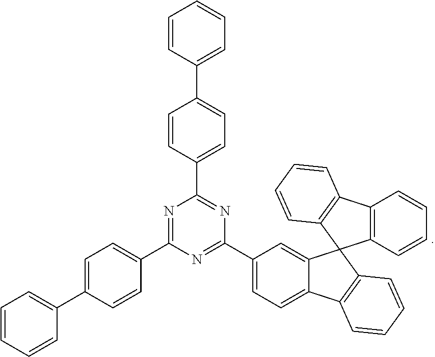 [SMILES: c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1]

In an embodiment of the present disclosure, the hole injection layer 310 is compose of HAT-CN.

Optionally, the electron blocking layer 322 includes one or more electron blocking materials, which may be selected from carbazole multimers or other types of compounds. It is not particularly limited in the present disclosure. For example, in an embodiment of the present disclosure, the electron blocking layer 322 is composed of Compound EB-01

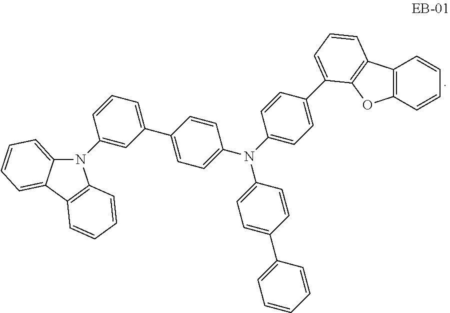 [SMILES: c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

Optionally, the organic light-emitting layer 330 may be composed of a single luminescent material or may comprise a host material and a guest material. Optionally, the organic light-emitting layer 330 is composed of a host material and a guest material. The holes injected into the organic light-emitting layer 330 and the electrons injected into the organic light-emitting layer 330 can recombine in the organic light-emitting layer 330 to form excitons. The excitons transmit energy to the host material, and the host material transmits the energy to the guest material, thereby enabling the guest material to emit light.

The host material of the organic light-emitting layer 330 may contain a metal chelating compound, a stilbene-based derivative, an aromatic amine derivative, a dibenzofuran derivative, or other types of materials. The host material of the organic light-emitting layer 330 may be one compound, or a combination of two or more compounds.

In an embodiment of the present disclosure, the host material of the organic light-emitting layer 330 is BH-01

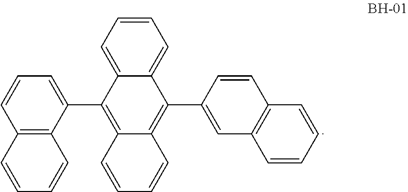 [SMILES: c1ccc2cc(-c3c4ccccc4c(-c4cccc5ccccc45)c4ccccc34)ccc2c1]

The guest material of the organic light-emitting layer 330 may be a compound having a condensed aryl ring or its derivative, a compound having a heteroaryl ring or its derivative, an aromatic amine derivative, or other materials. It is not particularly limited in the present disclosure. The guest material is also known as a doping material or a dopant. The specific examples of the dopant include but are not limited to,

In some specific embodiments of the present disclosure, the guest material of the organic light-emitting layer 330 is BD-01.

The electron transport layer 340 may be a single-layer structure or a multi-layer structure, which may comprise one or more electron transport material(s). The electron transport materials may be selected from, but not limited to, LiQ, benzimidazole derivatives, oxadiazole derivatives, quinoxaline derivatives, and other electron transport materials, and are not particularly limited in the present disclosure. The material of the electron transport layer 340 includes but is not limited to the following compounds:

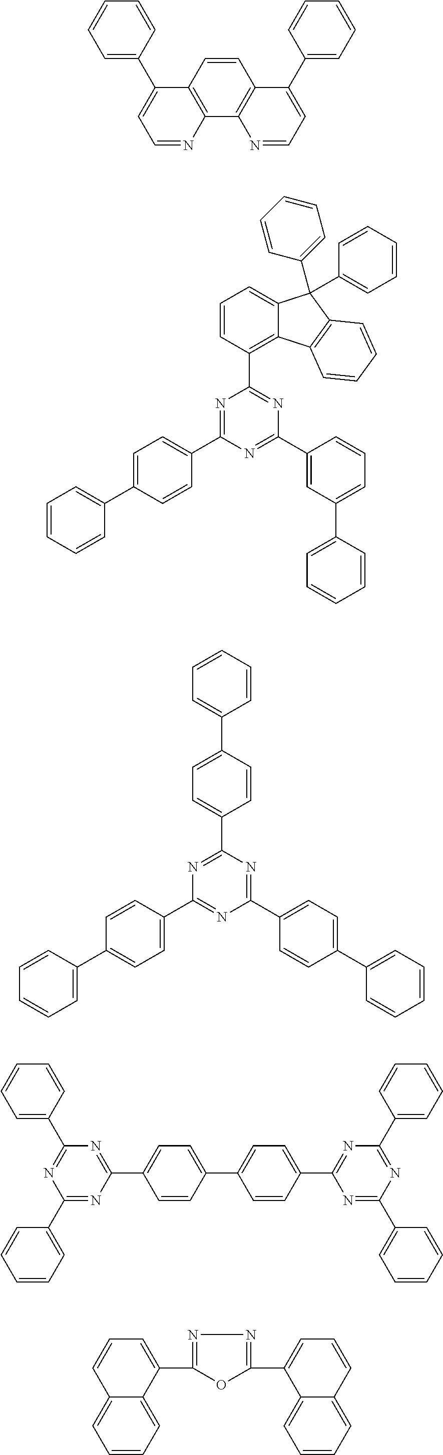 [SMILES: c1ccc(-c2ccnc3c2ccc2c(-c4ccccc4)ccnc23)cc1 ; c1ccc(-c2ccc(-c3nc(-c4cccc(-c5ccccc5)c4)nc(-c4cccc5c4-c4ccccc4C5(c4ccccc4)c4ccccc4)n3)cc2)cc1 ; c1ccc(-c2ccc(-c3nc(-c4ccc(-c5ccccc5)cc4)nc(-c4ccc(-c5ccccc5)cc4)n3)cc2)cc1 ; c1ccc(-c2nc(-c3ccccc3)nc(-c3ccc(-c4ccc(-c5nc(-c6ccccc6)nc(-c6ccccc6)n5)cc4)cc3)n2)cc1 ; c1ccc2c(-c3nnc(-c4cccc5ccccc45)o3)cccc2c1]

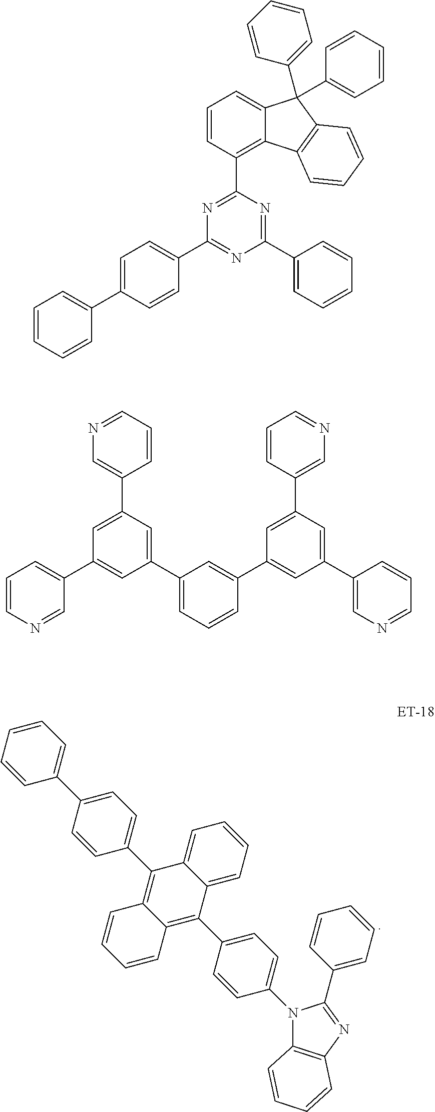 [SMILES: c1ccc(-c2ccc(-c3nc(-c4ccccc4)nc(-c4cccc5c4-c4ccccc4C5(c4ccccc4)c4ccccc4)n3)cc2)cc1 ; c1cncc(-c2cc(-c3cccnc3)cc(-c3cccc(-c4cc(-c5cccnc5)cc(-c5cccnc5)c4)c3)c2)c1 ; c1ccc(-c2ccc(-c3c4ccccc4c(-c4ccc(-n5c(-c6ccccc6)nc6ccccc65)cc4)c4ccccc34)cc2)cc1]

In an embodiment of the present disclosure, the electron transport layer 340 is composed of ETA 8 and LiQ.

In an embodiment, the cathode 200 comprises a cathode material, which is a low work function material contributing to injection of electrons into the functional layer. Specific examples of the cathode material include, but are not limited to, metals such as magnesium, calcium, sodium, potassium, titanium, indium, yttrium, lithium, gadolinium, aluminum, silver, tin, and lead, and alloys thereof; or multilayer materials such as LiF/Al, Liq/Al, LiO₂/Al, LiF/Ca, LiF/Al, and BaF₂/Ca. Optionally, a metal electrode comprising magnesium and silver as the cathode is included.

Optionally, an electron injection layer 350 may be further provided between the cathode 200 and the electron transport layer 340 to enhance the ability to inject electrons into the electron transport layer 340. The electron injection layer 350 may comprise an inorganic material such as an alkali metal sulfide and an alkali metal halide or may comprise a complex of an alkali metal and organics. In an embodiment of the present disclosure, the electron injection layer 350 comprises LiF.

In a third aspect, the present disclosure provides an electronic apparatus, comprising the organic electroluminescent device described in the second aspect of the present disclosure.

According to an embodiment, as shown in FIG. 2, the provided electronic apparatus is an electronic apparatus 400 comprising the above-described organic electroluminescent device. The electronic apparatus 400 may be a display device, a lighting device, an optical communication device, and other types of electronic devices, examples of which may include, but are not limited to, computer screens, mobile phone screens, televisions, electronic paper, emergency lamps, and optical modules, etc.

The synthesis method of the organic compound in the present disclosure will be demonstrated in detail with the following synthesis examples, but the present disclosure is not limited in any way by this.

### Synthesis Example

Professionals in their field should realize that the chemical reactions described in this present disclosure can be used to properly prepare many of the organic compounds in this present disclosure, and other methods used to prepare the compounds in this present disclosure are considered to be within the scope of this present disclosure. For example, according to the present disclosure, the synthesis of those non-exemplified compounds can be successfully completed by the technicians in the field through modification methods, such as appropriate protection of interfering groups, by using other known reagents in addition to the ones described in the present disclosure, or by making some conventional modifications to the reaction conditions. The compounds of which the synthetic methods not mentioned in this present disclosure are all raw material products commerically available.

The compounds of the present disclosure are prepared by reacting IM-I based and IM-II based intermediates (including some intermediates that can be directly purchased). Below are two types of synthesis methods for the intermediates:

### 1. Synthesis of IM-I-a1

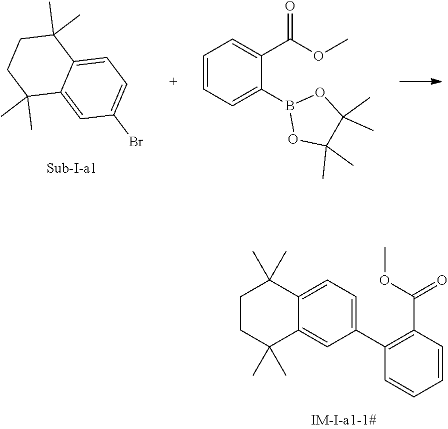 [SMILES: CC1(C)CCC(C)(C)c2cc(Br)ccc21 ; COC(=O)c1ccccc1B1OC(C)(C)C(C)(C)O1 ; COC(=O)c1ccccc1-c1ccc2c(c1)C(C)(C)CCC2(C)C]

(1) Under nitrogen protection, Sub-I-a1 (80.0 g, 299.39 mmol), 2-methoxycarbonylphenylboronic acid pinacol ester (78.5 g, 299.40 mmol), potassium carbonate (91.0 g, 658.65 mmol), toluene (700 mL), ethanol (350 mL), and water (150 mL) were added into a three-necked flask. The resulting mixture was stirred and heated to 50° C. to 60° C., followed by addition of tetrakis (triphenylphosphine) palladium (6.9 g, 5.98 mmol) and tetrabutylammonium bromide (TBAB, 19.3 g, 59.88 mmol), and after the addition, the resulting mixture was heated to 70° C. to 75° C. for 12 hours of reflux reaction. After the reaction was completed, the reaction solution was naturally cooled to the room temperature and was extracted with dichloromethane. After separation, the organic phase was washed with water until neutral, followed by drying, filtration, and then was concentrated under reduced pressure, to obtain a crude product. The crude product was recrystallizated with the mixture of ethyl acetate and n-heptane, and the resulting white solid is IM-I-a1-1 # (77.0 g, yield 79.8%).

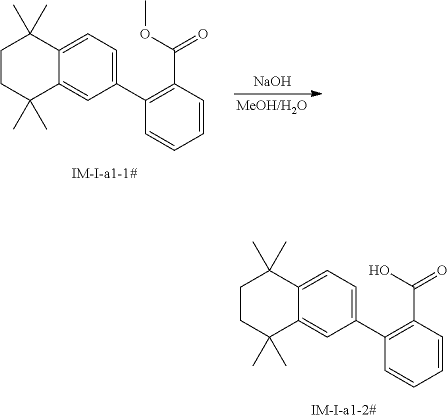

(2) At room temperature, NaOH (38.2 g, 955.19 mmol) was dissolved in 400 mL deionized water. After stirring thoroughly to completely dissolve, 400 mL methanol was added to the solution to obtain a mixed alkaline solution of NaOH/methanol/water. IM-I-a1-1 # (76.6 g, 237.56 mmol) and the above mixed alkaline solution were added into a three-necked flask. The resulting mixture was heated up to 50° C. with stirring for 24 hours of hydrolysis reaction. After the reaction was completed, methanol is removed by distillation under reduced pressure, and then 300 mL deionized water was added to dilute the reaction solution. 3M dilute hydrochloric acid was then added to adjust the pH to 2 to 3. Then, the reaction solution was extracted with dichloromethane and deionized water. After separation, the organic phase was dried with anhydrous magnesium sulfate and filtered, and then the solvent was removed by distillation under reduced pressure. The resulting white solid is IM-I-a1-2 # (60.5 g, yield 82.6%).

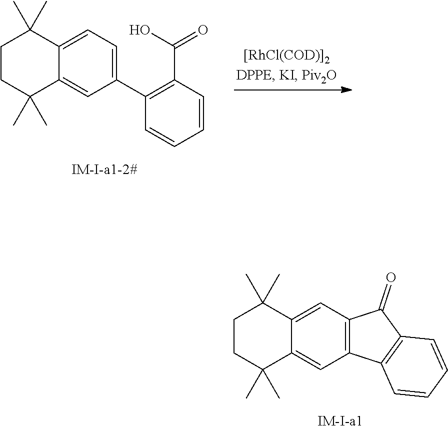

(3) Under nitrogen protection, IM-I-a1-2 # (60.0 g, 194.54 mmol), (1,5-cyclooctadiene) rhodium (I) chloride dimer (Cas: 12092-47-6, 2.4 g, 4.86 mmol), 1,2-diphenylphosphine ethane (DPPE, 3.9 g, 9.73 mmol), potassium iodide (16.1 g, 97.25 mmol), and trimethylacetic anhydride (Piv₂O, 108.7 g, 583.62 mmol) were sequentially added into a reaction flask. The reaction flask was placed in a microwave reactor and heated to 200° C. for 30 minutes of reaction. After the reaction is stopped, the reaction solution was cooled to room temperature, and was extracted with dichloromethane/deionized water. After separation, the organic phase was washed with water until neutral, and then followed by separation of aqueous and organic phases, drying, suction filtering, and concentration to obtain a yellow solid as crude product. The crude product was purified by silica gel column chromatography with dichloromethane/n-hexane (volume ratio of 1:5) as the mobile phase, followed by the removal of the solvent via distillation under reduced pressure to obtain IM-I-a1 as a white solid (53.2 g, yield 94.2%).

### 2. Synthesis of IM-I-a2

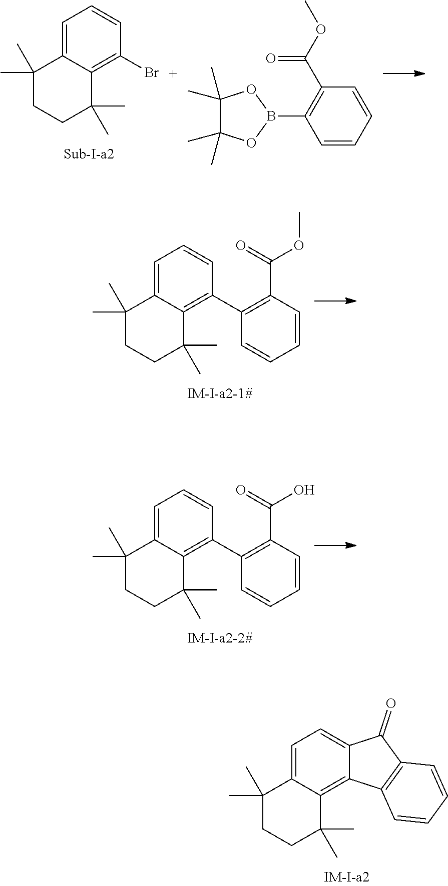

IM-I-a2 was synthesized using the same method as IM-I-a1, except that Sub-I-a2 (30.0 g, 112.27 mmol) was used instead of Sub-I-a1 in step (1), and all other raw materials remained unchanged, to obtain IM-I-a2-1 # (26.2 g, yield 72.4%); IM-I-a2-1 # (26.0 g, 80.63 mmol) was used instead of IM-I-a1-1 # in step (2) to obtain IM-I-a2-2 # (19.8 g, yield 79.5%); IM-I-a2-2 # (19.5 g, 63.23 mmol) was used instead of IM-I-a1-2 # in step (3) to obtain IM-I-a2 (16.7 g, yield 91.1%).

### 3. Synthesis of IM-I-b1

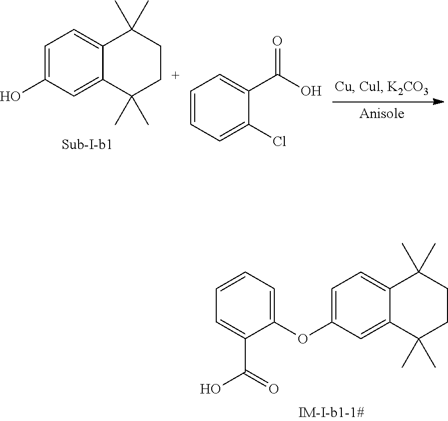

(1) Under nitrogen protection, Sub-I-b1 (40.0 g, 195.79 mmol), o-chlorobenzoic acid (32.2 g, 205.59 mmol), potassium carbonate (40.6 g, 293.68 mmol), copper powder (0.25 g, 3.92 mmol), cuprous iodide (0.75 g, 3.92 mmol), and anisole (350 mL) were sequentially added into a three-necked flask. The resulting mixture was stirred and refluxed for 5 hours of reaction. After the reaction was completed, the reaction solution was naturally cooled to room temperature and extracted with dichloromethane/deionized water. After separation, the organic phase was washed with water until neutral, and then was dried, filtered, and concentrated under reduced pressure. The resulting crude product as a light-yellow solid was purified by recrystallization using a mixture of ethyl acetate and n-hexane, and the resulting white solid was IM-I-b1-1 # (43.7 g, yield 68.8%).

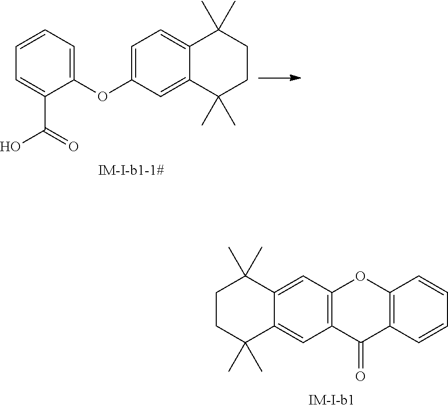 [SMILES: CC1(C)CCC(C)(C)c2cc(Oc3ccccc3C(=O)O)ccc21 ; CC1(C)CCC(C)(C)c2cc3c(=O)c4ccccc4oc3cc21]

(2) Under nitrogen protection, the intermediate IM-I-b1-1 # (43.2 g, 133.16 mmol) and 200 mL concentrated sulfuric acid (mass fraction ≥98%) were added into an enamel reactor with PTFE stirring rod and stirred at room temperature for 1 hour. After the reaction was completed, the reaction solution was slowly poured into ice water, followed by filtration to obtain a white solid, and the white solid was washed with deionized water until neutral, and after drying in an oven, and the resulting white solid is IM-I-b1 (26.8 g, yield 65.8%).

IM-I-xm in Table 1 were synthesized using the same method as IM-I-b1, except that the Raw material 1 in Table 1 were used to replace Sub-I-b1, and the Raw material 2 in Table 1 were used to replace o-chlorobenzoic acid, respectively. All other conditions remained unchanged.

TABLE 1

| Raw material 1 | Raw material 2 | IM-I-xm-1# | IM-I-xm | Yield/% |
|---|---|---|---|---|
| 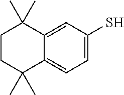 | 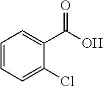 | 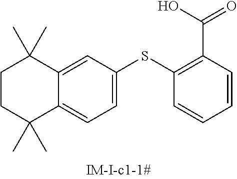 [SMILES: CC1(C)CCC(C)(C)c2cc(Sc3ccccc3C(=O)O)ccc21] | 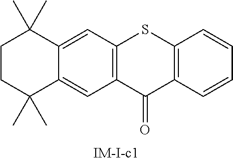 [SMILES: CC1(C)CCC(C)(C)c2cc3c(=O)c4ccccc4sc3cc21] | 57.0 |
| 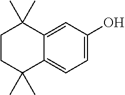 | 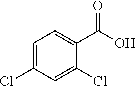 [SMILES: O=C(O)c1ccc(Cl)cc1Cl] | 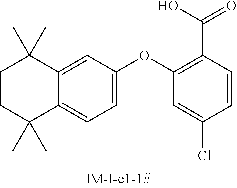 [SMILES: CC1(C)CCC(C)(C)c2cc(Oc3cc(Cl)ccc3C(=O)O)ccc21] | 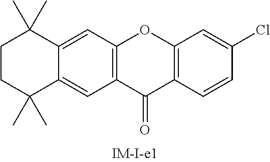 [SMILES: CC1(C)CCC(C)(C)c2cc3c(=O)c4ccc(Cl)cc4oc3cc21] | 62.2 |
| 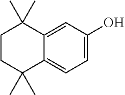 | 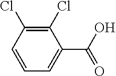 [SMILES: O=C(O)c1cccc(Cl)c1Cl] | 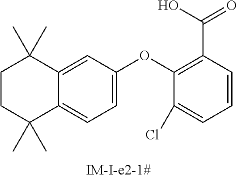 [SMILES: CC1(C)CCC(C)(C)c2cc(Oc3c(Cl)cccc3C(=O)O)ccc21] | 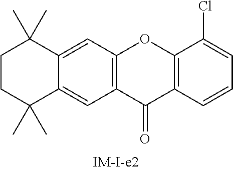 [SMILES: CC1(C)CCC(C)(C)c2cc3c(=O)c4cccc(Cl)c4oc3cc21] | 56.8 |
| 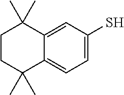 | 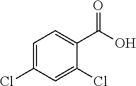 [SMILES: O=C(O)c1ccc(Cl)cc1Cl] | 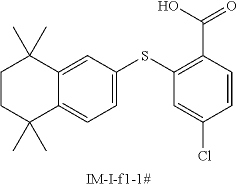 [SMILES: CC1(C)CCC(C)(C)c2cc(Sc3cc(Cl)ccc3C(=O)O)ccc21] | 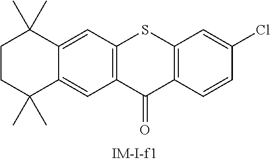 [SMILES: CC1(C)CCC(C)(C)c2cc3c(=O)c4ccc(Cl)cc4sc3cc21] | 54.2 |
| 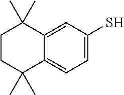 | 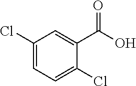 [SMILES: O=C(O)c1cc(Cl)ccc1Cl] | 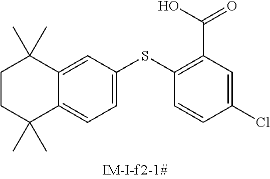 [SMILES: CC1(C)CCC(C)(C)c2cc(Sc3ccc(Cl)cc3C(=O)O)ccc21] | 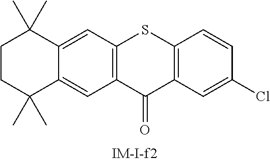 [SMILES: CC1(C)CCC(C)(C)c2cc3c(=O)c4cc(Cl)ccc4sc3cc21] | 51.4 |

### 4. Synthesis of IM-I-d1

Referring to the synthesis method of published patent CN109867652, the specific synthesis process is as follows:

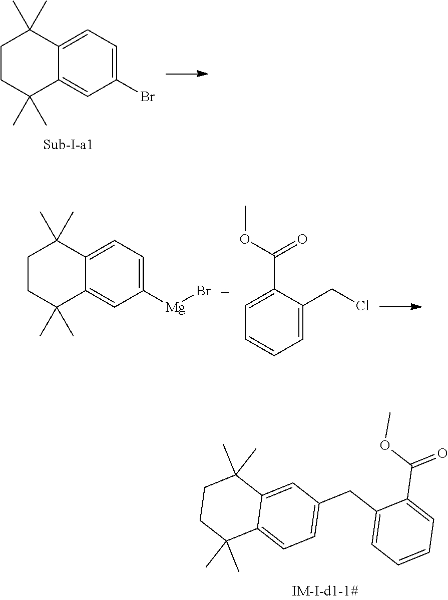

(1) Under nitrogen protection, the raw material Sub-I-a1 (50.0 g, 187.12 mmol) and 400 mL of dry THE were added into a 2 L three-necked flask. The mixture of the raw material was stirred at 0° C. for 15 minutes to completely dissolved. Then, magnesium chips (4.9 g, 204.22 mmol) were added and refluxed for 3 hours. After the reaction was completed, the newly prepared Grignard reagent as above mentioned was slowly dropped into 250 mL solution of methyl 2-chloromethylbenzoicate (34.5 g, 187.12 mmol) in tetrahydrofuran at 0° C. The resulting mixture was stirred for 30 minutes, then was naturally heated up to room temperature and stirred for 24 hours. After the reaction was completed, water was slowly dripped to the reaction bottle for quenching, and then the reaction solution was extracted with ethyl acetate and distilled water, followed by drying with anhydrous magnesium sulfate, and filtration. Purified by recrystallization using a mixture of ethyl acetate and n-hexane, the compound IM-I-d1-1 # (30.5 g, yield 72.0%) was obtained.

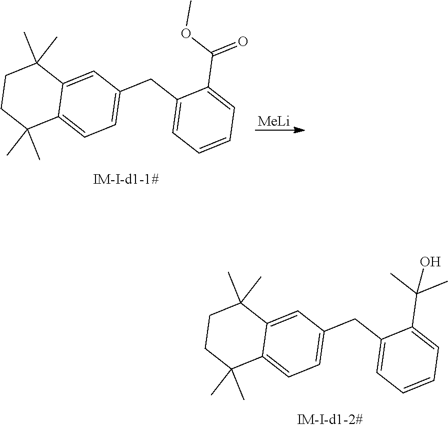 [SMILES: "MeLi" ; COC(=O)c1ccccc1Cc1ccc2c(c1)C(C)(C)CCC2(C)C ; CC(C)(O)c1ccccc1Cc1ccc2c(c1)C(C)(C)CCC2(C)C]

(2) The compound IM-I-d1-1 # (30.0 g, 132.46 mmol) and 450 mL ether were added into a 2 L reaction flask, and stirred and cooled to −78° C. Under nitrogen protection, 166 mL of 1.6M methyl lithium in ether (265.60 mmol) was slowly added dropwise to the reaction flask. After dropwise addition and 1 hour of reaction with the temperature being kept, the resulting mixture was naturally warmed to room temperature and stirred for 24 hours. After the completion of the reaction, methanol was slowly dripped to the reaction system, and the reaction solution was extracted with ethyl acetate, and then dried with anhydrous magnesium sulfate, followed by filtration. The filtrate was concentrated under reduced pressure to obtain a crude product. The crude product was purified by recrystallization using a mixture of dichloromethane and n-hexane, and the resulting white solid was IM-I-d1-2 # (34.1 g, yield 76.5%).

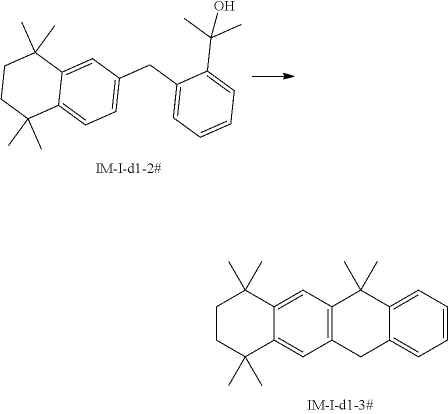

(3) IM-I-d1-2 # (32.3 g, 95.98 mmol) and 300 mL dichloromethane were added into a three-necked flask, with stirring to completely dissolve, then 6 mL concentrated sulfuric acid (mass fraction ≥98%) was added under nitrogen protection and stirred at room temperature for 2 hours. After the completion of reaction, the reactants were slowly added into ice water. After the reaction system was cooled to room temperature, a 5% NaOH aqueous solution was slowly added to adjust the pH to 7. The reaction solution was extracted with dichloromethane and distilled water and dried with anhydrous magnesium sulfate, followed by filtration. Then the filtrate was concentrated under reduced pressure to obtain a crude product. The crude product was purified by recrystallization using a mixture of dichloromethane and n-hexane, and the resulting white solid was IM-I-d1-3 #(26.3 g, yield 86.0%).

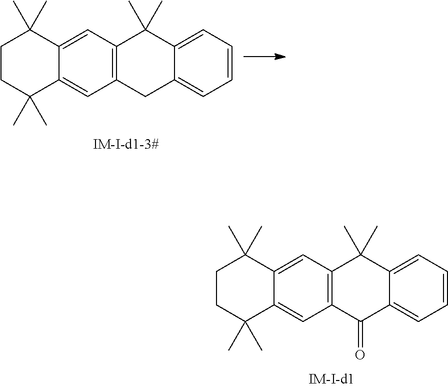

(4) IM-I-d1-3 # (26.0 g, 81.63 mmol) and 150 mL glacial acetic acid were added into a 2 L reaction flask, and stirred and heated to 55° C. to 58° C. Under nitrogen protection, 80 mL glacial acetic acid solution in which chromium trioxide (9.8 g, 97.96 mmol) was dissolved was added into the reaction flask, and the resulting mixture was stirred at a temperature of 55° C. to 58° C. for 4 hours. After the reaction was completed, the solvent was removed by distillation under reduced pressure, and 150 mL deionized water was added to the residue and stirred for 10 minutes to disperse the solid. After filtration, the filter cake was washed with deionized water to obtain a crude product as a light-yellow solid. The resulting light-yellow crude solid was recrystallized using a mixture of ethanol and water in a volume ratio of 3:1. The resulting white solid was IM-I-d1 (18.8 g, yield 69.3%).

IM-I-g1 and IM-I-g2 in Table 2 were synthesized using the same method as IM-I-d1, except that the Raw material 3 in Table 2 was used to replace methyl 2-chloromethylbenzoicate in step (1), and other conditions remained unchanged. The structures of the intermediates and products, as well as the yield (final step yield) obtained in each step, are listed in Table 2.

TABLE 2

| | 1# | 2# | 3# | | |
|---|---|---|---|---|---|
| Raw material 3 | Intermediate | Intermediate | Intermediate | Product | Yield/% |
| 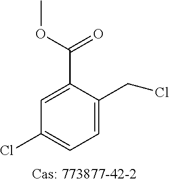 | 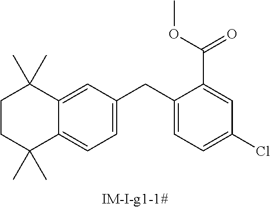 [SMILES: COC(=O)c1cc(Cl)ccc1Cc1ccc2c(c1)C(C)(C)CCC2(C)C] | 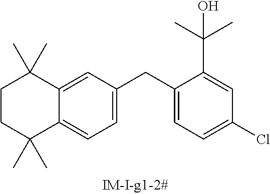 [SMILES: CC(C)(O)c1cc(Cl)ccc1Cc1ccc2c(c1)C(C)(C)CCC2(C)C] | 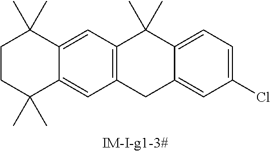 [SMILES: CC1(C)CCC(C)(C)c2cc3c(cc21)Cc1cc(Cl)ccc1C3(C)C] | 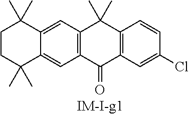 [SMILES: CC1(C)CCC(C)(C)c2cc3c(cc21)C(=O)c1cc(Cl)ccc1C3(C)C] | 65.4 |
| 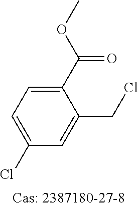 | 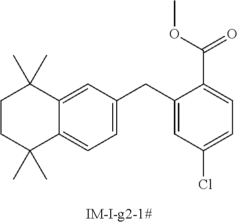 [SMILES: COC(=O)c1ccc(Cl)cc1Cc1ccc2c(c1)C(C)(C)CCC2(C)C] | 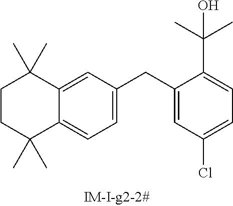 [SMILES: CC(C)(O)c1ccc(Cl)cc1Cc1ccc2c(c1)C(C)(C)CCC2(C)C] | 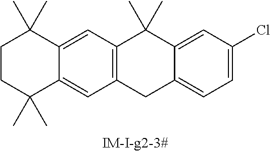 [SMILES: CC1(C)CCC(C)(C)c2cc3c(cc21)Cc1ccc(Cl)cc1C3(C)C] | 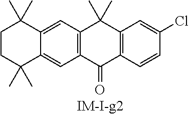 [SMILES: CC1(C)CCC(C)(C)c2cc3c(cc21)C(=O)c1ccc(Cl)cc1C3(C)C] | 64.2 |

### 5. Synthesis of IM-I-A1

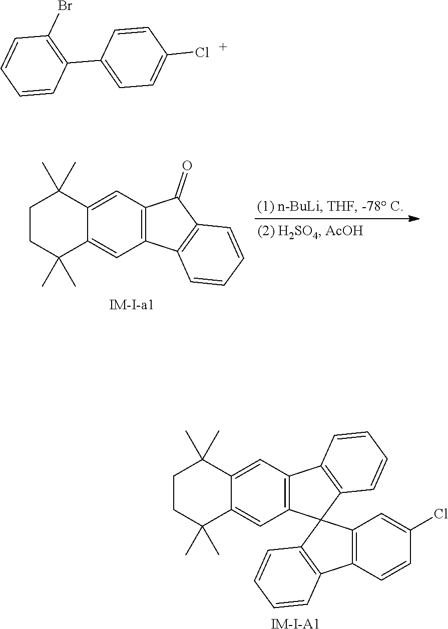

2-bromo-4′-chloro-1,1′-biphenyl (22.0 g, 82.23 mmol) and anhydrous tetrahydrofuran (200 mL) were added into a three-necked flask. The resulting mixture was stirred and cooled to −78° C. under nitrogen protection. Then, a 2M n-butyllithium in n-hexane (45 mL, 90.00 mmol) was slowly added dropwise. After the dropwise addition was completed, the resulting mixture was stirred for 2 hours with the temperature being kept, and then was added with 200 mL anhydrous tetrahydrofuran solution containing IM-I-a1 (23.9 g, 82.23 mmol), after that was stirred for 30 minutes with the temperature being kept. The resulting mixture was continuously stirred for 2 hours after the temperature was naturally warmed to room temperature. 1M hydrochloric acid solution (100 mL, 100 mmol) was slowly dripped to the reaction flask for thorough hydrolysis. The reaction solution was extracted with ethyl acetate, and the organic phase was dried with magnesium sulfate, followed by distillation under reduced pressure. The resulting crude product as a light-yellow solid was added to 200 mL glacial acetic acid and 4 mL concentrated sulfuric acid, heated and refluxed with stirring for 2 hours. The reaction solution was cooled to room temperature and then slowly poured into 400 mL deionized water to precipitate a white solid, followed by filtration, washing with deionized water until neutral. The resulting solid was purified by recrystallization using ethyl acetate to obtain IM-I-A1 (20.5 g, yield 44.48%).

IM-I-Xn in Table 3 were synthesized using the same method as IM-I-A1, except that the Raw material 4 were used to replace 2-bromo-4′-chloro-1,1′-biphenyl, and IM-I-xm in Table 3 were used to replace IM-I-a1, respectively. All other conditions remained unchanged, and the structures and yields of the obtained products are shown in Table 3.

TABLE 3

| Raw material 4 | IM-I-xm | IM-I-Xn | Yield/% |
|---|---|---|---|
| 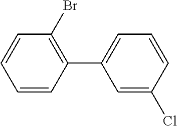 [SMILES: Clc1cccc(-c2ccccc2Br)c1] | 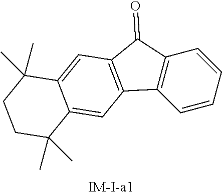 [SMILES: CC1(C)CCC(C)(C)c2cc3c(cc21)C(=O)c1ccccc1-3] | 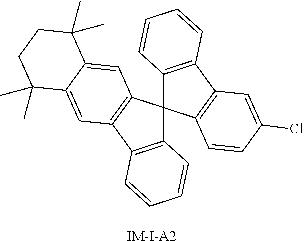 | 48.2 |
| 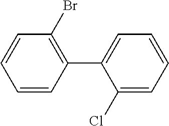 [SMILES: Clc1ccccc1-c1ccccc1Br] | 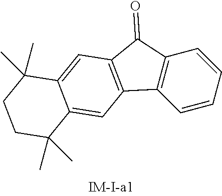 [SMILES: CC1(C)CCC(C)(C)c2cc3c(cc21)C(=O)c1ccccc1-3] | 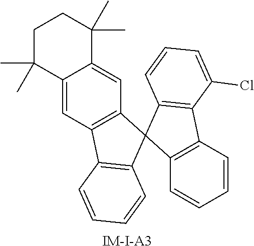 | 52.6 |
| 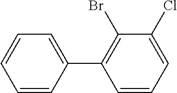 [SMILES: Clc1cccc(-c2ccccc2)c1Br] | 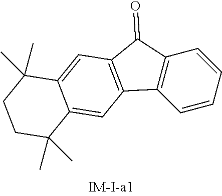 [SMILES: CC1(C)CCC(C)(C)c2cc3c(cc21)C(=O)c1ccccc1-3] | 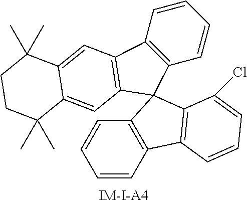 | 39.3 |
| 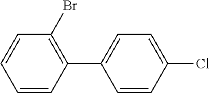 [SMILES: Clc1ccc(-c2ccccc2Br)cc1] | 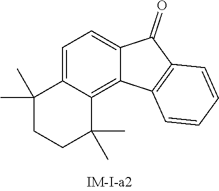 [SMILES: CC1(C)CCC(C)(C)c2c1ccc1c2-c2ccccc2C1=O] | 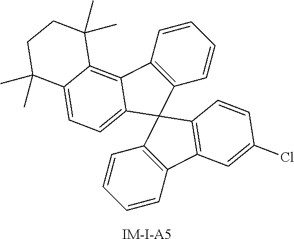 | 50.3 |
| 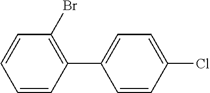 [SMILES: Clc1ccc(-c2ccccc2Br)cc1] | 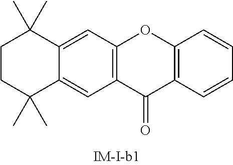 [SMILES: CC1(C)CCC(C)(C)c2cc3c(=O)c4ccccc4oc3cc21] | 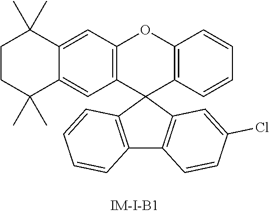 [SMILES: CC1(C)CCC(C)(C)c2cc3c(cc21)Oc1ccccc1C31c2ccccc2-c2ccc(Cl)cc21] | 48.5 |
| 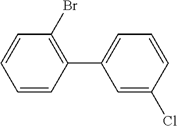 [SMILES: Clc1cccc(-c2ccccc2Br)c1] | 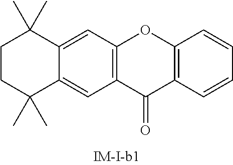 [SMILES: CC1(C)CCC(C)(C)c2cc3c(=O)c4ccccc4oc3cc21] | 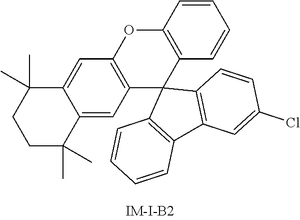 [SMILES: CC1(C)CCC(C)(C)c2cc3c(cc21)Oc1ccccc1C31c2ccccc2-c2cc(Cl)ccc21] | 45.5 |
| 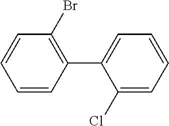 [SMILES: Clc1ccccc1-c1ccccc1Br] | 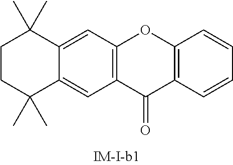 [SMILES: CC1(C)CCC(C)(C)c2cc3c(=O)c4ccccc4oc3cc21] | 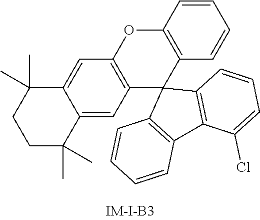 [SMILES: CC1(C)CCC(C)(C)c2cc3c(cc21)Oc1ccccc1C31c2ccccc2-c2c(Cl)cccc21] | 47.2 |
| 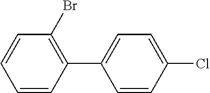 [SMILES: Clc1ccc(-c2ccccc2Br)cc1] | 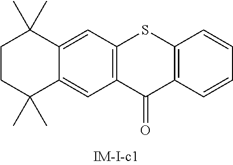 [SMILES: CC1(C)CCC(C)(C)c2cc3c(=O)c4ccccc4sc3cc21] | 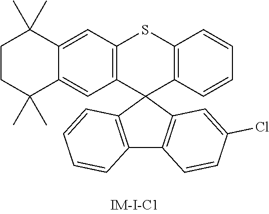 [SMILES: CC1(C)CCC(C)(C)c2cc3c(cc21)Sc1ccccc1C31c2ccccc2-c2ccc(Cl)cc21] | 47.7 |
| 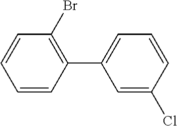 [SMILES: Clc1cccc(-c2ccccc2Br)c1] | 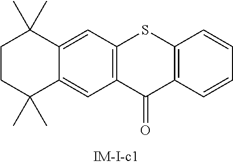 [SMILES: CC1(C)CCC(C)(C)c2cc3c(=O)c4ccccc4sc3cc21] | 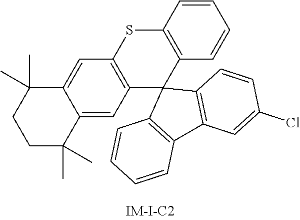 [SMILES: CC1(C)CCC(C)(C)c2cc3c(cc21)Sc1ccccc1C31c2ccccc2-c2cc(Cl)ccc21] | 44.2 |
| 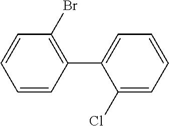 [SMILES: Clc1ccccc1-c1ccccc1Br] | 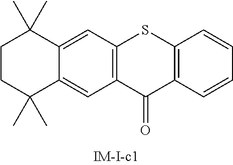 [SMILES: CC1(C)CCC(C)(C)c2cc3c(=O)c4ccccc4sc3cc21] | 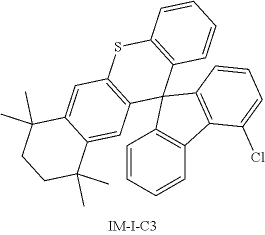 [SMILES: CC1(C)CCC(C)(C)c2cc3c(cc21)Sc1ccccc1C31c2ccccc2-c2c(Cl)cccc21] | 48.1 |
| 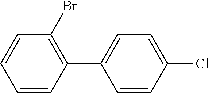 [SMILES: Clc1ccc(-c2ccccc2Br)cc1] | 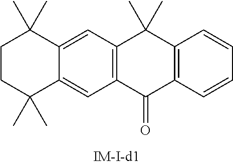 [SMILES: CC1(C)CCC(C)(C)c2cc3c(cc21)C(=O)c1ccccc1C3(C)C] | 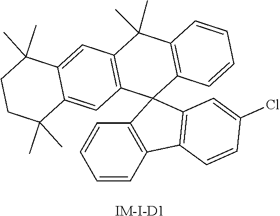 | 47.4 |

### 6. Synthesis of IM-I-A1-L

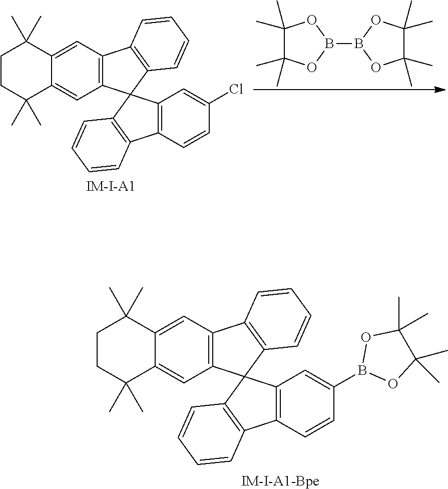

(1) IM-I-A1 (6.0 g, 13.04 mmol) and 1,4-dioxane (60 mL) were added into a 100 mL three-necked flask. Nitrogen was introduced to the flask, then bis(pinacolato)diboron (3.5 g, 13.82 mmol) and potassium acetate (2.56 g, 26.08 mol) were sequentially added. Pd₂(dba)₃ (0.12 g, 0.13 mmol) and X-PhOS (0.12 g, 0.26 mmol) were rapidly added upon the temperature was heated to 80° C. to 90° C. under mechanical stirring. The temperature was maintained at 80° C. to 90° C. for 12 hours of reaction. After the reaction was completed, the temperature was cooled to room temperature. The reaction solution was extracted with toluene, washed with water, and the separated organic phase was dried with anhydrous magnesium sulfate and concentrated under reduced pressure to obtain a crude product. The crude product was recrystallized using toluene to obtain IM-I-A1-Bpe (5.4 g, yield 74.9%).

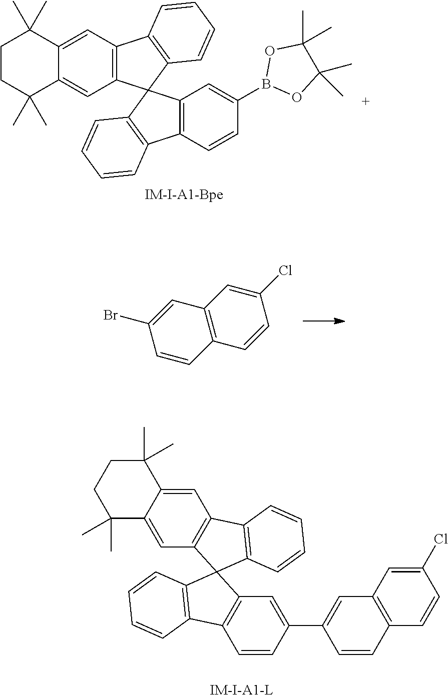

(2) IM-I-A1-L was synthesized using the same method as the intermediate IM-I-a1-1 #, except that IM-I-A1-Bpe (6.0 g, 10.86 mmol) was used to replace the Raw material Sub-I-a1, and 2-bromo-7-chloronaphthalene (2.6 g, 10.86 mmol) was used to replace 2-methoxycarbonylphenylboronic acid pinacol ester. The other raw materials and reaction conditions remained unchanged, resulting in IM-I-A1-L (4.8 g, yield 75.4%).

IM-I-Xn-L in Table 4 were synthesized according to the synthesis method of IM-I-A1-L, except that IM-I-Xn in Table 4 was used to replace IM-I-A1 to synthesize IM-I-Xn-Bpe; then IM-I-Xn-Bpe was used to replace IM-I-A1-Bpe, and the Raw material 5 was used to replace 2-bromo-7-chloronaphthalene. The other raw materials and conditions remained unchanged. The products and yields of the two-step reaction are shown in Table 4.

TABLE 4

| | | Yield at | | | Yield at |
|---|---|---|---|---|---|
| | | first | Raw material | | second |
| IM-I-Xn | IM-I-Xn-Bpe | step/% | 5 | IM-I-Xn-L | step/% |
| 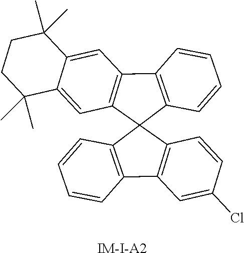 | 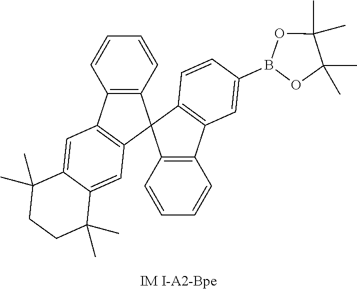 | 77.2 | 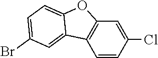 [SMILES: Clc1ccc2c(c1)oc1ccc(Br)cc12] | 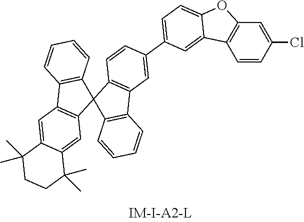 [SMILES: CC1(C)CCC(C)(C)c2cc3c(cc21)-c1ccccc1C31c2ccccc2-c2cc(-c3ccc4oc5cc(Cl)ccc5c4c3)ccc21] | 75.7 |
| 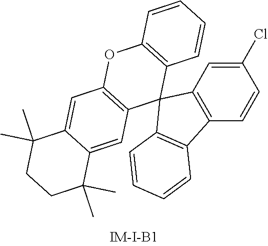 [SMILES: CC1(C)CCC(C)(C)c2cc3c(cc21)Oc1ccccc1C31c2ccccc2-c2ccc(Cl)cc21] | 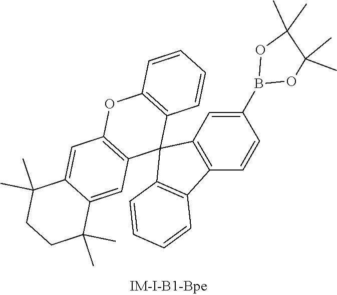 | 74.5 | 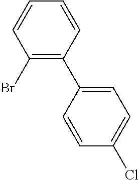 [SMILES: Clc1ccc(-c2ccccc2Br)cc1] | 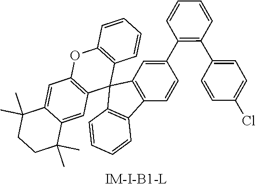 [SMILES: CC1(C)CCC(C)(C)c2cc3c(cc21)Oc1ccccc1C31c2ccccc2-c2ccc(-c3ccccc3-c3ccc(Cl)cc3)cc21] | 68.4 |
| 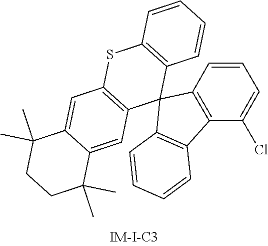 [SMILES: CC1(C)CCC(C)(C)c2cc3c(cc21)Sc1ccccc1C31c2ccccc2-c2c(Cl)cccc21] | 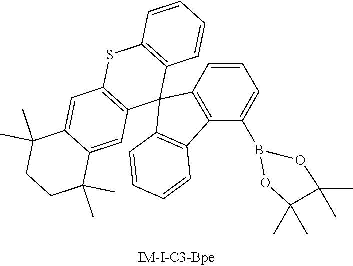 | 70.8 | 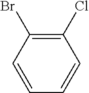 | 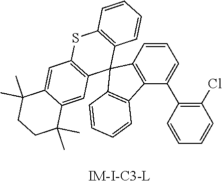 [SMILES: CC1(C)CCC(C)(C)c2cc3c(cc21)Sc1ccccc1C31c2ccccc2-c2c(-c3ccccc3Cl)cccc21] | 67.0 |

### 7. Synthesis of IM-11-N1

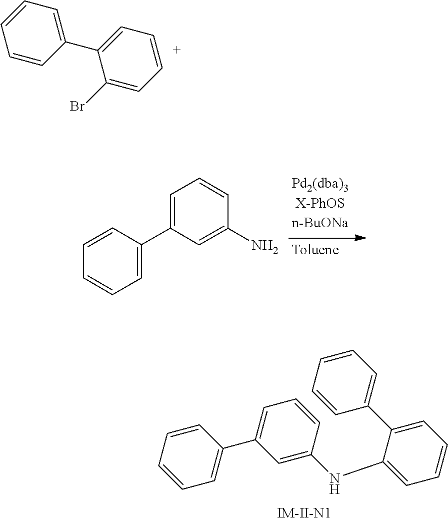 [SMILES: Brc1ccccc1-c1ccccc1 ; Nc1cccc(-c2ccccc2)c1 ; c1ccc(-c2cccc(Nc3ccccc3-c3ccccc3)c2)cc1]

Under nitrogen protection, 2-bromobiphenyl (10.0 g, 42.90 mmol), 3-aminobiphenyl (7.6 g, 45.04 mmol), tris(dibenzylidene acetone) dipalladium (0.4 g, 0.42 mmol), 2-dicyclohexylphosphino-2′,4′,6′-triisopropylbiphenyl (0.4 g, 0.85 mmol), and sodium tert butoxide (6.2 g, 64.35 mmol) were added into a reaction flask, and the resulting mixture was added to toluene (100 mL). The resulting mixture was heated to 108° C. under nitrogen protection and stirred for 3 hours; then the reaction solution was cooled to room temperature, washed with water, and added with magnesium sulfate for drying, followed by filtration. Then the solvent was removed from the filtrate under reduced pressure to obtain a crude product as a yellow solid; the crude product was purified by recrystallization using toluene/n-hexane, resulting in IM-II-N1 (10.4 g, yield 75.5%).

IM-II-Nx in Table 5 were synthesized according to the synthesis method of IM-II-N1, except that the Raw material 6 was used to replace 3-aminobiphenyl and the Raw material 7 was used to replace 2-bromobiphenyl, respectively. Other raw materials and conditions remained unchanged. The structures and yields of the obtained products are shown in Table 5.

TABLE 5

| Raw material 6 | Raw material 7 | IM-II-Nx | Yield/% |
|---|---|---|---|
| 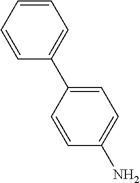 [SMILES: Nc1ccc(-c2ccccc2)cc1] | 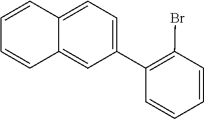 [SMILES: Brc1ccccc1-c1ccc2ccccc2c1] | 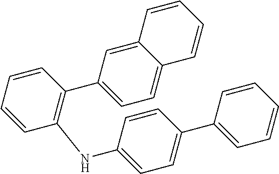 [SMILES: c1ccc(-c2ccc(Nc3ccccc3-c3ccc4ccccc4c3)cc2)cc1] IM-II-N2 | 78.6 |
| 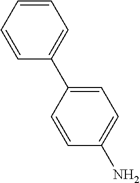 [SMILES: Nc1ccc(-c2ccccc2)cc1] | 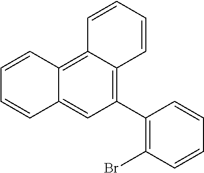 [SMILES: Brc1ccccc1-c1cc2ccccc2c2ccccc12] | 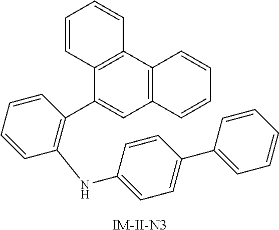 | 74.2 |
| 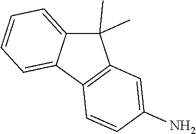 [SMILES: CC1(C)c2ccccc2-c2ccc(N)cc21] | 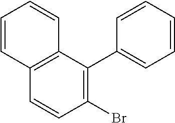 [SMILES: Brc1ccc2ccccc2c1-c1ccccc1] | 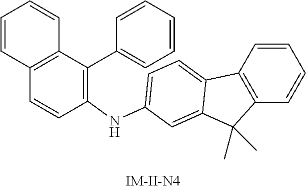 [SMILES: CC1(C)c2ccccc2-c2ccc(Nc3ccc4ccccc4c3-c3ccccc3)cc21] | 72.0 |
| 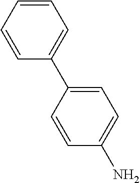 [SMILES: Nc1ccc(-c2ccccc2)cc1] | 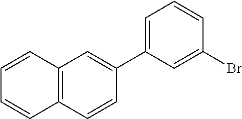 [SMILES: Brc1cccc(-c2ccc3ccccc3c2)c1] | 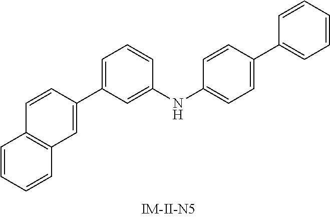 [SMILES: c1ccc(-c2ccc(Nc3cccc(-c4ccc5ccccc5c4)c3)cc2)cc1] | 76.8 |
| 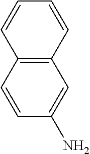 [SMILES: Nc1ccc2ccccc2c1] | 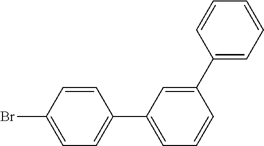 [SMILES: Brc1ccc(-c2cccc(-c3ccccc3)c2)cc1] | 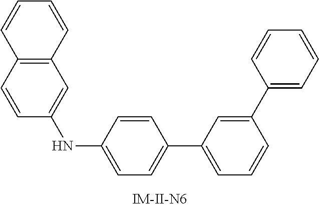 [SMILES: c1ccc(-c2cccc(-c3ccc(Nc4ccc5ccccc5c4)cc3)c2)cc1] | 79.3 |
| 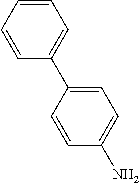 [SMILES: Nc1ccc(-c2ccccc2)cc1] | 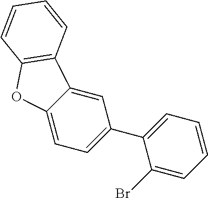 [SMILES: Brc1ccccc1-c1ccc2oc3ccccc3c2c1] | 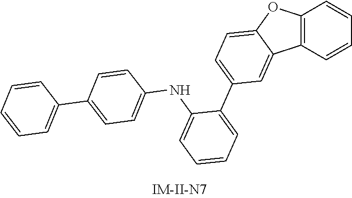 [SMILES: c1ccc(-c2ccc(Nc3ccccc3-c3ccc4oc5ccccc5c4c3)cc2)cc1] | 71.7 |
| 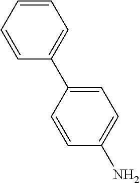 [SMILES: Nc1ccc(-c2ccccc2)cc1] | 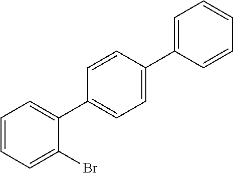 [SMILES: Brc1ccccc1-c1ccc(-c2ccccc2)cc1] | 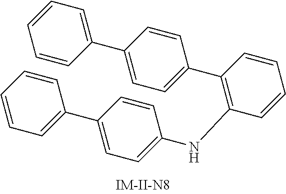 [SMILES: c1ccc(-c2ccc(Nc3ccccc3-c3ccc(-c4ccccc4)cc3)cc2)cc1] | 77.5 |

### Synthesis of Compound: Taking Compound A04 as an Example

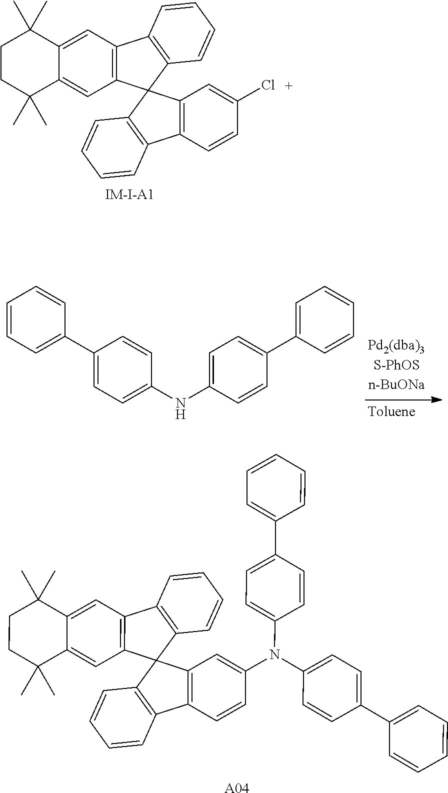

Under nitrogen protection, IM-I-A1 (4.7 g, 10.19 mmol), di(4-biphenyl)amine (3.3 g, 10.20 mmol), tri(dibenzylidene acetone) dipalladium (0.1 g, 0.11 mmol), 2-dicyclohexylphosphino-2′,6′-dimethoxybiphenyl (0.1 g, 0.22 mmol), and sodium tert butoxide (1.47 g, 15.29 mmol) were added into a rounded flask, and the resulting mixture was added to toluene (40 mL). The resulting mixture was heated to 108° C. under nitrogen protection and stirred for 4 hours; then the reaction solution was cooled to room temperature, washed with water, and added with magnesium sulfate for drying, followed by filtration. Then the solvent was removed from the filtrate under reduced pressure to obtain a crude product; the crude product was purified by recrystallization using toluene/n-hexane, resulting in the Compound A04 as a white solid (4.3 g, yield 56.6%). Mass Spectra LC-MS (ESI, pos. ion): m/z=746.4[M+H]⁺.

Compounds in Table 6 were synthesized using the same method as Compound A04, except that the Raw material 8 in Table 6 were used to replace IM-I-A1, and the Raw material 9 in Table 6 were used to replace di(4-biphenyl)amine, respectively. Other conditions remained unchanged. The structures of the raw materials used, as well as the structures and yields of the products, are listed in Table 6.

TABLE 6

| | | | | | Mass |
|---|---|---|---|---|---|
| | | | | | Spectra |
| | | | | | (m/z) |
| No. | Raw material 8 | Raw material 9 | Product | Yield/% | [M + H]⁺ |
| 2 | 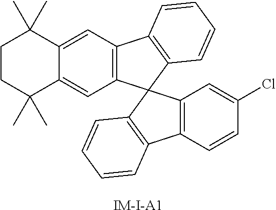 | 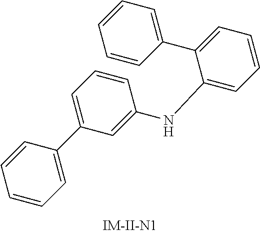 [SMILES: c1ccc(-c2cccc(Nc3ccccc3-c3ccccc3)c2)cc1] | 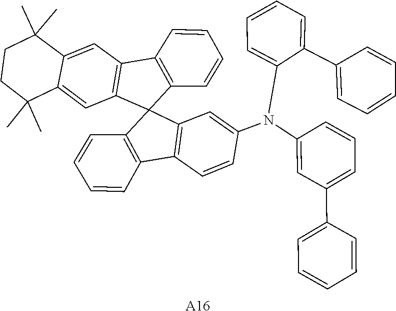 [SMILES: CC1(C)CCC(C)(C)c2cc3c(cc21)-c1ccccc1C31c2ccccc2-c2ccc(N(c3cccc(-c4ccccc4)c3)c3ccccc3-c3ccccc3)cc21 ; "A16"] | 45.2 | 746.4 |
| 3 | 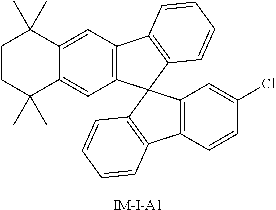 | 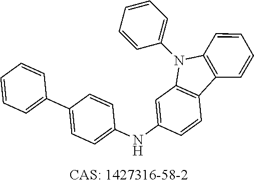 [SMILES: c1ccc(-c2ccc(Nc3ccc4c5ccccc5n(-c5ccccc5)c4c3)cc2)cc1] | 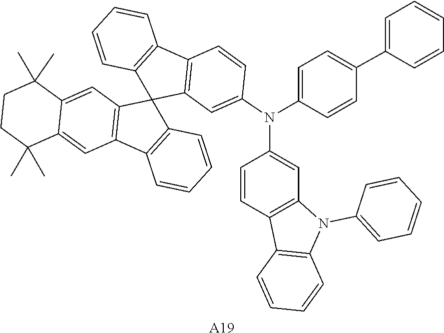 [SMILES: CC1(C)CCC(C)(C)c2cc3c(cc21)-c1ccccc1C31c2ccccc2-c2ccc(N(c3ccc(-c4ccccc4)cc3)c3ccc4c5ccccc5n(-c5ccccc5)c4c3)cc21 ; "A19"] | 50.3 | 835.4 |
| 4 | 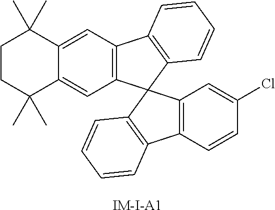 | 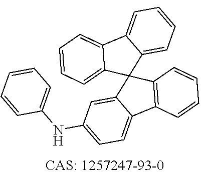 | 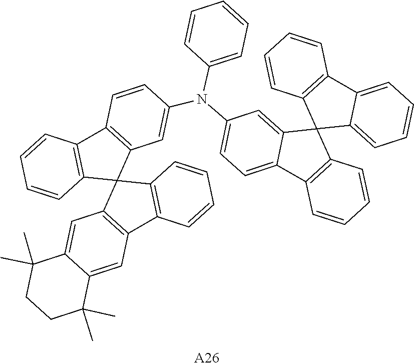 [SMILES: CC1(C)CCC(C)(C)c2cc3c(cc21)-c1ccccc1C31c2ccccc2-c2ccc(N(c3ccccc3)c3ccc4c(c3)C3(c5ccccc5-c5ccccc53)c3ccccc3-4)cc21 ; "A26"] | 48.8 | 832.4 |
| 5 | 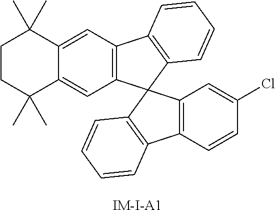 | 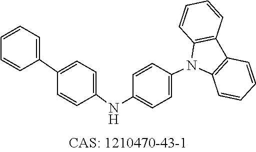 [SMILES: c1ccc(-c2ccc(Nc3ccc(-n4c5ccccc5c5ccccc54)cc3)cc2)cc1] | 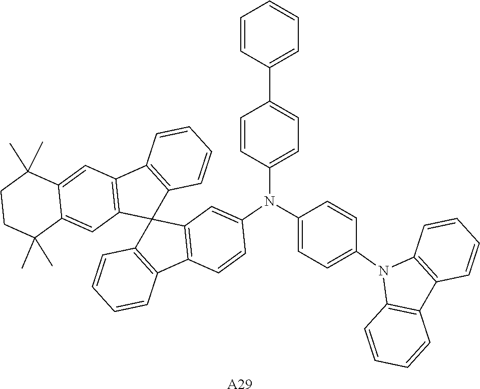 [SMILES: CC1(C)CCC(C)(C)c2cc3c(cc21)-c1ccccc1C31c2ccccc2-c2ccc(N(c3ccc(-c4ccccc4)cc3)c3ccc(-n4c5ccccc5c5ccccc54)cc3)cc21 ; "A29"] | 51.6 | 835.4 |
| 6 | 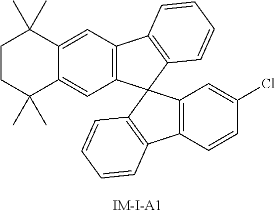 | 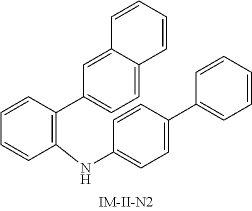 [SMILES: c1ccc(-c2ccc(Nc3ccccc3-c3ccc4ccccc4c3)cc2)cc1] | 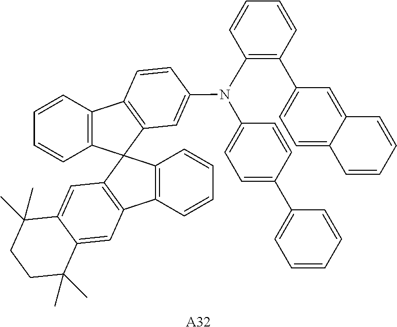 [SMILES: CC1(C)CCC(C)(C)c2cc3c(cc21)-c1ccccc1C31c2ccccc2-c2ccc(N(c3ccc(-c4ccccc4)cc3)c3ccccc3-c3ccc4ccccc4c3)cc21 ; "A32"] | 46.1 | 796.4 |
| 7 | 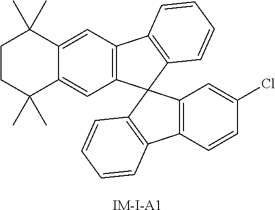 | 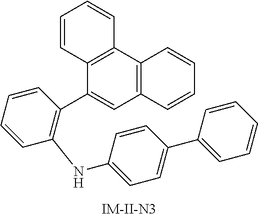 | 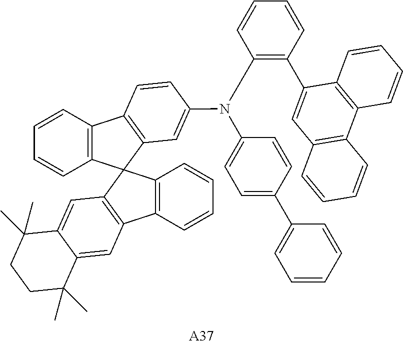 [SMILES: CC1(C)CCC(C)(C)c2cc3c(cc21)-c1ccccc1C31c2ccccc2-c2ccc(N(c3ccc(-c4ccccc4)cc3)c3ccccc3-c3cc4ccccc4c4ccccc34)cc21 ; "A37"] | 44.0 | 846.4 |
| 8 | 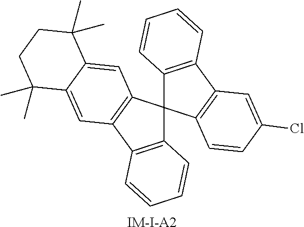 | 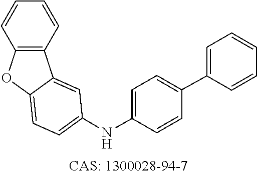 [SMILES: c1ccc(-c2ccc(Nc3ccc4oc5ccccc5c4c3)cc2)cc1] | 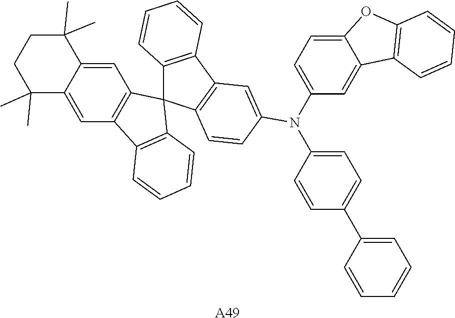 [SMILES: CC1(C)CCC(C)(C)c2cc3c(cc21)-c1ccccc1C31c2ccccc2-c2cc(N(c3ccc(-c4ccccc4)cc3)c3ccc4oc5ccccc5c4c3)ccc21 ; "A49"] | 49.8 | 760.3 |
| 9 | 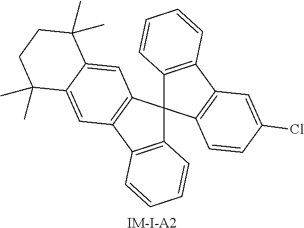 | 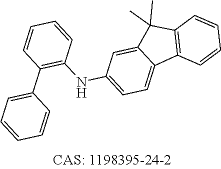 [SMILES: CC1(C)c2ccccc2-c2ccc(Nc3ccccc3-c3ccccc3)cc21] | 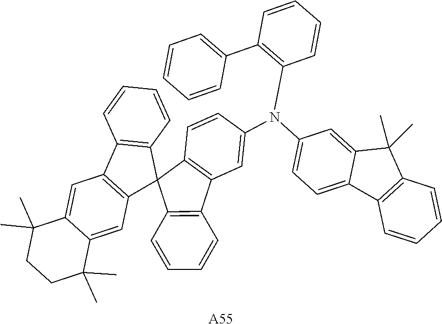 [SMILES: CC1(C)CCC(C)(C)c2cc3c(cc21)-c1ccccc1C31c2ccccc2-c2cc(N(c3ccc4c(c3)C(C)(C)c3ccccc3-4)c3ccccc3-c3ccccc3)ccc21 ; "A55"] | 46.4 | 786.4 |
| 10 | 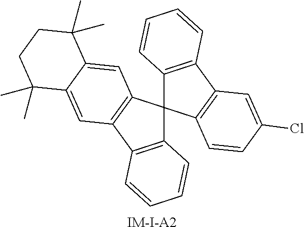 | 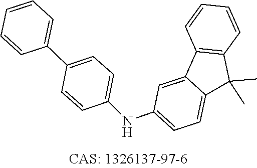 [SMILES: CC1(C)c2ccccc2-c2cc(Nc3ccc(-c4ccccc4)cc3)ccc21] | 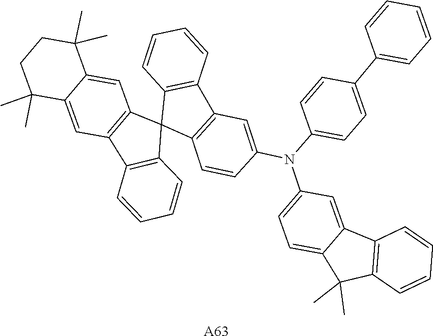 [SMILES: CC1(C)CCC(C)(C)c2cc3c(cc21)-c1ccccc1C31c2ccccc2-c2cc(N(c3ccc(-c4ccccc4)cc3)c3ccc4c(c3)-c3ccccc3C4(C)C)ccc21 ; "A63"] | 50.5 | 786.4 |
| 11 | 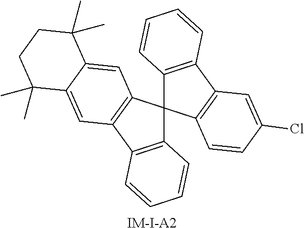 | 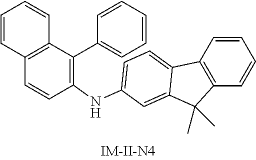 [SMILES: CC1(C)c2ccccc2-c2ccc(Nc3ccc4ccccc4c3-c3ccccc3)cc21] | 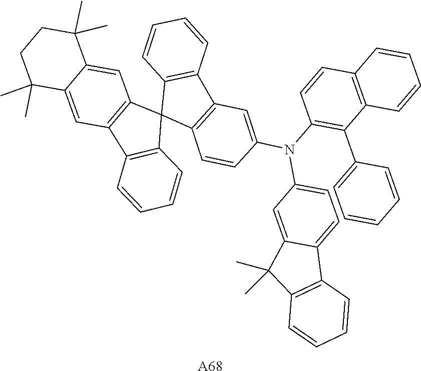 [SMILES: CC1(C)CCC(C)(C)c2cc3c(cc21)-c1ccccc1C31c2ccccc2-c2cc(N(c3ccc4c(c3)C(C)(C)c3ccccc3-4)c3ccc4ccccc4c3-c3ccccc3)ccc21 ; "A68"] | 45.0 | 836.4 |
| 12 | 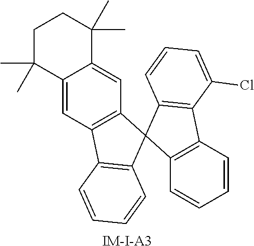 | 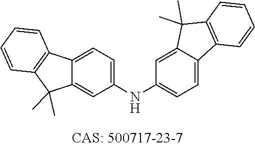 [SMILES: CC1(C)c2ccccc2-c2ccc(Nc3ccc4c(c3)C(C)(C)c3ccccc3-4)cc21] | 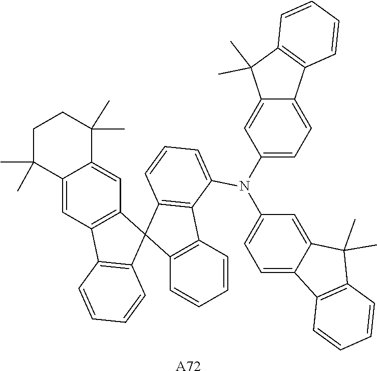 | 47.2 | 826.4 |
| 13 | 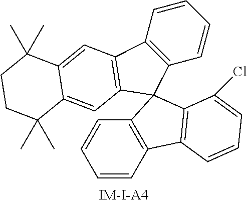 | CAS:500717-23-7 | 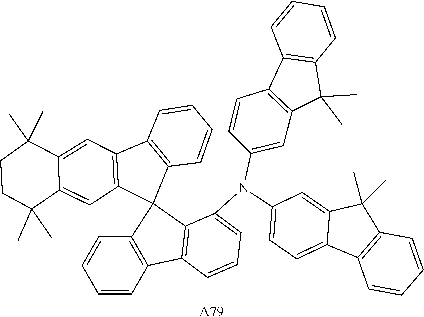 | 34.6 | 826.4 |
| 14 | 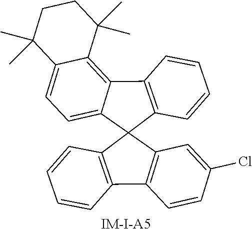 | 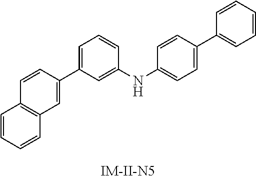 [SMILES: c1ccc(-c2ccc(Nc3cccc(-c4ccc5ccccc5c4)c3)cc2)cc1] | 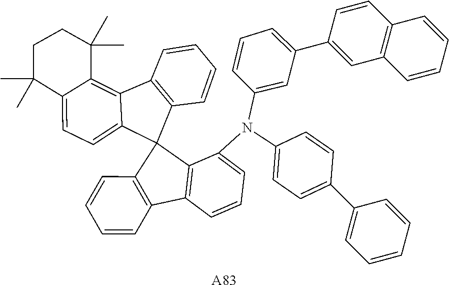 [SMILES: CC1(C)CCC(C)(C)c2c1ccc1c2-c2ccccc2C12c1ccccc1-c1cccc(N(c3ccc(-c4ccccc4)cc3)c3cccc(-c4ccc5ccccc5c4)c3)c12 ; "A83"] | 44.9 | 796.4 |
| 15 | 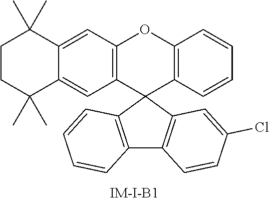 [SMILES: CC1(C)CCC(C)(C)c2cc3c(cc21)Oc1ccccc1C31c2ccccc2-c2ccc(Cl)cc21] | 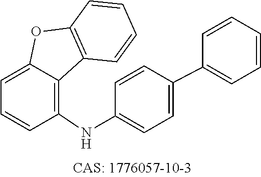 [SMILES: c1ccc(-c2ccc(Nc3cccc4oc5ccccc5c34)cc2)cc1] | 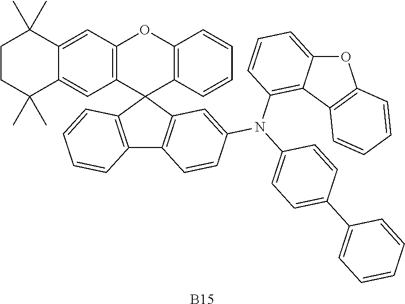 [SMILES: CC1(C)CCC(C)(C)c2cc3c(cc21)Oc1ccccc1C31c2ccccc2-c2ccc(N(c3ccc(-c4ccccc4)cc3)c3cccc4oc5ccccc5c34)cc21 ; "B15"] | 48.7 | 776.3 |
| 16 | 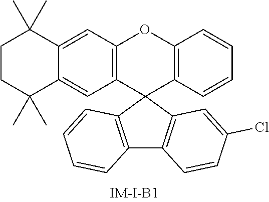 [SMILES: CC1(C)CCC(C)(C)c2cc3c(cc21)Oc1ccccc1C31c2ccccc2-c2ccc(Cl)cc21] | 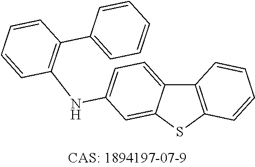 [SMILES: c1ccc(-c2ccccc2Nc2ccc3c(c2)sc2ccccc23)cc1] | 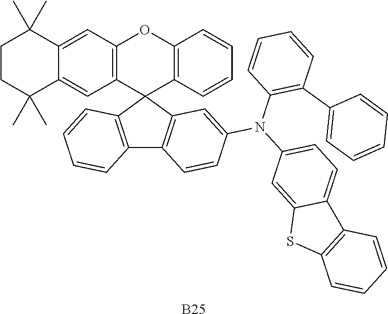 [SMILES: CC1(C)CCC(C)(C)c2cc3c(cc21)Oc1ccccc1C31c2ccccc2-c2ccc(N(c3ccc4c(c3)sc3ccccc34)c3ccccc3-c3ccccc3)cc21 ; "B25"] | 46.3 | 792.3 |
| 17 | 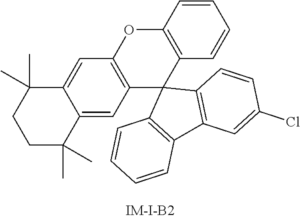 [SMILES: CC1(C)CCC(C)(C)c2cc3c(cc21)Oc1ccccc1C31c2ccccc2-c2cc(Cl)ccc21] | 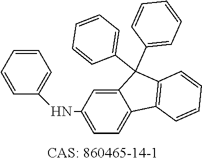 [SMILES: c1ccc(Nc2ccc3c(c2)C(c2ccccc2)(c2ccccc2)c2ccccc2-3)cc1] | 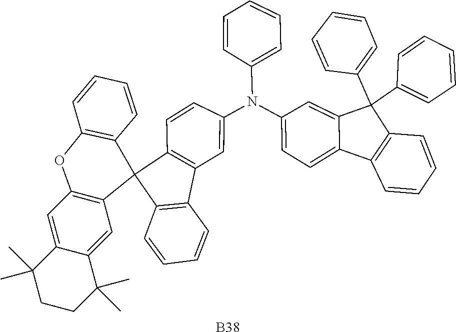 | 52.4 | 850.4 |
| 18 | 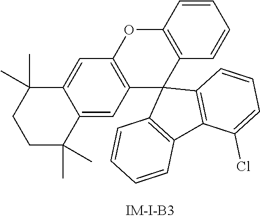 [SMILES: CC1(C)CCC(C)(C)c2cc3c(cc21)Oc1ccccc1C31c2ccccc2-c2c(Cl)cccc21] | 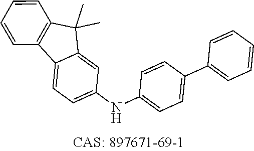 [SMILES: CC1(C)c2ccccc2-c2ccc(Nc3ccc(-c4ccccc4)cc3)cc21] | 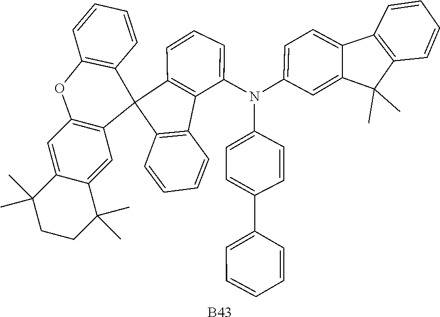 | 42.5 | 802.4 |
| 19 | 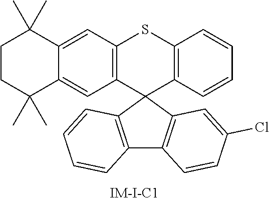 [SMILES: CC1(C)CCC(C)(C)c2cc3c(cc21)Sc1ccccc1C31c2ccccc2-c2ccc(Cl)cc21] | 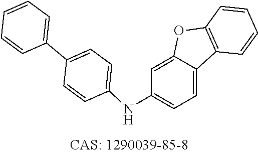 [SMILES: c1ccc(-c2ccc(Nc3ccc4c(c3)oc3ccccc34)cc2)cc1] | 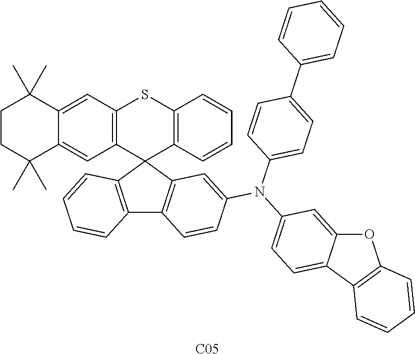 [SMILES: CC1(C)CCC(C)(C)c2cc3c(cc21)Sc1ccccc1C31c2ccccc2-c2ccc(N(c3ccc(-c4ccccc4)cc3)c3ccc4c(c3)oc3ccccc34)cc21 ; "C05"] | 53.7 | 792.3 |
| 20 | 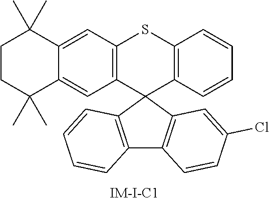 [SMILES: CC1(C)CCC(C)(C)c2cc3c(cc21)Sc1ccccc1C31c2ccccc2-c2ccc(Cl)cc21] | 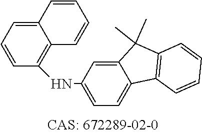 [SMILES: CC1(C)c2ccccc2-c2ccc(Nc3cccc4ccccc34)cc21] | 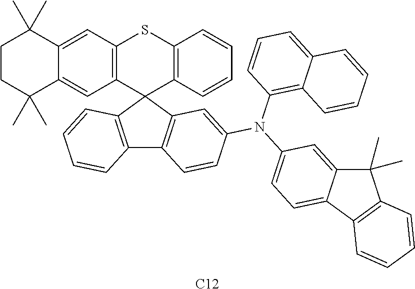 [SMILES: CC1(C)CCC(C)(C)c2cc3c(cc21)Sc1ccccc1C31c2ccccc2-c2ccc(N(c3ccc4c(c3)C(C)(C)c3ccccc3-4)c3cccc4ccccc34)cc21 ; "C12"] | 50.2 | 792.3 |
| 21 | 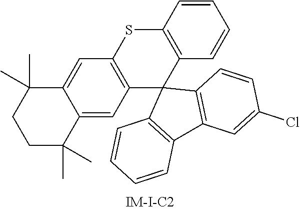 [SMILES: CC1(C)CCC(C)(C)c2cc3c(cc21)Sc1ccccc1C31c2ccccc2-c2cc(Cl)ccc21] | 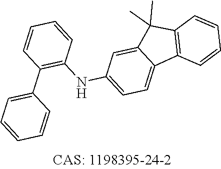 [SMILES: CC1(C)c2ccccc2-c2ccc(Nc3ccccc3-c3ccccc3)cc21] | 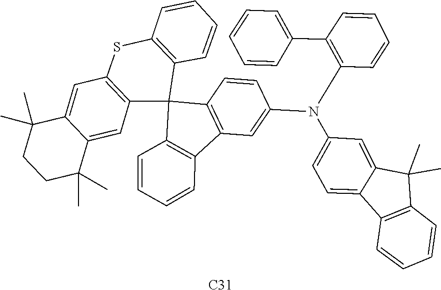 | 48.5 | 818.4 |
| 22 | 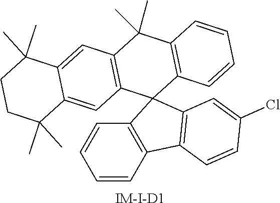 | 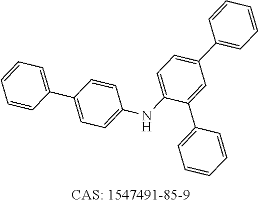 [SMILES: c1ccc(-c2ccc(Nc3ccc(-c4ccccc4)cc3-c3ccccc3)cc2)cc1] | 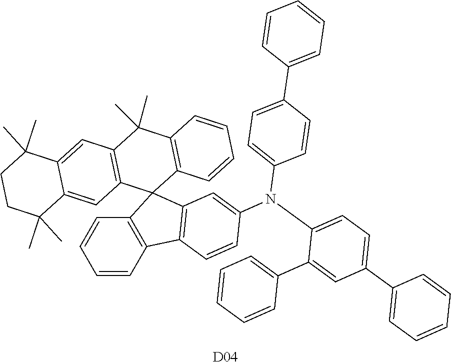 [SMILES: CC1(C)CCC(C)(C)c2cc3c(cc21)C(C)(C)c1ccccc1C31c2ccccc2-c2ccc(N(c3ccc(-c4ccccc4)cc3)c3ccc(-c4ccccc4)cc3-c3ccccc3)cc21 ; "D04"] | 47.0 | 864.5 |
| 23 | 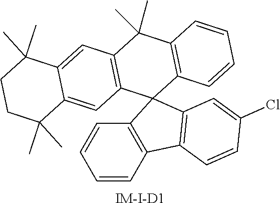 | 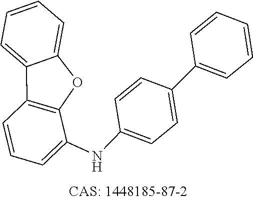 [SMILES: c1ccc(-c2ccc(Nc3cccc4c3oc3ccccc34)cc2)cc1] | 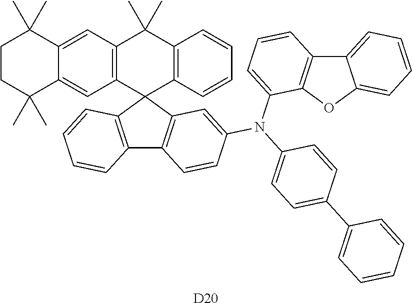 [SMILES: CC1(C)CCC(C)(C)c2cc3c(cc21)C(C)(C)c1ccccc1C31c2ccccc2-c2ccc(N(c3ccc(-c4ccccc4)cc3)c3cccc4c3oc3ccccc34)cc21 ; "D20"] | 49.3 | 802.4 |
| 24 | 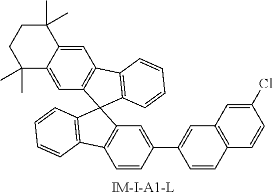 | 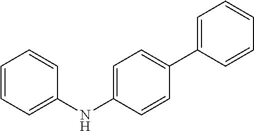 [SMILES: c1ccc(Nc2ccc(-c3ccccc3)cc2)cc1] | 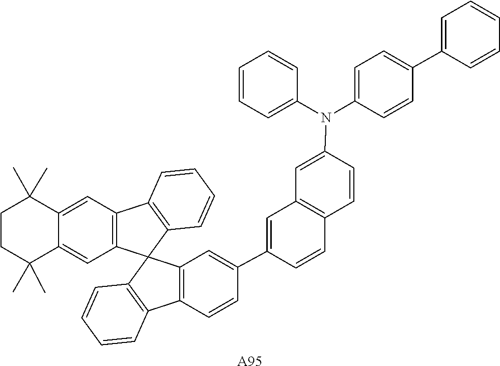 [SMILES: CC1(C)CCC(C)(C)c2cc3c(cc21)-c1ccccc1C31c2ccccc2-c2ccc(-c3ccc4ccc(N(c5ccccc5)c5ccc(-c6ccccc6)cc5)cc4c3)cc21 ; "A95"] | 43.4 | 796.4 |
| 25 | 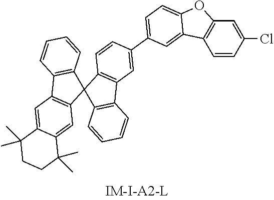 [SMILES: CC1(C)CCC(C)(C)c2cc3c(cc21)-c1ccccc1C31c2ccccc2-c2cc(-c3ccc4oc5cc(Cl)ccc5c4c3)ccc21] | 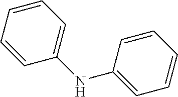 [SMILES: c1ccc(Nc2ccccc2)cc1] | 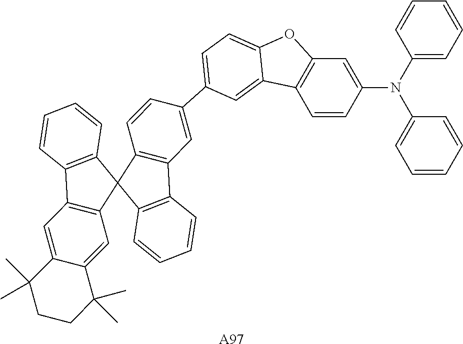 [SMILES: CC1(C)CCC(C)(C)c2cc3c(cc21)-c1ccccc1C31c2ccccc2-c2cc(-c3ccc4oc5cc(N(c6ccccc6)c6ccccc6)ccc5c4c3)ccc21 ; "A97"] | 68.3 | 760.3 |
| 26 | 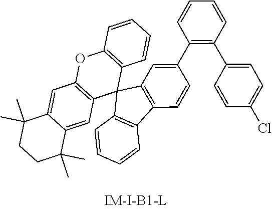 [SMILES: CC1(C)CCC(C)(C)c2cc3c(cc21)Oc1ccccc1C31c2ccccc2-c2ccc(-c3ccccc3-c3ccc(Cl)cc3)cc21] | 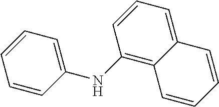 [SMILES: c1ccc(Nc2cccc3ccccc23)cc1] | 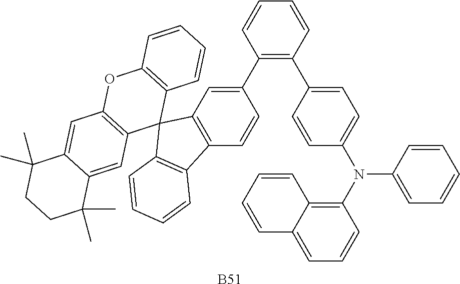 | 65.0 | 812.4 |
| 27 | 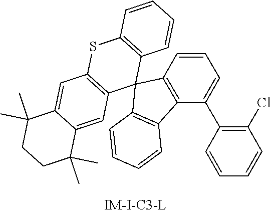 [SMILES: CC1(C)CCC(C)(C)c2cc3c(cc21)Sc1ccccc1C31c2ccccc2-c2c(-c3ccccc3Cl)cccc21] | 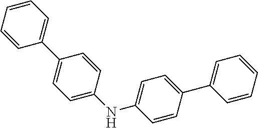 [SMILES: c1ccc(-c2ccc(Nc3ccc(-c4ccccc4)cc3)cc2)cc1] | 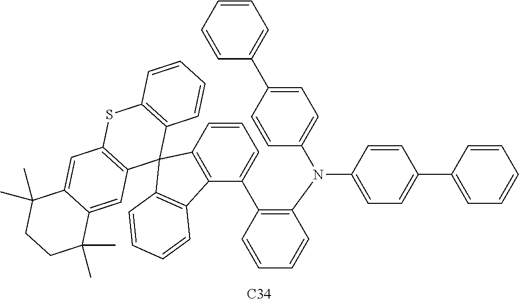 [SMILES: CC1(C)CCC(C)(C)c2cc3c(cc21)Sc1ccccc1C31c2ccccc2-c2c(-c3ccccc3N(c3ccc(-c4ccccc4)cc3)c3ccc(-c4ccccc4)cc3)cccc21 ; "C34"] | 48.6 | 854.4 |

NMR data of some Compounds are shown in Table 7 below:

TABLE 7

| Compound | ¹H-NMR (400 MHz, CD₂Cl₂) δ ppm |
|---|---|
| Compound | 7.91-7.88(m, 2H), 7.60-7.54(m, 8H), 7.51-7.48(m, 3H), |
| A04 | 7.45-7.38(m, 6H), 7.23-7.17(m, 2H), 7.12(t, 1H), |
| | 7.02-6.98(m, 3H), 6.88(d, 4H), 6.83(s, 1H), |
| | 6.76(s, 1H), 2.28-2.16(m, 2H), 2.09-1.97(m, 2H), |
| | 1.49(s, 6H), 1.46(s, 6H). |
| Compound | 8.22(d, 1H), 7.89(d, 1H), 7.76-7.68(m, 3H), |
| B51 | 7.62-7.44(m, 6H), 7.38-7.25(m, 7H), 7.21(t, 1H), |
| | 7.17-7.09(m, 6H), 7.02(d, 1H), 6.91 (s, 1H), |
| | 6.88-6.84(m, 3H), 6.78(d, 2H), 6.73(s, 1H), |
| | 2.29-2.17(m, 2H), 2.11-1.99(m, 2H), 1.49(s, 6H), |
| | 1.47(s, 6H). |
| Compound | 8.02-7.98(m, 2H), 7.88(d, 1H), 7.62-7.51(m, 8H), |
| C05 | 7.49-7.41(m, 3H), 7.33-7.28(m, 2H), 7.16(s, 1H), |
| | 7.14-7.10(m, 2H), 7.08-7.02(m, 4H), 6.91(s, 1H), |
| | 6.88(d, 1H), 6.81(d, 2H), 6.77(s, 1H), 6.68(s, 1H), |
| | 2.26-2.14(m, 2H), 2.07-1.95(m, 2H), 1.46(s, 6H), |
| | 1.43(s, 6H). |

### Example 1

This example provided an organic electroluminescent device, and the specific fabrication method was as follows:

The glass substrate coated with ITO transparent conductive layer was ultrasonically treated in a commercial cleaning agent, rinsed in deionized water, ultrasonically degreased in a mixed solvent of acetone: ethanol, baked in a clean environment until completely removal of moisture.

The surface was treated with ultraviolet ozone and O₂:N₂ plasma to enhance the work function of the anode and remove floating slag.

The glass substrate with an anode was placed in a vacuum chamber which is evacuated to less than 1×10⁻⁵ Pa, and the compound HAT-CN was deposited by vacuum evaporation on the anode layer film to form a hole injection layer with a thickness of 10 nm.

The Compound A04 was deposited by vacuum evaporation on the electron injection layer to form a hole transport layer with a thickness of 100 Å.

The Compound EB-01 was deposited by vacuum evaporation on the hole transport layer to form an electron blocking layer with a thickness of 80 Å.

The light-emitting layer of the device was deposited by vacuum evaporation on the electron blocking layer, and Compound BH-01 and Compound BD-01 were co-deposited in a mass ratio of 97%:3% using a multi-source co-evaporation method to form an organic light-emitting layer with a thickness of 300 Å.

Compound ET-18 and LiQ were deposited on the organic light-emitting layer in a weight ratio of 1:1 to form an electron transport layer with a thickness of 280 Å.

LiF with a thickness of 5 Å was deposited on the electron transport layer as the electron injection layer, and then magnesium (Mg) and silver (Ag) were co-deposited on the electron injection layer at a vapor deposition rate of 1:9 (mass ratio) to form a cathode with a thickness of 120 Å.

In addition, CP-01 was deposited by vacuum evaporation on the above cathode to form an organic cover layer with a thickness of 600 Å, thus completing the fabrication of the organic electroluminescent device.

### Example 2 to Example 27

Organic electroluminescent devices were fabricated using the same method as Example 1, except that the compounds in Table 8 were used instead of Compound A04 when forming a hole transport layer.

### Comparative Example 1 to Comparative Example 4

Organic electroluminescent devices were fabricated using the same method as Example 1, except that Compound A to Compound D in Table 8 were used instead of Compound A04 when forming a hole transport layer.

In the fabrication of organic electroluminescent devices, the structures of the materials used in the Comparative Examples and the Examples are as follows:

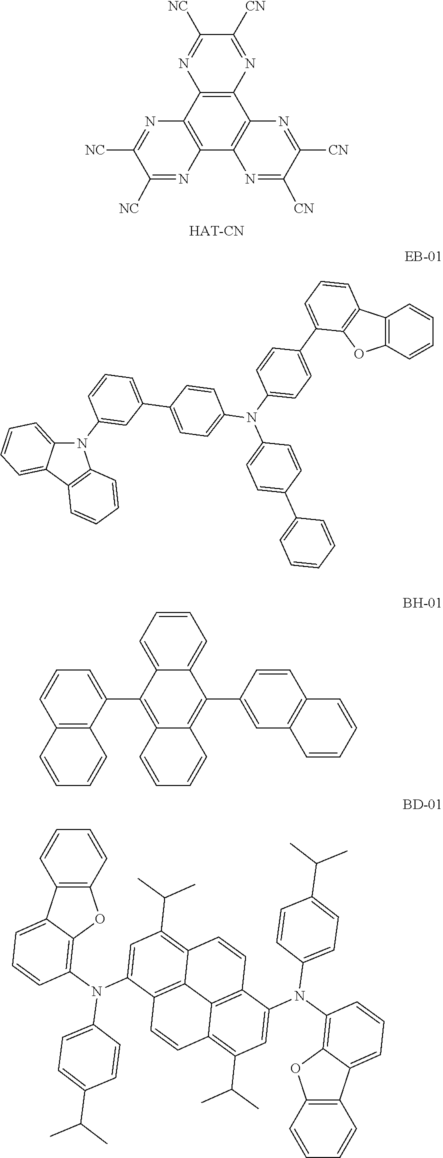

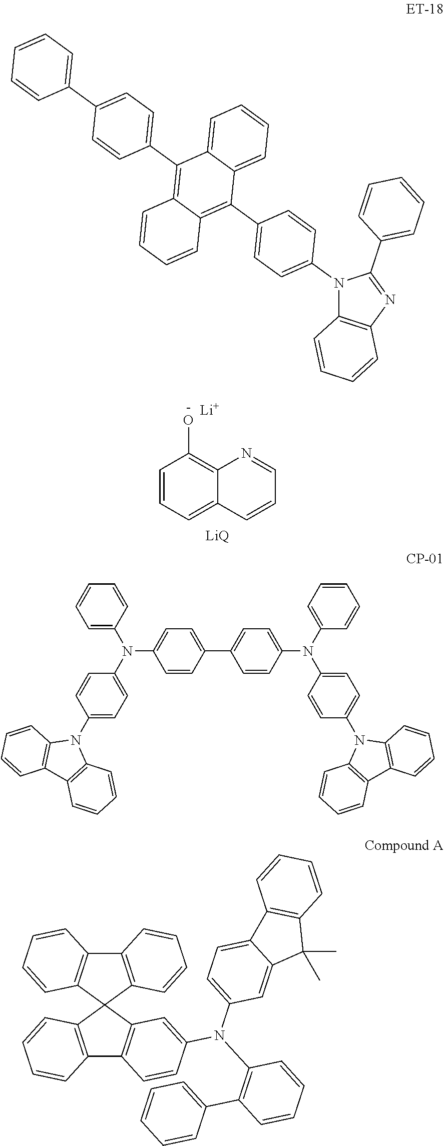

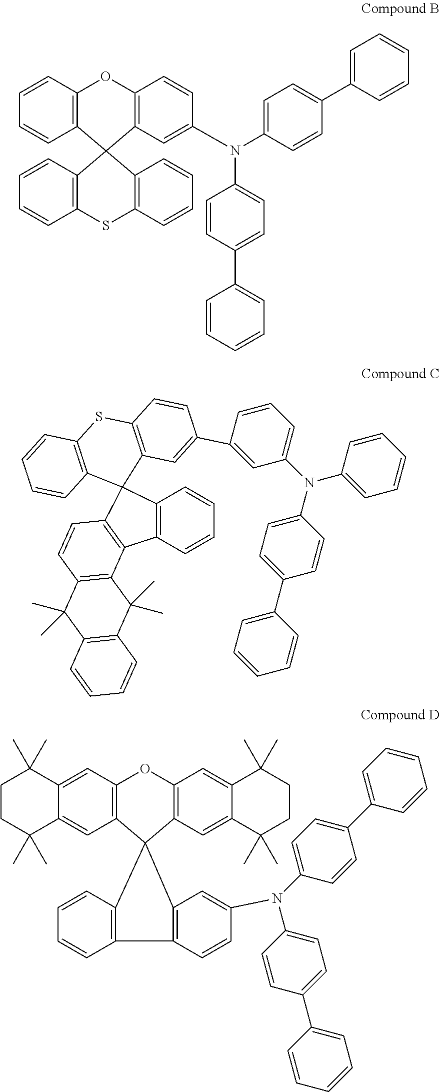 [SMILES: c1ccc(-c2ccc(N(c3ccc(-c4ccccc4)cc3)c3ccc4c(c3)C3(c5ccccc5O4)c4ccccc4Sc4ccccc43)cc2)cc1 ; CC1(C)c2ccccc2C(C)(C)c2c1ccc1c2-c2ccccc2C12c1ccccc1Sc1ccc(-c3cccc(N(c4ccccc4)c4ccc(-c5ccccc5)cc4)c3)cc12 ; CC1(C)CCC(C)(C)c2cc3c(cc21)Oc1cc2c(cc1C31c3ccccc3-c3ccc(N(c4ccc(-c5ccccc5)cc4)c4ccc(-c5ccccc5)cc4)cc31)C(C)(C)CCC2(C)C]

The performances of the organic electroluminescent devices prepared in Example 1 to Example 27 and Comparative Example 1 to Comparative Example 4 were tested. Specifically, the IVL performance of the devices was tested at 10 mA/cm², and the T₉₅ device lifetime was tested at 15 mA/cm². The test results are shown in Table 8 below.

TABLE 8

| | Hole | Driving | Current | Color | T₉₅ device |
|---|---|---|---|---|---|
| | Transport | Voltage | Efficiency | coordinates | lifetime |
| | Layer | (V) | (Cd/A) | CIEx, CIEy | (h) |
| Example 1 | Compound A04 | 3.87 | 6.94 | 0.141, 0.052 | 192 |
| Example 2 | Compound A16 | 3.90 | 6.83 | 0.141, 0.052 | 179 |
| Example 3 | Compound A19 | 3.81 | 6.88 | 0.141, 0.052 | 187 |
| Example 4 | Compound A26 | 3.93 | 6.72 | 0.141, 0.052 | 195 |
| Example 5 | Compound A29 | 3.93 | 6.78 | 0.141, 0.052 | 187 |
| Example 6 | Compound A32 | 3.86 | 6.85 | 0.141, 0.052 | 194 |
| Example 7 | Compound A37 | 3.90 | 6.96 | 0.141, 0.052 | 188 |
| Example 8 | Compound A49 | 3.95 | 6.89 | 0.141, 0.052 | 193 |
| Example 9 | Compound A55 | 3.86 | 6.79 | 0.141, 0.052 | 190 |
| Example 10 | Compound A63 | 3.82 | 6.82 | 0.141, 0.052 | 195 |
| Example 11 | Compound A68 | 3.89 | 6.73 | 0.141, 0.052 | 182 |
| Example 12 | Compound A72 | 3.91 | 7.00 | 0.141, 0.052 | 183 |
| Example 13 | Compound A79 | 3.81 | 6.84 | 0.141, 0.052 | 184 |
| Example 14 | Compound A83 | 3.82 | 6.96 | 0.141, 0.052 | 193 |
| Example 15 | Compound A95 | 3.94 | 6.91 | 0.141, 0.052 | 191 |
| Example 16 | Compound A97 | 3.95 | 6.98 | 0.141, 0.052 | 190 |
| Example 17 | Compound B15 | 3.92 | 6.48 | 0.141, 0.052 | 181 |
| Example 18 | Compound B25 | 3.89 | 6.42 | 0.141, 0.052 | 177 |
| Example 19 | Compound B38 | 3.83 | 6.50 | 0.141, 0.052 | 182 |
| Example 20 | Compound B43 | 3.96 | 6.47 | 0.141, 0.052 | 189 |
| Example 21 | Compound B51 | 3.98 | 6.54 | 0.141, 0.052 | 182 |
| Example 22 | Compound C05 | 3.86 | 6.24 | 0.141, 0.052 | 164 |
| Example 23 | Compound C12 | 3.86 | 6.06 | 0.141, 0.052 | 160 |
| Example 24 | Compound C31 | 3.88 | 6.10 | 0.141, 0.052 | 163 |
| Example 25 | Compound C34 | 3.90 | 6.22 | 0.141, 0.052 | 155 |
| Example 26 | Compound D04 | 3.90 | 6.05 | 0.141, 0.052 | 169 |
| Example 27 | Compound D20 | 3.86 | 6.17 | 0.141, 0.052 | 156 |
| Comparative | Compound A | 3.99 | 5.38 | 0.141, 0.052 | 131 |
| Example 1 | | | | | |
| Comparative | Compound B | 4.02 | 5.16 | 0.141, 0.052 | 125 |
| Example 2 | | | | | |
| Comparative | Compound C | 4.10 | 5.07 | 0.141, 0.052 | 119 |
| Example 3 | | | | | |
| Comparative | Compound D | 4.00 | 5.31 | 0.141, 0.052 | 122 |
| Example 4 | | | | | |

Referring to Table 8 above, it can be seen that compared to the organic electroluminescent devices of Comparative Example 1 to Comparative Example 4, the performances of the organic electroluminescent devices of Example 1 to Example 27 have been greatly improved, mainly manifested in an increase in luminous efficiency of at least 12.5% and an increase in T₉₅ lifetime of at least 18.3%. It can be seen that using the organic compound of the present disclosure in the hole transport layer of organic electroluminescent devices can significantly improve the luminous efficiency and T₉₅ lifetime of organic electroluminescent devices.

The preferred embodiments of the present disclosure are described in detail above in conjunction with the accompanying drawings. However, the present disclosure is not limited to the specific details of the above embodiments. Within the scope of the technical concept of the present disclosure, various simple modifications can be made to the technical solutions of the present disclosure, and these simple modifications all fall within the scope of protection of the present disclosure.

1. An organic compound, having a structure shown in Formula I: 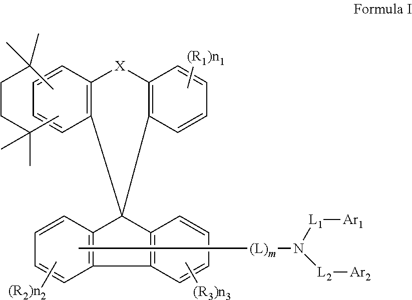
wherein,
X is selected from a single bond, C(R₄R₅), O, and S;
R₄ and R₅ are the same or different, and are each independently selected from a hydrogen, an alkyl having 1 to 10 carbon atoms, and an aryl having 6 to 20 carbon atoms;
R₁, R₂, and R₃ are the same or different, and are each independently selected from a deuterium, a cyano, a halogen group, an alkyl having 1 to 10 carbon atoms, a haloalkyl having 1 to 10 carbon atoms, a deuteroalkyl having 1 to 10 carbon atoms, a deuteroaryl having 6 to 12 carbon atoms, a trialkylsilyl having 3 to 12 carbon atoms, an aryl having 6 to 20 carbon atoms, a heteroaryl having 3 to 20 carbon atoms, and a cycloalkyl having 3 to 10 carbon atoms;
m is the number of sequentially linked L, and m is selected from 0, 1, and 2;
n₁ is the number of R₁, and n₁ is selected from 0, 1, 2, 3, and 4; when n₁ is greater than 1, any two R₁ are the same or different; optionally, any two adjacent R₁ form a ring;
n₂ is the number of R₂, and n₂ is selected from 0, 1, 2, 3, and 4; when n₂ is greater than 1, any two R₂ are the same or different; optionally, any two adjacent R₂ form a ring;
n₃ is the number of R₃, and n₃ is selected from 0, 1, 2, 3, and 4; when n₃ is greater than 1, any two R₃ are the same or different; optionally, any two adjacent R₃ form a ring;
L, L₁, and L₂ are the same or different, and are each independently selected from a single bond, a substituted or unsubstituted arylene having 6 to 30 carbon atoms, and a substituted or unsubstituted heteroarylene having 3 to 30 carbon atoms;
Ar₁ and Ar₂ are each independently selected from a substituted or unsubstituted aryl having 6 to 30 carbon atoms and a substituted or unsubstituted heteroaryl having 3 to 30 carbon atoms;
substituent(s) in L, L₁, L₂, Ar₁ and Ar₂ are the same or different, and are each independently selected from a deuterium, a cyano, a halogen group, an alkyl having 1 to 10 carbon atoms, a haloalkyl having 1 to 10 carbon atoms, a deuteroalkyl having 1 to 10 carbon atoms, a trialkylsilyl having 3 to 12 carbon atoms, an aryl having 6 to 20 carbon atoms, a heteroaryl having 3 to 20 carbon atoms, and a cycloalkyl having 3 to 10 carbon atoms; optionally, any two adjacent substituents in Ar₁ form a saturated or unsaturated 3-membered to 15-membered ring; optionally, any two adjacent substituents in Ar₂ form a saturated or unsaturated 3-membered to 15-membered ring.

2. The organic compound according to claim 1, wherein the organic compound has the structures represented by any one of Formula I-1 to Formula I-3: 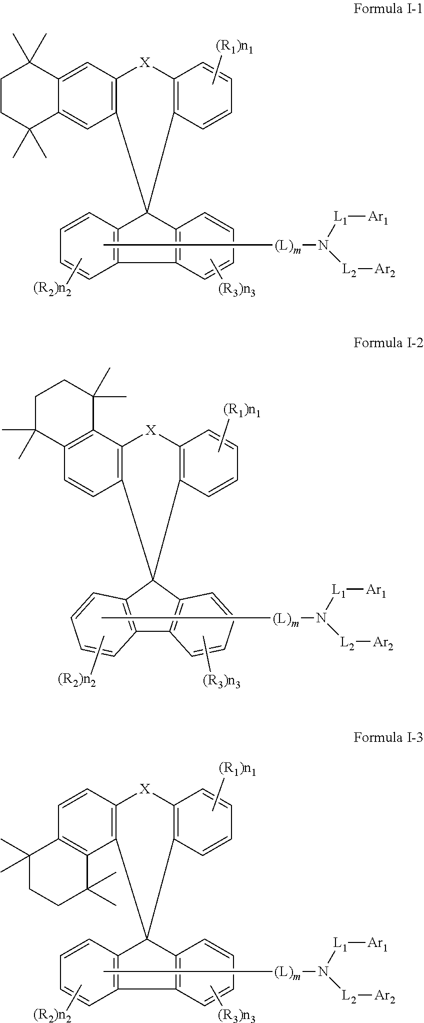
X, L, L₁, L₂, Ar₁, Ar₂, R₁, R₂, R₃, m, n₁, n₂, and n₃ have same definitions as those in Formula I.

3. The organic compound according to claim 1, wherein X is selected from a single bond, C(R₄R₅), O, and S; R₄ and R₅ are each independently selected from a methyl, an ethyl, a n-propyl, an isopropyl, and a phenyl.

4. The organic compound according to claim 1, wherein R₁, R₂, and R₃ are each independently selected from a deuterium, a fluorine, a cyano, a methyl, an ethyl, an isopropyl, a tert-butyl, a cyclohexyl, a phenyl, a deuterophenyl, and a naphthyl.

5. The organic compound according to claim 1, wherein, Ar₁ and Ar₂ are each independently selected from a substituted or unsubstituted aryl having 6 to 25 carbon atoms and a substituted or unsubstituted heteroaryl having 5 to 18 carbon atoms;
optionally, substituent(s) in Ar₁ and Ar₂ are the same or different, and are each independently selected from a deuterium, a fluorine, a cyano, a trimethylsilyl, an alkyl having 1 to 5 carbon atoms, a cycloalkyl having 5 to 10 carbon atoms, a haloalkyl having 1 to 5 carbon atoms, an aryl having 6 to 12 carbon atoms, and a heteroaryl having 5 to 12 carbon atoms; optionally, any two adjacent substituents in Ar₁ form a saturated or unsaturated 5-membered to 13-membered ring; and optionally, any two adjacent substituents in Ar₂ form a saturated or unsaturated 5-membered to 13-membered ring.

6. The organic compound according to claim 1, wherein Ar₁ and Ar₂ are each independently selected from a substituted or unsubstituted phenyl, a substituted or unsubstituted naphthyl, a substituted or unsubstituted biphenyl, a substituted or unsubstituted fluorenyl, a substituted or unsubstituted phenanthryl, a substituted or unsubstituted terphenyl, a substituted or unsubstituted dibenzofuranyl, a substituted or unsubstituted dibenzothienyl, a substituted or unsubstituted carbazolyl, and a substituted or unsubstituted spirodifluorenyl;
optionally, substituent(s) in Ar₁ and Ar₂ are each independently selected from a deuterium, a fluorine, a cyano, a trimethylsilyl, a trifluoromethyl, a methyl, an ethyl, an isopropyl, a tert-butyl, a phenyl, a naphthyl, a dibenzofuranyl, a dibenzothienyl, and a carbazolyl.

7. The organic compound according to claim 1, wherein L, L₁ and L₂ are each independently selected from a single bond, a substituted or unsubstituted phenylene, a substituted or unsubstituted naphthylene, a substituted or unsubstituted biphenylene, a substituted or unsubstituted terphenylene, a substituted or unsubstituted fluorenylene, a substituted or unsubstituted dibenzofuranylene, a substituted or unsubstituted dibenzothienylene, and a substituted or unsubstituted carbazolylene;
optionally, substituent(s) in L, L₁ and L₂ are each independently selected from a deuterium, a fluorine, a cyano, a trimethylsilyl, a trideuteromethyl, a trifluoromethyl, a methyl, an ethyl, an isopropyl, a tert-butyl, a phenyl, and a naphthyl.

8. The organic compound according to claim 1, wherein 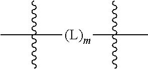
is selected from a single bond, and the group consisting of the following groups: 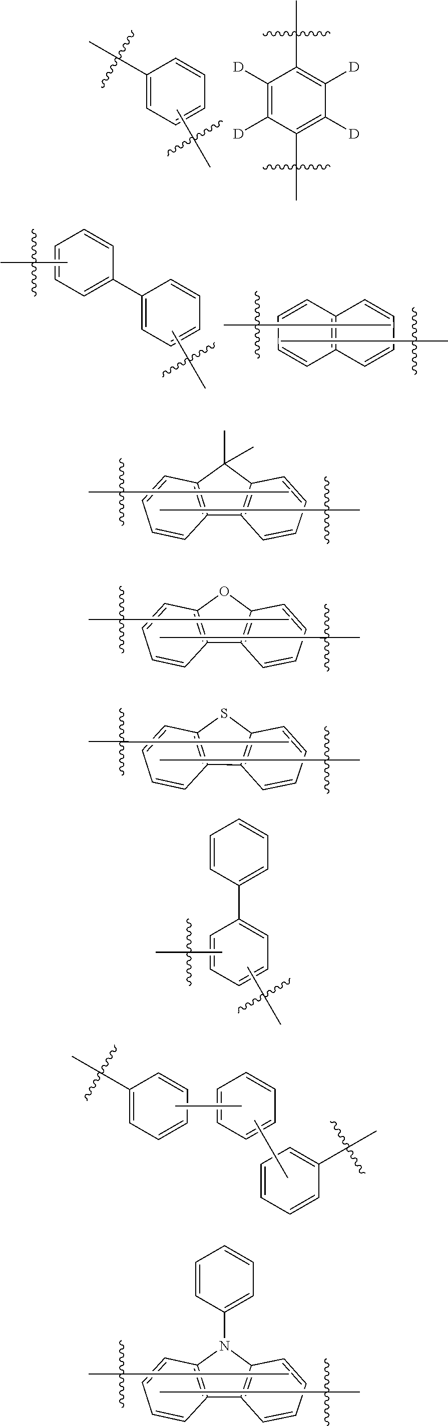 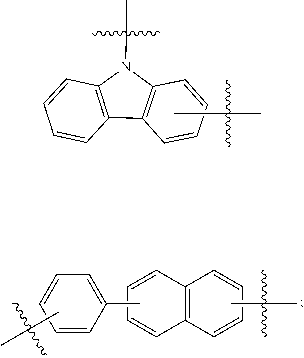
L₁ and L₂ are each independently selected from a single bond, and the group consisting of the following groups:

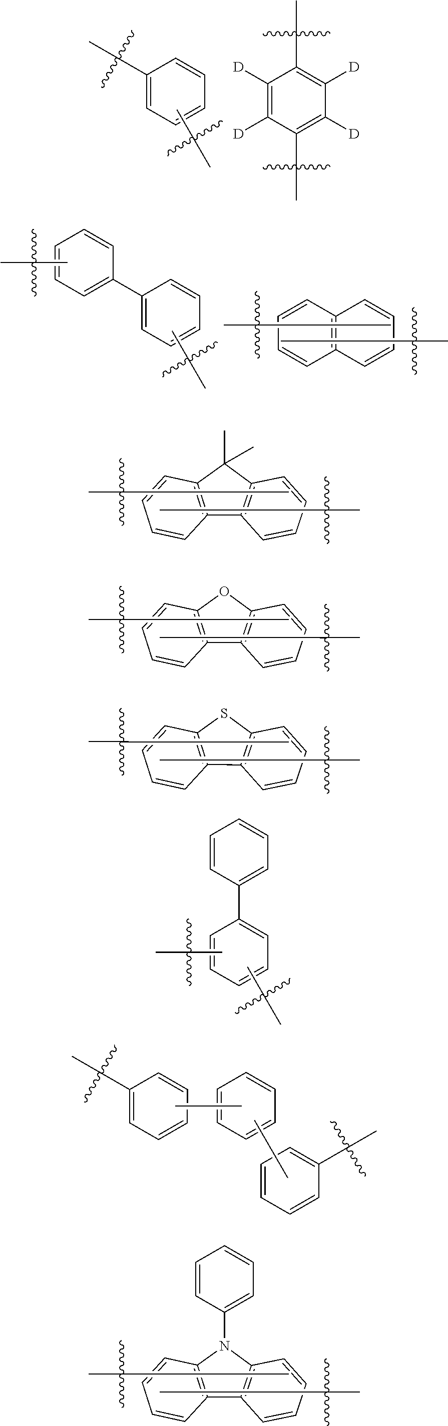

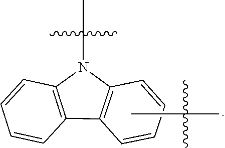

9. The organic compound according to claim 1, wherein 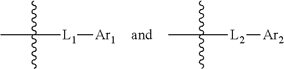
are each independently selected from the group consisting of the following groups:

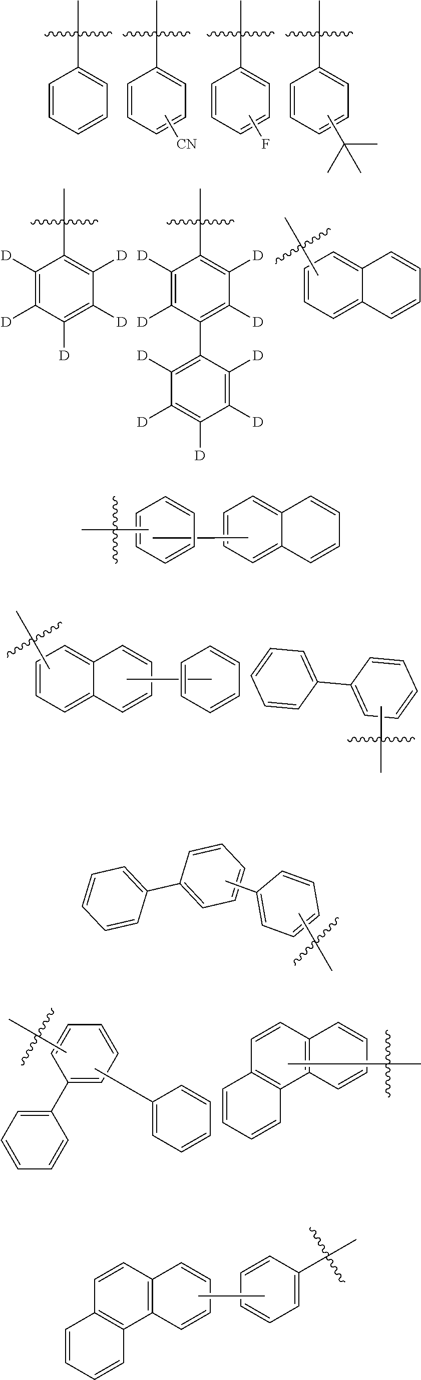

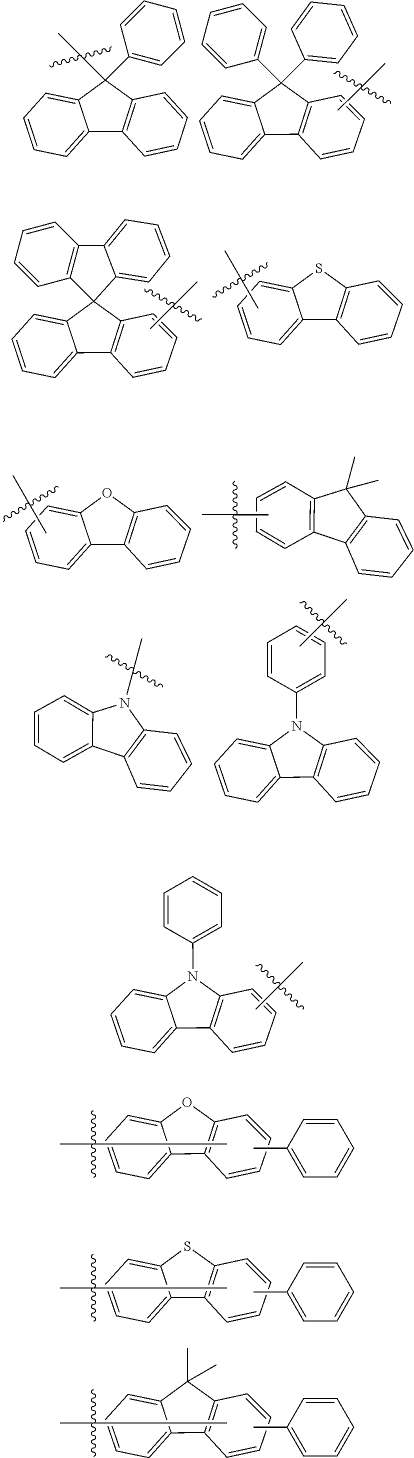

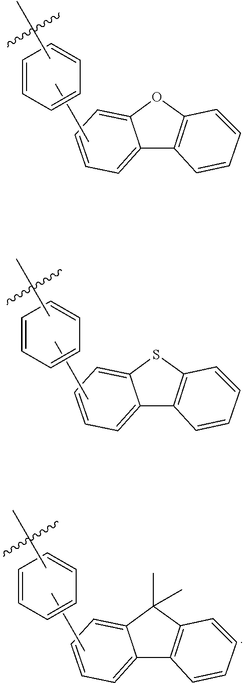

10. The organic compound according to claim 1, wherein the organic compound is selected from the group consisting of the following compounds:

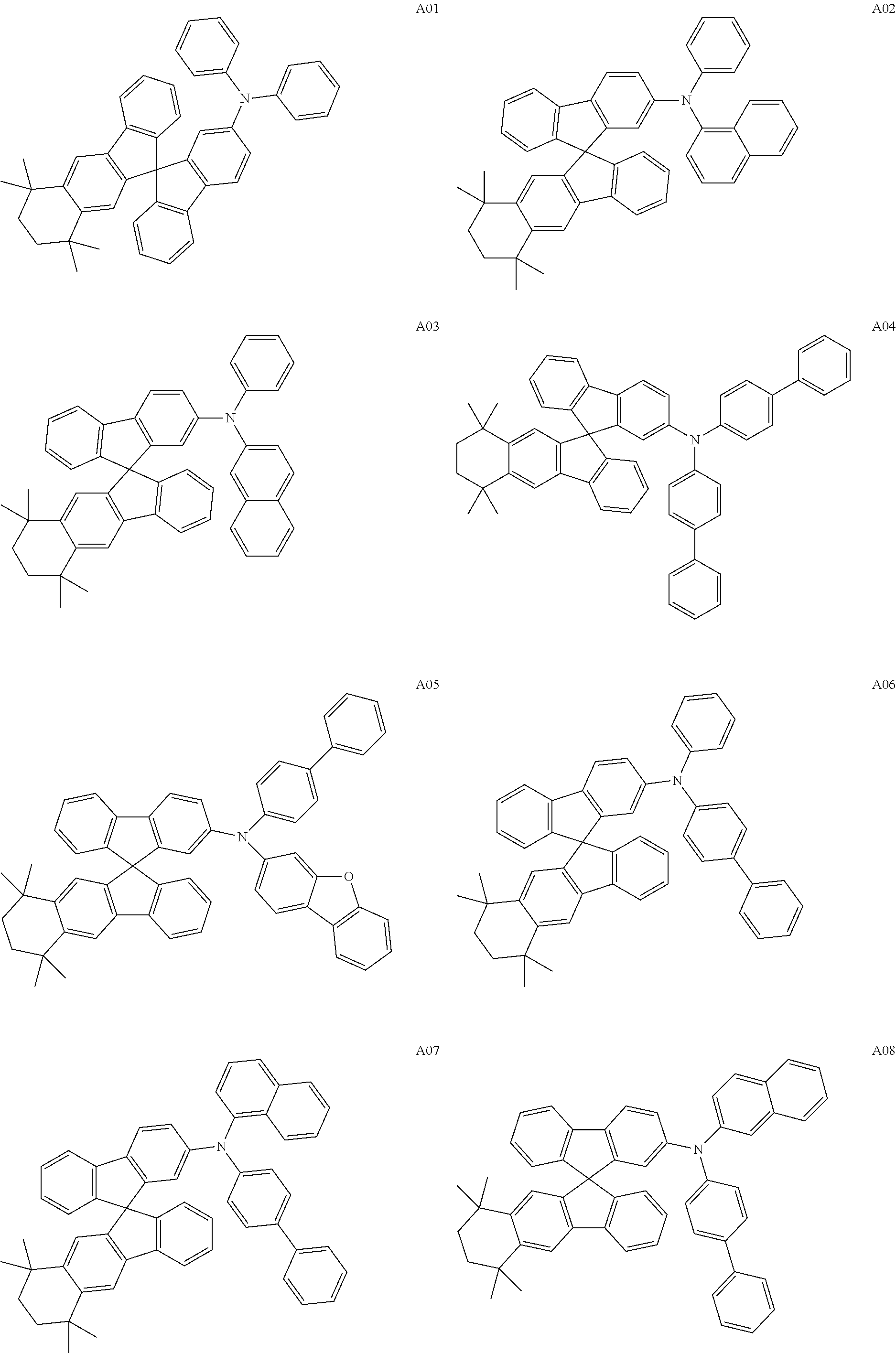

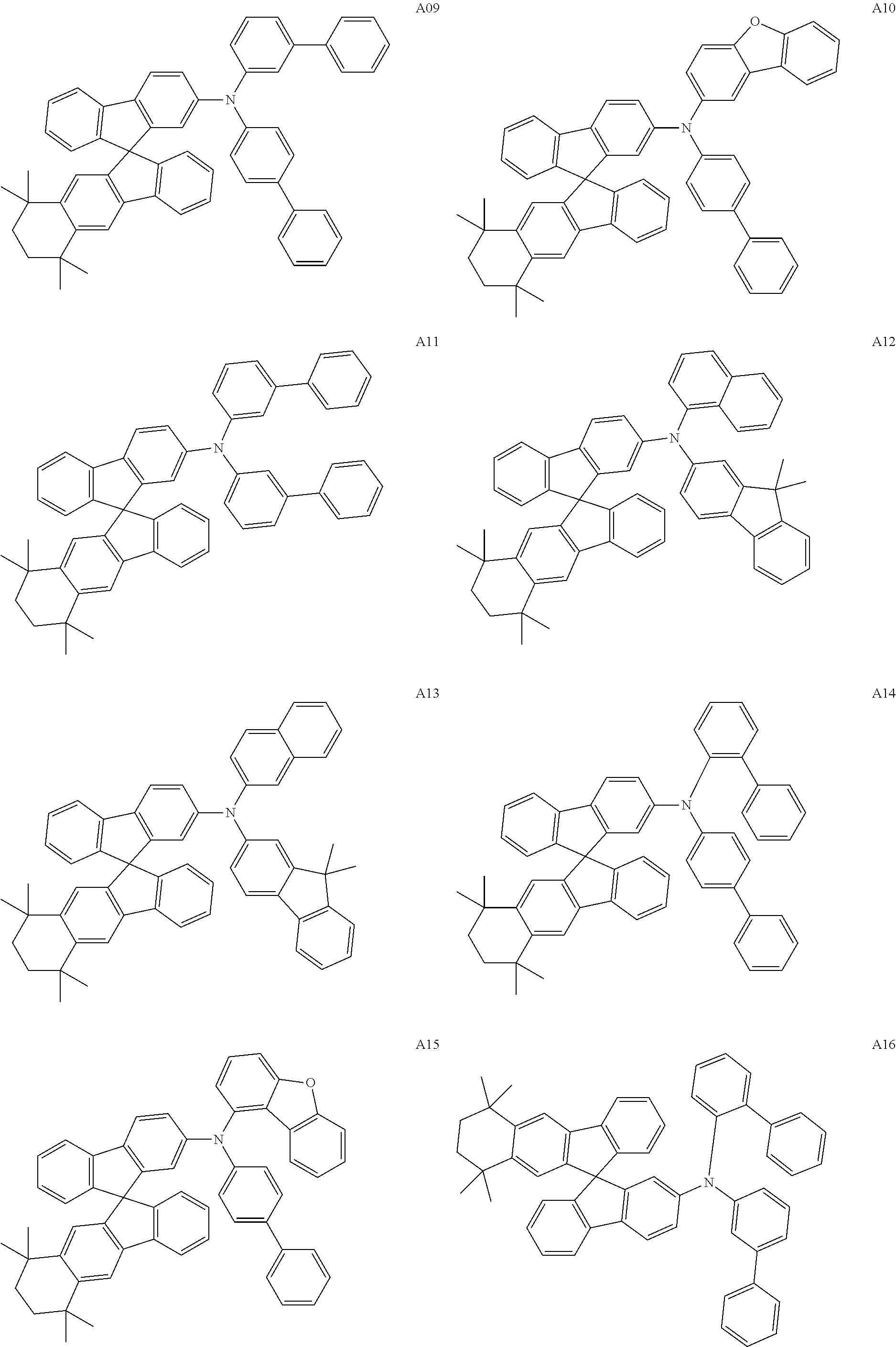

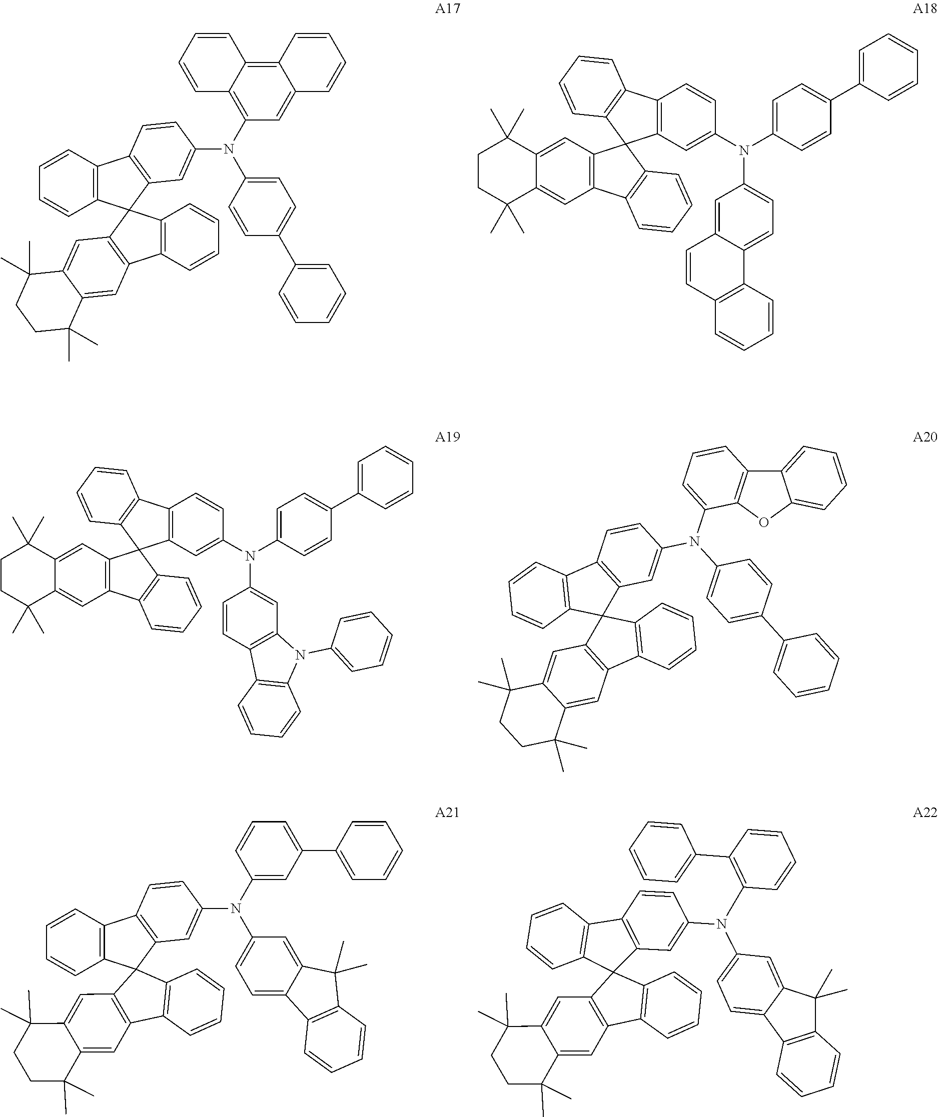

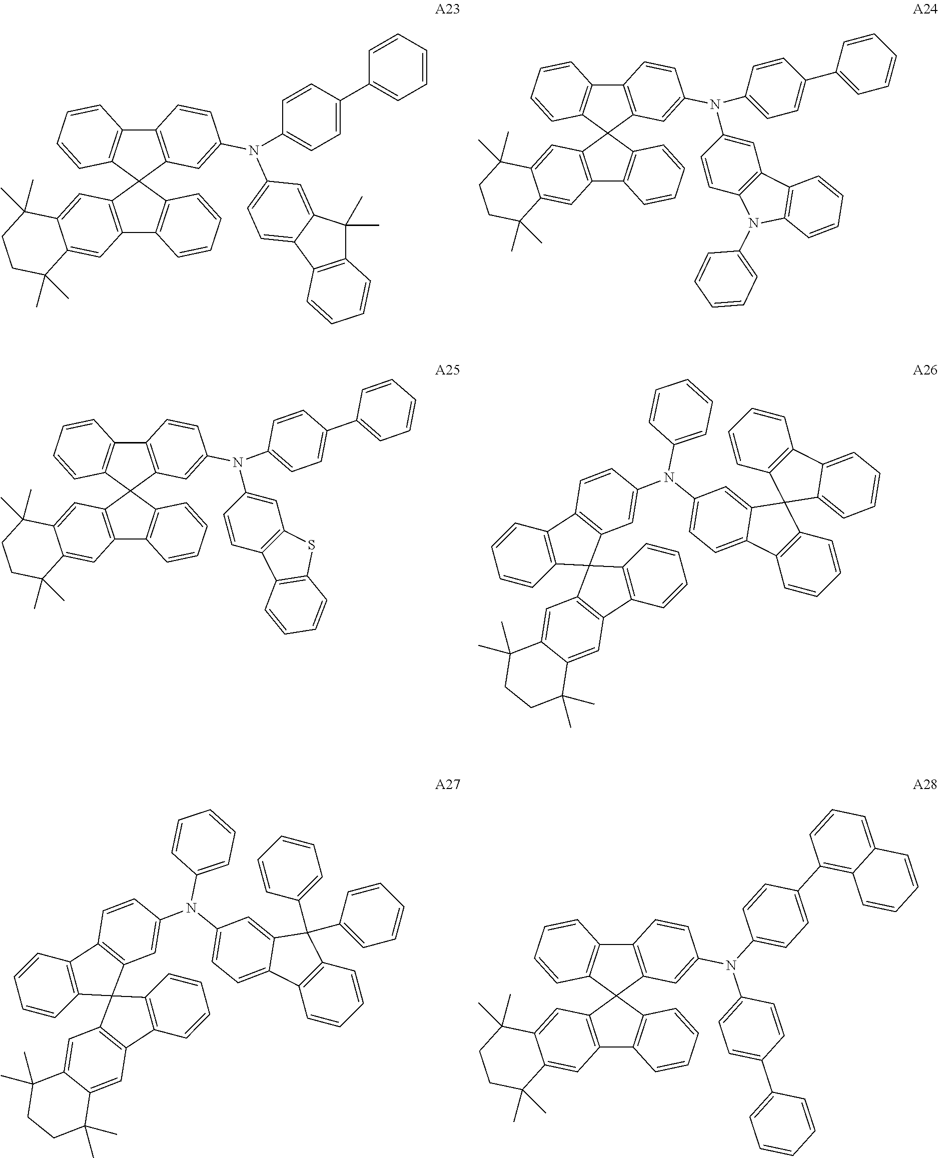

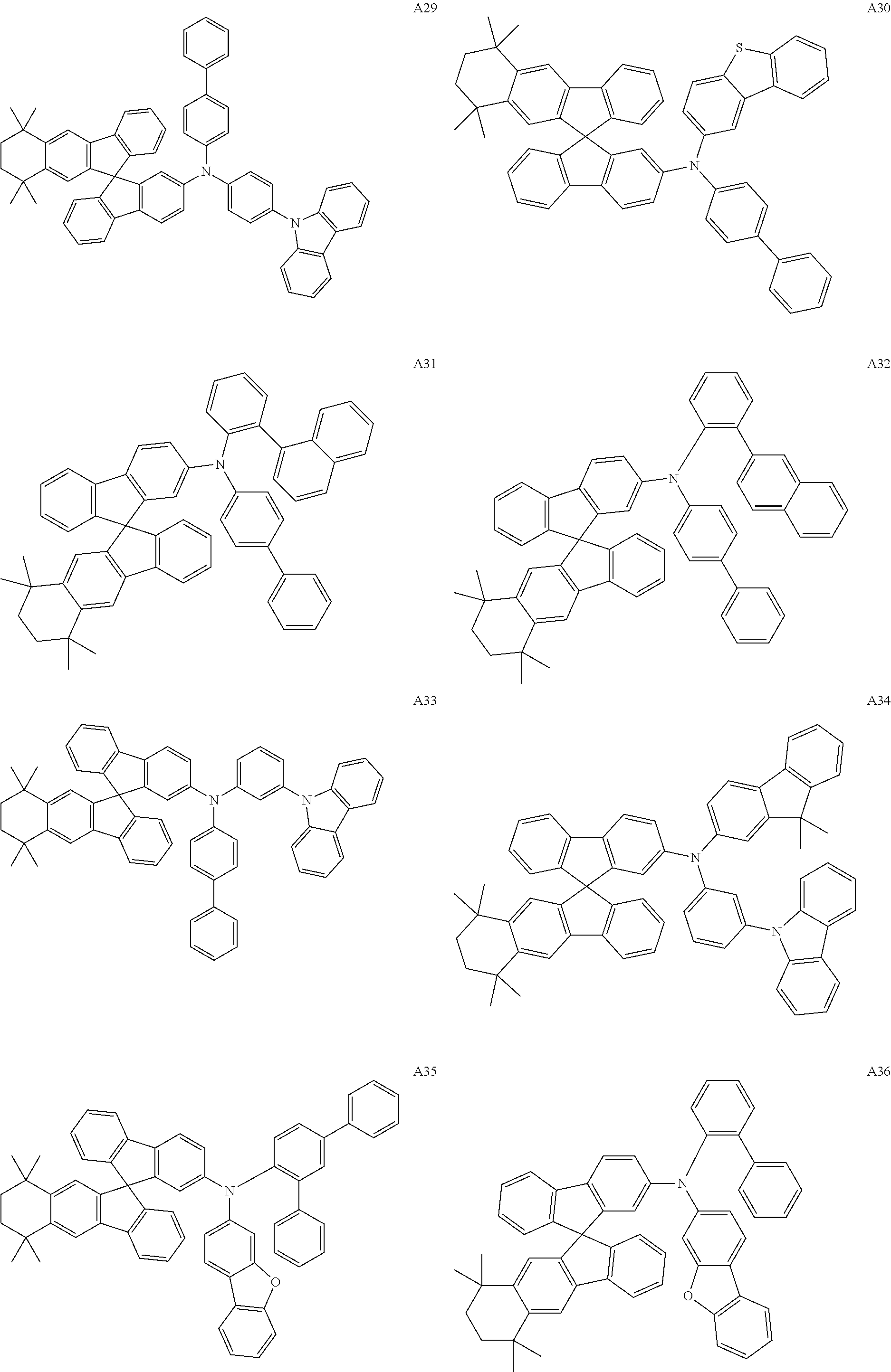

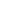

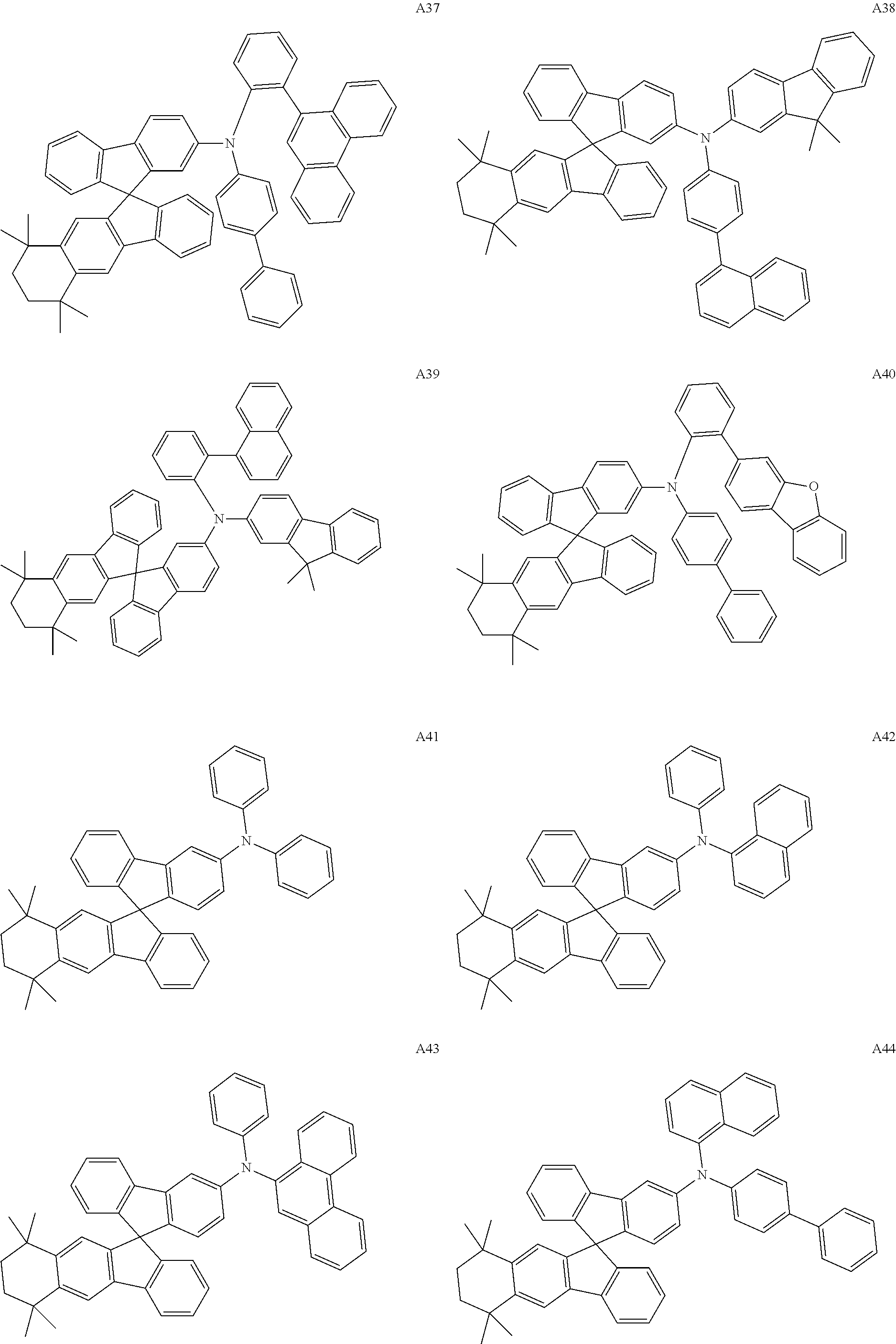

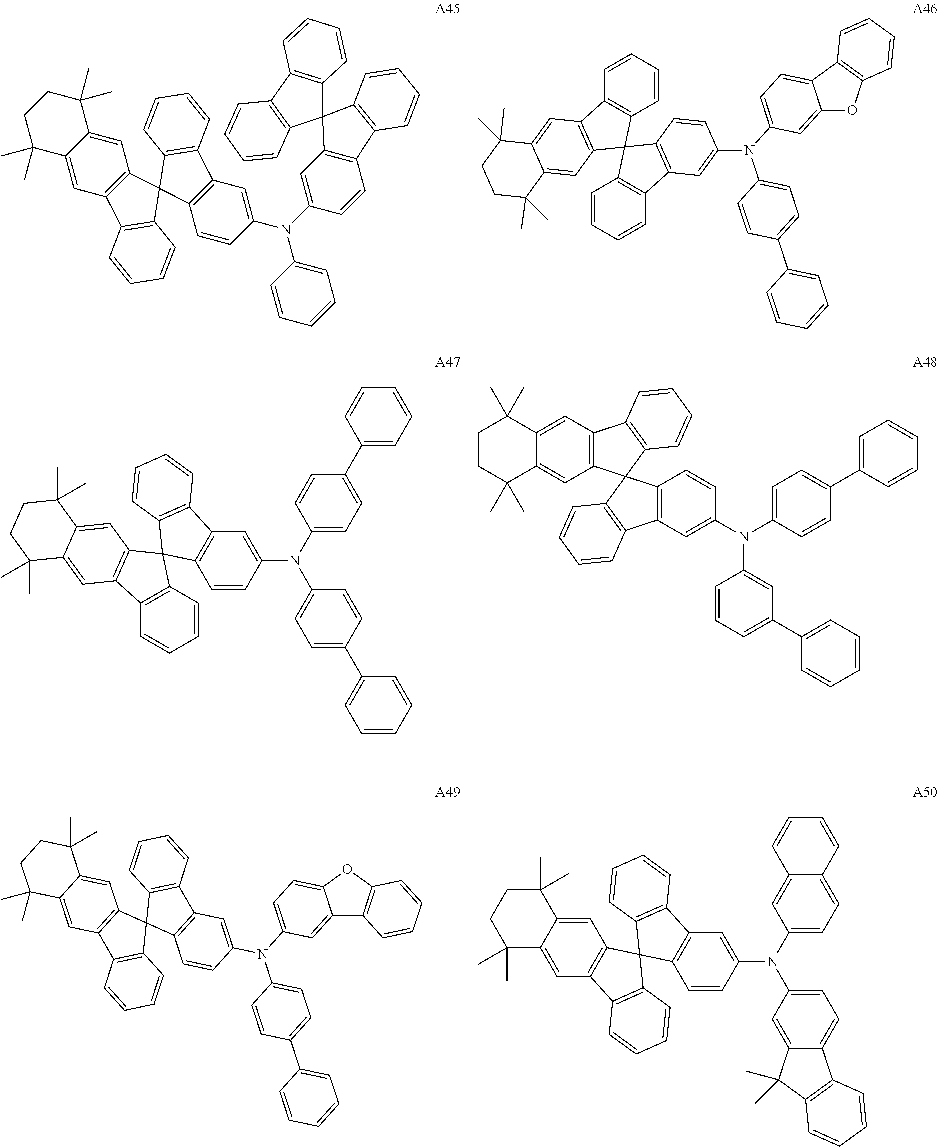

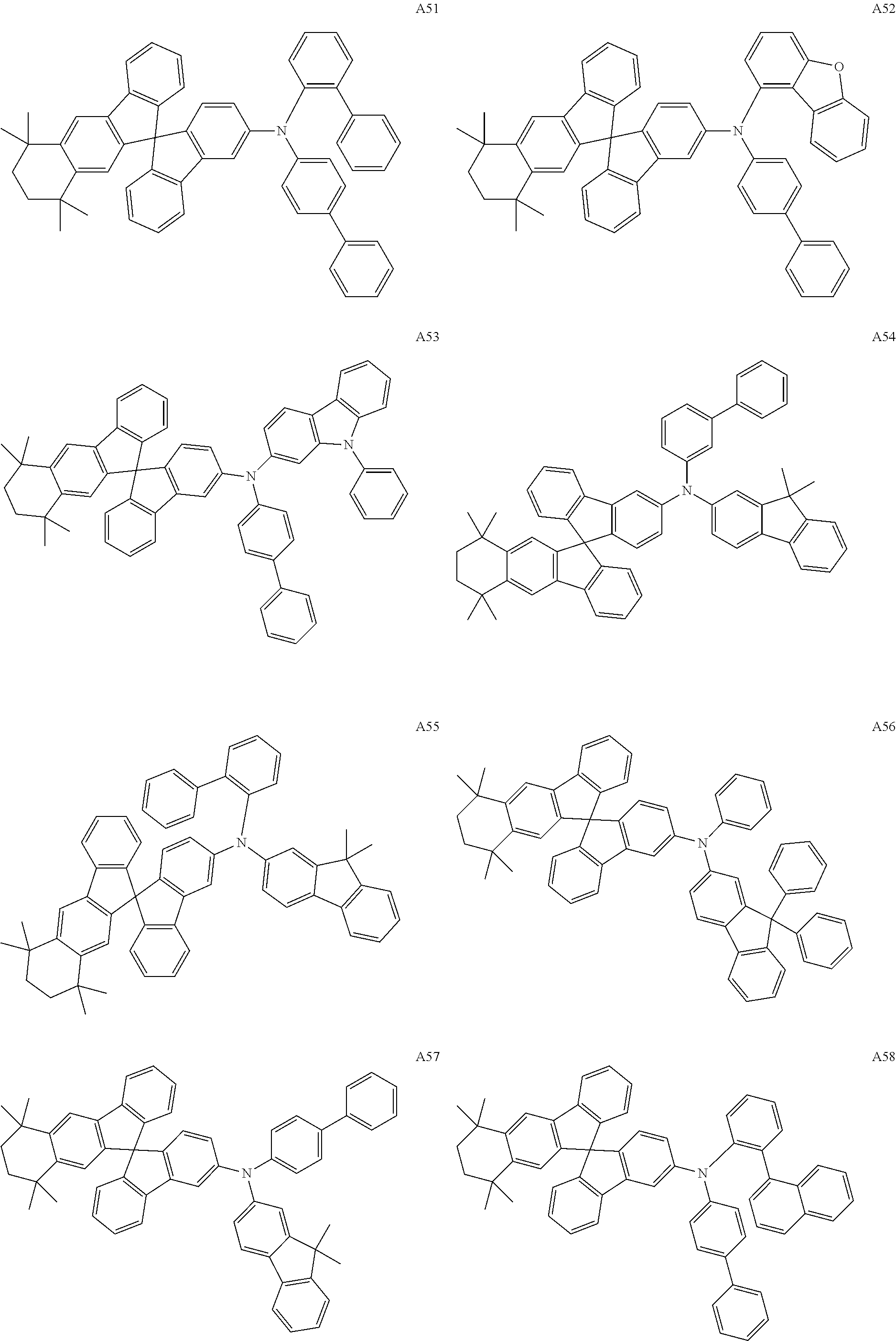

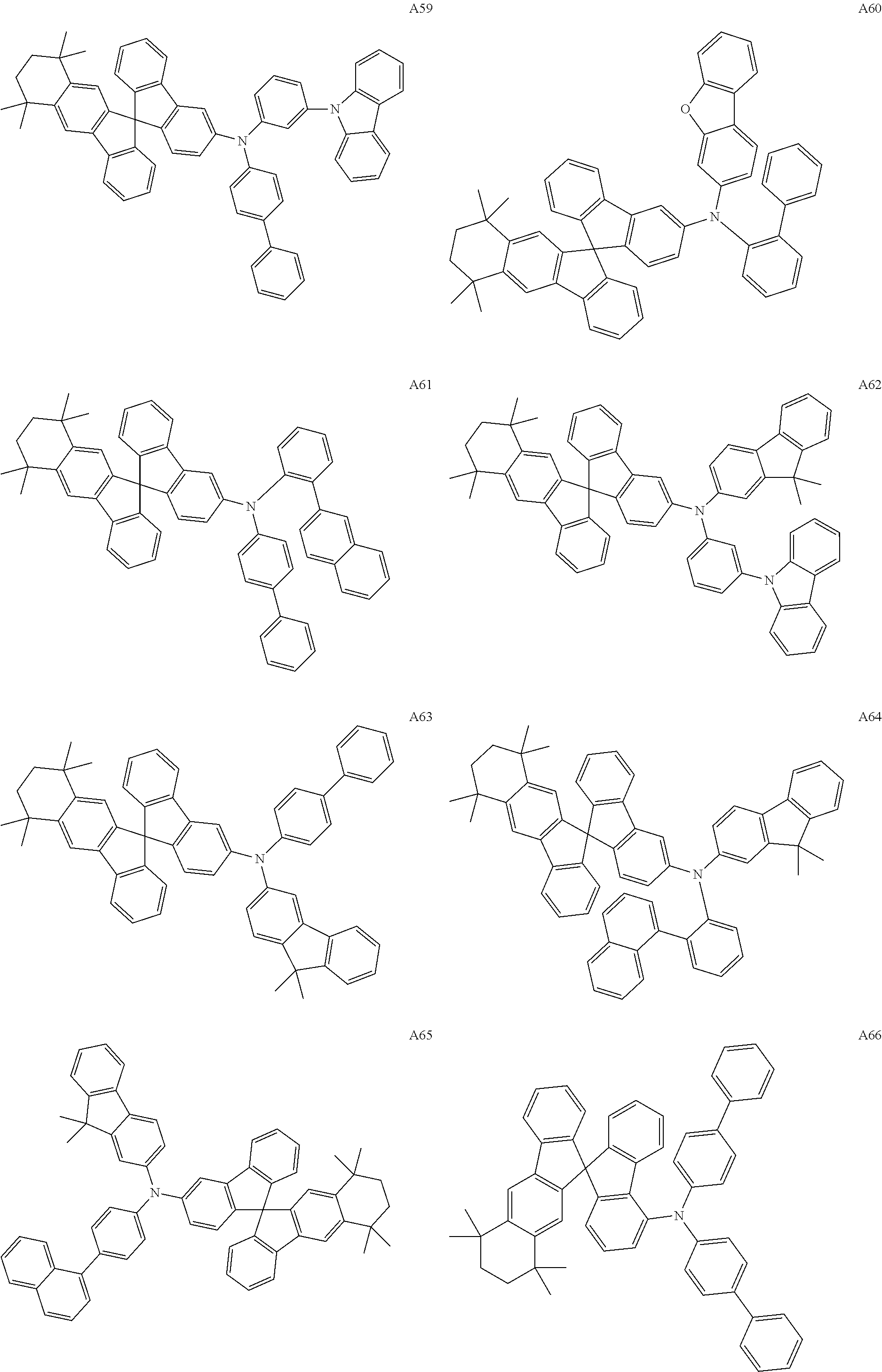

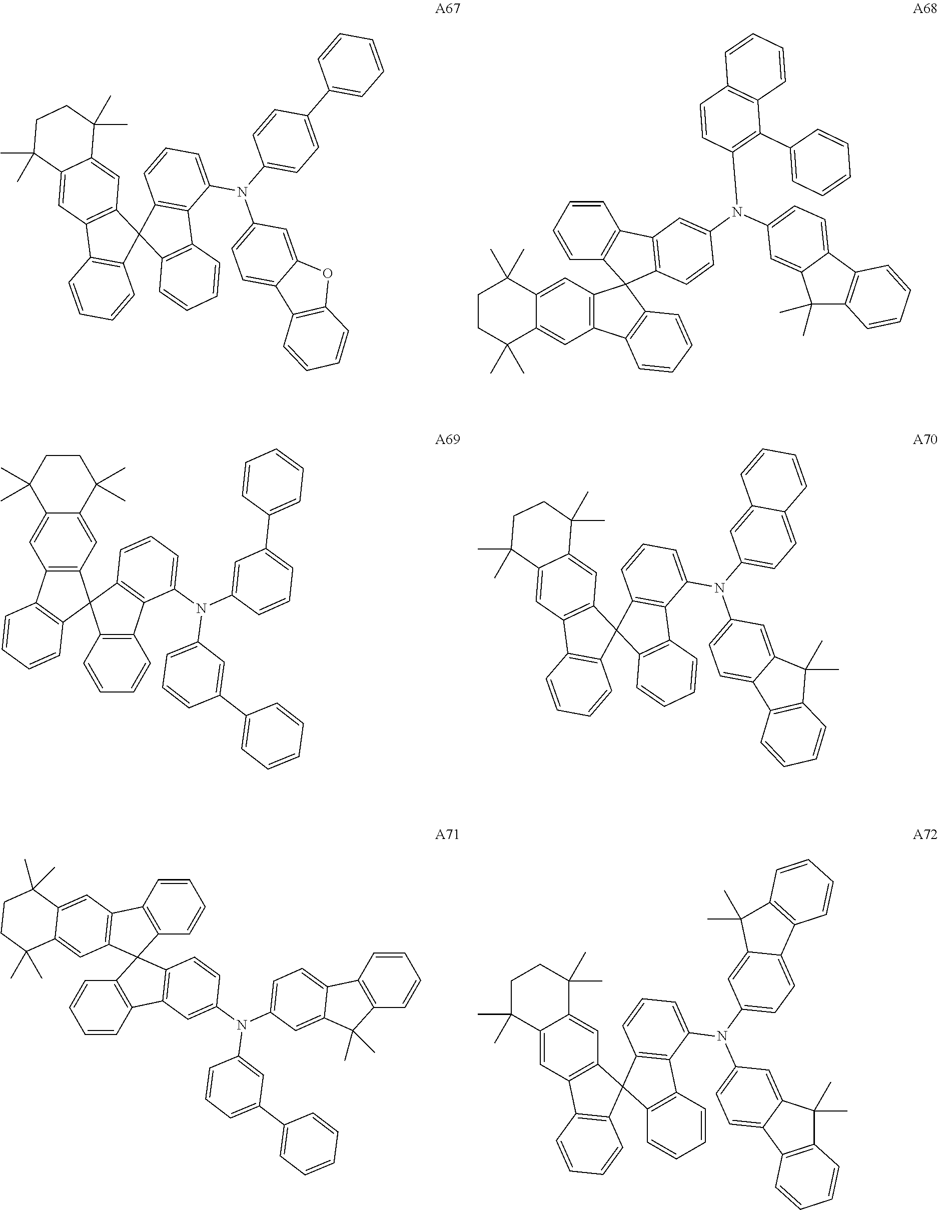

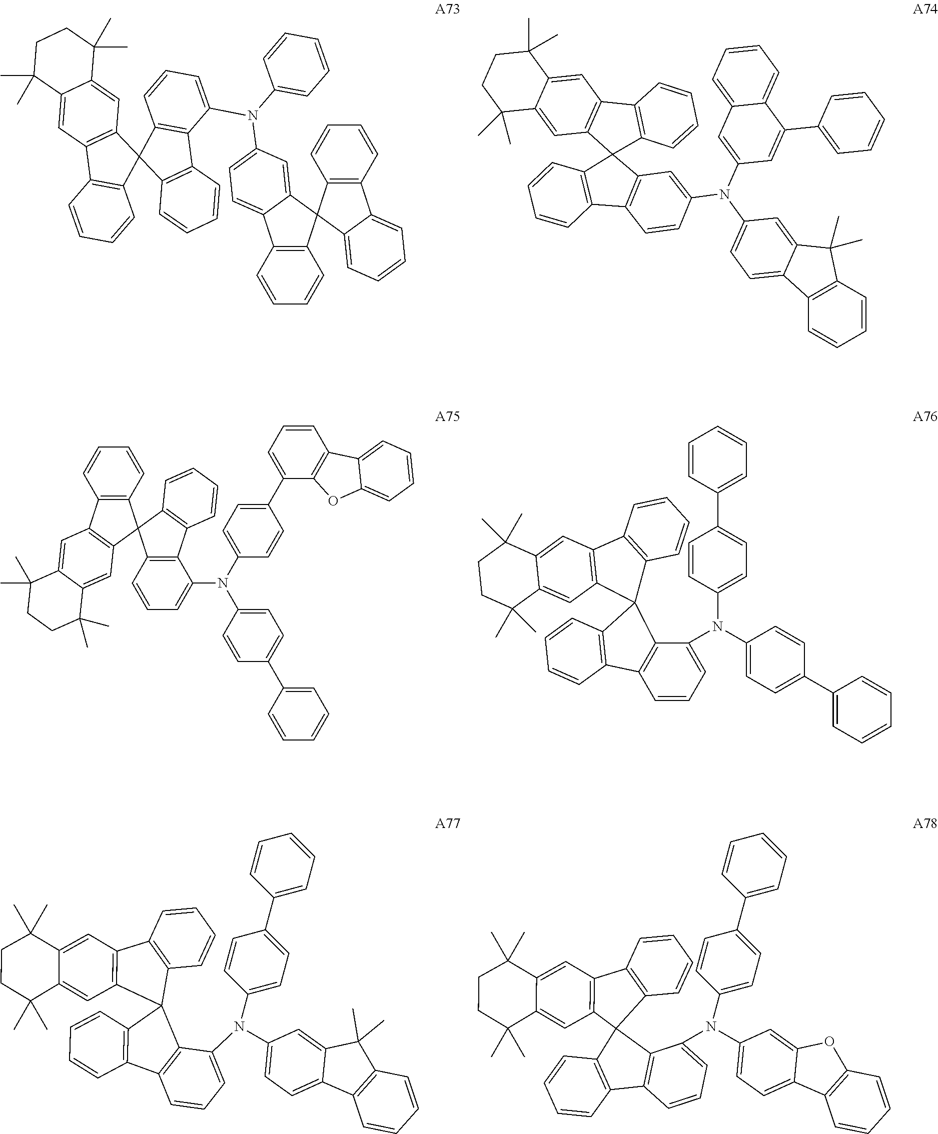

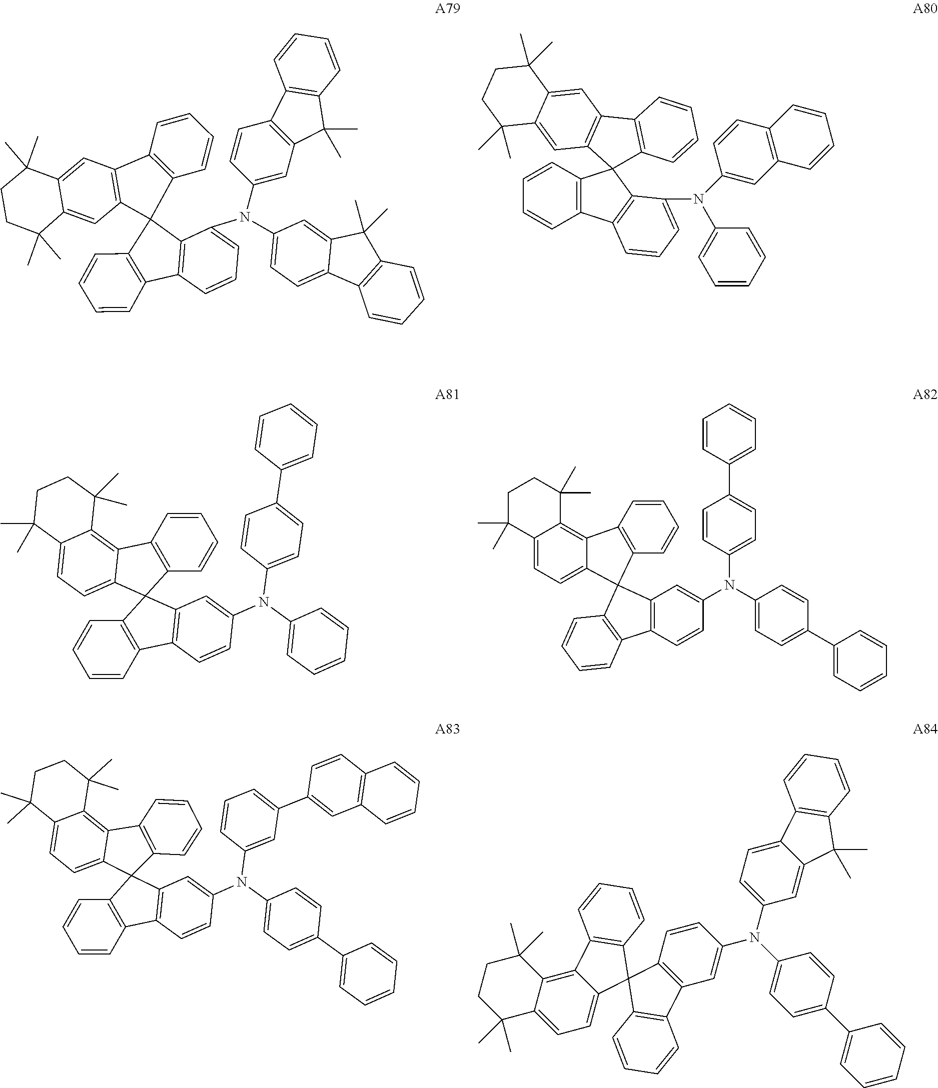

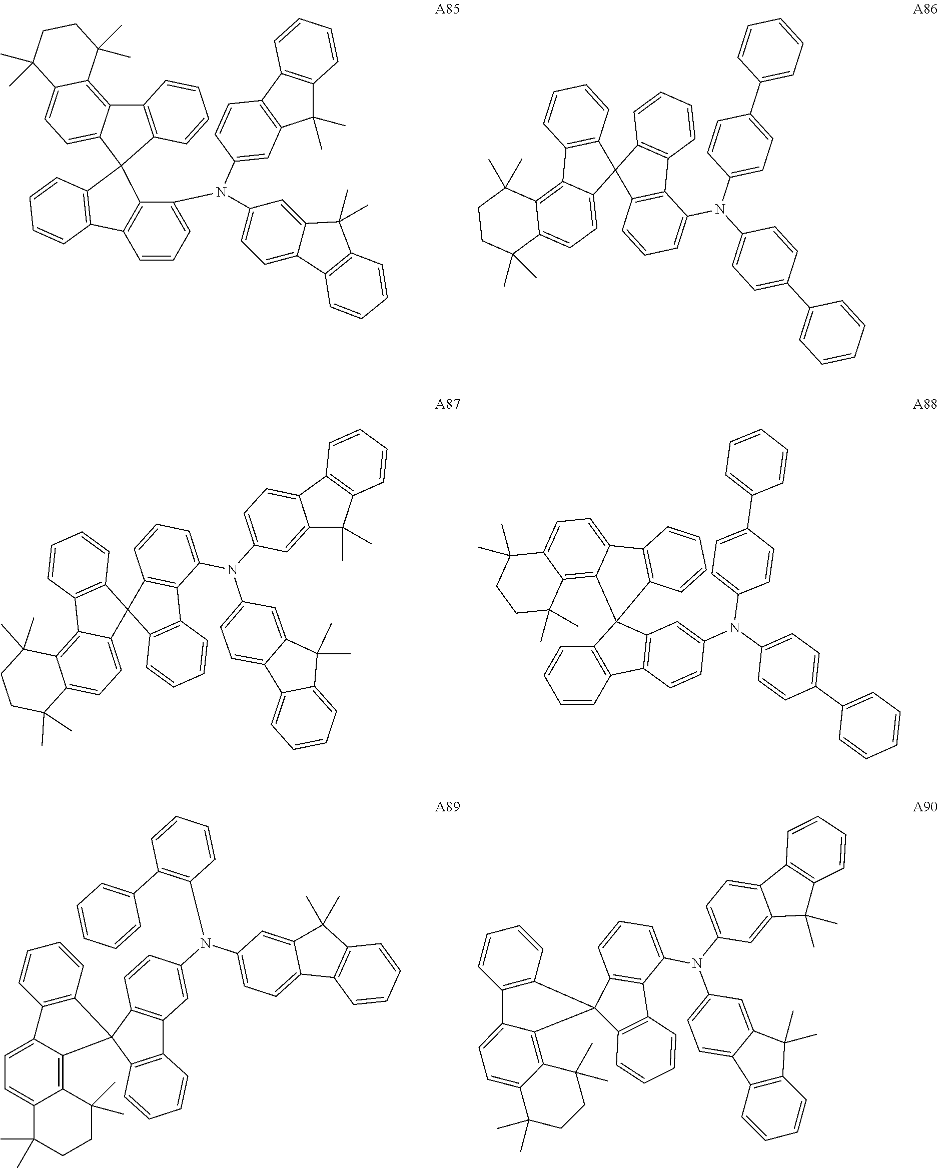

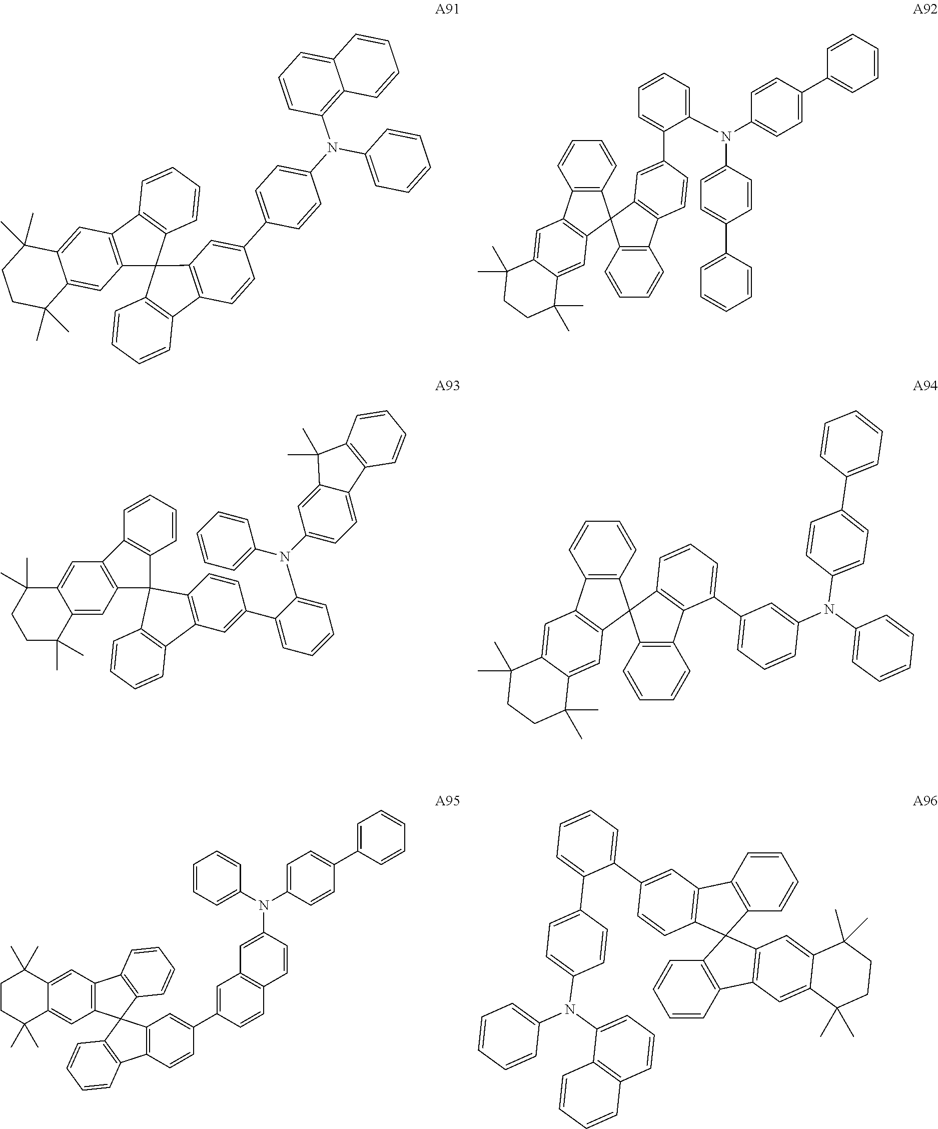

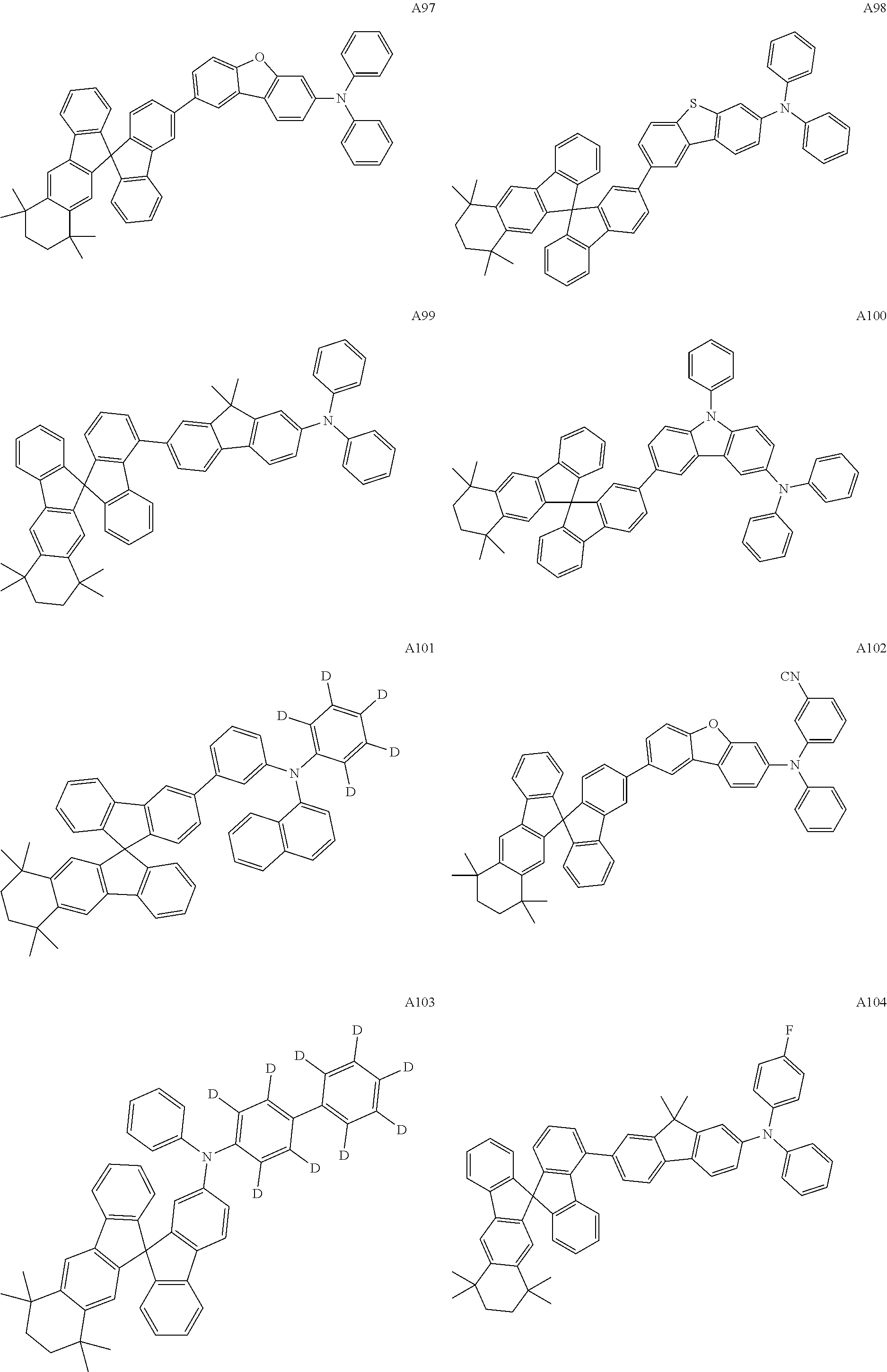

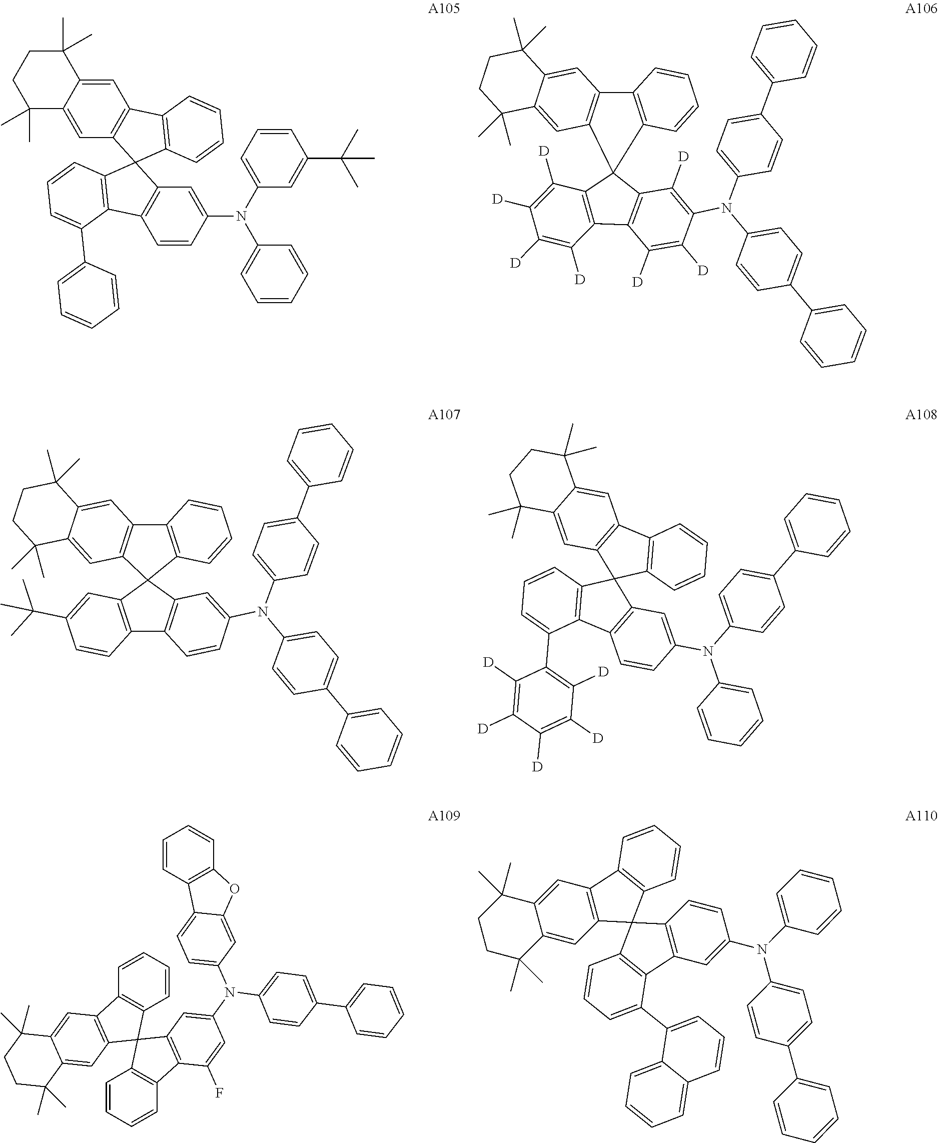

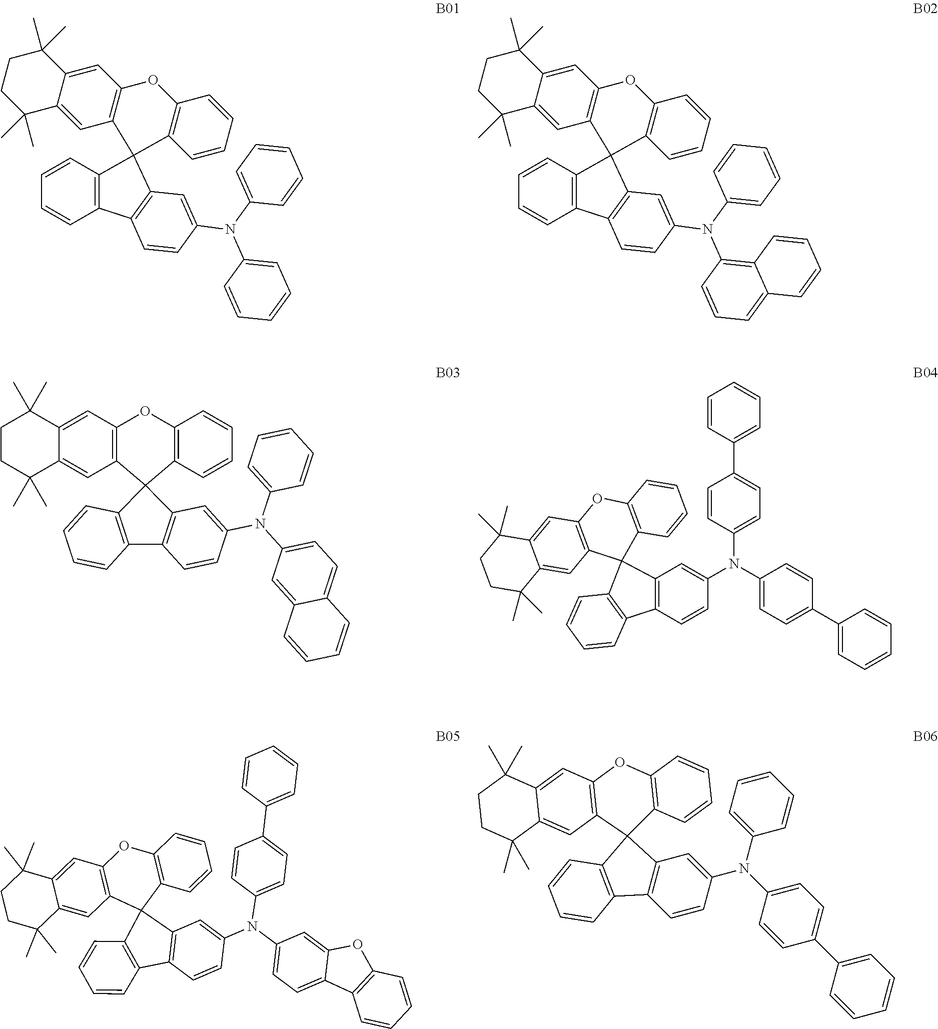

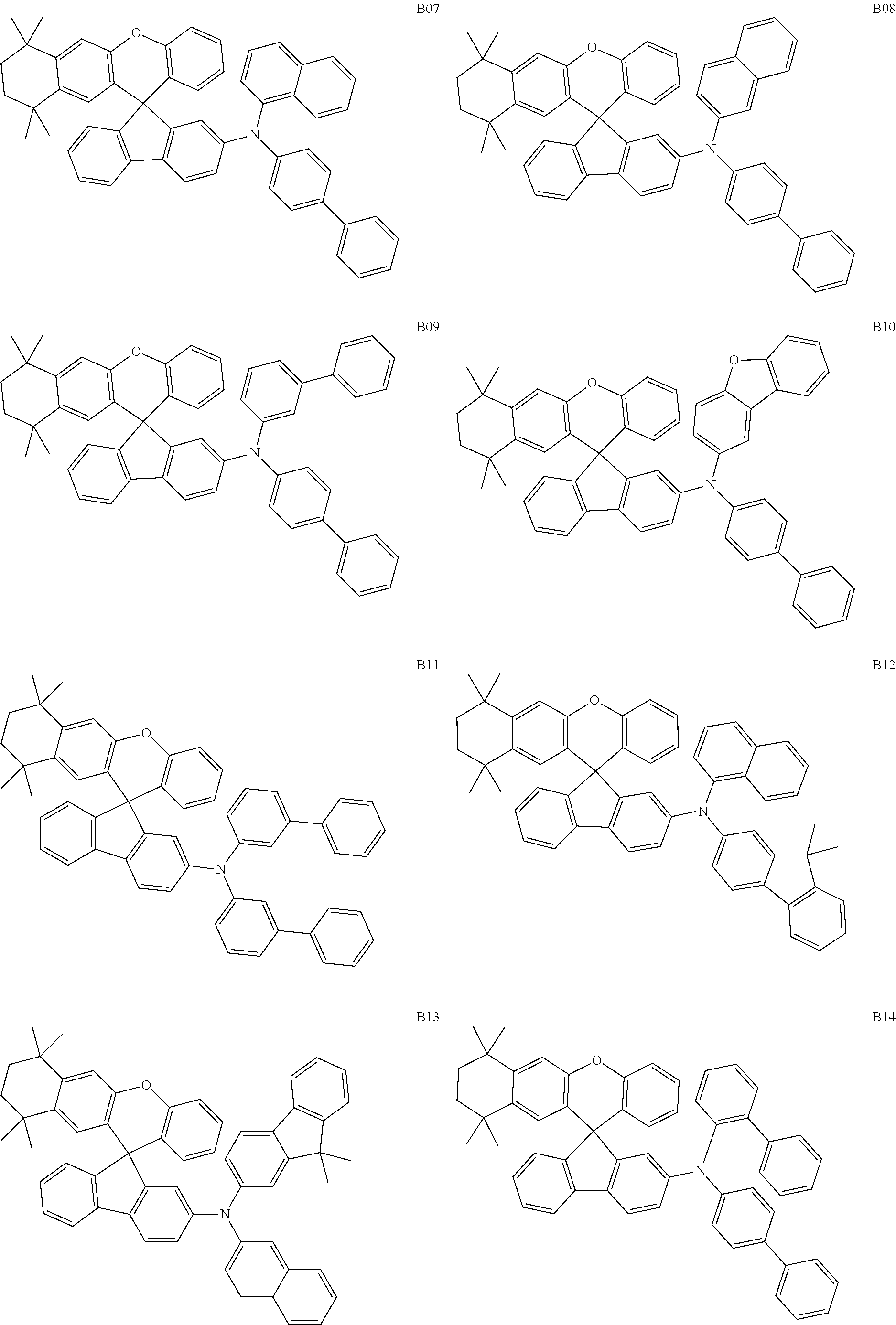

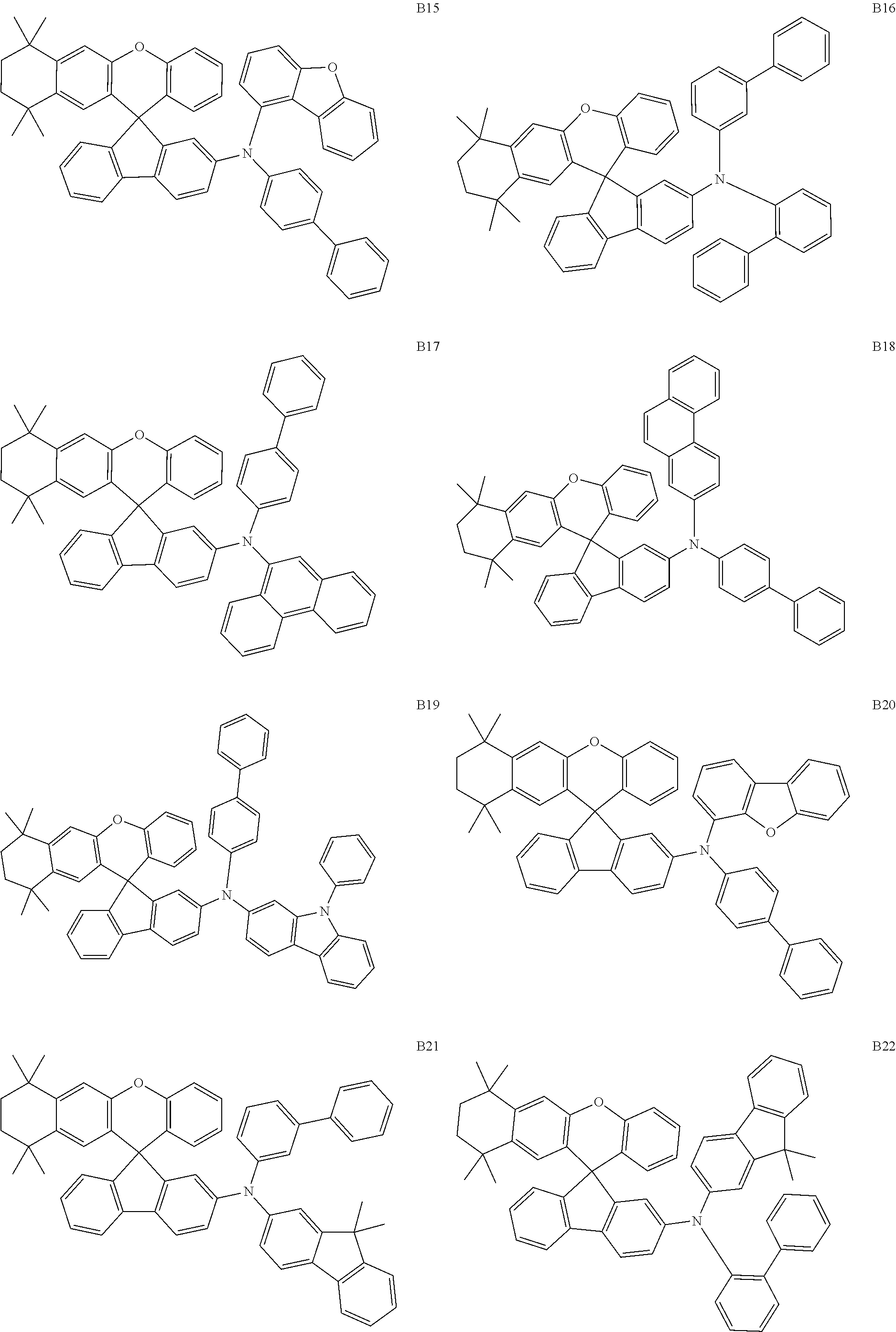

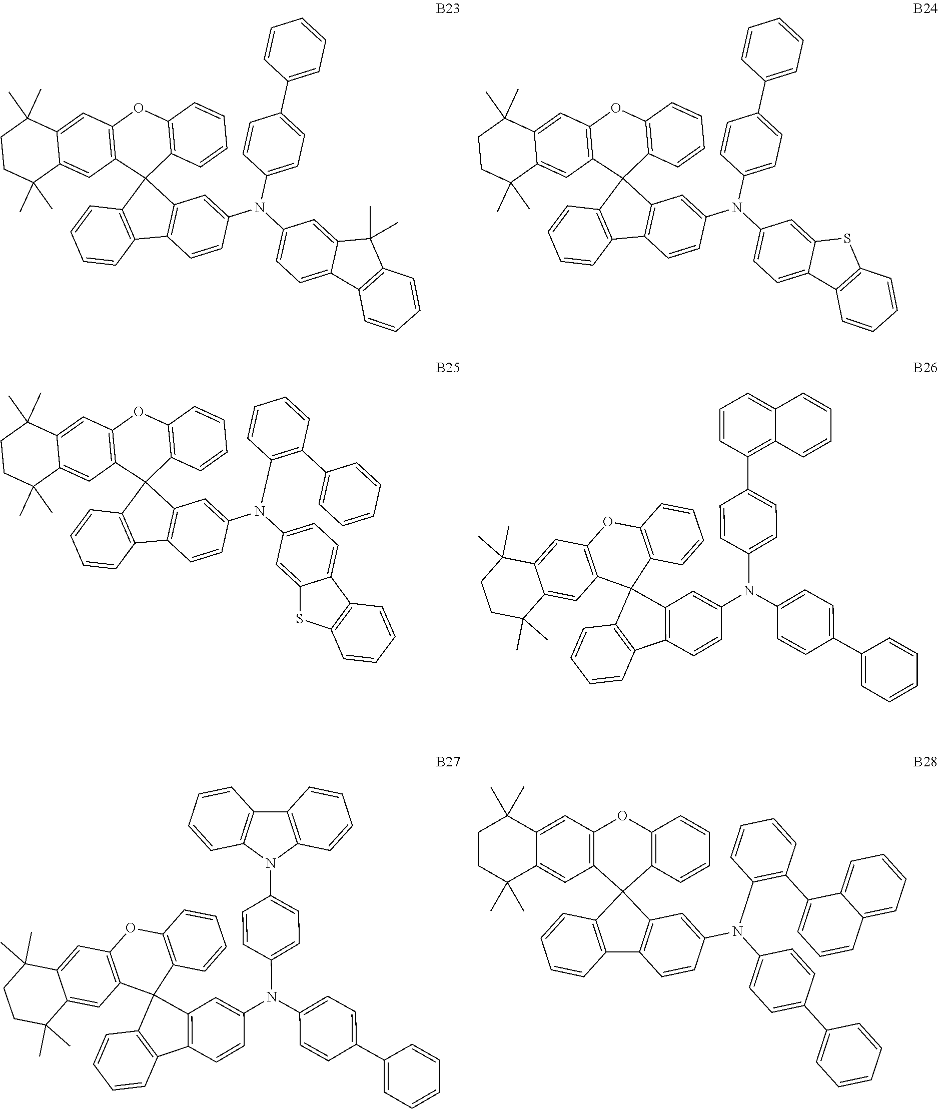

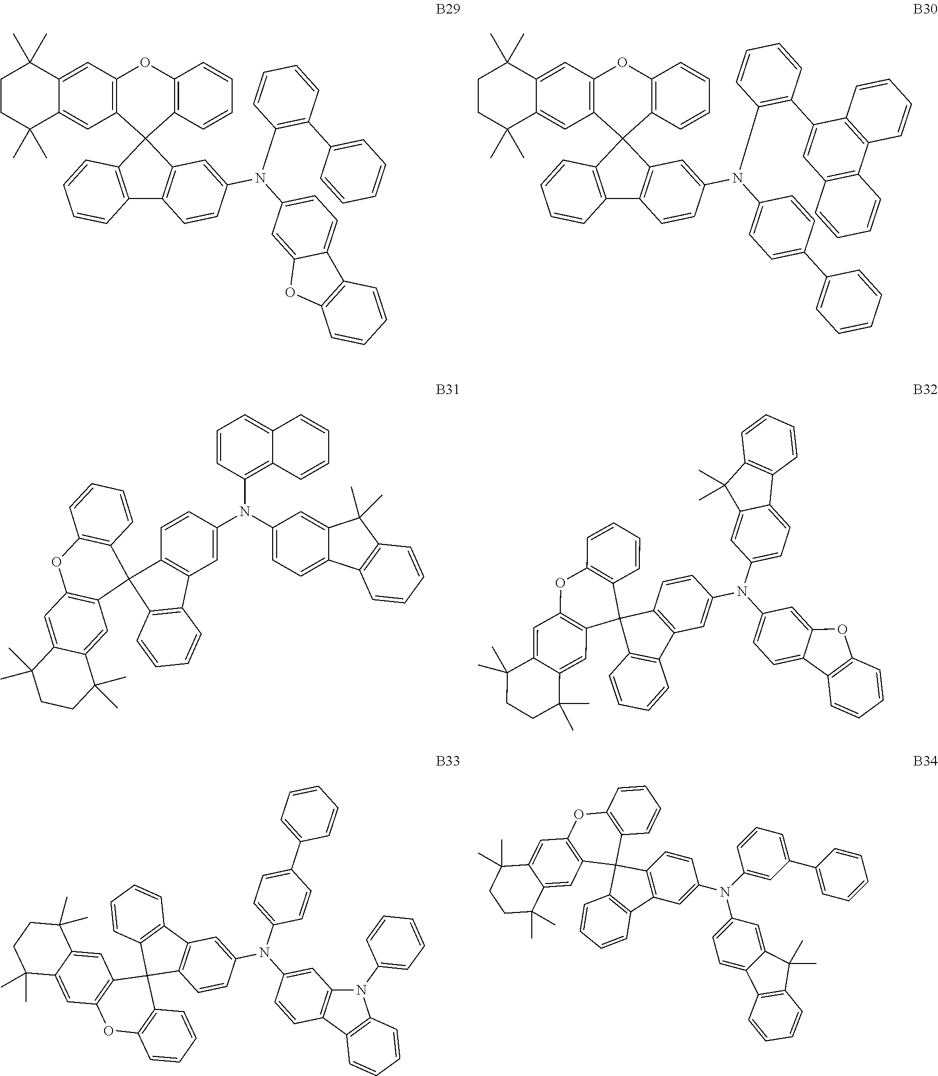

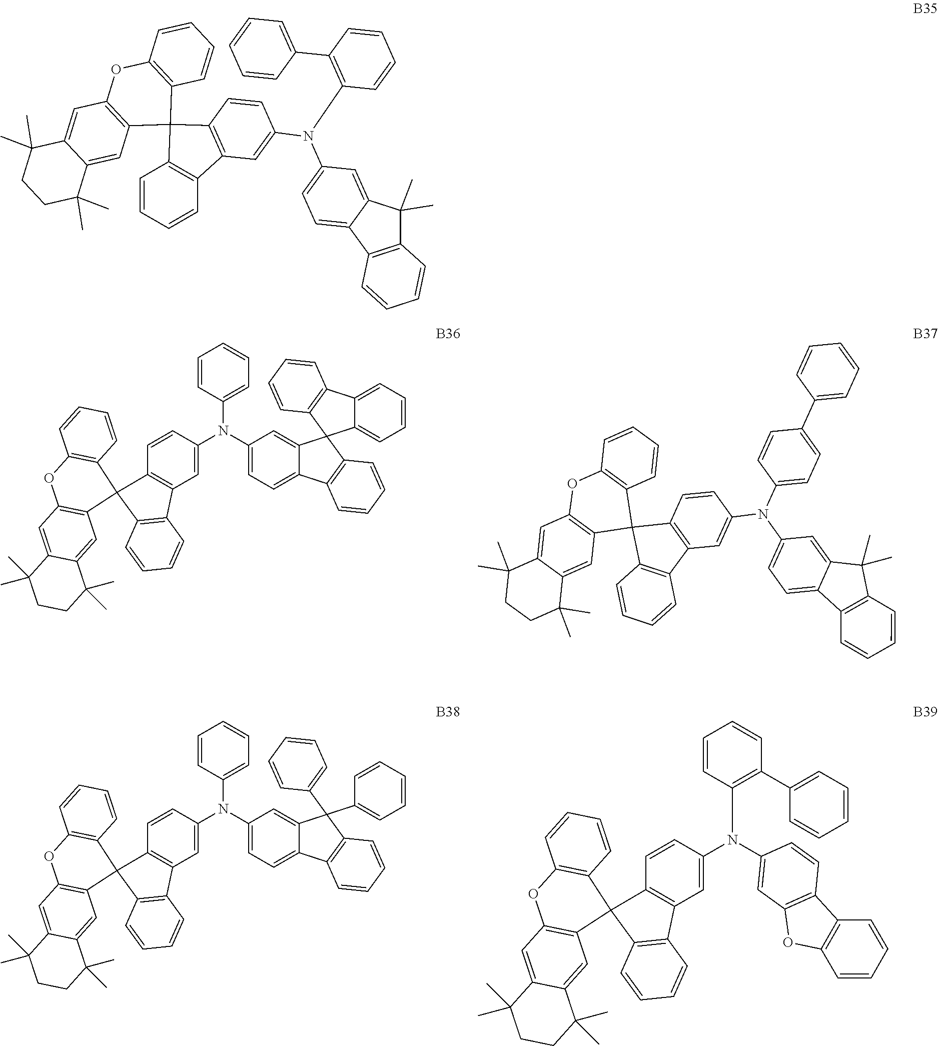

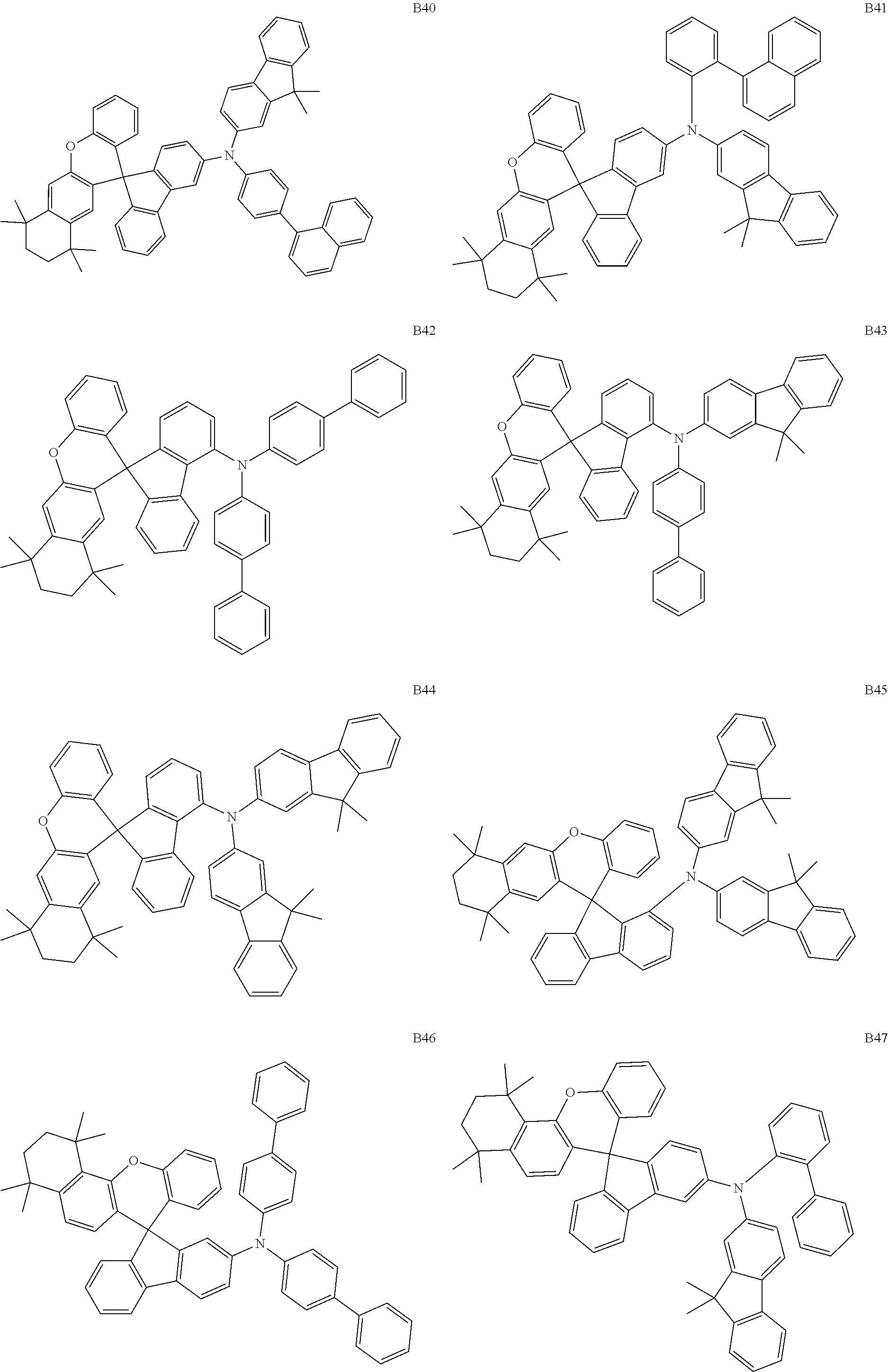

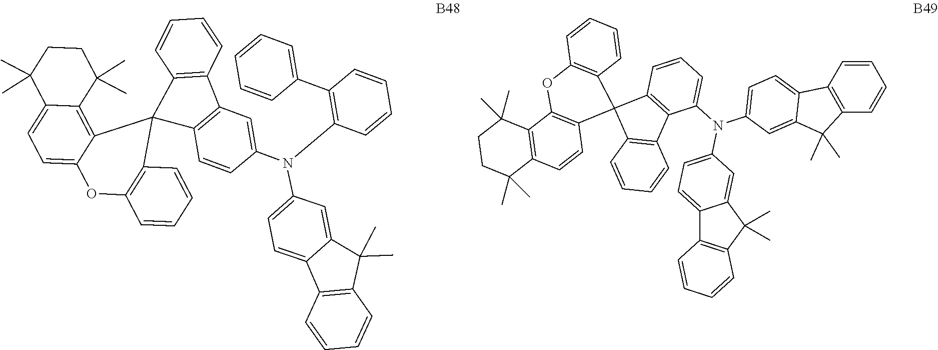

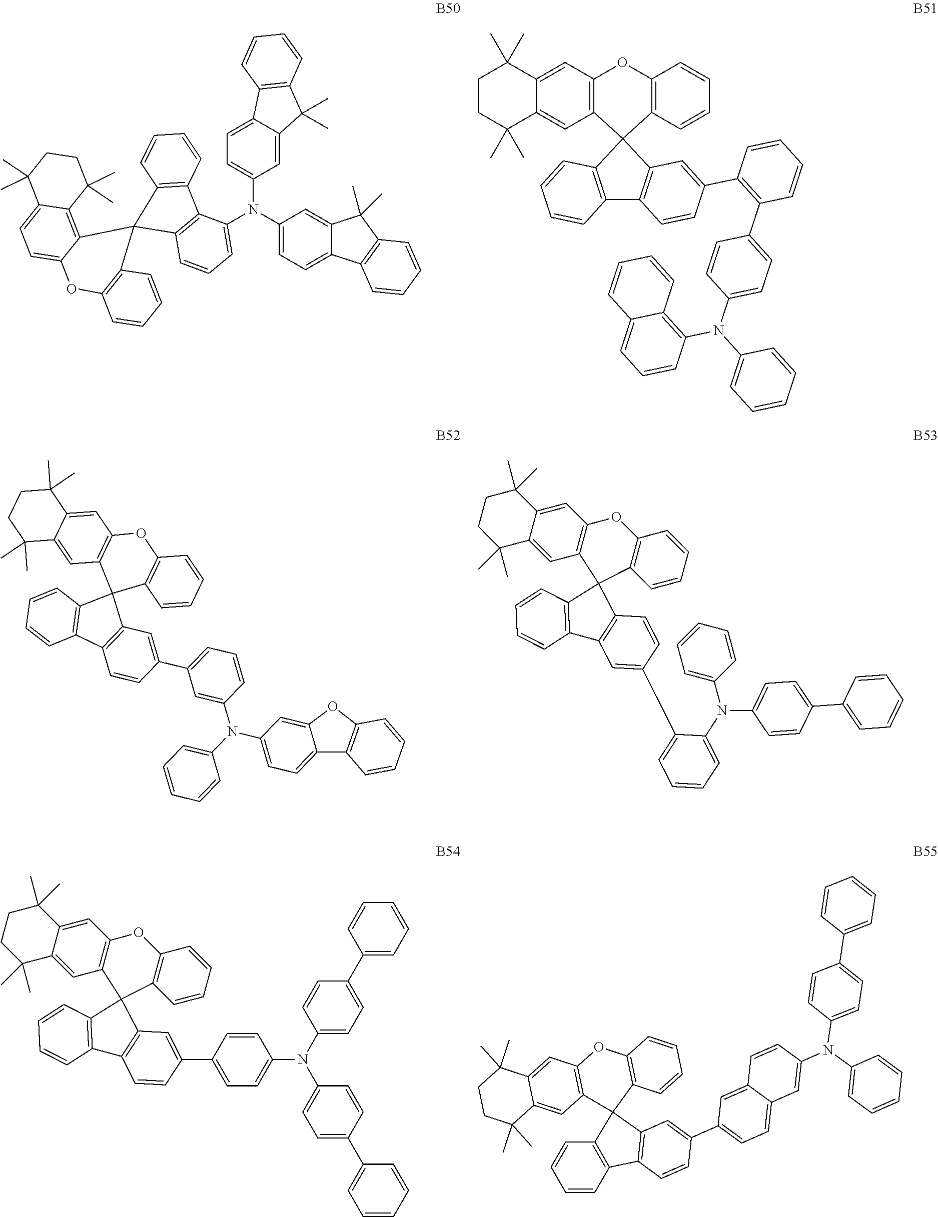

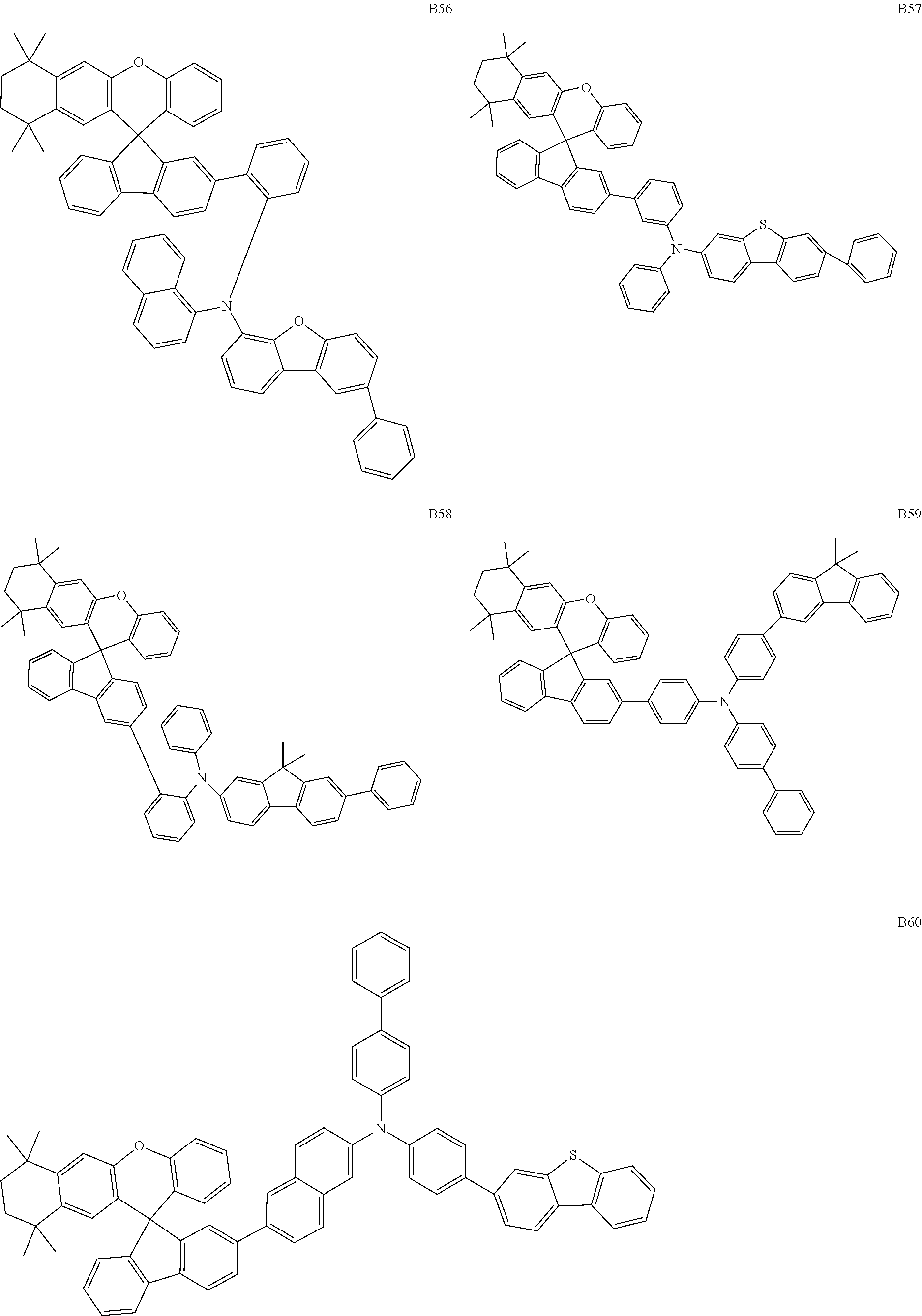

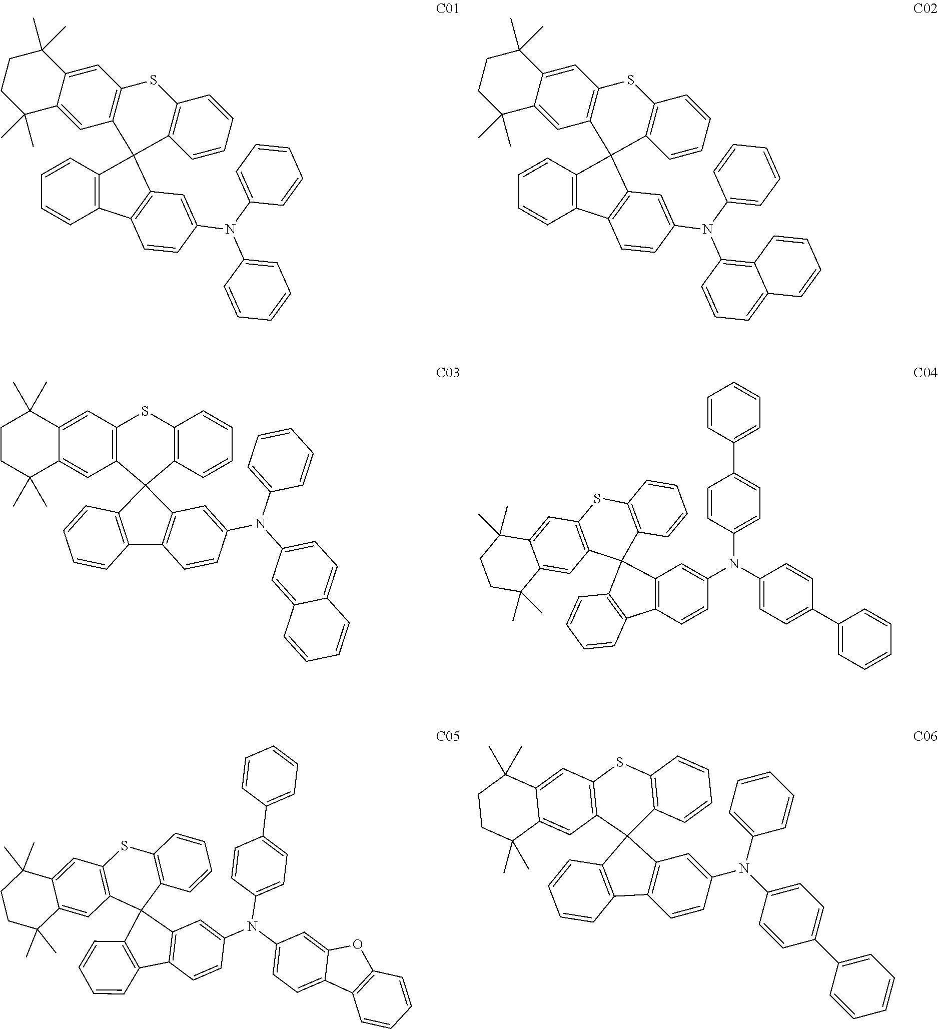

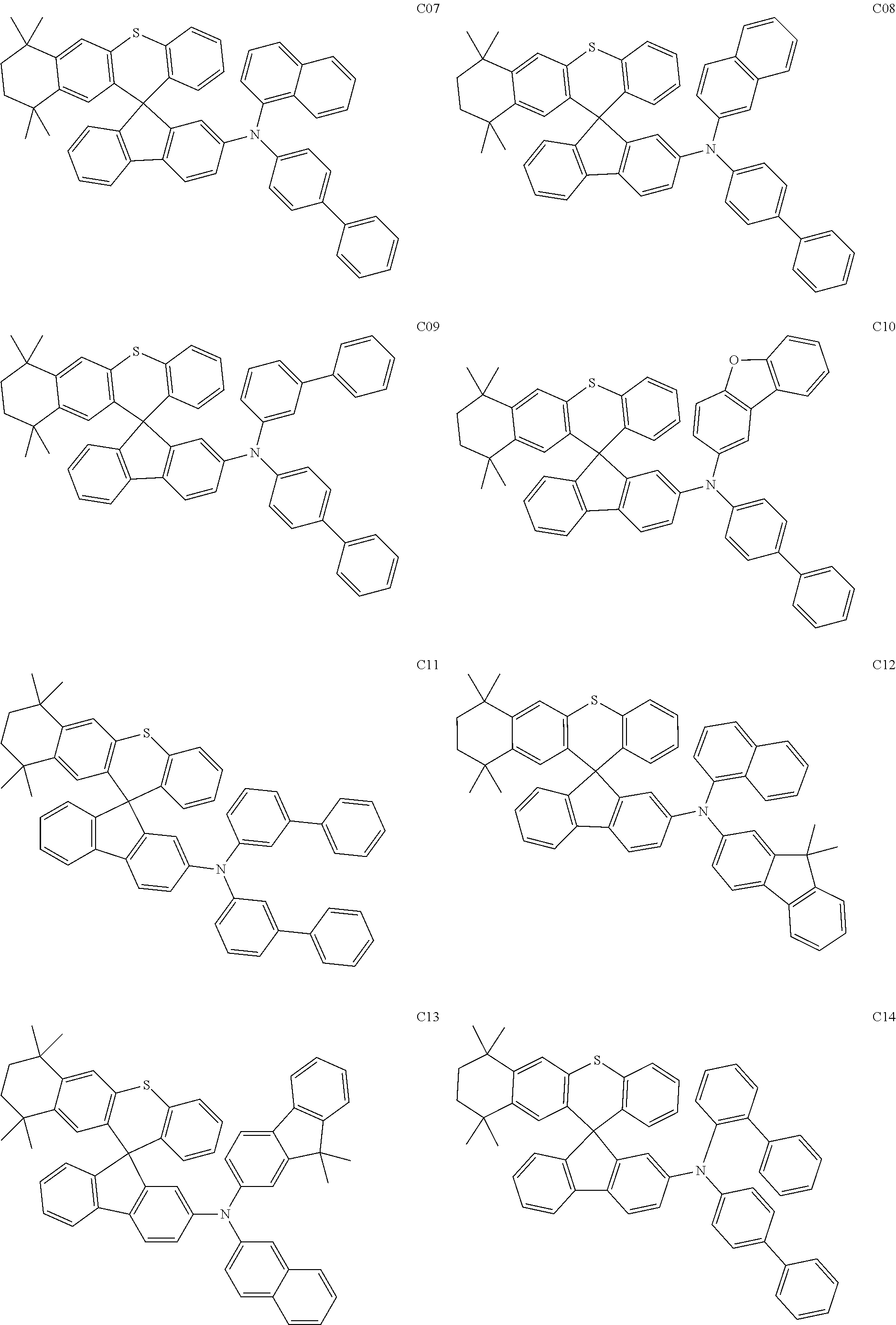

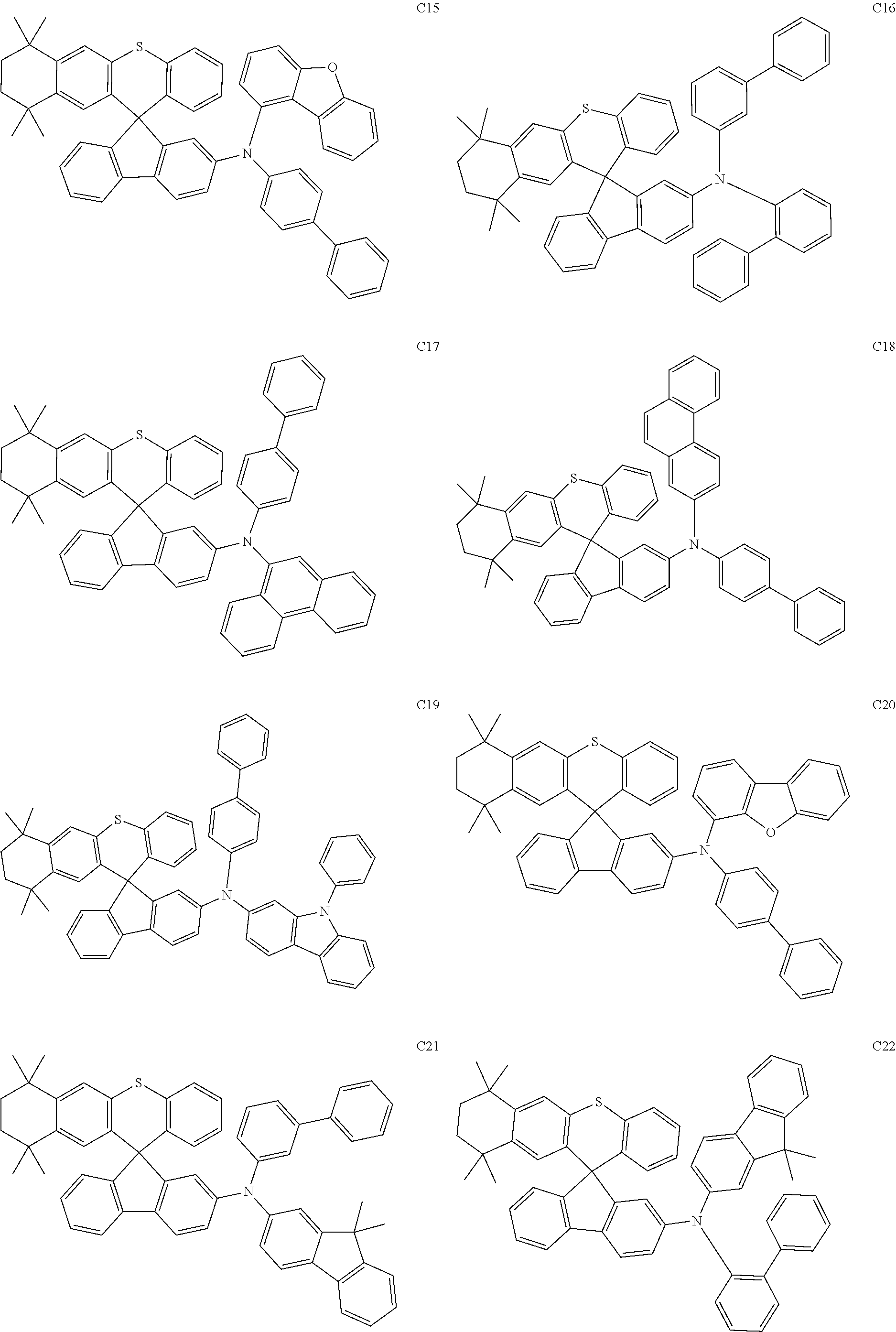

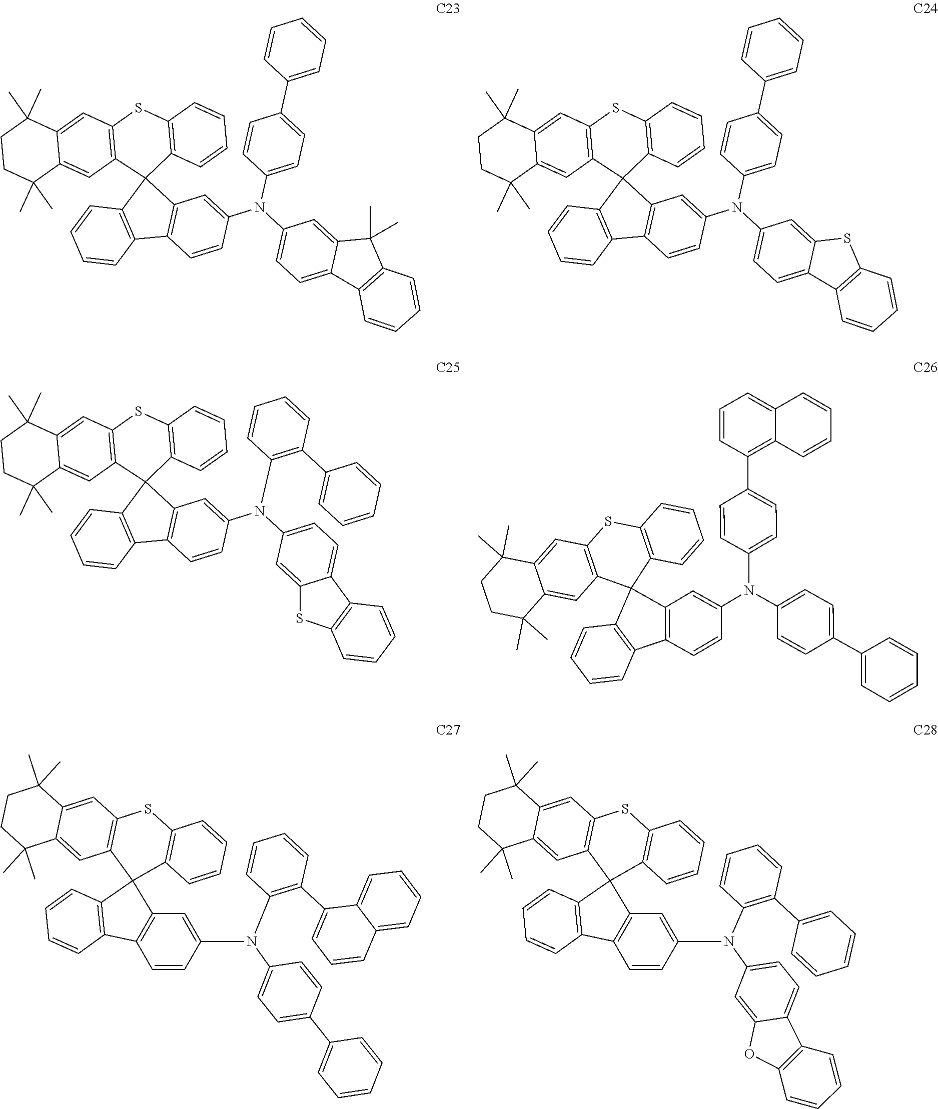

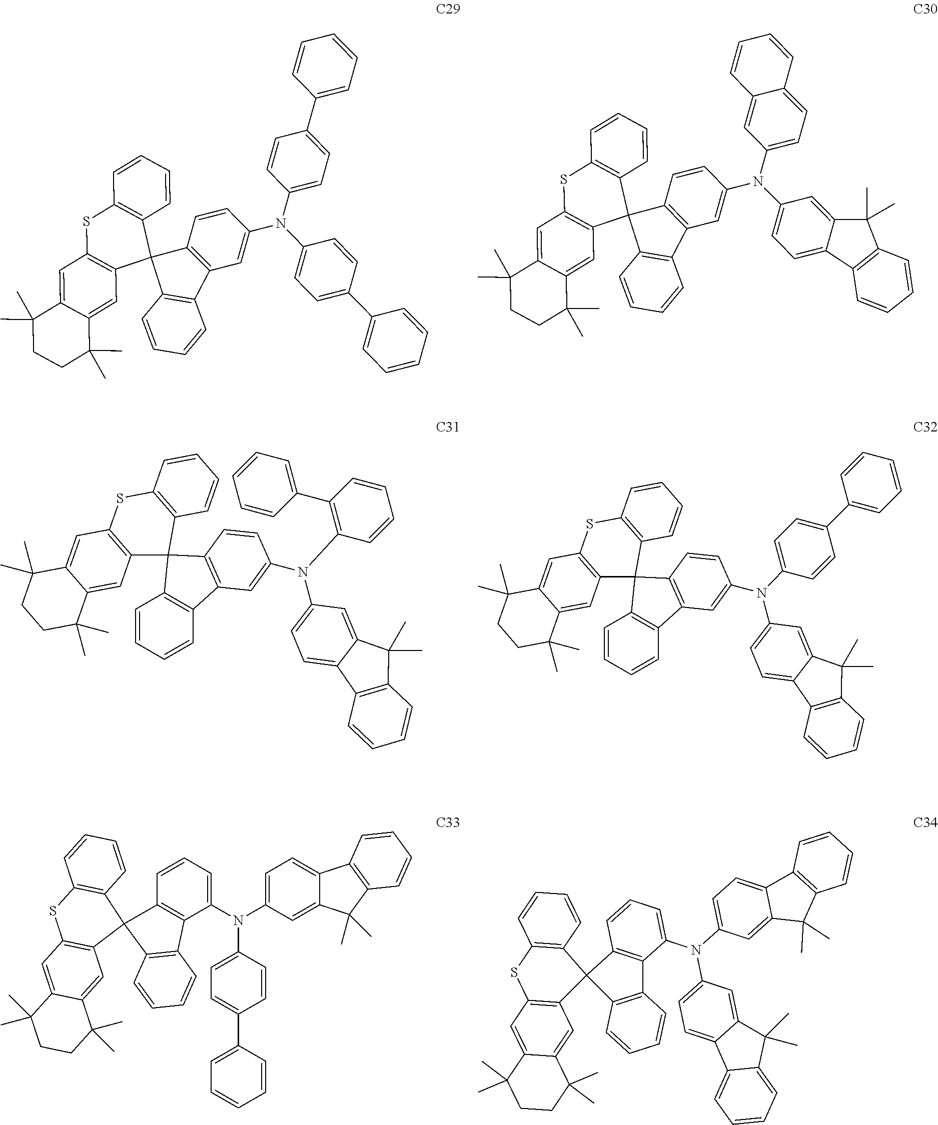

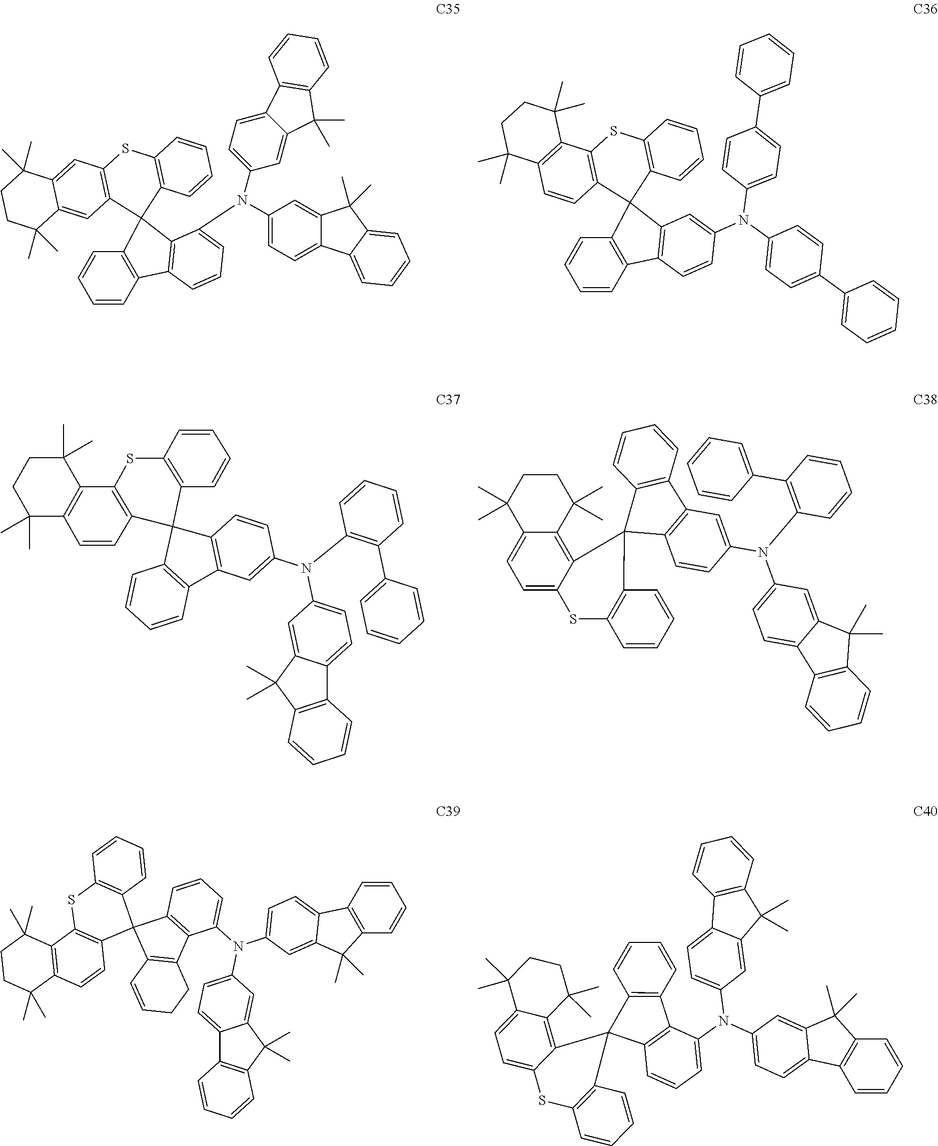

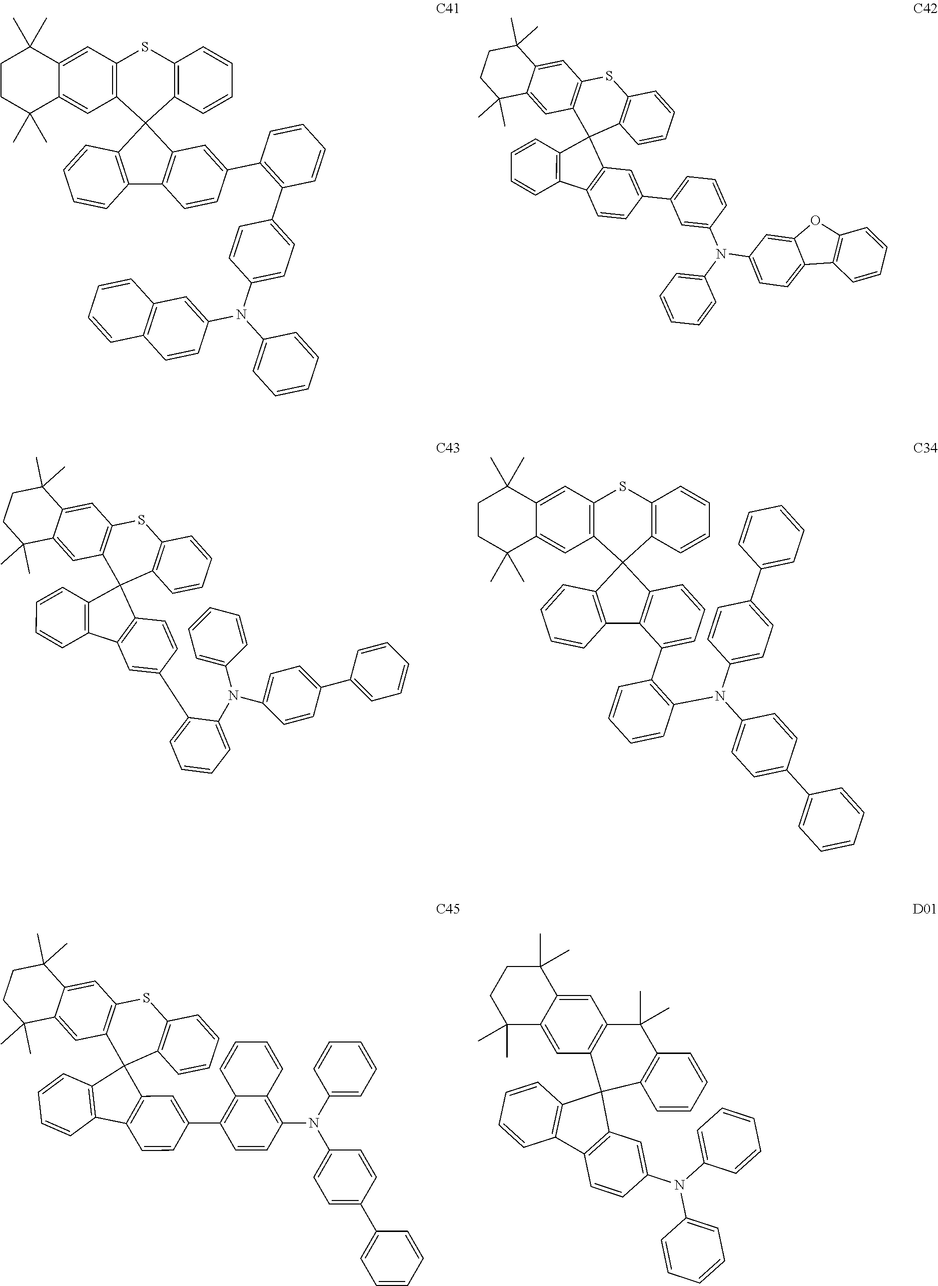

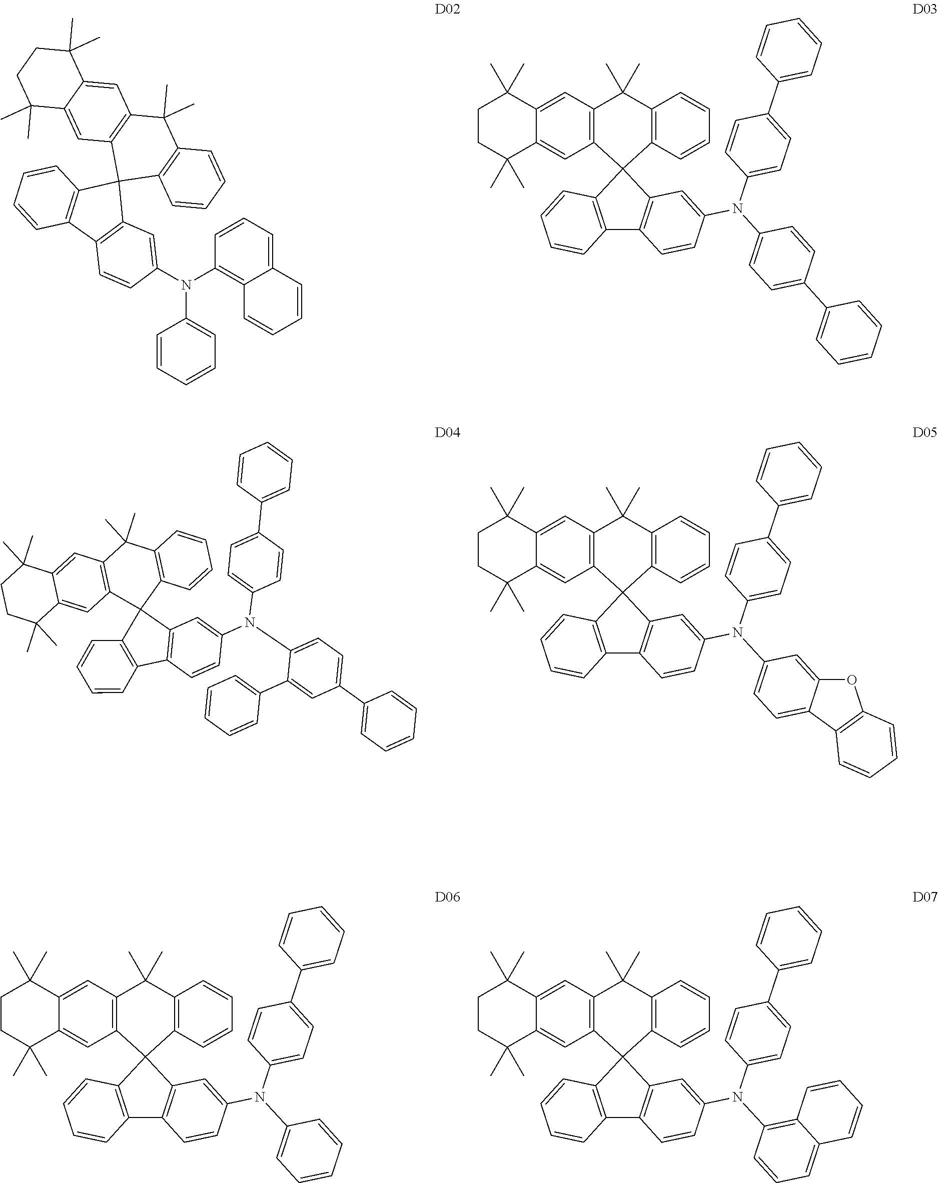

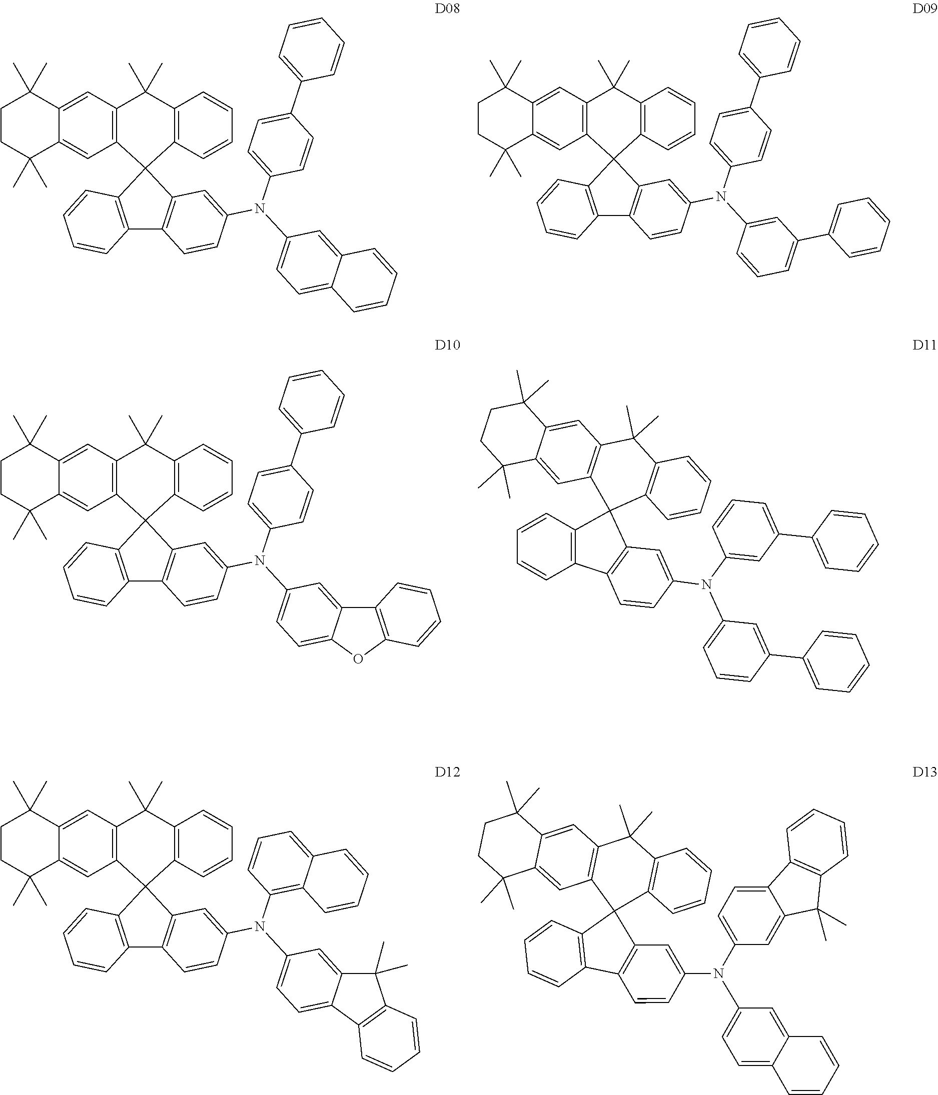

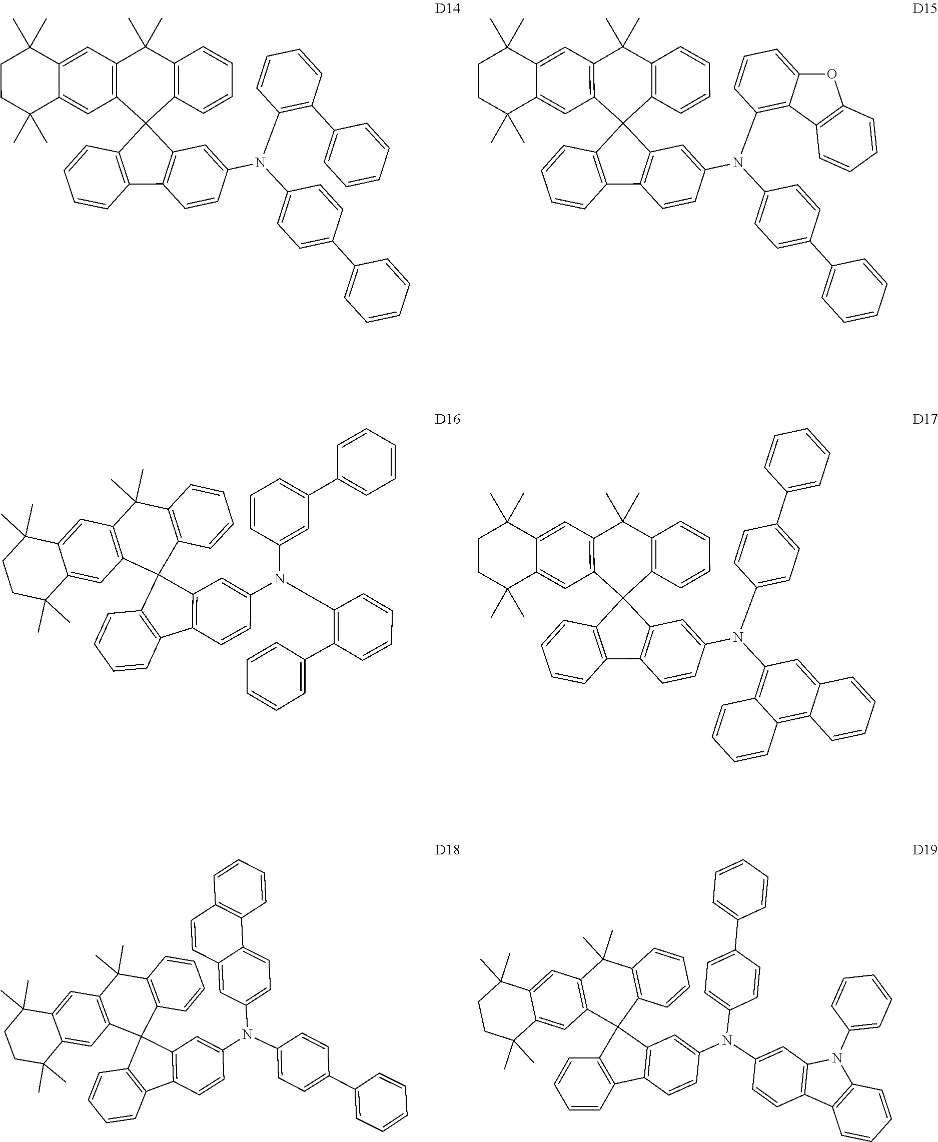

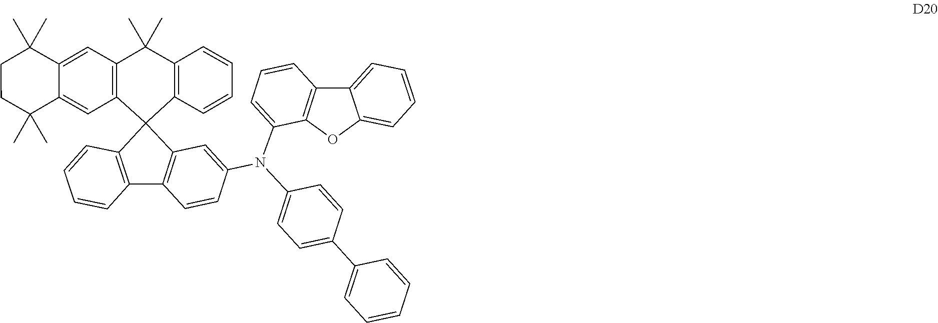 [SMILES: CC1(C)CCC(C)(C)c2cc3c(cc21)C(C)(C)c1ccccc1C31c2ccccc2-c2ccc(N(c3ccc(-c4ccccc4)cc3)c3cccc4c3oc3ccccc34)cc21]

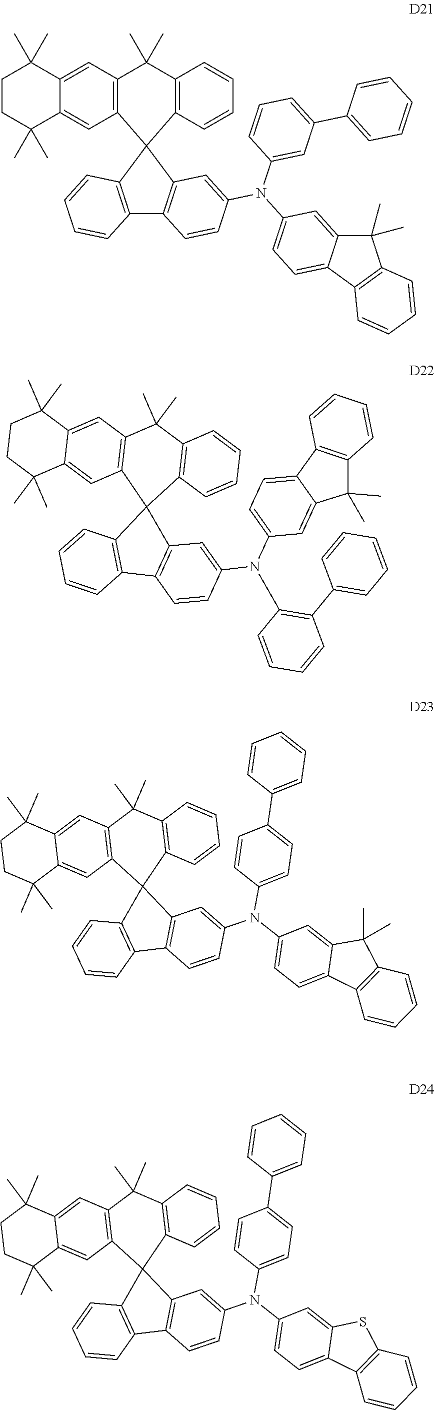 [SMILES: CC1(C)CCC(C)(C)c2cc3c(cc21)C(C)(C)c1ccccc1C31c2ccccc2-c2ccc(N(c3cccc(-c4ccccc4)c3)c3ccc4c(c3)C(C)(C)c3ccccc3-4)cc21 ; CC1(C)CCC(C)(C)c2cc3c(cc21)C(C)(C)c1ccccc1C31c2ccccc2-c2ccc(N(c3ccc4c(c3)C(C)(C)c3ccccc3-4)c3ccccc3-c3ccccc3)cc21 ; CC1(C)CCC(C)(C)c2cc3c(cc21)C(C)(C)c1ccccc1C31c2ccccc2-c2ccc(N(c3ccc(-c4ccccc4)cc3)c3ccc4c(c3)C(C)(C)c3ccccc3-4)cc21 ; CC1(C)CCC(C)(C)c2cc3c(cc21)C(C)(C)c1ccccc1C31c2ccccc2-c2ccc(N(c3ccc(-c4ccccc4)cc3)c3ccc4c(c3)sc3ccccc34)cc21]

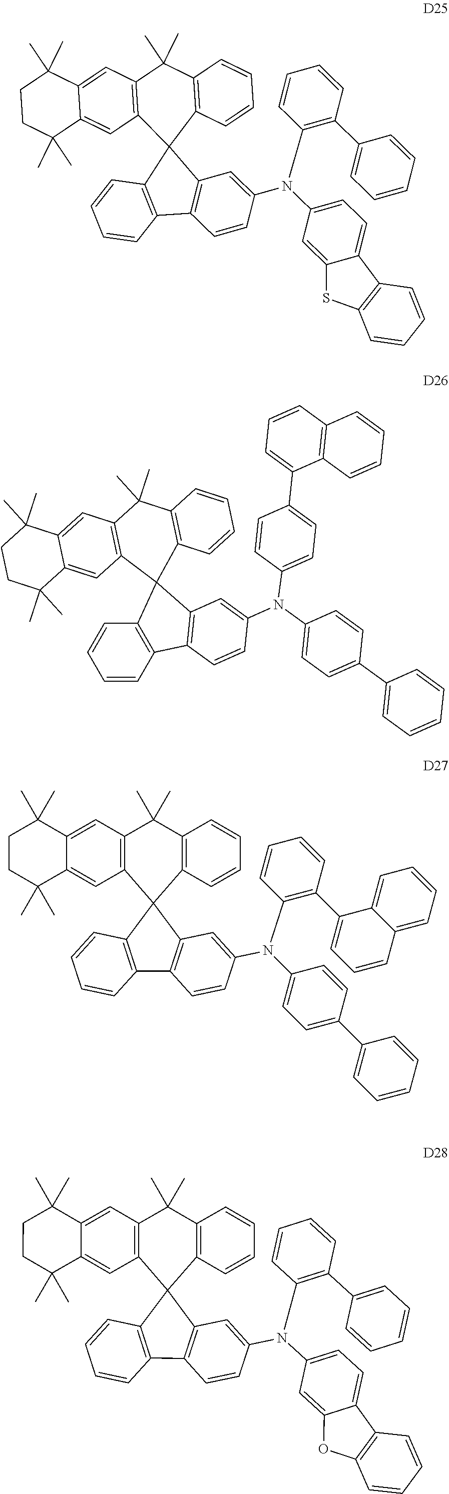 [SMILES: CC1(C)CCC(C)(C)c2cc3c(cc21)C(C)(C)c1ccccc1C31c2ccccc2-c2ccc(N(c3ccc4c(c3)sc3ccccc34)c3ccccc3-c3ccccc3)cc21 ; CC1(C)CCC(C)(C)c2cc3c(cc21)C(C)(C)c1ccccc1C31c2ccccc2-c2ccc(N(c3ccc(-c4ccccc4)cc3)c3ccc(-c4cccc5ccccc45)cc3)cc21 ; CC1(C)CCC(C)(C)c2cc3c(cc21)C(C)(C)c1ccccc1C31c2ccccc2-c2ccc(N(c3ccc(-c4ccccc4)cc3)c3ccccc3-c3cccc4ccccc34)cc21 ; CC1(C)CCC(C)(C)c2cc3c(cc21)C(C)(C)c1ccccc1C31c2ccccc2-c2ccc(N(c3ccc4c(c3)oc3ccccc34)c3ccccc3-c3ccccc3)cc21]

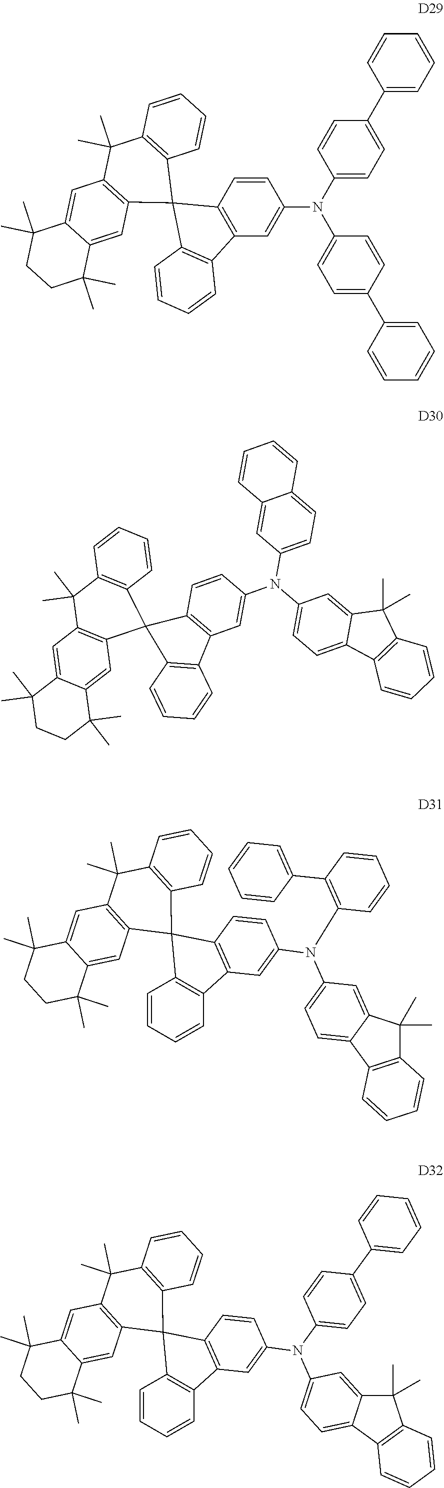

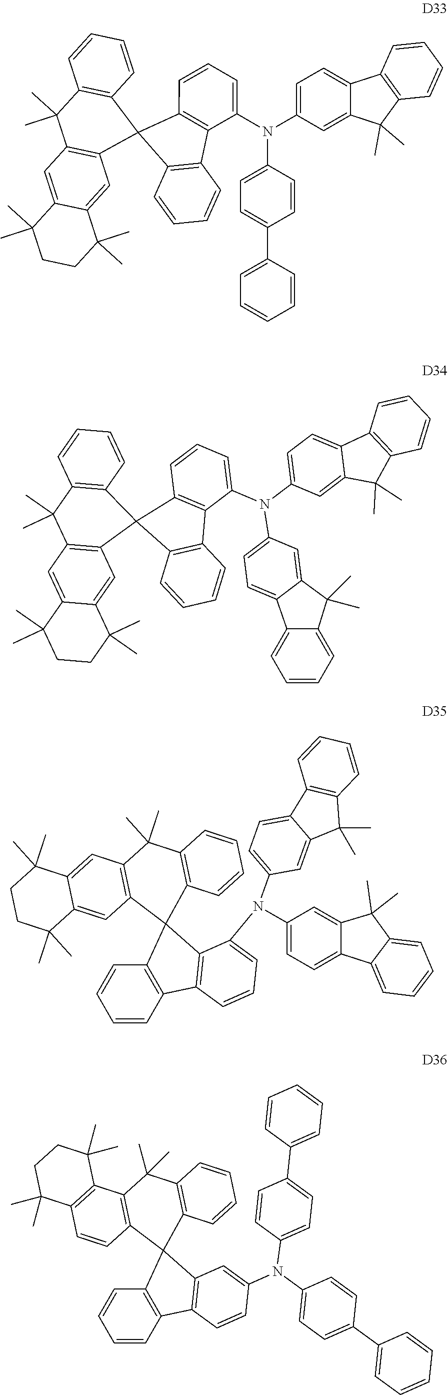 [SMILES: CC1(C)CCC(C)(C)c2cc3c(cc21)C(C)(C)c1ccccc1C31c2ccccc2-c2c(N(c3ccc(-c4ccccc4)cc3)c3ccc4c(c3)C(C)(C)c3ccccc3-4)cccc21 ; CC1(C)CCC(C)(C)c2cc3c(cc21)C(C)(C)c1ccccc1C31c2ccccc2-c2c(N(c3ccc4c(c3)C(C)(C)c3ccccc3-4)c3ccc4c(c3)C(C)(C)c3ccccc3-4)cccc21 ; CC1(C)CCC(C)(C)c2cc3c(cc21)C(C)(C)c1ccccc1C31c2ccccc2-c2cccc(N(c3ccc4c(c3)C(C)(C)c3ccccc3-4)c3ccc4c(c3)C(C)(C)c3ccccc3-4)c21 ; CC1(C)CCC(C)(C)c2c1ccc1c2C(C)(C)c2ccccc2C12c1ccccc1-c1ccc(N(c3ccc(-c4ccccc4)cc3)c3ccc(-c4ccccc4)cc3)cc12]

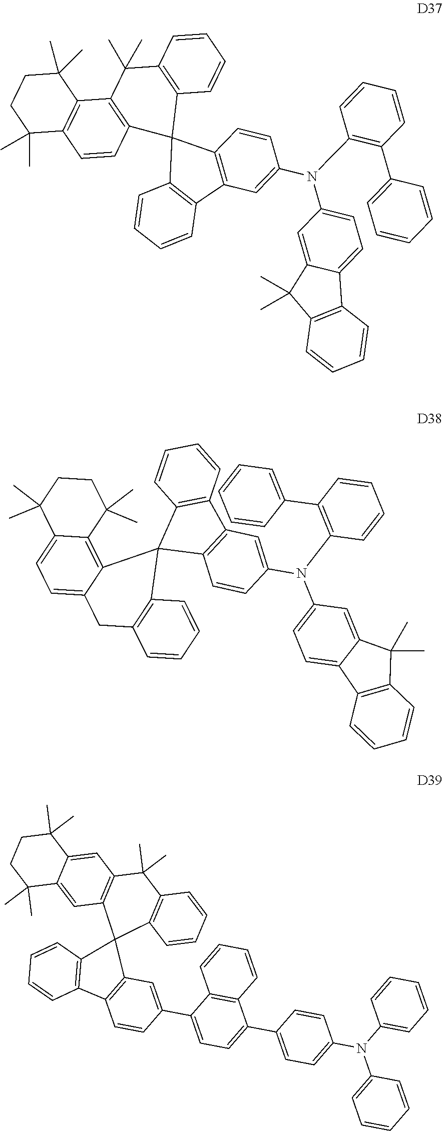

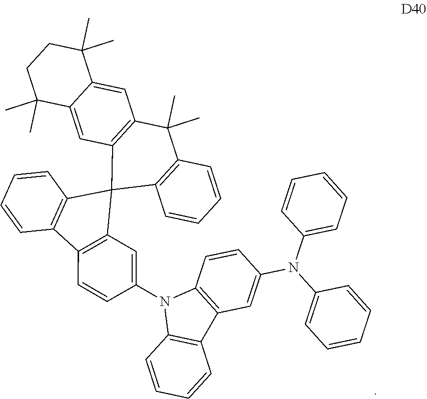 [SMILES: CC1(C)CCC(C)(C)c2cc3c(cc21)C(C)(C)c1ccccc1C31c2ccccc2-c2ccc(-n3c4ccccc4c4cc(N(c5ccccc5)c5ccccc5)ccc43)cc21]

11. An organic electroluminescent device, comprising an anode and a cathode disposed opposite to each other, and a functional layer disposed between the anode and the cathode; wherein the functional layer comprises the organic compound of claim 1.

12. The organic electroluminescent device according to claim 11, wherein the functional layer comprises a hole transport layer, and hole transport layer comprises the organic compound.

13. An electronic apparatus, comprising the organic electroluminescent device of claim 11.
